SEQUENCE LISTING (12 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - DNA, 1009 bp, Homo sapiens
  source 1..1009: mol_type = other DNA
gggggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatatagctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactcccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagt

SEQ ID NO: 3 - DNA, 21 bp, synthetic construct
  source 1..21: mol_type = other DNA
aagatgaagaagaagatgatg

SEQ ID NO: 4 - DNA, 21 bp, synthetic construct
  source 1..21: mol_type = other DNA
aagaagaagatgatgatgatg

SEQ ID NO: 5 - DNA, 21 bp, synthetic construct
  source 1..21: mol_type = other DNA
aaggaaagcctgatgcagcaa

SEQ ID NO: 6 - DNA, 21 bp, synthetic construct
  source 1..21: mol_type = other DNA
aaggctgcgaagctgaaggaa

SEQ ID NO: 7 - DNA, 21 bp, synthetic construct
  source 1..21: mol_type = other DNA
aagatgaagatgaagaagaag

SEQ ID NO: 8 - protein, 317 aa, Homo sapiens [sequence omitted]

SEQ ID NO: 9 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 10 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 11 - protein, 796 aa, Homo sapiens
MNQTASVSHHIKCQPSKTIKELGSNSPPQRNWKGIAIALLVILVVCSLITMSVILLTPDELTNSSETRLSLEDLFRKDFVLHDPEARWINDTDVVYKSENGHVIKLNIETNATTLLLENTTFVTFKASRHSVSPDLKYVLLAYDVKQIFHYSYTASYVIYNIHTREVWELNPPEVEDSVLQYAAWGVQGQQLIYIFENNIYYQPDIKSSSLRLTSSGKEEIIFNGIADWLYEEELLHSHIAHWWSPDGERLAFLMINDSLVPTMVIPRFTGALYPKGKQYPYPKAGQVNPTIKLYVVNLYGPTHTLELMPPDSFKSREYYITMVKWVSNTKTVVRWLNRAQNISILTVCETTTGACSKKYEMTSDTWLSQQNEEPVFSRDGSKFFMTVPVKQGGRGEFHHVAMFLIQSKSEQITVRHLTSGNWEVIKILAYDETTQKIYFLSTESSPRGRQLYSASTEGLLNRQCISCNFMKEQCTYFDASFSPMNQHFLLFCEGPRVPVVSLHSTDNPAKYFILESNSMLKEAILKKKIGKPEIKILHIDDYELPLQLSLPKDFMDRNQYALLLIMDEEPGGQLVTDKFHIDWDSVLIDMDNVIVARFDGRGSGFQGLKILQEIHRRLGSVEVKDQITAVKFLLKLPYIDSKRLSIFGKGYGGYIASMILKSDEKLFKCGSVVAPITDLKLYASAFSERYLGMPSKEESTYQAASVLHNVHGLKEENILIIHGTADTKVHFQHSAELIKHLIKAGVNYTMQVYPDEGHNVSEKSKYHLYSTILKFFSDCLKEEISVLPQEPEEDE

SEQ ID NO: 12 - DNA, 5964 bp, Homo sapiens
  source 1..5964: mol_type = other DNA
agaagcagcagaagcaacagcagtagcagcggcagcagcaacagcagcagcccctactgaagtccaatagaggagacttgatctctagttcattctggaactccgcctgggattgtgcactgtccagggtcctgaaacatgaaccaaactgccagcgtgtcccatcacatcaagtgtcaaccctcaaaaacaatcaaggaactgggaagtaacagccctccacagagaaactggaagggaattgctattgctctgctggtgattttagttgtatgctcactcatcactatgtcagtcatcctcttaaccccagatgaactcacaaattcgtcagaaaccagattgtctttggaagacctctttaggaaagactttgtgcttcacgatccagaggctcggtggatcaatgatacagatgtggtgtataaaagcgagaatggacatgtcattaaactgaatatagaaacaaatgctaccacattattattggaaaacacaacttttgtaaccttcaaagcatcaagacattcagtttcaccagatttaaaatatgtccttctggcatatgatgtcaaacagatttttcattattcgtatactgcttcatatgtgatttacaacatacacactagggaagtttgggagttaaatcctccagaagtagaggactccgtcttgcagtacgcggcctggggtgtccaagggcagcagctgatttatatttttgaaaataatatctactatcaacctgatataaagagcagttcattgcgactgacatcttctggaaaagaagaaataatttttaatgggattgctgactggttatatgaagaggaactcctgcattctcacatcgcccactggtggtcaccagatggagaaagacttgccttcctgatgataaatgactctttggtacccaccatggttatccctcggtttactggagcgttgtatcccaaaggaaagcagtatccgtatcctaaggcaggtcaagtgaacccaacaataaaattatatgttgtaaacctgtatggaccaactcacactttggagctcatgccacctgacagctttaaatcaagagaatactatatcactatggttaaatgggtaagcaataccaagactgtggtaagatggttaaaccgagctcagaacatctccatcctcacagtctgtgagaccactacaggtgcttgtagtaaaaaatatgagatgacatcagatacgtggctctctcagcagaatgaggagcccgtgttttctagagacggcagcaaattctttatgacagtgcctgttaagcaagggggacgtggagaatttcaccacgtagctatgttcctcatccagagtaaaagtgagcaaattaccgtgcggcatctgacatcaggaaactgggaagtgataaagatcttggcatacgatgaaactactcaaaaaatttactttctgagcactgaatcttctcccagaggaaggcagctgtacagtgcttctactgaaggattattgaatcgccaatgcatttcatgtaatttcatgaaagaacaatgtacatattttgatgccagttttagtcccatgaatcaacatttcttattattctgtgaaggtccaagggtcccagtggtcagcctacatagtacggacaacccagcaaaatattttatattggaaagcaattctatgctgaaggaagctatcctgaagaagaagataggaaagccagaaattaaaatccttcatattgacgactatgaacttcctttacagttgtcccttcccaaagattttatggaccgaaaccagtatgctcttctgttaataatggatgaagaaccaggaggccagctggttacagataagttccatattgactgggattccgtactcattgacatggataatgtcattgtagcaagatttgatggcagaggaagtggattccagggtctgaaaattttgcaggagattcatcgaagattaggttcagtagaagtaaaggaccaaataacagctgtgaaatttttgctgaaactgccttacattgactccaaaagattaagcatttttggaaagggttatggtggctatattgcatcaatgatcttaaaatcagatgaaaagctttttaaatgtggatccgtggttgcacctatcacagacttgaaattgtatgcctcagctttctctgaaagataccttgggatgccatctaaggaagaaagcacttaccaggcagccagtgtgctacataatgttcatggcttgaaagaagaaaatatattaataattcatggaactgctgacacaaaagttcatttccaacactcagcagaattaatcaagcacctaataaaagctggagtgaattatactatgcaggtctacccagatgaaggtcataacgtatctgagaagagcaagtatcatctctacagcacaatcctcaaattcttcagtgattgtttgaaggaagaaatatctgtgctaccacaggaaccagaagaagatgaataatggactgtatttatacagaactgaagggaatattgaggctcaatgaaacctgacaaagagactgtaatattgtagttgctccagaatgtcaagggcagcttacggagatgtcactggagcagcacgctcagagacagtgaactagcatttgaatacacaagtccaagtctactgtgttgctaggggtgcagaacccgtttctttgtatgagagaggtcaaagggttggtttcctgggagaaattagttttgcattaaagtaggagtagtgcatgttttcttctgttatccccctgtttgttctgtaactagttgctctcattttaatttcactggccaccatcatctttgcatataatgcacaatctatcatctgtcctacagtccctgatctttcatggctgagctgcaatctaacactttactgtacctttataataagtgcaattctttcattgtctattattatgcttaagaaaatattcagttaataaaaaacagagtattttatgtaatttctgtttttaaaaagacattattaaatgggtcaaaggacatatagaaatgtggatttcagcaccttccaaagttcagccagttatcagtagatacaatatctttaaatgaacacacgagtgtatgtctcacaatatatatacacaagtgtgcatatacagttaatgaaactatctttaaatgttattcatgctataaagagtaaacgtttgatgaattagaagagatgctcttttccaagctataatggatgctttgtttaatgagccaaatatgatgaaacattttttccaattcaaattctagctattgctttcctataaatgtttgggttgtgtttggtattgtttttagtggttaatagttttccagttgcatttaattttttgaatatgataccttgtcacatgtaaattagatacttaaatattaaattatagtttctgataaagaaattttgttaacaatgcaatgccactgagtgctattttgctcttttggtggagaaggcttttttcaaaactcttggtccttttacttctttctctcagtgcagaatcaattctcattttcatcgtaaaagcaaatagctggattatttcatttgccagtttctatttagtattccatgcctgcccaattcatctgttactgtttaatttcaattcttctggtgagaattagaaatgaaatattttttattcattggccaaaaagttcacagacagcagtgtttgctatttactttgaattgaaggcacaaaatgcatcaattcctgtgctgtgttgacttgcagtagtaagtaactgagagcataaaataaacctgactgtatgaagtcaatttaagtgatgagaacatttaactttggtgactaaagtcagaatatcttctcacttcacttaagggatcttccagaagatatctaaaagtctgtaataagcttagaagttcagataaatctaggcaggatactgcatttttgtggttttaaaaaagtccttaggacagactgaattatcataacttatggcatcaggaggaaactttaaaatatcaaggaatcactcagtcaccctcctgttttgttgaaggatcaaccccaaattctgggtatttgagtacatgtgaatcatggatttggtattcaactttttccctggatgctttggaatcgtgtcttccatgctccactgggttcaatttaaaataggagaggctttctcttctgaaagatccattttaggtctttttcaagaatagtgaacacattttttaacaaaataagttgtaattttaaaaggaaagttttgcctattttattaagatggaaatttctttttaggctaatttgaaatccaactgaagctttttaaccaatattttaaatttgaaccactagagttttttatgatgcaaatgattatgttgtctgaaaggtgtggttttattgaatgtctatttgagtatcatttaaaaagtatttgccttttactgtcatcatttctcttgttttattattattatcaatgtttatctatttttcaattaatttaatacagtttctaatgtgaaagacatttttctggaacccgttttccccttaaacactaaagagacctcaagtgaaagcatattgcttagtaggaaggtagaaaatgttaatccctgcgattctttgagttttaatgacagggtcattttcagtaaaggaaatgctcaccaacacatagtcaccaactattaaaggaatcatgtgattggattttcccctgtatacatgtacccttggtcataatcccactatttcatacatatttatgcattgctagattttcctaggactccaatagcatgctttccaagtgttattattcccttaatgttaaagaaaaaaatcaatatattgaattctttaattaaataggtaagcagtattaaatcatccacagagataaacaatctcttgaaactcttttgaaagagaaaagtgtttgttcactttcattgccttcattaagatctccaaatactcagagactaagcaggctattctcatctctttgagtttcctgacagaactaaaggaaggaatcactcttcaaaagatggatctcacttcactctgaaaatgtgcctttttttaattgggtgtttagcataaaaatcactattggggatcttacagatgtctgttgtagatctattacaatgaggtgacttagacattgggctttaccatattagagaattacttaagccattgtccttcaaatgttgggcttggagacagaagtaccttggactgaattgctttcaagtctctagaatcactgacagcacatagcttctatgaggagttatatatgaaataccacttgtgtccctcctcaagttctctgaccaccttcacatcctgcggccaccaacactgtatggatacaatctggcctctcagctctgctcactttcactttctgttctggggattctctgatactaattttggctacttggtagaaccaagagacaaatcgcttacatgctgcaaaccgatgtgaaggtgttactaacccgctaaaacacaattaatgaaagactgtagcaggtaactgatctttccctcagatattctacatggtccatggaagactctgtgggccgcagaagcccatagctgcatacccttctattttcctcttccccatttcactcttttaagctccctactcctattccctatgatcactttccaaaataaacggcaggtgtgctagctgttgtcttagactctgctttctggaaactatttttcccctccacaagaaattatcactgtgaatattacaggaggagaaataaaataagaataaatcagtggtttattttcaggttcgaataaactctttctaatgaaaa

(19) United States
(12) Patent Application Publication
(10) Pub. No.: US 2025/0262230 A1
(43) Pub. Date: Aug. 21, 2025

(54) HMGB1 Inhibitors for Treatment of APOE4-related Tauopathies including Alzheimer’s Disease

(71) Applicant: The J. David Gladstone Institutes, a testamentary trust established under the Will of J. David Gla, San Francisco, CA (US)

(72) Inventors: Yadong Huang, Danville, CA (US); Nicole Koutsodendris, Daly Cit, CA (US)

(73) Assignee: The Regents of the University of California, Oakland, CA (US)

(21) Appl. No.: 18/870,731

(22) PCT Filed: May 31, 2023
(86) PCT No.: PCT/US2023/067658

§ 371 Date: Dec. 2, 2024

Related U.S. Application Data
(60) Provisional application No. 63/354,728, filed on Jun. 23, 2022.

(51) Int. Cl. A61K 31/704; A61K 31/22; A61P 25/28

(52) U.S. Cl. CPC A61K 31/704; A61K 31/22; A61P 25/28

(57) ABSTRACT

As described herein, inhibitors of High mobility group box protein 1 (HMGB1) can significantly reduce HMGB1 nucleo-cytoplasmic translocation, gliosis, neurodegeneration, Tau pathologies, and myelin deficits, especially in subjects having an AP0E4 allele. Methods are therefore described herein that include administering one or more inhibitors of High mobility group box protein 1 (HMGB1) to a subject having at least one genomic AP0E4 allele.

### CROSS-REFERENCE TO RELATED APPLICATIONS

This application claims the benefit of the filing date of U.S. application No. 63/354,728, filed on Jun. 23, 2022, the disclosure of which is incorporated by reference herein.

### GOVERNMENT FUNDING

This invention was made with government support under R01AG071697 and P01AG073082 awarded by the National Institutes of Health, National Institute on Aging. The government has certain rights in the invention.

### INCORPORATION BY REFERENCE OF SEQUENCE LISTING

A Sequence Listing is provided herewith as an xml file, “2338683.xml” created on May 31, 2023, and having a size of 20,464 bytes. The content of the xml file is incorporated by reference herein in its entirety.

### BACKGROUND

Alzheimer's disease is a chronic neurodegenerative disease that is the cause of 60-70% of cases of dementia. See, e.g., Burns, A., et al. “Alzheimer's Disease,” BMJ 338 (2009); World Health Organization, “Dementia Fact Sheet,” 2017, available at website who.int/en/news-room/fact-sheets/detail/dementia. There are approximately 30-35 million people worldwide with Alzheimer's disease. See World Health Organization, supra. Alzheimer's disease affects about 6% of people who are 65 years of age and older. Burns, A., et al., supra. Alzheimer's disease is one of the most financially costly diseases in developed countries. See, e.g., Bonin-Guillaume, S., et al. “The Economical Impact of Dementia,” Presse Medicate, 34, 35 (2005). However, currently there is no known cure for Alzheimer's disease.

Thus, there remains a need for a treatment for Alzheimer's disease and other tauopathies.

### SUMMARY

As described herein, inhibitors of High mobility group box protein 1 (HMGB1) can significantly reduce HMGB1 nucleo-cytoplasmic translocation, gliosis, neurodegeneration, Tau pathologies, and myelin deficits, especially in subjects having an APOE4 allele. Methods are therefore described herein that include administering one or more inhibitors of High mobility group box protein 1 (HMGB1) to a subject having at least one genomic APOE4 allele. In some cases, the subject has two genomic APOE4 alleles. For example, the subject can express detectable levels of APOE4 protein. Examples of HMGB1 inhibitors that may be used include glycyrrhizic acid, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies (m2G7 or #10-22), recombinant HMGB1 box A protein, acetylcholine, the nicotinic acetylcholine receptor subtype alpha 7 agonist GTS-21, Peptide P5779, resveratrol, metformin, or a combination thereof. In some cases, the subject may exhibit symptoms of HMGB1 nucleo-cytosplasmic translocation, gliosis, neurodegeneration, tau pathology (buildup of tau protein), or myelin deficit. For example, the subject may exhibit symptoms of at least one tauopathy. Such a tauopathy is a disease characterized by the deposition of abnormal tau protein in the brain. For example, the tauopathy can be a neurodegenerative disorder, Alzheimer's disease, Pick disease, progressive supranuclear palsy, corticobasal degeneration, argyrophilic grain disease, primary age-related tauopathy, chronic traumatic encephalopathy, or frontotemporal dementia.

### DESCRIPTION OF THE FIGURES

FIG. 1A-1N illustrate that Tau pathology accumulation and propagation is reduced in PS19-fE4 mice after removal of APOE4 from neurons, but not astrocytes. FIG. 1A graphically illustrates ApoE levels as measured by ELISA in the hippocampal lysates of 10-month-old PS19-fE4 and PS19-fE3 mice with and without Cre (PS19-fE4: No Cre, n=21; PS19-fE4/Syn1-Cre, n=18; PS19-fE4/GFAP-Cre, n=17; PS19-fE3: No Cre, n=17; PS19-fE3/Syn1-Cre, n=11; PS19-fE3/GFAP-Cre, n=11). ApoE levels were normalized to the weight of the dissected hippocampal tissue for each individual mouse. Data are represented as mean±SEM, one-way ANOVA with Tukey's post hoc multiple comparisons test. FIG. 1B shows representative images of pTau staining with anti-AT8 in the hippocampus of 10-month-old PS19-fE4 and PS19-fE3 mice with and without Cre (scale bar, 500 μm). FIG. 1C graphically illustrates the quantified percentages of pTau (AT8) coverage area in the hippocampus of 10-month-old PS19-fE4 and PS19-fE3 mice with or without Cre (PS19-fE4: No Cre, n=25; Syn1-Cre, n=17; GFAP-Cre, n=16; PS19-fE3: No Cre, n=20; Syn1-Cre, n=15; GFAP-Cre, n=15). FIG. 1D shows representative images of anti-AT8 (green) and anti-TUJ1 (red) western blots in RAB fractions of hippocampal tissue lysates of 10-month-old PS19-fE4 and PS19-fE3 mice with or without Cre. FIG. 1E shows representative images of anti-AT8 (green) and anti-TUJ1 (red) western blots in RIPA fractions of hippocampal tissue lysates of 10-month-old PS19-fE4 and PS19-fE3 mice with or without Cre. FIG. 1F graphically illustrates quantified AT8-positive pTau levels relative to TUJ1 measured by western blot analysis in RAB fractions of the hippocampal lysates of 10-month-old PS19-fE4 and PS19-fE3 mice with or without Cre (PS19-fE4: No Cre, n=21; Syn1-Cre, n=18; GFAP-Cre, n=17; PS19-fE3: No Cre, n=17; Syn1-Cre, n=11; GFAP-Cre, n=11). FIG. 1G graphically illustrates quantified AT8-positive pTau levels relative to TUJ1 measured by western blot analysis in RIPA fractions of the hippocampal lysates of 10-month-old PS19-fE4 and PS19-fE3 mice with or without Cre (PS19-fE4: No Cre, n=21; Syn1-Cre, n=18; GFAP-Cre, n=17; PS19-fE3: No Cre, n=17; Syn1-Cre, n=11; GFAP-Cre, n=11). FIG. 1H is a schematic diagram illustrating the experimental design of a Tau propagation study involving a 12-week incubation period following unilateral hippocampal injection of AAV2-Tau-P301S (2.10E+13 vg/mL) in fE4 mice with and without Cre or fE3 mice without Cre. FIG. 1I shows a representative image of a human Tau-immunostained (HT7 antibody) fE4 mouse brain 12 weeks post-injection, with the injection site indicated by the black dot (X=+1.5; Y=−2.1; Z=−2.1) and a notch in the left hemisphere to distinguish the non-injected side (scale bar, 1 mm). FIG. 1J shows a representative image of a GFP-immunostained 10-month-old fE4 mouse 2 weeks after a unilateral injection with AAV2-GFP (1.0E+13 vg/mL) into the right hippocampus, illustrating there is no viral spread to the non-injected hippocampal side (scale bar, 900 μm). FIG. 1K shows representative images of the non-injected hippocampal side of 13-month-old fE mice, with and without Cre, after immunostaining for human Tau (HT7 antibody) (scale bar, 500 μm). FIG. 1L shows representative images of the non-injected hippocampal side of 13-month-old fE mice with or without Cre after immunostaining with pTau (AT8 antibody) (scale bar, 500 μm). FIG. 1M graphically illustrates the average number of HT7 (human Tau)-positive cells in each hippocampal slice on the non-injected hippocampal side of 13-month-old fE mice with or without Cre. FIG. 1N graphically illustrates the average number of AT8 (pTau)-positive cells in each hippocampal slice on the non-injected hippocampal side of 13-month-old fE mice with or without Cre. Quantified data in FIG. 1K-1M are n=8 mice per genotype group and data in FIG. 1B, 1E, 1F, 1K, 1M are represented as mean±SEM, one-way ANOVA with Tukey's post hoc multiple comparisons test.

FIG. 2A-2B illustrate that removing neuronal, but not astrocytic, APOE4 ameliorates neuronal hyperexcitability in the hippocampus of PS19-fE4 mice. FIG. 2A graphically illustrates average normalized fPSP slopes in CA1 stratum radiatum in response to incremental stimulation of Schaffer collaterals. Neuronal APOE4 expression renders CA3-CA1 network hyperexcitable as evidenced by augmented response to synaptic stimulation. FIG. 2B graphically illustrates calculated individual fPSP slope gain values for all experiments in FIG. 2A. PS19-fE3: n=13, N=2; PS19-fE4: n=20, N=4; PS19-fE4/Syn1-Cre: n=25, N=4; PS19-fE4/GFAP-Cre: n=13, N=2. Data is represented as mean±SEM, one-way ANOVA with Tukey's post hoc multiple comparisons test.

FIG. 3A-3J illustrate that neurodegeneration is reduced whether APOE4 is removed from neurons or astrocytes. FIG. 3A shows representative images of the ventral hippocampus of 10-month-old PS19-fE4 and PS19-fE3 mice with and without Cre after staining with Sudan Black to enhance hippocampal visualization (scale bar, 1 mm). FIG. 3B graphically illustrates quantification of hippocampal volume in 10-month-old PS19-fE4 and PS19-fE3 mice with and without Cre. FIG. 3C graphically illustrates quantification of posterior lateral ventricle volume in 10-month-old PS19-fE4 and PS19-fE3 mice with and without Cre. FIG. 3D shows representative images of the CA1 hippocampal subfield of 10-month-old PS19-fE4 and PS19-fE3 mice with and without Cre after immunostaining for neuronal marker NeuN (scale bar, 50 μm). FIG. 3E graphically illustrates the thickness of the CA1 neuronal cell layer of 10-month-old PS19-fE4 and PS19-fE3 mice with and without Cre. FIG. 3F shows representative images of the hippocampal dentate gyrus of 10-month-old PS19-fE4 and PS19-fE3 mice with and without Cre after immunostaining for neuronal marker NeuN (scale bar, 100 μm). FIG. 3G graphically illustrates the thickness of the dentate gyrus granule cell layer of 10-month-old PS19-fE4 and PS19-fE3 mice with and without Cre. For all quantifications in FIGS. 3B, 3C, 3E, and 3G, PS19-fE4: No Cre, n=25; Syn1-Cre, n=17; GFAP-Cre, n=16 and PS19-fE3: No Cre, n=20; Syn1-Cre, n=15; GFAP-Cre, n=15 mice. All data are represented as mean±SEM, one-way ANOVA with Tukey's post hoc multiple comparisons test. FIG. 3H graphically illustrates correlations between hippocampal volume (mm³) and AT8 coverage area (%) in PS19-fE4 mice (n=25). Pearson's correlation analysis (two-sided). LV, lateral ventricle; DG, dentate gyrus. FIG. 3I graphically illustrates correlations between hippocampal volume (mm³) and posterior lateral ventricle volume (mm³) in PS19-fE4 mice (n=25). Pearson's correlation analysis (two-sided). LV, lateral ventricle; DG, dentate gyrus. FIG. 3J graphically illustrates correlations between hippocampal volume (mm³) and CA1 neuronal cell layer thickness (μm) (j) in PS19-fE4 mice (n=25). Pearson's correlation analysis (two-sided). LV, lateral ventricle; DG, dentate gyrus.

FIG. 4A-4O illustrate that microgliosis and astrogliosis are reduced whether APOE4 is removed from neurons or astrocytes. FIG. 4A graphically illustrates the percent Iba1 coverage area in the hippocampus of 10-month-old PS19-fE4 and PS19-fE3 mice with and without Cre. FIG. 4B graphically illustrates a correlation between percent Iba1 coverage area and hippocampal volume of PS19-fE4 mice (n=25). FIG. 4C graphically illustrates the percent CD68 coverage area in the hippocampus of 10-month-old PS19-fE4 and PS19-fE3 mice with and without Cre. FIG. 4D graphically illustrates a correlation between percent CD68 coverage area and hippocampal volume of PS19-fE4 mice (n=25). in FIG. 4D, n=8 mice per genotype group, and all data are represented as mean±SEM, one-way ANOVA with Tukey's post hoc multiple comparisons test. FIG. 4E graphically illustrates the percent GFAP coverage area in the hippocampus of 10-month-old PS19-fE4 and PS19-fE3 mice with and without Cre. FIG. 4F graphically illustrates the percent S100β coverage area in the hippocampus of 10-month-old PS19-fE4 and PS19-fE3 mice with and without Cre. FIG. 4G graphically illustrates the correlation between percent GFAP coverage area and hippocampal volume of PS19-fE4 mice (n=25). FIG. 4H graphically illustrates the correlation between percent S100β coverage area and hippocampal volume of PS19-fE4 mice (n=25). For quantifications, PS19-fE4: No Cre, n=25; Syn1-Cre, n=17; GFAP-Cre, n=16 and PS19-fE3: No Cre, n=20; Syn1-Cre, n=15; GFAP-Cre, n=15 mice. All data are represented as mean±SEM, one-way ANOVA with Tukey's post hoc multiple comparisons test. Pearson's correlation analysis (two-sided). FIG. 4I graphically illustrates the percent Iba1 coverage area in the non-injected hippocampal side of 13-month-old fE mice with and without Cre 12 weeks after unilateral injection of AAV2-Tau-P301S. FIG. 4J graphically illustrates the percent GFAP coverage area in the non-injected hippocampal side of 13-month-old fE mice with and without Cre 12 weeks after unilateral injection of AAV2-Tau-P301S. AAV2 virus does not directly penetrate the non-injected hippocampal side and microgliosis is reduced after removal of neuronal or astrocytic APOE4 in the non-injected hippocampal side of fE4 mice that received unilateral injections of AAV2-Tau-P301S. For quantifications FIG. 4K graphically illustrates the percent MBP coverage area in the hippocampal CA1 subregion of 10-month-old PS19-fE4 and PS19-fE3 mice with and without Cre. FIG. 4L graphically illustrates the percent NG2 coverage area in the hippocampus of 10-month-old PS19-fE4 and PS19-fE3 mice with and without Cre. FIG. 4M graphically illustrates APOE protein levels measured by ELISA in the cell lysates of 14-day cultured primary neurons from the hippocampus and cortex of PS19-fE4 and PS19-fE4/GFAP-Cre mice, which were normalized to total cellular protein levels. n=3 wells of neurons for each group, unpaired and two tailed t-test. FIG. 4N graphically illustrates APOE protein levels measured by ELISA in the cell lysates of 14-day cultured primary neurons from PS19-fE4/GFAP-Cre mouse hippocampus. After culturing for 14 days the neurons were then treated with different recombinant proteins (Nrg3, HMGB1, and DPP10 all at 10 μg/ml) for 24 hours, and APOE4 protein levels in cell lysates were measured by ELISA and normalized to total cellular protein levels. n=3 wells of neurons for each group. FIG. 4O graphically illustrates APOE protein levels measured by ELISA in the cell lysates of 14-day cultured primary neurons from PS19-fE4 mouse hippocampus were cultured for 14 days and then treated with different recombinant proteins (Nrg3, HMGB1, and DPP10 all at 10 μg/ml) for 24 hours, and APOE4 protein levels in cell lysates were measured by ELISA and normalized to total cellular protein levels. n=3 wells of neurons for each group, One-way ANOVA with Tukey's post hoc multiple comparisons test. All data are represented as mean±SEM. Ex Neuron, excitatory neuron; In Neuron, inhibitory neuron; OPC, oligodendrocyte precursor cell.

FIG. 5A-5J illustrate that HMGB1 nucleo-cytoplasmic translocation and release from hippocampal neurons is reduced drastically by removing neuronal APOE4 and to a lesser extent by removing astrocytic APOE4. FIG. 5A shows representative images of immunostaining with anti-HMGB1 and DAPI in the dentate gyrus of 10-month-old PS19-fE4 and PS19-fE3 mice with and without Cre (scale bar, 40 μm). FIG. 5B shows representative high magnification images (60×+3× zoom) of immunostaining with anti-HMGB1 and DAPI in the dentate gyrus of 10-month-old PS19-fE4 and PS19-fE3 mice with and without Cre (scale bar, 10 μm). FIG. 5C graphically illustrates nuclear integrated density of HMGB1 immunostaining in the dentate gyrus of 10-month-old PS19-fE4 and PS19-fE3 mice with and without Cre. FIG. 5D graphically illustrates cytoplasmic integrated density of HMGB1 immunostaining in the dentate gyrus of 10-month-old PS19-fE4 and PS19-fE3 mice with and without Cre. FIG. 5E graphically illustrates the average number of nuclear HMGB1⁺ puncta in the dentate gyrus of 10-month-old PS19-fE4 and PS19-fE3 mice with and without Cre. FIG. 5F graphically illustrates the average number of cytoplasmic HMGB1⁺ puncta in the dentate gyrus of 10-month-old PS19-fE4 and PS19-fE3 mice with and without Cre. For quantifications in c-f, n=6 mice per group, and all data are represented as mean±SEM, one-way ANOVA with Tukey's post hoc multiple comparisons test. FIG. 5G graphically illustrates total HMGB1 protein levels measured by ELISA in the hippocampal interstitial fluid (ISF) of 8.5-month-old PS19-fE4 mice with and without Cre. FIG. 5H graphically illustrates HMGB1 protein levels measured by ELISA in each collected ISF fraction of 8.5-month-old PS19-fE4 mice with and without Cre. Fractions 1-2 were excluded from analyses in FIG. 5G-5H since artificial CSF was circulated at a higher flow rate for the first two hours to prevent clogging of the tubing. Data in FIG. 5G-5H include n=2 mice per genotype and are represented as mean±SEM, one-way ANOVA with Tukey's post hoc multiple comparisons test. FIG. 5I graphically illustrates the quantified ratios of percent Iba1 coverage area between the injected:non-injected hippocampal sides 6 days post-injection. HMGB1-absent (n=3) or HMGB1-enriched (n=3) interstitial fluids (ISFs) were injected into the hippocampus of 8.5-month-old wildtype mice and the levels of Iba1 were measured 6 days following injection. FIG. 5J graphically illustrates the quantified ratios of percent GFAP coverage area between the injected:non-injected hippocampal sides 6 days post-injection. HMGB1-absent (n=3) or HMGB1-enriched (n=3) interstitial fluids (ISFs) were injected into the hippocampus of 8.5-month-old wildtype mice and the levels of Iba1 were measured 6 days following injection. For quantifications in FIGS. 5I-5J, n=3 mice were used for control (HMGB1-absent) and experimental (HMGB1-enriched) experimental groups.

FIG. 6A-6R illustrate that treatment with HMGB1 inhibitors blocks nucleo-cytoplasmic translocation of HMGB1 and ameliorates gliosis, Tau pathology, myelin deficits, and neurodegeneration in PS19-fE4 mice. FIG. 6A is a schematic diagram illustrating the experimental design of an HMGB1 inhibitor study occurring over a 12-week period where PS19-fE4 and PS19-fE3 mice were treated with either a 0.9% saline vehicle or HMGB1 inhibitors (a mixture of 80 mg/kg of ethyl pyruvate and 20 mg/kg of glycyrrhizic acid dissolved in 0.9% saline), starting at 6.5 months of age and ending at 9.5 months of age. FIG. 6B shows representative high magnification images (60×+3× zoom) of immunostaining with anti-HMGB1 and DAPI in the dentate gyrus of 9.5-month-old PS19-fE4 and PS19-fE3 mice following treatment with saline or HMGB1 inhibitors (scale bar, 10 μm). FIG. 6C graphically illustrates quantification of nuclear integrated density of HMGB1 immunostaining in the dentate gyrus of 9.5-month-old treated PS19-fE4 and PS19-fE3 mice. FIG. 6D graphically illustrates quantification of cytoplasmic integrated density of HMGB1 immunostaining in the dentate gyrus of 9.5-month-old treated PS19-fE4 and PS19-fE3 mice. FIG. 6E shows representative images of microglia immunostaining with anti-Iba1 in the hippocampus of 9.5-month-old treated PS19-fE4 and PS19-fE3 mice (scale bar, 500 μm). FIG. 6F graphically illustrates quantification of the percent Iba1 coverage area in the hippocampus of 9.5-month-old treated PS19-fE4 and PS19-fE3 mice. FIG. 6G shows representative images of astrocyte immunostaining with anti-GFAP in the hippocampus of 9.5-month-old treated PS19-fE4 and PS19-fE3 mice (scale bar, 500 μm). FIG. 6H graphically illustrates quantification of percent GFAP coverage area in the hippocampus of 9.5-month-old treated PS19-fE4 and PS19-fE3 mice. FIG. 6I shows representative images of pTau immunostaining with anti-AT8 in the hippocampus of 9.5-month-old treated PS19-fE4 and PS19-fE3 mice (scale bar, 500 μm). FIG. 6J graphically illustrates quantification of percent pTau (AT8) coverage area in the hippocampus of 9.5-month-old treated PS19-fE4 and PS19-fE3 mice. FIG. 6K shows representative images of myelin sheath staining with anti-MBP and DAPI in the stratum radiatum of the hippocampus underneath the pyramidal cell layer of CA1 in 9.5-month-old treated PS19-fE4 and PS19-fE3 mice (scale bar, 50 μm). FIG. 6L graphically illustrates quantification of the percent MBP coverage area in the hippocampal CA1 subregion of 9.5-month-old treated PS19-fE4 and PS19-fE3 mice. FIG. 6M shows representative images of the ventral hippocampus of 9.5-month-old treated PS19-fE4 and PS19-fE3 mice after staining with Sudan Black (scale bar, 1 mm). FIG. 6N graphically illustrates quantification of hippocampal volume in 9.5-month-old treated PS19-fE4 and PS19-fE3 mice. FIG. 6O graphically illustrates quantification of posterior lateral ventricle volume in 9.5-month-old treated PS19-fE4 and PS19-fE3 mice. For quantifications in FIGS. 6C, 6D, 6F, 6H, 6J, 6L, 6N, 6O, PS19-fE4 saline: n=16; PS19-fE4 HMGB1 inhibitors: n=18; PS19-fE3 saline: n=16; PS19-fE3 HMGB1 inhibitors: n=14. All data are represented as mean±SEM, one-way ANOVA with Tukey's post hoc multiple comparisons test. FIG. 6P graphically illustrates quantification of nuclear puncta number of HMGB1 immunostaining in the dentate gyrus of 9.5-month-old PS19-fE4 and PS19-fE3 mice following treatment with saline or HMGB1 inhibitors, ethyl pyruvate (EP) and glycyrrhizic acid (GA) (HMGB1-In). FIG. 6Q graphically illustrates quantification of cytoplasmic puncta number of HMGB1 immunostaining in the dentate gyrus of 9.5-month-old PS19-fE4 and PS19-fE3 mice following treatment with saline or HMGB1 inhibitors, ethyl pyruvate (EP) and glycyrrhizic acid (GA) (HMGB1-In). FIG. 6R graphically illustrates quantification of percent CD68 coverage area in the hippocampus of 9.5-month-old treated PS19-fE4 and PS19-fE3. For quantifications in FIG. 6A, 6B, 6D, PS19-fE4 saline: n=16; PS19-fE4 HMGB1 inhibitors: n=18; PS19-fE3 saline: n=16; PS19-fE3 HMGB1 inhibitors: n=14. All data are represented as mean±SEM, one-way ANOVA with Tukey's post hoc multiple comparisons test.

FIG. 7A-7D illustrate characterization of PS19-fE mouse models with the APOE gene specifically removed from neurons or astrocytes by cell type-specific Cre expression. FIG. 7A. Representative images of the cell type-specificity of Cre recombinase expression as determined by coimmunostaining with anti-Cre, anti-GFAP, and anti-NeuN in the hippocampus of 10-month-old PS19-fE4 mice with Syn1-Cre or GFAP-Cre (scale bar, 50 m). FIG. 7B. Representative images of the cell type-specificity of APOE expression as determined by coimmunostaining with anti-ApoE, anti-GFAP, and anti-NeuN in the hippocampus of 10-month-old PS19-fE4 mice with and without Cre (scale bar, 50 m). FIG. 7C. Representative image of APOE⁺ microglia in the hippocampus of 10-month-old PS19-fE4/GFAP-Cre mice as determined by co-immunostaining with anti-ApoE and anti-Iba1 (scale bar, 50 m). FIG. 7D. ApoE levels measured by ELISA in the hippocampal lysates of 10-month-old PS19-fE4 and PS19-fE3 mice with and without Cre (PS19-fE4: No Cre, n=21; Syn1-Cre, n=18; GFAP-Cre, n=17; PS19-fE3: No Cre, n=17; Syn1-Cre, n=11; GFAP-Cre, n=11). ApoE levels are normalized to the weight of the dissected hippocampal tissue for each individual mouse. Data are represented as mean±SEM, one-way ANOVA with Tukey's post hoc multiple comparisons test.

FIG. 8A-8F illustrate AAV2 virus does not directly penetrate the non-injected hippocampal side and microgliosis is reduced after removal of neuronal or astrocytic APOE4 in the non-injected hippocampal side of fE4 mice that received unilateral injections of AAV2-Tau-P301S. FIG. 8A. Representative images of the injected and non-injected hippocampal sides of a 10-month-old fE4 mouse 2 weeks after a unilateral injection with an AAV2-GFP virus (1.0E+13 vg/mL) and immunostained with anti-GFP (scale bar, 500 m). FIG. 8B. Representative high magnification images of the dentate gyrus of the injected and non-injected hippocampal sides of a 10-month-old fE4 mouse 2 weeks after a unilateral injection with an AAV2-GFP virus and immunostained with anti-GFP and DAPI (scale bar, 100 μm). FIG. 8C. Representative images of microglia immunostaining with anti-Iba1 in the non-injected hippocampal side of 13-month-old fE4 mice with and without Cre or fE3 mice without Cre 12 weeks after unilateral injection of AAV2-Tau-P301S (2.10E+13 vg/mL) (scale bar, 500 μm). FIG. 8D. Quantification of the percent Iba1 coverage area in the non-injected hippocampal side of 13-month-old fE mice with and without Cre 12 weeks after unilateral injection of AAV2-Tau-P301S. FIG. 8E. Representative images of astrocyte immunostaining with anti-GFAP in the non-injected hippocampal side of 13-month-old fE mice with and without Cre 12 weeks after unilateral injection of AAV2-Tau-P301S (scale bar, 500 μm). FIG. 8F. Quantification of the percent GFAP coverage area in the non-injected hippocampal side of 13-month-old fE mice with and without Cre 12 weeks after unilateral injection of AAV2-Tau-P301S. For quantifications in FIG. 8D,F n=8 mice per genotype group, and all data are represented as mean±SEM, one-way ANOVA with Tukey's post hoc multiple comparisons test.

FIG. 9A-9B illustrate removing neuronal, but not astrocytic, APOE4 ameliorates neuronal hyperexcitability in the hippocampus of PS19-fE4 mice. FIG. 9A. Average normalized fPSP slopes in CA1 stratum radiatum in response to incremental stimulation of Schaffer collaterals. Neuronal APOE4 expression renders CA3-CA1 network hyperexcitable as evidenced by augmented response to synaptic stimulation. FIG. 9B. Calculated individual fPSP slope gain values for all experiments in (a). PS19-fE3: n=13, N=2; PS19-fE4:n=20, N=4; PS19-fE4/Syn1-Cre: n=25, N=4; PS19-fE4/GFAP-Cre: n=13, N=2. Data is represented as mean±SEM, one-way ANOVA with Tukey's post hoc multiple comparisons test.

FIG. 10A-10F illustrate myelin deficits and OPC pool depletion are significantly reduced after removal of APOE4 from neurons, but not astrocytes. FIG. 10A. Representative images of myelin sheath staining with anti-MBP and DAPI in the stratum radiatum of the hippocampus underneath the pyramidal cell layer of CA1 in 10-month-old PS19-fE4 and PS19-fE3 mice with and without Cre (scale bar, 50 m). FIG. 10B. Quantification of the percent MBP coverage area in the hippocampal CA1 subregion of 10-month-old PS19-fE4 and PS19-fE3 mice with and without Cre. FIG. 10C. Representative images of OPCs by immunostaining with anti-NG2 in the hippocampus of 10-month-old PS19-fE4 and PS19-fE3 mice with and without Cre (scale bar, 500 μm). FIG. 10D. Quantification of the percent NG2 coverage area in the hippocampus of 10-month-old PS19-fE4 and PS19-fE3 mice with and without Cre. FIG. 10E. Representative images of myelin sheath staining with anti-MBP and DAPI in the stratum radiatum of the hippocampus in 10-month-old fE4 and fE3 mice. FIG. 10F. Representative images of OPC staining with anti-NG2 in the hippocampus of 10-month-old fE4 and fE3 mice. For quantifications in FIGS. 10B and D, n=6 mice per genotype group, and all data are represented as mean±SEM, one-way ANOVA with Tukey's post hoc multiple comparisons test.

FIG. 11A-11J. illustrate snRNA-seq analysis characterizing hippocampal cell clusters and the associations of some of the clusters with pathologies. FIG. 11A. Dot-plot showing the normalized average expression of selected marker genes for all 33 distinct hippocampal cell clusters. FIG. 11B. Feature plot illustrating the relative levels of normalized human APOE gene expression across all 33 hippocampal cell clusters in PS19-fE4 mice with no Cre (n=4), Syn1-Cre (n=4), or GFAP-Cre (n=3) and PS19-fE3 mice with no Cre (n=3). FIG. 11C. UMAP plot highlighting cells in hippocampal cell clusters 17 and 25 for each mouse genotype group. FIG. 11D. Box plot of the proportion of oligodendrocyte cluster 17 per sample by mouse genotype group. Each dot represents one mouse. FIG. 11E. Dot-plot of the top 20 KEGG pathways significantly enriched for the differentially expressed genes of oligodendrocyte cluster 17 vs other oligodendrocyte clusters 1 and 2. P-values are based on a hypergeometric test and are adjusted for multiple testing using the Benjamini-Hochberg method. The size of the dots is proportional to the number of genes in the given gene set. Gene Ratio represents the proportion of genes in the respective gene set that are deemed to be differentially expressed using the FindMarkers function in Seurat. FIG. 11F. Box plot of the proportion of excitatory neuron cluster 25 per sample by mouse genotype group. Each dot represents one mouse. FIG. 11G-J. Scatter plots of proportion of cells per sample for oligodendrocyte cluster 17 (FIG. 11G,H) and excitatory neuron cluster 25 (FIG. 11I,J) versus measurement of hippocampus volume (mm³) and pTau (AT8) coverage area (%). The samples are colored by the mouse genotype group they belong to and genotype-specific best-fit lines are included.

FIG. 12A-12S illustrate snRNA-seq analysis showing astrocyte and microglia subclusters and the associations of some of the subclusters with pathologies. FIG. 12A. UMAP plot highlighting cells in astrocyte subclusters 4 and 7 in PS19-fE4 mice with no Cre (n=4), Syn1-Cre (n=4), or GFAP-Cre (n=3) and PS19-fE3 mice with no Cre (n=3). FIG. 12B. Feature plot illustrating the relative levels of normalized human APOE gene expression across all astrocyte subclusters for each mouse genotype group. FIG. 12C. Box plot of the proportion of cells per sample within each mouse genotype group for astrocyte subcluster 4. Each dot represents one sample. FIG. 12D,E. Scatter plot of hippocampus volume (mm3) (d) and pTau (AT8) coverage area (%) (e) versus proportion of cells per sample in astrocyte subcluster 4. The samples are colored by mouse genotype groups they belong to and genotype-specific best-fit lines are included. FIG. 12F. Box plot of the proportion of cells per sample within each mouse genotype group for astrocyte subcluster 7. FIG. 12G,H. Scatter plot of hippocampus volume (mm³) (g) and pTau (AT8) coverage area (%) (h) versus proportion of cells per sample in astrocyte subcluster 7. FIG. 12I. UMAP plot highlighting cells in microglia subclusters 4 and 8 for each mouse genotype group. FIG. 12J. Feature plot illustrating the relative levels of normalized human APOE gene expression across all microglia subclusters for each mouse genotype group. FIG. 12K. Box plot of the proportion of cells per sample within each mouse genotype group for microglia subcluster 4. FIG. 12L,M. Scatter plot of hippocampus volume (mm³) (1) and pTau (AT8) coverage area (%) (m) versus proportion of cells per sample in microglia subcluster 4. FIG. 12N. Box plot of the proportion of cells per sample within each mouse genotype group for microglia subcluster 8. FIG. 12O,P. Scatter plot of hippocampus volume (mm³) (o) and pTau (AT8) coverage area (%) (p) versus proportion of cells per sample in microglia subcluster 8. FIG. 12Q,R. Dot-plot of the top 20 KEGG pathways significantly enriched for the differentially expressed genes of astrocyte subcluster 4 vs all other astrocyte subclusters (q) and microglia subcluster 4 vs all other microglia subclusters (r). P-values are based on a hypergeometric test and are adjusted for multiple testing using the Benjamini-Hochberg method. The size of the dots is proportional to the number of genes in the given gene set. Gene Ratio represents the proportion of genes in the respective gene set that are deemed to be differentially expressed using the FindMarkers function in Seurat. FIG. 12S. Principal component analysis plot for clusters 17 and 25 and astrocyte and microglia subclusters. PC1 and PC2 showed the overall relationship between the clusters based on the similarity of the estimated log odds ratio per unit changes in eight pathologies per cluster/subcluster. AS, astrocyte; MG, microglia.

FIG. 13A-13I illustrate istrocytic APOE regulation of neuronal APOE expression. FIG. 13A. UMAP plot of 22 distinct hippocampal cell clusters from the GEO:GSE164507 dataset. FIG. B. Feature plot illustrating the relative levels of normalized human APOE gene expression across all 22 hippocampal cell clusters in floxed APOE3 (FE3) mice, floxed APOE4 (FE4) mice, P301S-Tau/Aldh111-Cre/APOE3flox/flox mice treated with oil (TAFE3_oil), P301S-Tau/Aldh111-Cre/APOE4^{flox/flox} treated with oil (TAFE4_oil), P301S-Tau/Aldh111-Cre/APOE3^{flox/flox} mice treated with tamoxifen (TAFE3_tam), P301S-Tau/Aldh111-Cre/APOE4^{flox/flox} treated with tamoxifen (TAFE4_tam). FIG. 13C,D. Histogram of APOE expression in cluster 3 excitatory (Ex) neurons (c) and cluster 5 inhibitory (In) neurons (d) from FE3, FE4, TAFE3_oil, and TAFE4_oil mice. For Log FC and p-values. FIG. 13E,F. Histogram of APOE expression in cluster 3 excitatory (Ex) neurons (e) and cluster 5 inhibitory (In) neurons (f) from TAFE3_oil, TAFE4_oil, TAFE3_tam, and TAFE4_tam mice. For Log FC and p-values. FIG. 13G. APOE protein levels were measured by ELISA in the cell lysates of 14-day cultured primary neurons from the hippocampus and cortex of PS19-fE4 and PS19-fE4/GFAP-Cre mice, which were normalized to total cellular protein levels. n=3 wells of neurons for each group, unpaired and two tailed t-test. FIG. 13H,I. Primary neurons from PS19-fE4/GFAP-Cre (h) or PS19-fE4 (i) mouse hippocampus were cultured for 14 days and then treated with different recombinant proteins (Nrg3, HMGB1, and DPP10 all at 10 μg/ml) for 24 hours. After the treatment, APOE4 protein levels in cell lysates were measured by ELISA and normalized to total cellular protein levels. n=3 wells of neurons for each group, One-way ANOVA with Tukey's post hoc multiple comparisons test. All data are represented as mean±SEM. Ex Neuron, excitatory neuron; In Neuron, inhibitory neuron; OPC, oligodendrocyte precursor cell.

FIG. 14A-14F illustrate HMGB1-enriched ISF fractions elicit microgliosis in wildtype mice following a unilateral injection. FIG. 14A. Representative high magnification images (60×+3× zoom) of immunostaining with anti-HMGB1 and DAPI in the dentate gyrus of 10-month-old fE4 and fE3 mice (scale bar, 10 μm). FIG. 14B. Representative images of immunostaining with anti-HMGB1 and DAPI in the dentate gyrus of 10-month-old PS19-fE4 and PS19-fE3 mice with and without Cre (scale bar, 40 μm). FIG. 14C. Representative images of microglia stained with anti-Iba1 in the hippocampus of 8.5-month-old wildtype mice following a unilateral injection of HMGB1-absent (n=3) or HMGB1-enriched (n=3) ISF (scale bar, 500 μm). FIG. 14D. Quantification of the ratio of percent Iba1 coverage area between the injected:non-injected hippocampal sides 6 days post-injection. FIG. 14E. Representative images of astrocytes stained with anti-GFAP in the hippocampus of 8.5-month-old wildtype mice following a unilateral injection of injection of HMGB1-absent or HMGB1-enriched ISF (scale bar, 500 μm). FIG. 14F. Quantification of the ratio of percent GFAP coverage area between the injected:non-injected hippocampal sides 6 days post-injection. For quantifications in d and f, n=3 mice were used for control (HMGB1-absent) and experimental (HMGB1-enriched) experimental groups. All data are represented as mean±SEM, one-way ANOVA with Tukey's post hoc multiple comparisons test.

FIG. 15A-15D illustrate treatment with HMGB1 inhibitors reduces HMGB1 puncta formation and ameliorates microglia activation in PS19-fE4 mice. FIG. 15A,B. Quantification of nuclear puncta number (a) and cytoplasmic puncta number (b) of HMGB1 immunostaining in the dentate gyrus of 9.5-month-old PS19-fE4 and PS19-fE3 mice following treatment with saline or HMGB1 inhibitors. FIG. 15C) Representative images of activated microglia immunostaining with anti-CD68 in the hippocampus of 9.5-month-old treated PS19-fE4 and PS19-fE3 mice (scale bar, 500 μm). FIG. 15D) Quantification of percent CD68 coverage area in the hippocampus of 9.5-month-old treated PS19-fE4 and PS19-fE3. For quantifications in a,b,d, PS19-fE4 saline: n=16; PS19-fE4 HMGB1 inhibitors: n=18; PS19-fE3 saline: n=16; PS19-fE3 HMGB1 inhibitors: n=14. All data are represented as mean±SEM, one-way ANOVA with Tukey's post hoc multiple comparisons test.

FIG. 16A-16D illustrate “APOE4-HMGB1-inflammation-degeneration” cascade hypothesis/model of APOE4-related AD and other tauopathies. FIG. 16A. Schematic overview of the pathogenic mechanisms of cell type-specific APOE4 pathogenesis. In the presence of both neuronal and astrocytic APOE4, as shown in PS19-fE4 mice, a pathogenic cascade initiates with neuronal expression of APOE4, which can be induced by various neuronal stressors. Neuronal APOE4 has a potent effect on the accumulation and propagation of Tau pathology, which can further induce neuronal APOE4 expression. Elevated neuronal APOE4, in concert with Tau pathology, triggers the nucleo-cytoplasmic translocation and release of HMGB1 from neurons. Astrocytic APOE4 does not have a direct effect on Tau pathology accumulation and propagation, however, it indirectly enhances APOE4 expression in neurons by promoting the release of astrocytic factors, such as DPP10, capable of regulating neuronal APOE4 expression, thus, secondarily promoting APOE4/Tau pathology-induced HMGB1 translocation and release from neurons. Upon its release from neurons, HMGB1 acts as an inflammatory cytokine that induces gliosis. The extensive gliosis, especially the accumulation of toxic astrocytic and microglial subtypes, results in the aberrant engulfment of neurons, synapses, and oligodendrocyte-derived myelin sheaths and subsequent neurodegeneration, neurodysfunction, and oligodendrocyte degeneration. FIG. 16B. Schematic diagram illustrating the protective mechanisms of removing neuronal APOE4 on the proposed cell type-specific APOE4 pathogenic mechanism. Removal of neuronal APOE4, as shown in PS19-fE4/Syn1-Cre mice, leads to a drastic reduction of the accumulation and propagation of Tau pathology in neurons. The reduced Tau pathology and the absence of neuronal APOE4 together present as a blockade for HMGB1 release from neurons. Since astrocytic APOE4 contributes to HMGB1 release by indirectly enhancing neuronal APOE4 expression, the lack of neuronal APOE4 blocks the indirect effect of astrocytic APOE4 on triggering neuronal HMGB1 release. Without adequate neuronal HMGB1 release, there is no induction of gliosis, particularly of the toxic microglial and astrocytic subtypes, consequently leading to reduced neurodegeneration, neurodysfunction, and oligodendrocyte degeneration. Thus, removal of neuronal APOE4 leads to a complete prevention or rescue of all observed pathologies. FIG. 16C. Schematic diagram illustrating the protective mechanisms of removing astrocytic APOE4 on the proposed cell type-specific APOE4 pathogenic mechanism. Removal of astrocytic APOE4, as shown in the PS19-fE4/GFAP-Cre mice, leads to a significant, but not complete, reduction in neuronal APOE4 expression. The reduced levels of neuronal APOE4 are still sufficient to induce significant Tau pathology accumulation and propagation, which, together with the low level of neuronal APOE4, triggers low levels of HMGB1 release. Without high enough HMGB1 release, there is a large reduction in the overall levels of gliosis. Still, the low levels of neuronal APOE4 and HMGB1 release are sufficient to promote the presence of toxic astrocytic and microglial subtypes. The overall reduction of gliosis leads to reduced neurodegeneration, although a subpopulation of disease-associated neurons is still present due to the low levels of neuronal APOE4. The presence of toxic glial subtypes still leads to oligodendrocyte deficits, probably due to higher sensitivity of oligodendrocytes to lower levels of toxic glial subtypes. The high levels of Tau pathology and the presence of toxic glial subtypes may continuously contribute to neurodysfunction. Thus, removal of astrocytic APOE4 leads to a partial prevention or rescue of the observed pathologies. FIG. 16D. Schematic diagram illustrating the protective mechanisms of HMGB1 inhibitor treatment on the proposed ell type-specific APOE4 pathogenic mechanism. In the presence of HMGB1 inhibitors, the release of HMGB1 from neurons is blocked. Without released HMGB1 to act as an inflammatory cytokine, gliosis is significantly reduced, consequently leading to a significant reduction in neurodegeneration and oligodendrocyte degeneration. The lack of gliosis and likely toxic glial subtypes also leads to a significant reduction in Tau pathology, which, together with reduced neurodegeneration, likely ameliorates neurodysfunction. Thus, HMGB1 inhibitor treatment also leads to a complete prevention or rescue of all observed pathologies.

FIG. 17A-17M illustrate APOE4 promotes the cellular release of HMGB1 to induce acute and persistent gliosis in mouse hippocampus. FIG. 17A. HMGB1 protein levels measured by ELISA in the hippocampal interstitial fluid (ISF) of 10-month-old PS19-E4 and PS19-E3 mice. FIG. 17B. HMGB1 protein levels measured by ELISA in each collected ISF fraction of 10-month-old PS19-E4 mice. Fractions 1 and 2 were excluded from analyses in A,B since artificial CSF was circulated at a higher flow rate for the first two hours to prevent clogging of the tubing. FIG. 17C. HMGB1 protein levels measured by ELISA in each collected ISF fraction of one PS19-E4 mouse used for experiments in D and F-I. Fractions 3-7 were designated as HMGB1-absent (HMGB1⁻) fractions. Fractions 19-22 were designated as HMGB1-enriched (HMGB1⁺) fractions. FIG. 17D. Experimental design of a study involving the injection of HMGB1-enriched or HMGB1-absent ISF into the hippocampus of 10-month-old WT mice and assessment of acute gliosis 6 days after the initial injection. FIG. 17E. Experimental design of a study involving two injections of recombinant HMGB1 (rHMGB1) or saline into the hippocampus of 3-month-old PS19-E4 mice and assessment of gliosis 8-weeks after the initial injection. FIG. 17F. Representative images of microglia stained with anti-Iba1 in the hippocampus of 10-month-old WT mice following a unilateral injection of HMGB1-absent (HMGB1⁻) or HMGB1-enriched (HMGB1⁺) ISF collected from a 10-month-old PS19-E4 mouse (scale bar, 500 μm). FIG. 17G. Quantification of the ratio of the percent Iba1 coverage area between the injected and non-injected hippocampal sides 6 days post-injection. FIG. 17H. Representative images of astrocytes stained with anti-GFAP in the hippocampus of 10-month-old wildtype mice following a unilateral injection of HMGB1-absent or HMGB1-enriched ISF collected from a 10-month-old PS19-E4 mouse (scale bar, 500 μm). FIG. 17I. Quantification of the ratio of the percent GFAP coverage area between the injected and non-injected hippocampal sides 6 days post-injection. FIG. 17J. Representative images of microglia stained with anti-Iba1 in the hippocampus of 5-month-old PS19-E4 mice following two unilateral injections of rHMGB1 or saline (scale bar, 500 μm). FIG. 17K. Quantification of the ratio of the percent Iba1 coverage area between the injected and non-injected hippocampal sides 8-weeks after the initial injection. FIG. 17L. Representative images of astrocytes stained with anti-GFAP in the hippocampus of 3-month-old PS19-E4 mice following two unilateral injections of rHMGB1 or saline (scale bar, 500 μm). FIG. 17M. Quantification of the ratio of the percent GFAP coverage area between the injected and non-injected hippocampal sides 8-weeks after the initial injection. Data in A,B include n=7 mice per genotype. For quantifications in G,I, n=9 mice were used for control (HMGB1⁻) and experimental (HMGB1⁺) groups. For quantifications in K,M, n=8 mice were used for rHMGB1 and saline control groups. All data are represented as mean±SEM, unpaired two-sided t test. ISF, interstitial fluid; WT, wildtype; rHMGB1, recombinant HMGB1.

FIG. 18A-18B illustrate treatment of PS19-E4 mice with HMGB1 inhibitors drastically reduces HMGB1 release to ISF. Experimental design of the HMGB1 inhibitor study includes the treatment of PS19-E4 and PS19-E3 mice with either a 0.9% saline vehicle or HMGB1 inhibitors (a mixture of 80 mg/kg of ethyl pyruvate and 20 mg/kg of glycyrrhizic acid dissolved in 0.9% saline), three doses per week (Monday, Wednesday, and Friday), for three weeks. After the treatment, micro-dialysis was performed to collect the interspatial fluid (ISF) from each mouse hippocampus. FIG. 18A. HMGB1 protein levels measured by ELISA in the ISF of HMGB1 inhibitor-treated or saline-treated PS19-E4 mice. FIG. 18B. HMGB1 protein levels measured by ELISA in each collected ISF fraction of HMGB1 inhibitor-treated PS19-E4 mice. Fractions 1 and 2 were excluded from analyses in E,F. Quantified data (PS19-E4, n=5; PS19-E4 HMGB1-In, n=5) are represented as mean±SEM, unpaired two-sided t test. HMGB1-In, HMGB1-Inhibitors.

FIG. 19A-19C illustrate enhanced nucleo-cytoplasmic translocation of HMGB1 in neurons of human apoE4/4 AD brains versus human apoE3/3 AD brains. FIG. 19A. Immunofluorescent staining of HMGB1 protein on sections from human apoE4/4 AD brains and human apoE3/3 AD brains. Note the enhanced nucleo-cytoplasmic translocation of HMGB1 in neurons from human apoE4/4 AD brains versus those from human apoE3/3 AD brains. FIGS. 19B and C. Quantification of the nuclear integrated density (B) and extranuclear integrated density (C) of HMGB1 immunostaining in neurons from human apoE4/4 AD brains and human apoE3/3 AD brains. Quantified data (n=5 per genotype group) are represented as mean±SEM, unpaired two-sided t test.

### DETAILED DESCRIPTION

As described herein, inhibitors of High mobility group box protein 1 (HMGB1) can significantly reduce HMGB1 nucleo-cytosplasmic translocation, gliosis, neurodegeneration, Tau pathologies, and myelin deficits in subjects having an APOE4 allele. For example, the methods can be used for treatment of tauopathies. Tauopathies are neurodegenerative disorders characterized by the deposition of abnormal tau protein in the brain. Such tauopathies include Alzheimer's disease, Pick disease, progressive supranuclear palsy, corticobasal degeneration, and argyrophilic grain disease, primary age-related tauopathy, chronic traumatic encephalopathy, or frontotemporal dementia.

As illustrated herein, neuronal APOE4 promotes the release of HMGB1 from neurons, which initiates and/or exacerbates inflammation within the brain, induces gliosis, and leads to neurodegeneration and myelin deficits. In subjects with the APOE4 allele, HMGB1 protein is aberrantly translocated from the nucleus to the cytosol of hippocampal neurons and released from neurons into the hippocampal interstitial fluid. Experiments described herein show that administration of two HMGB1 inhibitors, ethyl pyruvate and glycyrrhizic acid, blocked HMGB1 nucleo-cytoplasmic translocation in mice and drastically reduced the extent of gliosis, neurodegeneration, Tau pathology, and myelin deficits in APOE4 mice, while having no discernable effects on the APOE3 mice.

Methods are described herein that can include administering a HMGB1 inhibitor to a subject. The subject can be a subject whose genome includes the APOE4 allele. For example, the subject to be treated is one who expresses an APOE4 protein. In some cases, the methods include administering a therapeutically effective amount of a HMGB1 inhibitor to the subject. Such a therapeutically effective amount of a HMGB1 inhibitor can reduce gliosis, neurodegeneration, Tau deposition, and myelin deficits. For example, such a therapeutically effective amount of a HMGB1 inhibitor can reduce one or more of gliosis, neurodegeneration, Tau deposition, or myelin deficits by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 99%, or at least 99%.

### High Mobility Group Box Protein 1 (HMGB1)

High mobility group box protein 1 (HMGB1) is a ubiquitous nuclear protein released by glia and neurons upon inflammasome activation and activates receptor for advanced glycation end products (RAGE) and toll-like receptor (TLR) 4 on the target cells. HMGB1/TLR4 axis is a key initiator of neuroinflammation. As shown herein, neuronal APOE4 promotes the release of HMGB1 from neurons, which initiates and/or exacerbates inflammation within the brain, induces gliosis, and leads to neurodegeneration and myelin deficits.

A sequence for a human HMGB1 protein is shown below as SEQ ID NO: 1 (UNIPROT accession no. P09429).

```
                10 20 30 40 50
MGKGDPKKPR GKMSSYAFFV QTCREEHKKK HPDASVNFSE FSKKCSERWK
                60 70 80 90 100
TMSAKEKGKF EDMAKADKAR YEREMKTYIP PKGETKKKFK DPNAPKRPPS
              110 120 130 140 150
AFFLFCSEYR PKIKGEHPGL SIGDVAKKLG EMWNNTAADD KQPYEKKAAK
              160 170 180 190 200
LKEKYEKDIA AYRAKGKPDA AKKGVVKAEK SKKKKEEEED EEDEEDEEEE
              210
EDEEDEDEEE DDDDE
```

A cDNA sequence encoding such a HMGB1 protein is shown below as SEQ ID NO:2 (NCBI accession no. X12597.1).

| | |
|---|---|
| 1 | GGGGGACGGG CACTGGGCGA CTCTGTGCCT CGCTGAGGAA |
| 41 | AAATAACTAA ACATGGGCAA AGGAGATCCT AAGAAGCCGA |
| 81 | GAGGCAAAAT GTCATCATAT GCATTTTTTG TGCAAACTTG |
| 121 | TCGGGAGGAG CATAAGAAGA AGCACCCAGA TGCTTCAGTC |
| 161 | AACTTCTCAG AGTTTTCTAA GAAGTGCTCA GAGAGGTGGA |
| 201 | AGACCATGTC TGCTAAAGAG AAAGGAAAAT TTGAAGATAT |
| 241 | GGCAAAAGCG GACAAGGCCC GTTATGAAAG AGAAATGAAA |
| 281 | ACCTATATCC CTCCCAAAGG GGAGACAAAA AAGAAGTTCA |
| 321 | AGGATCCCAA TGCACCCAAG AGGCCTCCTT CGGCCTTCTT |
| 361 | CCTCTTCTGC TCTGAGTATC GCCCAAAAAT CAAAGGAGAA |
| 401 | CATCCTGGCC TGTCCATTGG TGATGTTGCG AAGAAACTGG |
| 441 | GAGAGATGTG GAATAACACT GCTGCAGATG ACAAGCAGCC |
| 481 | TTATGAAAAG AAGGCTGCGA AGCTGAAGGA AAAATACGAA |
| 521 | AAGGATATAG CTGCATATCG AGCTAAAGGA AAGCCTGATG |
| 561 | CAGCAAAAAA GGGAGTTGTC AAGGCTGAAA AAAGCAAGAA |
| 601 | AAAGAAGGAA GAGGAGGAAG ATGAGGAAGA TGAAGAGGAT |
| 641 | GAGGAGGAGG AGGAAGATGA AGAAGATGAA GATGAAGAAG |
| 681 | AAGATGATGA TGATGAATAA GTTGGTTCTA GCGCAGTTTT |
| 721 | TTTTTTCTTG TCTATAAAGC ATTTAACCCC CCTGTACACA |
| 761 | ACTCACTCCC TITTAAAGAA AAAAATTGAA ATGTAAGGCT |
| 801 | GTGTAAGATT TGTTTTTAAA CTGTACAGTG TCTTTTTTTG |
| 841 | TATAGTTAAC ACACTACCGA ATGTGTCTTT AGATAGCCCT |
| 881 | GTCCTGGTGG TATTTTCAAT AGCCACTAAC CTTGCCTGGT |
| 921 | ACAGTATGGG GGTTGTAAAT TGGCATGGAA ATTTAAAGCA |
| 961 | GGTTCTTGTT GGTGCACAGC ACAAATTAGT TATATATGGG |
| 1001 | GATGGTAGT |

The human HMGB1 gene resides on chromosome 13 (location 13q12.3; NC_000013.11 (30456704 . . . 30617597, complement)).

Variants and homologs of any of the sequences described here can also be relevant to the methods and compositions described herein. For example, such variants and homologs can have less than 100% sequence identity to any of the sequences described herein. The variants and homologs can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of sequences described herein.

### HMGB1 Inhibitors

The HMGB1 inhibitors refer to any compounds or molecules that are capable of inhibiting the expression, function, or extracellular release of HMGB1. Such HMGB1 inhibitors can include chemical compounds, antibodies, proteins, small RNA molecules, small DNA molecules, and the like that target HMGB1 proteins or HMGB1 nucleic acids.

The chemical compounds may be synthesized compounds or a naturally occurring compounds. One example of an HMGB1 inhibitor that can be used is glycyrrhizin, also called glycyrrhizic acid (structure shown below).

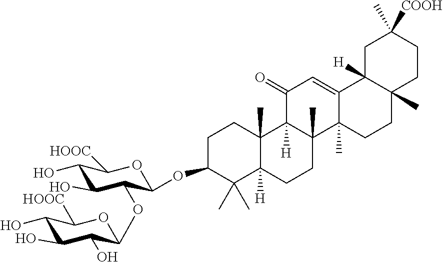

Glycyrrhizin is an HMGB1 inhibitor that can bind one or both HMG boxes in HMGB1 and that can suppress chemoattractant and mitogenic activities of HMGB1. After oral ingestion, glycyrrhizin is hydrolyzed to 18β-glycyrrhetinic acid (enoxolone) by intestinal bacteria and after absorption from the gut, 18β-glycyrrhetinic acid is metabolized to 3β-monoglucuronyl-18β-glycyrrhetinic acid in the liver. The 3β-monoglucuronyl-18β-glycyrrhetinic acid metabolite circulates in the bloodstream. Consequently, its oral bioavailability can be poor. Accordingly, glycyrrhizin, 18β-glycyrrhetinic acid (enoxolone), and/or 3β-monoglucuronyl-18β-glycyrrhetinic acid can be administered intravenously, or locally into selected target tissues, for example, into the brain.

In other examples, HMGB1 inhibitors include, but are not limited to, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies (m2G7 or #10-22), recombinant HMGB1 box A protein, acetylcholine, the nicotinic acetylcholine receptor subtype alpha 7 agonist GTS-21, Peptide P5779, resveratrol, metformin, and a derivative thereof, may also be used in the present invention. These compounds are found to have inhibitory effects on HMGB1 secretion and can be administered by any convenient method (e.g., orally, locally or intravenously). One or more of these compounds may be administered in combination. Glycyrrhizin can also be administered with one or more of these compounds.

In some cases, the HMGB1 inhibitor may be a RNA molecule such as a siRNA, short hairpin RNA, microRNA, antisense RNA, or guide RNA. The RNA molecule may bind to an HMGB1 nucleic acid sequence such as SEQ ID NO: 2. The RNA molecule may be modified in accordance with practical need. Other suitable RNA molecules capable of targeting HMGB1 gene may also be used in the present invention. Examples of HMGB1 siRNAs that are shown below useful for targeting human HMGB1.

| | | Start Position | |
|---|---|---|---|
| | Target Sequence | in the HMGB1 cDNA | SEQ ID NO: |
| 1 | AAGATGAAGAAGAAGATGATG | 671 | SEQ ID NO: 3 |
| 2 | AAGAAGAAGATGATGATGATG | 677 | SEQ ID NO: 4 |
| 3 | AAGGAAAGCCTGATGCAGCAA | 548 | SEQ ID NO: 5 |
| 4 | AAGGCTGCGAAGCTGAAGGAA | 493 | SEQ ID NO: 6 |
| 5 | AAGATGAAGATGAAGAAGAAG | 665 | SEQ ID NO: 7 |

### Apolipoprotein E4

The APOE gene encodes apolipoprotein E, which is a protein that combines with lipids including cholesterol to form lipoproteins. There are a number of different APOE but the major alleles are the e2, e3, and e4 alleles. The most common allele is e3, which is found in more than half of the general population. All apoE isoforms have about 299 amino acids—the only differences being single amino acid changes. However, there are clearly functional differences because apoE3, the common isoform, is not associated with Alzheimer's disease.

Apolipoprotein E4 (APOE4) is the strongest risk factor for sporadic late-onset Alzheimer's disease (AD), which accounts for the vast majority of Alzheimer's disease cases. APOE4 differs from APOE2 and APOE3 at amino acid positions 112 and 158 and has a unique conformation that influences its lipid-binding and receptor-binding properties. ApoE4 contains an arginine residue at position 112, whereas apoE3 has a cysteine at this position.

A sequence for Homo sapiens apolipoprotein E isoform b precursor is shown below as SE ID NO:8 NCBI accession no. NP_000032.1).

| | |
|---|---|
| 1 | MKVLWAALLV TFLAGCQAKV EQAVETEPEP ELRQQTEWQS |
| 41 | GQRWELALGR FWDYLRWVQT LSEQVQEELL SSQVTQELRA |
| 81 | LMDETMKELK AYKSELEEQL TPVAEETRAR LSKELQAAQA |
| 121 | RLGADMEDVC GRLVQYRGEV QAMLGQSTEE LRVRLASHLR |
| 161 | KLRKRLLRDA DDLQKRLAVY QAGAREGAER GLSAIRERLG |
| 201 | PLVEQGRVRA ATVGSLAGQP LQERAQAWGE RLRARMEEMG |
| 241 | SRTRDRLDEV KEQVAEVRAK LEEQAQQIRL QAEAFQARLK |
| 281 | SWFEPLVEDM QRQWAGLVEK VQAAVGTSAA PVPSDNH |

The SEQ ID NO:8 apolipoprotein E sequence includes an eighteen amino acid signal peptide (highlighted above in bold and with underlining). An amino acid sequence for the Homo sapiens apolipoprotein E isoform b precursor without the signal sequence is shown below as SEQ ID NO:9.

| | |
|---|---|
| 1 | KVEQAVETEP EPELRQQTEW QSGQRWELAL GRFWDYLRWV |
| 41 | QTLSEQVQEE LLSSQVTQEL RALMDETMKE LKAYKSELEE |
| 81 | QLTPVAEETR ARLSKELQAA QARLGADMED VCGRLVQYRG |
| 121 | EVQAMLGQST EELRVRLASH LRKLRKRLLR DADDLQKRLA |
| 161 | VYQAGAREGA ERGLSAIRER LGPLVEQGRV RAATVGSLAG |
| 201 | QPLQERAQAW GERLRARMEE MGSRTRDRLD EVKEQVAEVR |
| 241 | AKLEEQAQQI RLQAEAFQAR LKSWFEPLVE DMQRQWAGLV |
| 281 | EKVQAAVGTS AAPVPSDNH |

As noted above, ApoE4 contains an arginine residue at position 112, whereas apoE3 has a cysteine at this position (highlighted above in bold and with underlining).

Hence, one sequence for Homo sapiens apolipoprotein E4 is shown below as SEQ ID NO: 10.

| | |
|---|---|
| 1 | KVEQAVETEP EPELRQQTEW QSGQRWELAL GRFWDYLRWV |
| 41 | QTLSEQVQEE LLSSQVTQEL RALMDETMKE LKAYKSELEE |
| 81 | QLTPVAEETR ARLSKELQAA QARLGADMED VRGRLVQYRG |
| 121 | EVQAMLGQST EELRVRLASH LRKLRKRLLR DADDLQKRLA |
| 161 | VYQAGAREGA ERGLSAIRER LGPLVEQGRV RAATVGSLAG |
| 201 | QPLQERAQAW GERLRARMEE MGSRTRDRLD EVKEQVAEVR |
| 241 | AKLEEQAQQI RLQAEAFQAR LKSWFEPLVE DMQRQWAGLV |
| 281 | EKVQAAVGTS AAPVPSDNH |

The human APOE gene resides on chromosome 19 (location 19q13.32; NC_000019.10 (44905796 . . . 44909393) or NC_060943.1 (47730492 . . . 47734089)). An APOE4 allele is found in about 10-15% of the population. Subjects with other APOE (non-APOE4) alleles typically have a lower probability of developing Alzheimer's disease or other tauopathies. Everyone has two copies of the APOE gene: people with APOE E2/E2 alleles have the lowest overall risk for Alzheimer's and those with APOE E4/E4 have the highest risk. The other combinations of APOE—E2/E3, E2/E4, E3/E3 and E3/E4—fall in between.

A variety of tests are available for determining whether a subject has an APOE4 allele. Such tests can involve genomic sequencing, polymerase chain reaction amplification, restriction enzyme mapping, single nucleotide polymorphism detection, (see, e.g., any of the following websites: 23andme.com; empowerdxlab.com; nebula.org; alzheimersorganization.org; testing.com). Any such tests can be used to determine whether a subject has at least one APOE4 allele.

### Dipeptidyl Peptidase 10 (DPP10)

Dipeptidyl peptidase 10 is a protein that in humans is encoded by the DPP10 gene. The DPP10 protein is a single-pass type II membrane protein that is a member of the S9B family in clan SC of the serine proteases. This protein has no detectable protease activity, most likely due to the absence of the conserved serine residue normally present in the catalytic domain of serine proteases. However, it does bind specific voltage-gated potassium channels and alters their expression and biophysical properties.

A sequence for a human DPP10 protein is available from the UNIPROT databases as accession no. Q8N608, shown below as SEQ ID NO: 11.

```
                10 20 30 40 50
MNQTASVSHH IKCQPSKTIK ELGSNSPPQR NWKGIAIALL VILVVCSLIT
                60 70 80 90 100
MSVILLTPDE LTNSSETRLS LEDLFRKDFV LHDPEARWIN DTDVVYKSEN
              110 120 130 140 150
GHVIKLNIET NATTLLLENT TFVTFKASRH SVSPDLKYVL LAYDVKQIFH
              160 170 180 190 200
YSYTASYVIY NIHTREVWEL NPPEVEDSVL QYAAWGVQGQ QLIYIFENNI
              210 220 230 240 250
YYQPDIKSSS LRLTSSGKEE IIFNGIADWL YEEELLHSHI AHWWSPDGER
              260 270 280 290 300
LAFLMINDSL VPTMVIPRFT GALYPKGKQY PYPKAGQVNP TIKLYVVNLY
              310 320 330 340 350
GPTHTLELMP PDSFKSREYY ITMVKWVSNT KTVVRWLNRA QNISILTVCE
              360 370 380 390 400
TTTGACSKKY EMTSDTWLSQ QNEEPVFSRD GSKFFMTVPV KQGGRGEFHH
              410 420 430 440 450
VAMFLIQSKS EQITVRHLTS GNWEVIKILA YDETTQKIYF LSTESSPRGR
              460 470 480 490 500
QLYSASTEGL LNRQCISCNF MKEQCTYFDA SFSPMNQHFL LFCEGPRVPV
              510 520 530 540 550
VSLHSTDNPA KYFILESNSM LKEAILKKKI GKPEIKILHI DDYELPLQLS
              560 570 580 590 600
LPKDFMDRNQ YALLLIMDEE PGGQLVTDKF HIDWDSVLID MDNVIVARFD
              610 620 630 640 650
GRGSGFQGLK ILQEIHRRLG SVEVKDQITA VKFLLKLPYI DSKRLSIFGK
              660 670 680 690 700
GYGGYIASMI LKSDEKLFKC GSVVAPITDL KLYASAFSER YLGMPSKEES
              710 720 730 740 750
TYQAASVLHN VHGLKEENIL IIHGTADTKV HFQHSAELIK HLIKAGVNYT
              760 770 780 790
MQVYPDEGHN VSEKSKYHLY STILKFFSDC LKEEISVLPQ EPEEDE
```

A cDNA encoding the foregoing human DPP10 protein has the following nucleotide sequence (NCBI accession no. NM_020868.6; SEQ ID NO:12).

| | |
|---|---|
| 1 | AGAAGCAGCA GAAGCAACAG CAGTAGCAGC GGCAGCAGCA |
| 41 | ACAGCAGCAG CCCCTACTGA AGTCCAATAG AGGAGACTTG |
| 81 | ATCTCTAGTT CATTCTGGAA CTCCGCCTGG GATTGTGCAC |
| 121 | TGTCCAGGGT CCTGAAACAT GAACCAAACT GCCAGCGTGT |
| 161 | CCCATCACAT CAAGTGTCAA CCCTCAAAAA CAATCAAGGA |
| 201 | ACTGGGAAGT AACAGCCCTC CACAGAGAAA CTGGAAGGGA |
| 241 | ATTGCTATTG CTCTGCTGGT GATTTTAGTT GTATGCTCAC |
| 281 | TCATCACTAT GTCAGTCATC CTCTTAACCC CAGATGAACT |
| 321 | CACAAATTCG TCAGAAACCA GATTGTCTTT GGAAGACCTC |
| 361 | TTTAGGAAAG ACTTTGTGCT TCACGATCCA GAGGCTCGGT |
| 401 | GGATCAATGA TACAGATGTG GTGTATAAAA GCGAGAATGG |
| 441 | ACATGTCATT AAACTGAATA TAGAAACAAA TGCTACCACA |
| 481 | TTATTATTGG AAAACACAAC TTTTGTAACC TTCAAAGCAT |
| 521 | CAAGACATTC AGTTTCACCA GATTTAAAAT ATGTCCTTCT |
| 561 | GGCATATGAT GTCAAACAGA TTTTTCATTA TTCGTATACT |
| 601 | GCTTCATATG TGATTTACAA CATACACACT AGGGAAGTTT |
| 641 | GGGAGTTAAA TCCTCCAGAA GTAGAGGACT CCGTCTTGCA |
| 681 | GTACGCGGCC TGGGGTGTCC AAGGGCAGCA GCTGATTTAT |
| 721 | ATTTTTGAAA ATAATATCTA CTATCAACCT GATATAAAGA |
| 761 | GCAGTTCATT GCGACTGACA TCTTCTGGAA AAGAAGAAAT |
| 801 | AATTTTTAAT GGGATTGCTG ACTGGTTATA TGAAGAGGAA |
| 841 | CTCCTGCATT CTCACATCGC CCACTGGTGG TCACCAGATG |
| 881 | GAGAAAGACT TGCCTTCCTG ATGATAAATG ACTCTTTGGT |
| 921 | ACCCACCATG GTTATCCCTC GGTTTACTGG AGCGTTGTAT |
| 961 | CCCAAAGGAA AGCAGTATCC GTATCCTAAG GCAGGTCAAG |
| 1001 | TGAACCCAAC AATAAAATTA TATGTTGTAA ACCTGTATGG |
| 1041 | ACCAACTCAC ACTTTGGAGC TCATGCCACC TGACAGCTTT |
| 1081 | AAATCAAGAG AATACTATAT CACTATGGTT AAATGGGTAA |
| 1121 | GCAATACCAA GACTGTGGTA AGATGGTTAA ACCGAGCTCA |
| 1161 | GAACATCTCC ATCCTCACAG TCTGTGAGAC CACTACAGGT |
| 1201 | GCTTGTAGTA AAAAATATGA GATGACATCA GATACGTGGC |
| 1241 | TCTCTCAGCA GAATGAGGAG CCCGTGTTTT CTAGAGACGG |
| 1281 | CAGCAAATTC TTTATGACAG TGCCTGTTAA GCAAGGGGGA |
| 1321 | CGTGGAGAAT TTCACCACGT AGCTATGTTC CTCATCCAGA |
| 1361 | GTAAAAGTGA GCAAATTACC GTGCGGCATC TGACATCAGG |
| 1401 | AAACTGGGAA GTGATAAAGA TCTTGGCATA CGATGAAACT |
| 1441 | ACTCAAAAAA TTTACTTTCT GAGCACTGAA TCTTCTCCCA |
| 1481 | GAGGAAGGCA GCTGTACAGT GCTTCTACTG AAGGATTATT |
| 1521 | GAATCGCCAA TGCATTTCAT GTAATTTCAT GAAAGAACAA |
| 1561 | TGTACATATT TTGATGCCAG TTTTAGTCCC ATGAATCAAC |
| 1601 | ATTTCTTATT ATTCTGTGAA GGTCCAAGGG TCCCAGTGGT |
| 1641 | CAGCCTACAT AGTACGGACA ACCCAGCAAA ATATTTTATA |
| 1681 | TTGGAAAGCA ATTCTATGCT GAAGGAAGCT ATCCTGAAGA |
| 1721 | AGAAGATAGG AAAGCCAGAA ATTAAAATCC TTCATATTGA |
| 1761 | CGACTATGAA CTTCCTTTAC AGTIGTCCCT TCCCAAAGAT |
| 1801 | TTTATGGACC GAAACCAGTA TGCTCTTCTG TTAATAATGG |
| 1841 | ATGAAGAACC AGGAGGCCAG CTGGTTACAG ATAAGTTCCA |
| 1881 | TATTGACTGG GATTCCGTAC TCATTGACAT GGATAATGTC |
| 1921 | ATTGTAGCAA GATTTGATGG CAGAGGAAGT GGATTCCAGG |
| 1961 | GTCTGAAAAT TTTGCAGGAG ATTCATCGAA GATTAGGTTC |
| 2001 | AGTAGAAGTA AAGGACCAAA TAACAGCTGT GAAATTTTTG |
| 2041 | CTGAAACTGC CTTACATTGA CTCCAAAAGA TTAAGCATTT |
| 2081 | TTGGAAAGGG TTATGGTGGC TATATTGCAT CAATGATCTT |
| 2121 | AAAATCAGAT GAAAAGCTTT TTAAATGTGG ATCCGTGGTT |
| 2161 | GCACCTATCA CAGACTTGAA ATTGTATGCC TCAGCTTTCT |
| 2201 | CTGAAAGATA CCTTGGGATG CCATCTAAGG AAGAAAGCAC |
| 2241 | TTACCAGGCA GCCAGTGTGC TACATAATGT TCATGGCTTG |
| 2281 | AAAGAAGAAA ATATATTAAT AATTCATGGA ACTGCTGACA |
| 2321 | CAAAAGTTCA TTTCCAACAC TCAGCAGAAT TAATCAAGCA |
| 2361 | CCTAATAAAA GCTGGAGTGA ATTATACTAT GCAGGTCTAC |
| 2401 | CCAGATGAAG GTCATAACGT ATCTGAGAAG AGCAAGTATC |
| 2441 | ATCTCTACAG CACAATCCTC AAATTCTTCA GTGATTGTTT |
| 2481 | GAAGGAAGAA ATATCTGTGC TACCACAGGA ACCAGAAGAA |
| 2521 | GATGAATAAT GGACTGTATT TATACAGAAC TGAAGGGAAT |
| 2561 | ATTGAGGCTC AATGAAACCT GACAAAGAGA CTGTAATATT |
| 2601 | GTAGTTGCTC CAGAATGTCA AGGGCAGCTT ACGGAGATGT |
| 2641 | CACTGGAGCA GCACGCTCAG AGACAGTGAA CTAGCATTTG |
| 2681 | AATACACAAG TCCAAGTCTA CTGTGTTGCT AGGGGTGCAG |
| 2721 | AACCCGTTTC TTTGTATGAG AGAGGTCAAA GGGTTGGTTT |
| 2761 | CCTGGGAGAA ATTAGTTTTG CATTAAAGTA GGAGTAGTGC |
| 2801 | ATGTTTTCTT CTGTTATCCC CCTGTTTGTT CTGTAACTAG |
| 2841 | TTGCTCTCAT TTTAATTTCA CTGGCCACCA TCATCTTTGC |
| 2881 | ATATAATGCA CAATCTATCA TCTGTCCTAC AGTCCCTGAT |
| 2921 | CTTTCATGGC TGAGCTGCAA TCTAACACTT TACTGTACCT |
| 2961 | TTATAATAAG TGCAATTCTT TCATTGTCTA TTATTATGCT |
| 3001 | TAAGAAAATA TTCAGTTAAT AAAAAACAGA GTATTTTATG |
| 3041 | TAATTTCTGT TTTTAAAAAG ACATTATTAA ATGGGTCAAA |
| 3081 | GGACATATAG AAATGTGGAT TTCAGCACCT TCCAAAGTTC |
| 3121 | AGCCAGTTAT CAGTAGATAC AATATCTTTA AATGAACACA |
| 3161 | CGAGTGTATG TCTCACAATA TATATACACA AGTGTGCATA |
| 3201 | TACAGTTAAT GAAACTATCT TTAAATGTTA TTCATGCTAT |
| 3241 | AAAGAGTAAA CGTTTGATGA ATTAGAAGAG ATGCTCTTTT |
| 3281 | CCAAGCTATA ATGGATGCTT TGTTTAATGA GCCAAATATG |
| 3321 | ATGAAACATT TTTTCCAATT CAAATTCTAG CTATTGCTTT |
| 3361 | CCTATAAATG TTTGGGTTGT GTTTGGTATT GTTTTTAGTG |
| 3401 | GTTAATAGTT TTCCAGTTGC ATTTAATTTT TTGAATATGA |
| 3441 | TACCTTGTCA CATGTAAATT AGATACTTAA ATATTAAATT |
| 3481 | ATAGTTTCTG ATAAAGAAAT TTTGTTAACA ATGCAATGCC |
| 3521 | ACTGAGTGCT ATTTTGCTCT TTTGGTGGAG AAGGCTTTTT |
| 3561 | TCAAAACTCT TGGTCCTTTT ACTTCTTTCT CTCAGTGCAG |
| 3601 | AATCAATTCT CATTTTCATC GTAAAAGCAA ATAGCTGGAT |
| 3641 | TATTTCATTT GCCAGTTTCT ATTTAGTATT CCATGCCTGC |
| 3681 | CCAATTCATC TGTTACTGTT TAATTTCAAT TCTTCTGGTG |
| 3721 | AGAATTAGAA ATGAAATATT TTTTATTCAT TGGCCAAAAA |
| 3761 | GTTCACAGAC AGCAGTGTTT GCTATTTACT TTGAATTGAA |
| 3801 | GGCACAAAAT GCATCAATTC CTGTGCTGTG TTGACTTGCA |
| 3841 | GTAGTAAGTA ACTGAGAGCA TAAAATAAAC CTGACTGTAT |
| 3881 | GAAGTCAATT TAAGTGATGA GAACATTTAA CTTTGGTGAC |
| 3921 | TAAAGTCAGA ATATCTTCTC ACTTCACTTA AGGGATCTTC |
| 3961 | CAGAAGATAT CTAAAAGTCT GTAATAAGCT TAGAAGTTCA |
| 4001 | GATAAATCTA GGCAGGATAC TGCATTTTTG TGGTTTTAAA |
| 4041 | AAAGTCCTTA GGACAGACTG AATTATCATA ACTTATGGCA |
| 4081 | TCAGGAGGAA ACTTTAAAAT ATCAAGGAAT CACTCAGTCA |
| 4121 | CCCTCCTGTT TTGTTGAAGG ATCAACCCCA AATTCTGGGT |
| 4161 | ATTTGAGTAC ATGTGAATCA TGGATTTGGT ATTCAACTTT |
| 4201 | TTCCCTGGAT GCTTTGGAAT CGTGTCTTCC ATGCTCCACT |
| 4241 | GGGTTCAATT TAAAATAGGA GAGGCTTTCT CTTCTGAAAG |
| 4281 | ATCCATTTTA GGTCTTTTTC AAGAATAGTG AACACATTTT |
| 4321 | TTAACAAAAT AAGTTGTAAT TTTAAAAGGA AAGTTTTGCC |
| 4361 | TATTTTATTA AGATGGAAAT TTCTTTTTAG GCTAATTTGA |
| 4401 | AATCCAACTG AAGCTTTTTA ACCAATATTT TAAATTTGAA |
| 4441 | CCACTAGAGT TTTTTATGAT GCAAATGATT ATGTTGTCTG |
| 4481 | AAAGGTGTGG TTTTATTGAA TGTCTATTTG AGTATCATTT |
| 4521 | AAAAAGTATT TGCCTTTTAC TGTCATCATT TCTCTTGTTT |
| 4561 | TATTATTATT ATCAATGTTT ATCTATTTTT CAATTAATTT |
| 4601 | AATACAGTTT CTAATGTGAA AGACATTTTT CTGGAACCCG |
| 4641 | TTTTCCCCTT AAACACTAAA GAGACCTCAA GTGAAAGCAT |
| 4681 | ATTGCTTAGT AGGAAGGTAG AAAATGTTAA TCCCTGCGAT |
| 4721 | TCTTTGAGTT TTAATGACAG GGTCATTTTC AGTAAAGGAA |
| 4761 | ATGCTCACCA ACACATAGTC ACCAACTATT AAAGGAATCA |
| 4801 | TGTGATTGGA TTTTCCCCTG TATACATGTA CCCTTGGTCA |
| 4841 | TAATCCCACT ATTTCATACA TATTTATGCA TTGCTAGATT |
| 4881 | TTCCTAGGAC TCCAATAGCA TGCTTTCCAA GTGTTATTAT |
| 4921 | TCCCTTAATG TTAAAGAAAA AAATCAATAT ATTGAATTCT |
| 4961 | TTAATTAAAT AGGTAAGCAG TATTAAATCA TCCACAGAGA |
| 5001 | TAAACAATCT CTTGAAACTC TTTTGAAAGA GAAAAGTGTT |
| 5041 | TGTTCACTTT CATTGCCTTC ATTAAGATCT CCAAATACTC |
| 5081 | AGAGACTAAG CAGGCTATTC TCATCTCTTT GAGTTTCCTG |
| 5121 | ACAGAACTAA AGGAAGGAAT CACTCTTCAA AAGATGGATC |
| 5161 | TCACTTCACT CTGAAAATGT GCCTTTTTTT AATTGGGTGT |
| 5201 | TTAGCATAAA AATCACTATT GGGGATCTTA CAGATGTCTG |
| 5241 | TTGTAGATCT ATTACAATGA GGTGACTTAG ACATTGGGCT |
| 5281 | TTACCATATT AGAGAATTAC TTAAGCCATT GTCCTTCAAA |
| 5321 | TGTTGGGCTT GGAGACAGAA GTACCTTGGA CTGAATTGCT |
| 5361 | TTCAAGTCTC TAGAATCACT GACAGCACAT AGCTTCTATG |
| 5401 | AGGAGTTATA TATGAAATAC CACTTGTGTC CCTCCTCAAG |
| 5441 | TTCTCTGACC ACCTTCACAT CCTGCGGCCA CCAACACTGT |
| 5481 | ATGGATACAA TCTGGCCTCT CAGCTCTGCT CACTTTCACT |
| 5521 | TTCTGTTCTG GGGATTCTCT GATACTAATT TTGGCTACTT |
| 5561 | GGTAGAACCA AGAGACAAAT CGCTTACATG CTGCAAACCG |
| 5601 | ATGTGAAGGT GTTACTAACC CGCTAAAACA CAATTAATGA |
| 5641 | AAGACTGTAG CAGGTAACTG ATCTTTCCCT CAGATATTCT |
| 5681 | ACATGGTCCA TGGAAGACTC TGTGGGCCGC AGAAGCCCAT |
| 5721 | AGCTGCATAC CCTTCTATTT TCCTCTTCCC CATTTCACTC |
| 5761 | TTTTAAGCTC CCTACTCCTA TTCCCTATGA TCACTTTCCA |
| 5821 | AAATAAACGG CAGGTGTGCT AGCTGTTGTC TTAGACTCTG |
| 5861 | CTTTCTGGAA ACTATTTTTC CCCTCCACAA GAAATTATCA |
| 5881 | CTGTGAATAT TACAGGAGGA GAAATAAAAT AAGAATAAAT |
| 5921 | CAGTGGTTTA TTTTCAGGTT CGAATAAACT CTTTCTAATG |
| 5961 | AAAA |

Variants, isoforms, and homologs of any of the DPP10 sequences described here can also be relevant to the methods and compositions described herein. For example, such variants and homologs can have less than 100% sequence identity to any of the sequences described herein. The variants and homologs can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of sequences described herein.

As illustrated herein, increased levels of DPP10 are indicative of and can lead to increased levels of APOE4. Hence, increased DPP10 levels in samples from a subject indicate that the subject would benefit from treatment with at least one HMGB1 inhibitor, at least one DPP10 inhibitor, or a combination of one or more HMGB1 inhibitors and one or more DPP10 inhibitors. For example, subjects may benefit from treatment with such HMGB1 inhibitors and/or DPP10 inhibitors when samples from the subject has DPP10 levels that are increased by at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 1.1 fold, at least 1.2 fold, at least 1.25 fold, at least 1.5 fold, at least 2 fold, at least 2.5 fold, or at least 3 fold.

DPP10 can be detected using antibodies that specifically bind to DPP10 protein, and/or probes/primers that bind specifically to DPP10 mRNA. Antibodies that bind to DPP10 protein are available, for example from Alomone Labs, Invitrogen, SigmaAldrich, and ThermoFisher Scientific. Probes and primers that bind specifically to DPP10 mRNA can include segments of about 15-100 nucleotides that have at least 90% sequence identity or complementarity to a DPP10 coding region (e.g., to a DPP10 cDNA with the SEQ ID NO: 12 sequence).

DPP10 inhibitors include any compounds or molecules that are capable of inhibiting the expression, function, or extracellular release of DPP10. Such DPP10 inhibitors can include chemical compounds, antibodies, proteins, small RNA molecules, small DNA molecules, and the like that target DPP10 proteins or DPP10 nucleic acids.

### Compositions

The invention also relates to compositions containing one or more active agents such as any of the HMGB1 inhibitor compounds described herein. Such active agents can include a polypeptide, a nucleic acid encoding a polypeptide (e.g., within an expression cassette or expression vector), a modified cell, an inhibitory nucleic acid, a small molecule, a compound identified by a method described herein, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form.

In some embodiments, the active agents are administered in a “therapeutically effective amount.” Such a therapeutically effective amount is an amount sufficient to obtain the desired physiological effect, such a reduction of at least one symptom of a tauopathy disease. Such symptoms can include HMGB1 nucleo-cytosplasmic translocation, gliosis, neurodegeneration, Tau pathologies, and myelin deficits.

For example, active agents can reduce the symptoms of HMGB1 nucleo-cytosplasmic translocation, gliosis, neurodegeneration, Tau pathologies, or myelin deficits by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules or compounds chosen for administration, the disease, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the composition, the small molecules or compounds are synthesized or otherwise obtained and purified as necessary or desired. These small molecules or compounds, and other agents can be suspended in a pharmaceutically acceptable carrier and/or lyophilized or otherwise stabilized. These active agents can be adjusted to an appropriate concentration, and optionally combined with other agents. The absolute weight of a given small molecule or compound, and/or other agents included in a unit dose can vary widely. For example, about 0.01 to about 2 g, or about 0.1 to about 500 mg, of at least one molecule, compound, and/or other agent, or a plurality of molecules, compounds, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the nature of the disease or condition being treated, and the age and condition of the subject. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the active agent(s) and/or other agents can sometimes be administered in an oral dosage form, that oral dosage form can be formulated so as to protect the small molecules, compounds, polypeptides, other agents, and combinations thereof from degradation or breakdown before the small molecules, compounds, other agents, and combinations thereof provide therapeutic utility. For example, in some cases the small molecules, compounds, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as active agents, anti-viral agents, antibacterial agents, antimicrobial agents and/or preservatives.

The present description is further illustrated by the following examples, which should not be construed as limiting in any way.

### Example 1: Materials and Methods

This Example illustrates the materials and methods employed in the development of the invention.

### Mice

Human LoxP-floxed APOE knock-in (fE) mice with conditional deletion of the human APOE gene were generated as described by Knoferle et al. (J. Neurosci. 34, 14069-14078 (2014)). Briefly, homozygous fE3 and fE4 mice (Wang et al. Neuron 109, 1657-1674.e7 (2021) were crossbred with Synapsin 1-Cre transgenic mice [B6.Cg-Tg(Syn1-Cre)671Jxm/J](The Jackson Laboratory) (Zhu et al., Genes Dev. 15, 859-876 (2001)) or GFAP-Cre transgenic mice [B6.Cg-Tg(GFAP-Cre)8Gtm](National Cancer Institute Mouse Repository) (Bajenaru et al. Mol. Cell Biol. 22, 5100-5113 (2002); Uhlmann et al., Ann. Neurol. 52, 285-296 (2002)). The fE/Cre mice were further crossbred with Tau-P301S (PS19) transgenic mice [B6;C3-Tg(Prnp-MAPT*P301S)PS19Vle/J](The Jackson Laboratory) expressing human P301S 1N4R Tau driven by the PrP promoter to generate PS19-fE4 and PS19-fE3 mice with no Cre, Syn1-Cre, or GFAP-Cre. Littermates that were negative for Syn1-Cre or GFAP-Cre were used as PS19-fE controls. For generation of the PS19-fE/Syn1-Cre line, only female Syn1-Cre mice were used for breeding purposes because germline recombination has been reported to occur in the progeny of male Syn1-Cre mice (Rempe et al., Genesis 44, 44-49 (2006)). Wildtype (WT) mice [C57BL/6J] were obtained from the Jackson Laboratory. All mice were on a pure C57BL/6 genetic background and were housed in a pathogen-free barrier facility on a 12 hour light cycle at 19-23° C. and 30-70% humidity. Animals were identified by ear punch under brief isoflurane anesthesia and genotyped by polymerase chain reaction (PCR) of a tail clipping. All animals otherwise received no procedures except those reported in this study. All animal experiments were conducted in accordance with the guidelines and regulation of the National Institutes of Health, the University of California, and the Gladstone Institutes under the protocol AN176773.

For brain tissue collections, mice were deeply anesthetized with intraperitoneal injections of avertin (Henry Schein) and transcardially perfused for 1 min with 0.9% saline. Brains were either fixed as whole brains or hemi-brains, depending on the study. Right hemi-brains were drop-fixed for 48 h in 4% paraformaldehyde (16% PFA diluted in MilliQ H₂O) (Electron Microscopy Sciences), rinsed in 1×PBS (Corning) for 24 h, and cryoprotected in 30% sucrose (Sigma) for 48 h at 4° C. The fixed hemi-brains were cut into 30 μm thick coronal sections on a freeze sliding microtome (Leica) and stored in cryoprotectant solution (30% Ethylene Glycol, 30% Glycerol, 40% 1×PBS) at −20° C. Left hemi-brains were snap frozen on dry ice and stored at −80° C.

### Immunohistochemistry

For immunofluorescent staining, several sections from each mouse (˜300 μm apart) were transferred to a 12-well plate in 1×PBS-T (PBS+0.1% Tween-20) (Millipore Sigma) and were washed 3×5 min in PBS-T to remove cryoprotectant solution. Sections were incubated for 5 min in boiling antigen retrieval buffer (Tris buffer, pH 7.6) (TEKNOVA) and washed 2×5 min in PBS-T. Sections were then incubated in blocking solution (5% normal donkey serum (Jackson Labs), 0.2% Triton-X (Millipore Sigma) in 1×PBS) for 1 h at room temperature to prevent non-specific antibody binding. After blocking, sections were washed 1×5 min in PBS-T and incubated in Mouse-on-Mouse (M.O.M.) Blocking Buffer (1 drop M.O.M IgG/4 mL PBS-T) (Vector Labs) for 1 h at room temperature. After M.O.M. block, sections were incubated in primary antibody at 4° C. overnight after being diluted to optimal concentrations (anti-APOE 1:200 (Cell Signaling); anti-CD68 1:100 (Bio-Rad); anti-Cre 1:800 (Cell Signaling); anti-GFAP 1:800 (Millipore Sigma); anti-GFP 1:5000 (Thermofischer); anti-HMGB1 1:100 (Abcam); anti-Iba1 (rbt) 1:200 (Wako); anti-Iba1 (gt) 1:200 (Abcam); anti-MBP 1:500 (Abcam); anti-NeuN 1:500 (Millipore Sigma); anti-NG2 1:500 (Abcam); anti-S100β 1:200 (Abcam)). Following primary antibody incubation, sections were washed 3×5 min in PBS-T and then incubated in fluorescence-labeled secondary antibodies (Abcam, Jackson Immuno, 1:1000 in PBS-T) for 1 hour at room temperature protected from light after being diluted in PBS-T. Sections were then washed 2×5 minutes in PBS-T and incubated in DAPI (1:50,000 in PBS-T) (Thermofisher) for 8 minutes at room temperature protected from light. Sections were then washed 2×5 minutes in PBS-T, mounted onto microscope slides (Fisher Scientific), cover-slipped with ProLong Gold mounting media (Vector Laboratories), and sealed with clear nail polish. Images were taken using an FV3000 confocal laser scanning microscope (Olympus) or Aperio VERSA slide scanning microscope (Leica) at 10×, 20×, 40×, or 60× magnifications depending on the stain. Image analyses of percent coverage area were performed using the open-source Fiji (ImageJ) software after setting a standard threshold value that is applied to all images. Researchers were also blinded to samples to exclude the possibility of bias.

For DAB (3,3′-diaminobenzidine) staining, several sections from each mouse (˜300 μm apart) were transferred to a 12-well plate in 1×PBS-T and then washed 3×5 min in PBS-T to remove cryoprotectant solution. Sections were then incubated for 5 min in boiling antigen retrieval buffer (1×PBS, 0.1M sodium citrate, 0.1M citric acid) (Fisher Scientific, Fluka) and washed 2×5 min in PBS-T. Next, sections were incubated for 15 min in endogenous peroxidase buffer (1×PBS, 10% methanol (Fisher Scientific), 3% H₂O₂ (Sigma) and washed 3×5 min in PBS-T. Sections were then incubated in blocking solution (1×PBS-T, 5% normal donkey serum, 1% non-fat dry milk) for 1 hour at room temperature. After blocking, sections were washed 2×5 min in PBS-T and then incubated in Avidin/Biotin blockage (4 drops of each block) (Vector Laboratories) for 15 minutes and then washed 2×5 minutes in PBS-T. Sections were incubated in M.O.M. Blocking Buffer (1 drop M.O.M IgG/4 mL PBS-T) (Vector Labs) for 1 hour at room temperature. Following M.O.M. block, sections were washed 2×5 minutes and incubated in primary antibody at 4° C. overnight after being diluted in PBS-T to optimal concentrations (anti-pTau (AT8) 1:100 (Invitrogen); anti-HT7 1:200 (Peter Davies)). After primary antibody incubation, sections were washed 3×5 minutes in PBS-T and then incubated in biotinylated secondary antibody (1:200; Jackson Immuno) at room temperature for 1 hour. Next, sections were washed 3×5 minutes in PBS-T and incubated in ABC buffer (Vector Laboratories) that was prepared 10 minutes prior to the incubation step. Sections were washed for 2×5 minutes in PBS-T and 1×5 minutes in Tris buffer (pH 7.6). Sections were incubated in DAB buffer (5 mL 1×PBS, 2 drops Buffer Stock Solution, 2 drops DAB, 2 drops H₂O₂) (Vector Laboratories) for precisely 2 minutes. Staining was halted by washing sections 3×5 minutes in Tris buffer (pH 7.6) and 2×5 minutes in PBS-T. Sections were mounted onto microscope slides and dried at room temperature overnight. Next, mounted sections were submerged into Xylene (Fisher Scientific) 2×5 minutes and cover-slipped with DPX mounting media (Sigma-Aldrich). Images were taken using an Aperio VERSA slide scanning microscope (Leica) at 10× magnification.

### Volumetric Analysis.

Serial coronal hippocampal brain sections (7 sections per mouse, 30 μm thick, 300 μm apart) were mounted onto microscope slides (Fisher Scientific) and dried at room temperature for 1 hour. The 0.1% Sudan Black solution was prepared by adding the appropriate amount of Sudan Black powder (Sigma) to 70% ethanol (KOPTEC) and mixing the solution using a magnetic stirrer while and protected from light. The solution was then centrifuged at 3,000 RPM for 10 minutes and the collected supernatant was filtered using a 0.2 μm filter syringe (Thermo Scientific) to remove undissolved dye. Sections were then stained with the 0.1% Sudan Black solution at room temperature for 10 minutes and washed 3×2 minutes in 70% ethanol and 3×5 minutes in Milli-Q water. Sections were then cover-slipped with ProLong Gold mounting media (Invitrogen) and imaged on an Aperio VERSA slide scanning microscope (Leica) at 10× magnification. For hippocampal and posterior lateral ventricle volumetric analyses, the areas of interest were traced in ImageJ using the segmented line tool and the volume was calculated using the formula: volume=(sum of area)*0.3 mm (Uhlmann et al. Ann. Neurol. 52, 285-296 (2002)). The sum of area value was obtained by taking a sum of the quantified area measurements of all 7 brain sections per mouse, roughly between coordinates AP=−1.2 and AP=−3.4.

### Neuronal Layer Thickness Measurements

Two brain sections (30 μm thick, 300 μm apart) underwent immunofluorescence staining as described above using the primary antibody NeuN (1:500) to visualize the neuronal cell layers of the hippocampus. Sections were imaged at 20× magnification using an FV3000 confocal laser scanning microscope (Olympus). The thickness of the CA1 pyramidal cell layer and dentate gyrus granular cell layer of the hippocampus were measured on the Fiji (ImageJ) software by drawing a straight line perpendicular to the NeuN+ cell layers at two points per hippocampal subfield and taking the average value for each mouse.

### Nuclear-Cytoplasmic Localization of HMGB1 Measurements

Two brain sections (30 μm thick, 300 μm apart) were immunostained with anti-HMGB1 (1:100) and DAPI (1:50,000) as described above. Sections were imaged at 40× and 60× magnification using an FV3000 confocal laser scanning microscope (Olympus). All image processing and quantification was performed on the Fiji (ImageJ) software. Briefly, a 1-pixel median filter was applied to the DAPI channel and an appropriate threshold was set to create a mask of DAPI. The image calculator function was then used to overlay the DAPI mask and HMGB1 channel, which provided the HMGB1 staining that was only localized to the nucleus. After obtaining values for integrated density and particles, the image calculator was used to subtract the DAPI mask from HMGB1, which provided HMGB1 staining that was excluded from the nucleus.

### Biochemical Extraction of Brain Tissue

The hippocampus was dissected from snap frozen mouse hemi-brains after thawing on ice. The hippocampal tissue was weighed and homogenized using a Polytron immersion disperser Polytron homogenizer (Kinematica AG) in ice-cold RAB buffer (G Biosciences) at 10 μL/mg tissue, supplemented by phosphatase inhibitors (Roche) and protease inhibitors (Roche). Samples were then centrifuged using an Optima TLX ultracentrifuge (Beckman Coulter) at 50,000 g for 20 minutes at 4° C. and the supernatant was collected as the RAB-soluble fraction. The pellets were resuspended in ice-cold RIPA buffer (Thermo Scientific) at 10 μL/mg tissue and centrifuged at 50,000 g for 20 minutes at 4° C. The supernatant was collected as the RIPA-soluble fraction and the pellet was stored at −80° C. for further use. All fractions were stored at −80° C. until further analyses.

### Western Blot Analysis

Biochemically extracted mouse hippocampal tissue lysates were loaded onto 12% Bis-Tris SDS-PAGE gels (Invitrogen) and separated by gel electrophoresis at 160V using MOPS buffer. The separated proteins were transferred onto nitrocellulose membranes at 18V for 60 minutes (Trans-Blot Turbo Transfer System (Bio-rad). Membranes were washed 3×5 minutes in PBS-T and then incubated in Intercept blocking buffer (LI-COR) for 1 hour at room temperature to block non-specific binding sites. After blocking, membranes were washed 3×5 minutes in PBS-T and incubated with primary antibody overnight at 4° C. (AT8 1:3,000 (Invitrogen), TUJ1 1:15,000 (Biolegend)). Membranes were washed 3×5 minutes in PBS-T and incubated in fluorescently-labeled secondary antibody (1:20,000; LI-COR) for 1 hour in the dark at room temperature. Resulting bands were detected with the Odyssey CLx infrared imaging system (LI-COR), and the fluorescence intensity of the bands was quantified as a ratio of AT8:TUJ1 signal using the Image Studio software.

### Sandwich ELISA

Biochemically extracted mouse hippocampal tissue lysates were diluted in Milli-Q H₂O to the appropriate concentration and were run according to the provided manufacturer protocols (human APOE (Abcam); mouse HMGB1 (Novus Biologicals). Reactions of samples were read on a SpectraMaX M5 spectrophotometer (Molecular Devices) and protein concentrations were determined after interpolating a standard curve and adjusting for dilutions.

### Primary Neuron Cultures and Recombinant Protein Treatment

Primary cultures of neurons were prepared from prenatal E20 pups of various genotypes. After collecting pup brains, the cortex plus hippocampus were isolated and placed in ice-cold dissociation media+kyneurenic acid media (DM/KY) (DM:Na₂SO₄ (81.8 mM); K₂SO₄ (30 mM); MgCl₂ (5.8 mM), CaCl₂) (0.25 mM); HEPES (1 mM); glucose (20 mM); phenol red (0.001%); NaOH (0.16 mM) in distilled H₂O) (KY: kynurenic acid (10 mM); phenol red (0.0025%); HEPES (5 mM); MgCl₂ (100 mM); NaOH (add dropwise until pH 7.4). The resulting DM/KY media was made by combining 90% DM with 10% KY media. The isolated tissue was finely minced and then submerged in pre-warmed Papain solution (1 mL per brain) for 13 minutes while gently inverting and then submerged in trypsin inhibitor solution for 5 minutes (5 mL for up to 10 brains) while gently inverting. The tissue pellet was washed with Optimem/Glucose solution (20 mM glucose, 1 mL per brain) while gently inverting. Then, fresh Optimem/Glucose solution was added and tissue was gently triturated until separated into single cells. After filtering the cells through a 40 um cell strainer, the dissociated cells were plated at 1×10⁶ cells/well of a 12-well plate or 3×10⁵ cells/well of a 24-well plate in Neurobasal medium supplemented with B27, 100 U/mL⁻¹ of penicillin G, 100 g/mL⁻¹ of streptomycin, and 1% GlutaMAX (B27/Neurobasal). Every 3-4 days, half of the media was removed and replaced with fresh B27/Neurobasal media. In some experiments, primary neurons were treated with either a Dulbecco's PBS (dPBS) vehicle or a recombinant protein of interest (10 μg/mL for one well of a 12-well plate) for 24 hours at day 14 in vitro. Following treatment, the media was collected and the cultures were harvested for analysis. Total protein levels present in cell lysates were obtained thru BCA analysis (Pierce).

### Stereotaxic Surgery on Mice

Mice were anesthetized with an intraperitoneal injection of ketamine (60 mg/kg) and xylazine (30 mg/kg) and maintained on 0.8%-1.0% isofluorane (Henry Schein). Mice were secured in a stereotaxic alignment system model 940 using earbars and a tooth bar (Kopf Instruments). The scalp was prepared by removing hair using Nair and sterilizing with 70% ethanol. The scalp was then cut open using a scalpel and sterilized with 70% ethanol. The cranial sutures were better visualized using 3% hydrogen peroxide. Following identification of Bregma, a unilateral stereotaxic site was drilled with a 0.5 mm microburr (Fine Science Tools) using coordinates X=+1.5, Y=−2.1, Z=−2.1, with Z measured from the surface of the brain. Mice were injected with 2 μL of the respective virus (AAV2(Y444F)-smCBA-human_P301S_Tau-WPRE, 2.10E+13 vg/mL, Virovek); AAV2-Synapsin-GFP, 1.0E+13 vg/mL, SignaGen) or interstitial fluid (ISF) fraction at a rate of 500 nL/min and allowed to diffuse for 3 min. Following surgery, mice were sutured with nylon monofilament non-absorbable 6-0 sutures (Henry Schein) and administered analgesics buprenorphine (0.0375 mg/kg intraperitoneally), ketophen (5 mg/kg subcutaneously), and saline (500 μL intraperitoneally). Mice were monitored on a heating pad until ambulatory and provided Hydrogel for hydration.

### Brain Slices Electrophysiological Recordings and Data Analyses

For electrophysiological recording study, 8-month-old PS19-fE3 mice and PS19-fE4 mice with no Cre, Syn1-Cre, or GFAP-Cre were anaesthetized with isoflurane and decapitated. The brain was rapidly removed from the skull and placed in ice-cold (2-5° C.) slicing solution. Slicing solution contained (in mM): 110 choline chloride, 2.5 KCl, 26 NaHCO₃, 10 MgCl₂, 1.25 NaH₂PO₄, 0.5 CaCl₂), 10 glucose, 3 Na Pyruvate, 1 L-Ascorbic acid, pH 7.4. 350 μm-thick sagittal slices were cut from both hemispheres using a vibratome (VT1200, Leica) and transferred to a 95% O₂—CO₂ vapor interface holding chamber (BSK5, Scientific Systems Design) containing artificial cerebrospinal fluid (ACSF) where they were allowed to recover at 34° C. for one hour and held at room temperature (20-22° C.) afterwards. ACSF contained (in mM): 126 NaCl, 2.5 KCl, 1.5 CaCl₂), 1.5 MgCl₂, 26 NaHCO₃, 1.25 NaH₂PO₄, 10 glucose, and 1.5 L-Ascorbic acid, pH 7.4.

For input/output recording studies, local field post-synaptic potentials (fPSPs) were elicited by orthodromic stimulation of Schaffer collaterals by concentric bipolar stimulating electrode (FHC) connected to a constant voltage isolated stimulator (DS2A-MKII, Digitimer North America) and placed in CA2 stratum radiatum. fPSPs were recorded with a glass borosilicate microelectrode filled with ACSF and placed in CA1 stratum radiatum. Signals were sampled and digitized by MultiClamp 700B amplifier and Digidata 1550B1 acquisition system with pClamp10 software (Molecular Devices), and analyzed using IgorPro6 software (Wavemetrics) running custom macros. fPSP slopes were analyzed as the linear fit slope values between 10% and 90% of fPSP peak. Input-output relationships were recorded as the fPSP slope values in response to increasing stimulation intensity (20-60 pA), with fPSP slope gain calculated as the linear slope of the resulting input-output curve.

### Microdialysis of Mouse Hippocampus

Brain interstitial fluid was collected using in vivo microdialysis of the hippocampus. Surgical procedures, including pre- and post-operative care, were conducted as described above for stereotaxic surgeries. During the surgery, a unilateral stereotaxic site was drilled with a 1.2 mm bone drill bit (BASi) and an AtmosLM guide cannula PEG-4 (Amuza) was stereotaxically implanted above the right hippocampus at coordinates X=+1.5, Y=−2.1, Z=−1.1. The cannula was secured in place using dental cement (GC America), and a temporary PEG-4 AtmosLM dummy probe (Amuza) was inserted and fixed with an AC-5 cap nut screw (Eicom). Two days post-surgery, mice were placed in a microdialysis stand-alone system (BASi) overnight to assimilate, and the following afternoon a 1000 kDa AtmosLM collection probe (Eicom) was inserted through the guide cannula into the hippocampus, which extends 1 mm father down to Z=−2.1 to target the dentate gyrus. Artificial CSF (Harvard Apparatus) made with 0.15% BSA (Thermo Scientific) was circulated through the system at a rate of 0.5 μL/min using a push-pull method, and interstitial fluid (ISF) was collected in a refrigerated fraction collector (BASI) each hour for roughly 24 hours. To prevent clogging of the tubing, pumps were operated at 10× collection speed for the first two hours before being adjusted to a 0.5 μL/min flow rate. Following completion of ISF collection, mice were euthanized and perfused with 0.9% saline, as described above. The brain was dissected into hemispheres, with the right hemi-brain postfixed for 48 hours in 4% PFA and the left hemi-brain fresh frozen. Interstitial fluid (ISF) fractions were frozen at −80° C. for further analysis.

Treatment with HMGB1 Inhibitors

At 6.5 months of age, male and female PS19-fE4 and PS19-fE3 mice were randomly assigned to the control or treatment group. Mice received intraperitoneal injections with either sterile grade 0.9% saline (Fisher Scientific) or a mixture of HMGB1 inhibitors: ethyl pyruvate (80 mg/kg) (Sigma-Aldrich) and glycyrrhizic acid (20 mg/kg) (Sigma-Aldrich) dissolved in 0.9% saline. The mice received three injections per week for 12 weeks, starting at 6.5 months of age until they reached 9.5 months of age. All mice were monitored for weight changes, grooming changes, and posture during the experiments and no changes were observed. Following treatment, the animals were perfused and their brain tissue was processed for histopathological analysis, as described above.

### Single-Nuclei Preparation for 10× Loading

The mouse hippocampus was dissected on ice and placed into a pre-chilled 2 mL Dounce with 1 mL of cold 1× Homogenization Buffer (1×HB) (250 mM Sucrose, 25 mM KCL, 5 mM MgCl₂, 20 mM Tricine-KOH pH7.8, 1 mM DTT, 0.5 mM Sermidine, 0.15 mM Sermine, 0.3% NP40, 0.2 units/μL RNase inhibitor, 0.2 units/μL Protease inhibitor). Dounce with “A” loose pestle (˜10 strokes) and then with “B” tight pestle (A15 strokes). The homogenate was filtered using a 70 μM Flowmi strainer (Eppendorf) and transferred to a pre-chilled 2 mL LoBind tube (Fischer Scientific). Nuclei were pelleted by spinning for 5 min at 4° C. at 350 RCF. The supernatant was removed and the nuclei were resuspended in 400 μL 1×HB. Next, 400 μL of 50% Iodixanol solution was added to the nuclei and then slowly layered with 600 μL of 30% Iodixanol solution under the 25% mixture, then layered with 600 μL of 40% Iodixanol solution under the 30% mixture. The nuclei were then spun for 20 min at 4° C. at 3,000 g in a pre-chilled swinging bucket centrifuge. 200 μL of the nuclei band at the 30%-40% interface was collected and transferred to a fresh tube. Then, 800 μL of 2.5% BSA in PBS plus 0.2 units/μL of RNase inhibitor was added to the nuclei and then were spun for 10 minutes at 500 RCF at 4° C. The nuclei were resuspended with 2% BSA in PBS plus 0.2 units/μL RNase inhibitor to reach ˜500 nuclei/μL. The nuclei were then filtered with a 40 μM Flowmi stainer. The nuclei were counted and then ˜13,000 nuclei per sample were loaded onto 10× Genomics Next GEM chip G. The snRNA-seq libraries were prepared using the Chromium Next GEM Single Cell 3′ Library and Gel Bead kit v3.1 (10× Genomics) according to the manufacturer's instructions. Libraries were sequenced on an Illumina NovaSeq 6000 sequencer at the UCSF CAT Core.

### Custom Reference Genome

PS19 tau mutant floxed APOE knock-in mouse model (Bien-Ly et al., J. Neurosci. 32, 4803-4811 (2012)) was used for single-nucleus RNA-sequencing (snRNA-seq). The Homo sapiens microtubule associated protein tau (MAPT) (NCBI Reference Sequence: NM_001123066.4) (Agarwala et al. Nucleic Acids Res. 44, D7-D19 (2016)) and the Homo sapiens APOE are genes of interest for this study. These genes are not expected to be a part of the mouse reference genome, so to quantify the reads aligning to these genes of interest, a custom mouse reference genome was made using the reference mouse genome sequence (GRCm38) from Ensembl (release 98) (Howe et al. Ensembl 2021. Nucleic Acids Res. 49, D884-D891 (2021)) and the mouse gene annotation file from GENCODE (release M23) (Frankish et al. GENCODE 2021. Nucleic Acids Res. 49, D916-D923 (2021)), similar to those used in 10× Genomics Cell Ranger mouse reference package mm10 2020-A. The headers of the Ensembl reference mouse genome sequence fasta file with the chromosome names were modified to match the chromosome names in a fasta file from GENCODE. The annotation GTF file contains entries from non-polyA transcripts that overlap with the protein coding genes. These reads are flagged as multi-mapped and are not counted by the 10× Genomics Cell Ranger v6.1.1 count pipeline (Zheng et al. Nat. Commun. 8, 14049 (2017)). To avoid this, the GTF file was modified to (1) remove version suffixes from transcript, gene, and exon ids to match the Cell Ranger reference packages, (2) remove non-polyA transcripts. The Homo sapiens MAPT sequence and Homo sapiens APOE sequence were appended as separate chromosomes to the end of the mouse reference genome sequence and the corresponding gene annotations were appended to the filtered mouse reference gene annotation GTF file. The 10× Genomics Cell Ranger v6.1.1 mkref pipeline was used to build the custom reference genome using the modified fasta and GTF file.

Pre-Processing and Clustering of Mouse snRNA-Seq Samples

The snRNA-seq samples included a total of 16 samples with four mice from each of the four genotype groups (PS19-fE4, PS19-fE4 Syn1-Cre, PS19-fE4 GFAP-Cre, and PS19-fE3). Each group of four mice had two male and two female mice. The demultiplexed fastq files for these samples were aligned to the custom mouse reference genome (See custom reference genome methods for additional descriptions) using the 10× Genomics Cell Ranger v6.1.1 count pipeline (Zheng et al. Nat. Commun. 8, 14049 (2017)), as described in the Cell Ranger documentation. The include-introns flag for the count pipeline was set to true to count the reads mapping to intronic regions. The Cell Ranger count web summaries showed a “Low Fraction Reads in Cells” error for two samples—one from the PS19-fE4 GFAP-Cre group and one from the PS19-fE3 group. These two samples had only ˜40% reads assigned to cell-associated barcodes and <80% reads mapped to the genome. These metrics were much higher for other 14 samples. Checking the experimental record indicated that these two samples had issues at the nuclear isolation step and lower cDNA was recovered due to the use of an expired old batch of sample preparation reagents. All other 14 samples were prepared with a new batch of sample preparation reagents. So, these two samples were excluded and only the remaining 14 samples were used for the downstream analyses with Seurat.

The filtered count matrices generated by the Cell Ranger count pipeline for 14 samples were processed using the R package for single-nucleus analysis Seurat v4.0.5 (Hao et al. Cell 184, 3573-3587.e29 (2021)). Each sample was pre-processed as a Seurat object and the top 1% of cells per sample with a high number of unique genes, cells with <=200 unique genes, and cells >=0.25% mitochondrial genes were filtered out for each sample. The 14 samples were merged into a single Seurat object and normalization and variance stabilization was performed using sctransform (Hafemeister & Satija, Genome Biol. 20, 296 (2019)) with the “glmGamPoi” (Bioconductor package version 1.6.0) method (Ahlmann-Eltze & Huber, Bioinformatics 36, 5701-5702 (2021)) for initial parameter estimation.

Graph-based clustering was performed using the Seurat v4.0.5 functions FindNeighbors and FindClusters. First, the cells were embedded in a k-nearest neighbor (KNN) graph based on the Euclidean distance in the PCA space. The edge weights between two cells were further modified using Jaccard similarity. Next, clustering was performed using the Louvain algorithm implementation in the FindClusters Seurat function. Clustering was performed for all combinations of 10, 15 and 20 PCs with 0.4, 0.5, 0.6, 0.7, 0.8 and 0.9 resolutions. Clustering with 15 PCs and 0.7 resolution resulted in 33 distinct biologically relevant clusters, which was used for further analyses.

### Cell Type Assignment

Data visualization using Seurat v4.0.5 in the UMAP space for the 14 samples revealed no batch effects by age, sex, genotype, date of birth, or nuclear isolation date. The marker genes for each cluster were identified using the FindAllMarkers Seurat function on the SCT assay data. This algorithm uses the Wilcoxon Rank Sum test to iteratively identify differentially expressed genes in a cluster against all the other clusters. Marker genes were filtered to keep only positively expressed genes, detected in at least 25% of the cells in either population, and with at least 0.5 log 2 fold change. Identities were assigned to cell clusters by matching the cell clusters to known cell types with the expression of canonical cell-type-specific genes, the expression of genes identified in publicly available mouse hippocampal single-cell RNA-seq datasets, and the expression of each cluster's marker genes in a publicly available resource of brain-wide in situ hybridization images, as we reported previously (Zalocusky et al., Nat. Neurosci. 24, 786-798 (2021)).

### Subclustering of Astrocytic and Microglial Sn-RNA-Seq Data

The hippocampal cell cluster 10 was annotated as the astrocyte cells and hippocampal cell clusters 11, 21 and 29 were annotated as the microglial cells. Both these cell types were sub-clustered. Normalization and variance stabilization was performed using sctransform⁸² with the “glmGamPoi” (Bioconductor package version 1.6.0) method (Ahlmann-Eltze & Huber, Bioinformatics 36, 5701-5702 (2021)) for initial parameter estimation. Graph-based clustering was performed using the Seurat v4.0.5 functions FindNeighbors and FindClusters. First, the cells were embedded in a k-nearest neighbor (KNN) graph based on the Euclidean distance in the PCA space. The edge weights between two cells were further modified using Jaccard similarity. Next, clustering was performed using the Louvain algorithm implementation in the FindClusters Seurat function. Clustering was performed for all combinations of 10, 15, 20, 25 and 30 PCs with 0.4, 0.5, 0.6, 0.7, 0.8 and 0.9 resolutions. Sub-clustering with 15 PCs and 0.9 resolution resulted in 18 distinct biologically relevant subclusters for astrocytes. Sub-clustering with 15 PCs and 0.9 resolution resulted in 18 distinct biologically relevant microglia subclusters.

### Gene-Set Enrichment Analysis

Differentially expressed genes between clusters of interest were identified using FindMarkers Seurat function on the SCT assay data. This algorithm uses the Wilcoxon Rank Sum test to identify differentially expressed genes between two populations. Differentially expressed genes were limited to genes detected in at least 10% of the cells in either population and with at least 0.1 log 2 fold change. Over-representation (or enrichment) analysis was performed using clusterProfiler v4.2.1 (Wu et al. Innov. (N Y) 2, 100141 (2021)) to find gene sets in the KEGG database (Kanehisa et al. Nucleic Acids Res. 44, D457-D462 (2016)) for mouse associated with the differentially expressed genes. The p-values are based on a hypergeometric test and are adjusted for multiple testing using the Benjamini-Hochberg method (Benjamini et al., J R Stat. Soc. Ser. B Stat. Methodol. 57, 289-300 (1995)). Significantly enriched gene sets were filtered to have an adjusted p-value less than 0.8 and at least 10 differentially expressed genes present in the gene set. The same method was used for gene-set enrichment analysis of astrocyte subclusters and microglia subclusters.

### Association Between Clusters and Genotype

A Generalized Linear Mixed-Effects Model to assess association with Animal Models (GLMM_AM) was implemented in the lme4 (v1.1-27.1) R package (Bates et al., J. Stat. Soft. 67, 1-48 (2015)) and used to estimate the associations between cluster membership and the mouse model. These models were run separately for each cluster of cells. The GLM model was performed with the family argument set to the binomial probability distribution and with the bobyqa control optimizer used for the maximum likelihood estimation. Cluster membership for each cell was modeled as a 0-1 response variable according to whether or not the cell belongs to the cluster under consideration. The corresponding mouse id from which the cell was derived was the random effect variable and the animal model for this mouse id was included as the fixed variable. The reference animal model was set to PS19 fE4. The resulting p-values for the estimated log odds ratio across the three animal models (with respect to the PS19 fE4) and clusters were adjusted for multiple testing using the Benjamini-Hochberg method (Benjamini et al., J R Stat. Soc. Ser. B Stat. Methodol. 57, 289-300 (1995)). The same method was used for estimating the between cluster association with genotype for astrocyte subclusters and microglia subclusters.

### Association Between Proportion of Cell Types and Histopathological Parameters

A Generalized Linear Mixed-Effects Model to assess association with histopathology (GLMM_histopathology) was implemented in the lme4 (v1.1-27.1) R package (Bates et al., J. Stat. Soft. 67, 1-48 (2015)) and used to identify cell types whose proportions are significantly associated with changes in histopathology across the samples. These models were performed separately for each combination of the cluster of cells and the eight histological parameters: hippocampal volume (mm³), the percent of AT8 coverage area, the percent of GFAP coverage area, the percent of S1000 coverage area, the percent of IBA1 coverage area, the percent of CD68 coverage area, the percent of MBP coverage area, and the percent of oligodendrocyte progenitor cell (OPC) coverage area. The GLM model was performed with the family argument set to the binomial probability distribution family and with the ‘bobyqa’ control optimizer used for the maximum likelihood estimation. Cluster membership for each cell was modeled as a 0-1 response variable according to whether or not the cell belongs to the cluster under consideration. The corresponding mouse model from which the cell was derived was included as a random effect and further the mouse id within the given mouse model was modeled as a random effect as well. Note, this represents the hierarchical nature of this data for the GLMM, and the mouse models are first assumed to be sampled from an “universe” of mouse models, this is then followed by sampling mice within each mouse model. The modeling choice of including the mouse model as a random effect as opposed to a fixed effect is meant to increase the degrees of freedom (or maximize the statistical power) to detect the association of interest, particularly in light of the relatively small number of replicates (3-4) per animal model. The histological parameter under consideration was modeled as a fixed effect in this model.

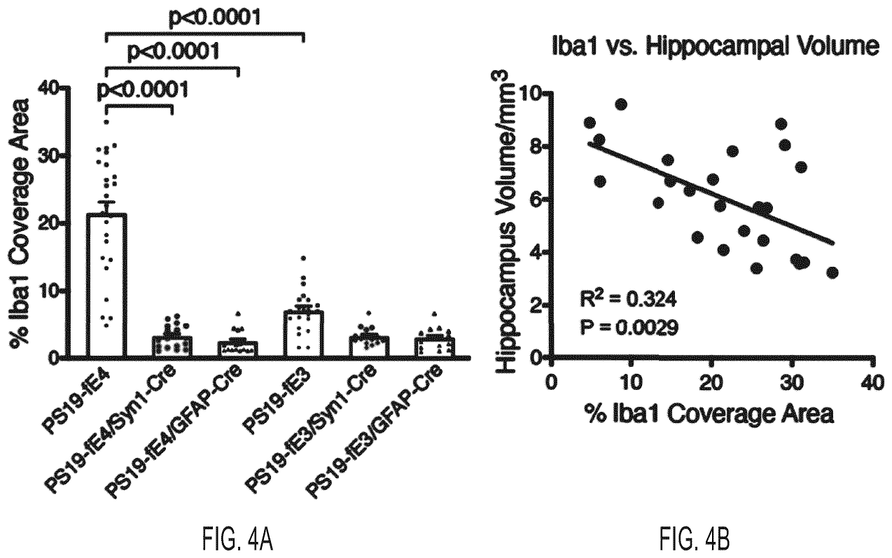

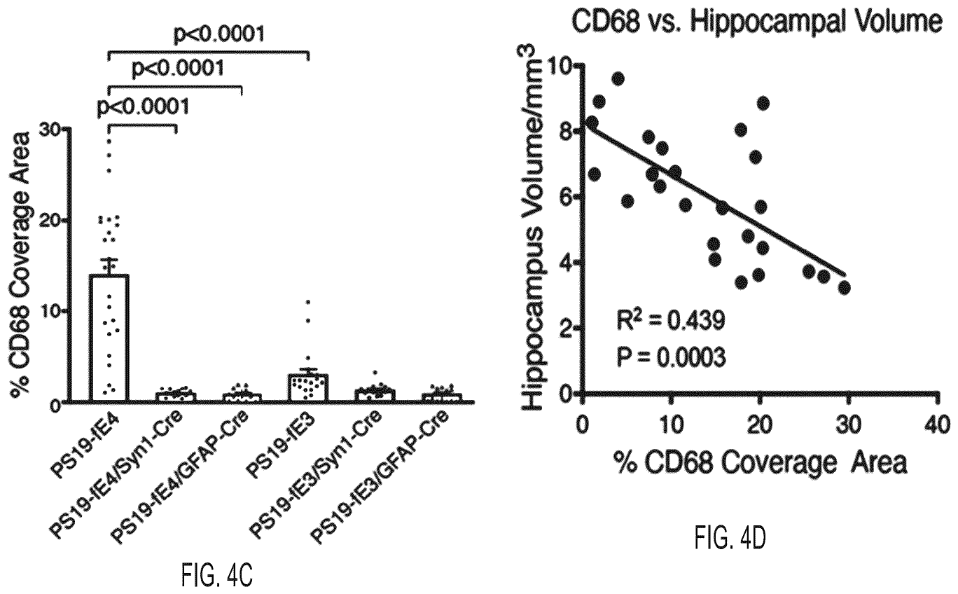

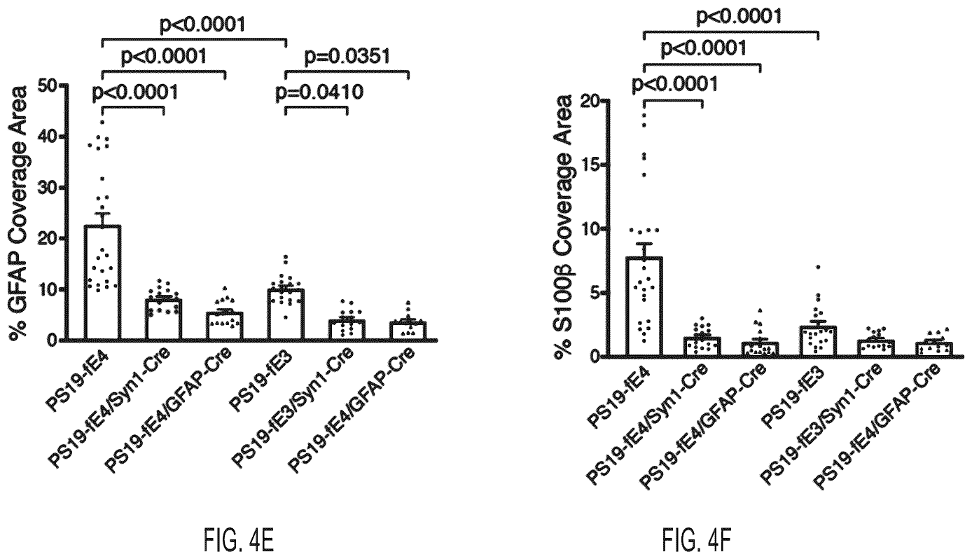

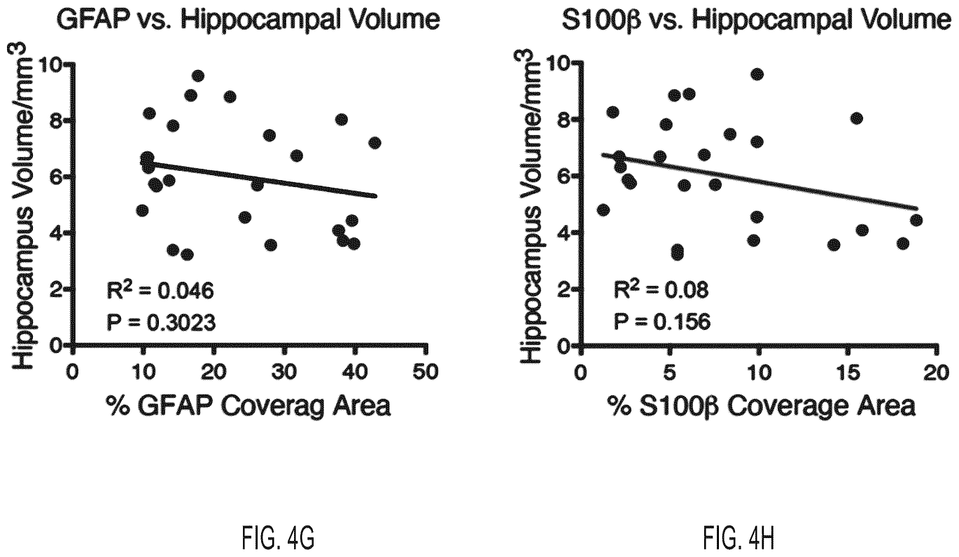

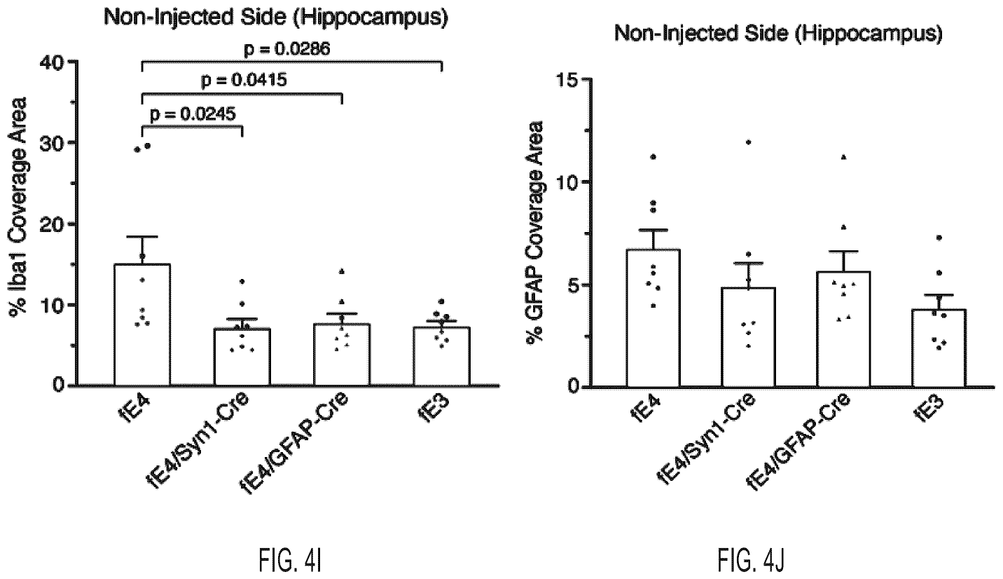

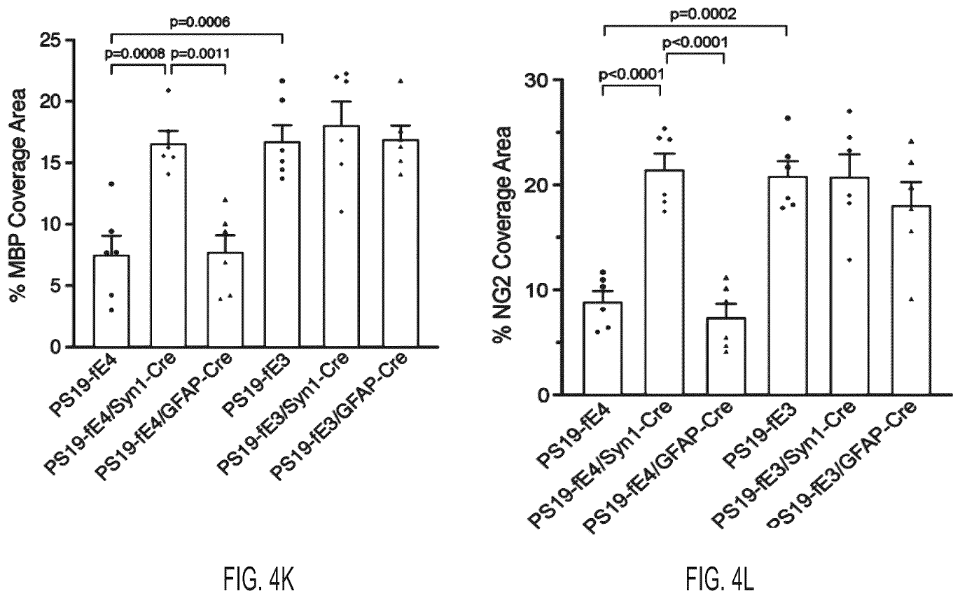

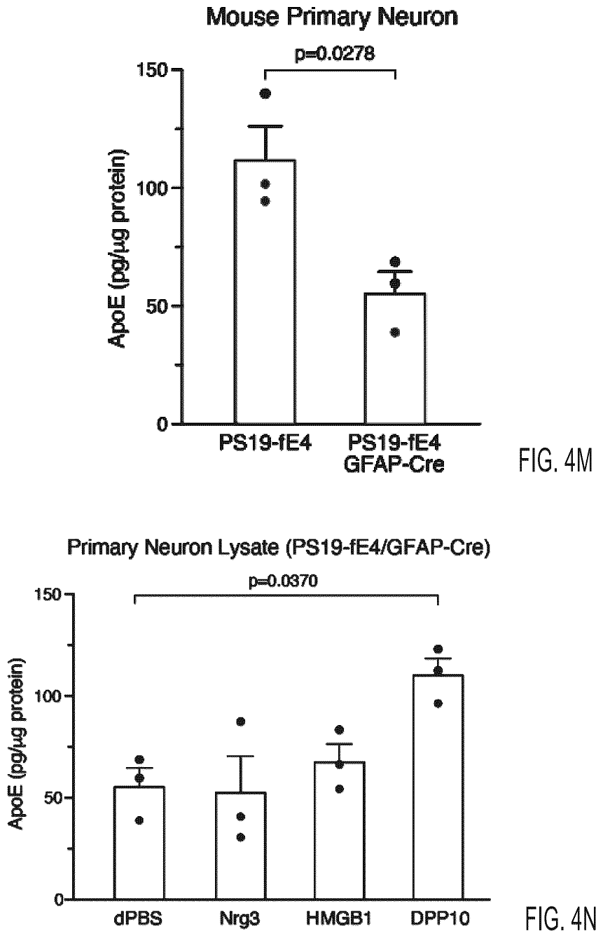

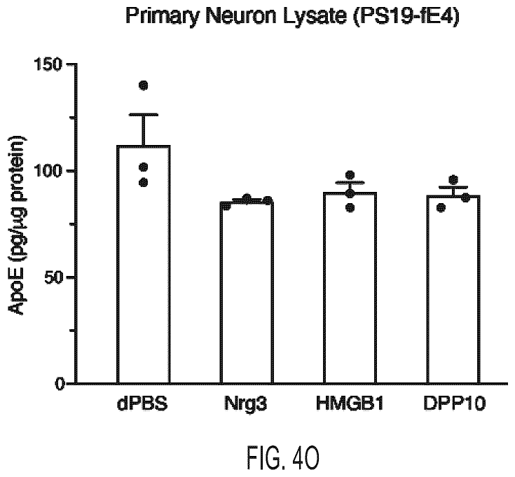

A subset of cell types of interest were selected and visualized the log Odds ratio estimates (derived from the GLMM fits) in a heatmap (FIG. 4d) using pheatmap package 1.0.12 after adjusting the p-values distribution across histopathological parameters across cell types with Benjamini-Hochberg multiple testing correction (Benjamini et al., J R Stat. Soc. Ser. B Stat. Methodol. 57, 289-300 (1995)). The pipeline was applied to the astrocyte and microglia subtypes and visualized the associations between astrocyte and microglia subtypes of interest and the eight histopathological parameters in FIG. 4h and FIG. 4i, respectively. The first 5 principal component coordinates were estimated using the eight log Odds ratio for unit change of the histopathological parameters for each of the cell types and astrocyte and microglia sub-cell types of interest. This was implemented using prcomp(scale=T, center=T) in stats R package. We visualized the first two PCs using fviz_pca_ind( ) implemented in factoextra 1.0.7 R package.

Mouse snRNA-Seq Dataset from GEO:GSE164507

Mouse snRNAseq data were reanalyzed that are available on the Gene Expression Omnibus database at ncbi.nlm.nih.gov/geo (accession no. GSE164507). The publicly available data set was downloaded which, for each sample, includes a filtered matrix of gene by cell expression, file with barcodes, and file with expressed genes. Full details of the study are available in the original publication (Wang et al., Neuron 109, 1657-1674.e7 (2021)). Briefly, the study examined P301S mutant Tau transgenic mice carrying floxed APOE-ε4 or APOE-ε3 alleles. These mice were crossed with mice expressing a Cre recombinase under the regulation of a tamoxifen-inducible ER element and the Aldh111 astrocyte-specific promoter. These Aldh111-CreERT2 mice were administered either tamoxifen, to induce Cre recombinase expression, or a vehicle at 5.5 months of age, after the onset of tau pathology. Isolated single nuclei from the hippocampus of these mice were sequenced using 10× Genomics Chromium Single Cell sequencing and the data was processed using Cell Ranger Single Cell Software Suite (v3.0.2). The filtered count matrices generated by the Cell Ranger count pipeline for all 8 samples were processed using Seurat v4.0.4 (Hao et al. Cell 184: 3573-3587.e29 (2021)). Samples were filtered to include only cells with 500-2,000 genes detected and <5% mitochondrial reads. The filtered samples were merged into a single Seurat object containing a matrix of 33,457 genes by 63,248 nuclei. Normalization and variance stabilization were performed using sctransform (Hafemeister & Satija, Genome Biol. 20, 296 (2019)). Clustering was determined as implemented in Seurat v4.0.4 with the RunPCA( ), FindNeighbors( ), and FindClusters( ) functions. Nearest neighbor distances were computed using up to the first 15 PCs. This algorithm embeds cells in a k-nearest neighbor graph, based on Euclidean distance in PCA space. The edge weights between any two cells are further refined using Jaccard similarity. Clustering was implemented using the Louvain algorithm with the default settings and a resolution of 0.7, resulting in a set of 22 distinct clusters. Differential gene expression was detected using the Wilcoxon Rank Sum test in Seurat, implemented via the FindMarkers( ) function with min.pct=0.1, test.use=“wilcox”, and logfc.threshold=0.05.

### General Statistical Analysis

The differences between genotype groups were evaluated by ordinary one-way ANOVA with Tukey's multiple comparisons test, where the mean of each column was compared with the mean of every other column. All plotted data are presented as the mean±SEM. The correlations between two data in the same genotype group were analyzed using simple linear regression and plotted as the mean±SEM. The analyses were performed and plots were created with GraphPad Prism version 9.2.0.

### Example 2: Neuron- or Astrocyte-Specific Removal of the APOE Gene in Human APOE-Knock-In Mice Expressing Human Mutant Tau

The inventors and co-workers had previously generated mouse lines expressing a floxed human APOE3 or APOE4 gene (Bien-Ly et al., J. Neurosci. 32, 4803-4811 (2012)) and a Cre recombinase gene under the control of a neuron-specific Synapsin-1 promoter (Syn1-Cre) (Zhu et al. Genes Dev. 15, 859-876 (2001)) or an astrocyte-specific Glial Fibrillary Acidic Protein promoter (GFAP-Cre) (Bajenaru et al., Mol. Cell Biol. 22, 5100-5113 (2002); Uhlmann et al., Ann. Neurol. 52, 285-296 (2002)).

These floxed APOE-K1 (fE) mice express homozygous human APOE3 or APOE4 in place of the endogenous mouse Apoe. The human APOE gene was flanked by a pair of LoxP sites to allow for its precise excision in the presence of cell-type-specific Cre recombinase expression (Knoferle et al., J. Neurosci. 34, 14069-14078 (2014)). The fE mice with no Cre, Syn1-Cre, or GFAP-Cre were crossbred with mice expressing mutant 1N4R human microtubule-associated protein Tau (MAPT) encoding the disease-associated P301S mutation (PS19 line), which has been widely utilized as a tauopathy mouse model (Yoshiyama et al., Neuron 53, 337-351 (2007)). The resulting compound mice are referred to as PS19-fE, PS19-fE/Syn1-Cre or PS19-fE/GFAP-Cre mice.

Rigorous characterization of the fE/Syn1-Cre and fE/GFAP-Cre mice was performed to validate the specificity of Cre recombinase expression under the neuron-specific Syn1 or the astrocyte-specific GFAP promoter (Knoferle et al. J. Neurosci. 34, 14069-14078 (2014)). The PS19-fE/Cre mice were further validated by performing immunohistochemical analysis of brain sections from 10-month-old PS19-fE4/Syn1-Cre and PS19-fE4/GFAP-Cre mice. To confirm the specificity of Cre recombinase expression in hippocampal neurons and astrocytes when driven under the Syn1 or GFAP promoter, respectively, co-immunostaining with NeuN and GFAP antibodies was performed along with a Cre recombinase antibody. In PS19-fE4/Syn1-Cre mice, Cre recombinase was expressed exclusively in NeuN-positive neurons and was not expressed in GFAP-positive astrocytes. In PS19-fE4/GFAP-Cre mice, Cre recombinase was expressed exclusively in GFAP-positive astrocytes and was not expressed in NeuN-positive neurons. Co-immunostaining using antibodies against APOE, GFAP, and NeuN illustrated that PS19-fE4 mice without Cre have APOE expression in GFAP-positive astrocytes and some NeuN-positive neurons, whereas PS19-fE4/Syn1-Cre mice have APOE expression in astrocytes and lack APOE expression in neurons while PS19-fE4/GFAP-Cre mice have APOE expression in some neurons and lack APOE expression in astrocytes. The PS19-fE4/GFAP-Cre mice exhibited some APOE-positive cells that were negative for NeuN and GFAP but positive for the microglial marker Iba1, indicating that they were APOE-expressing microglia.

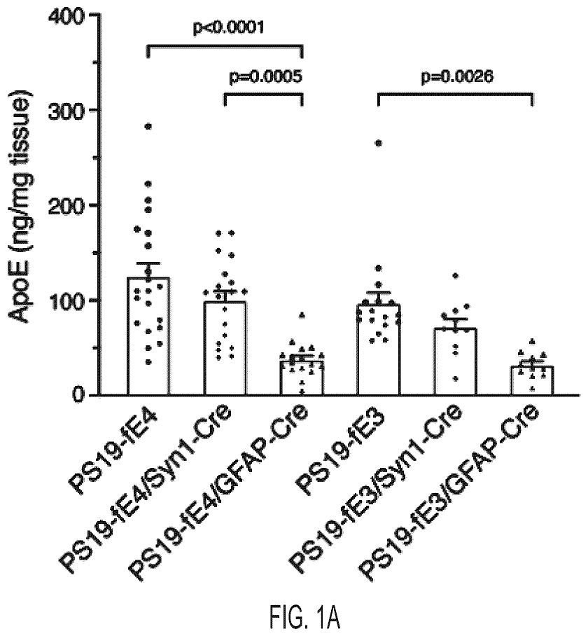

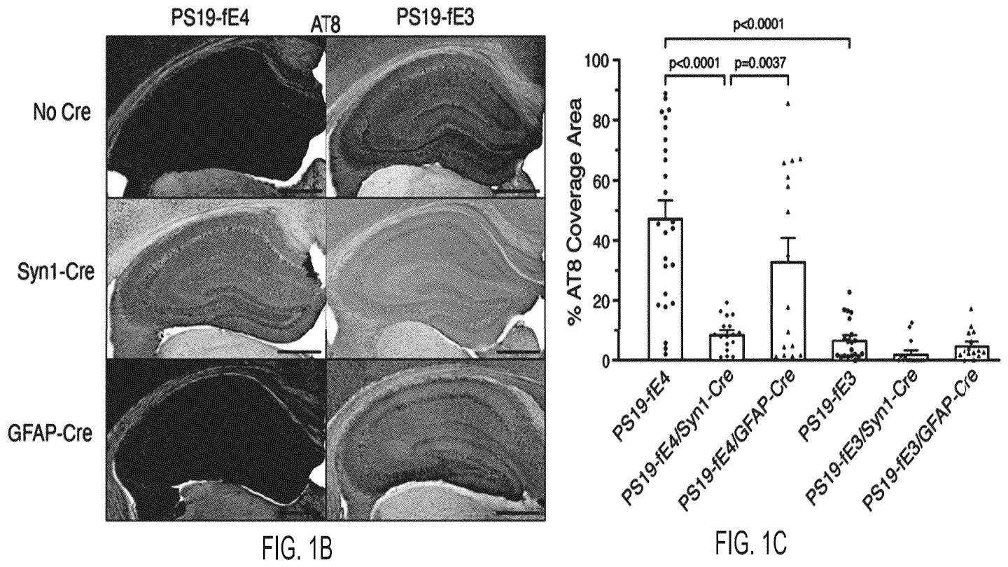

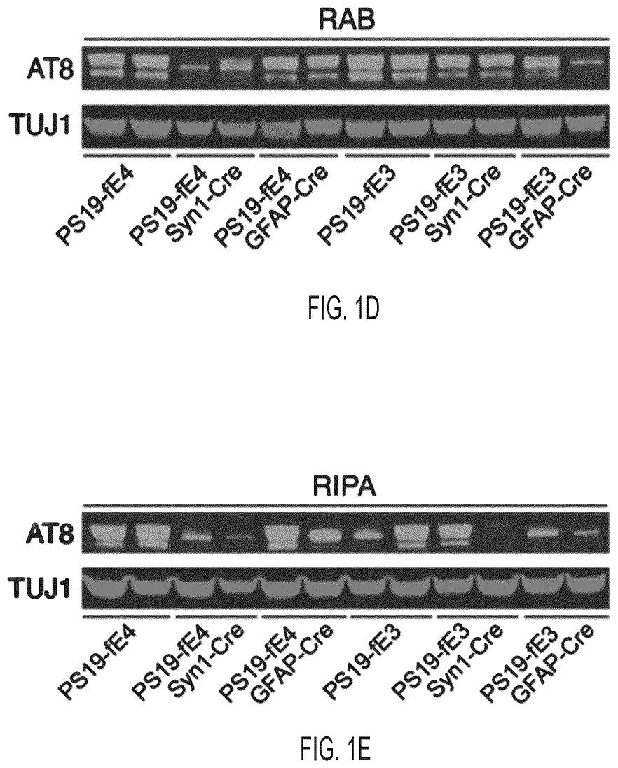

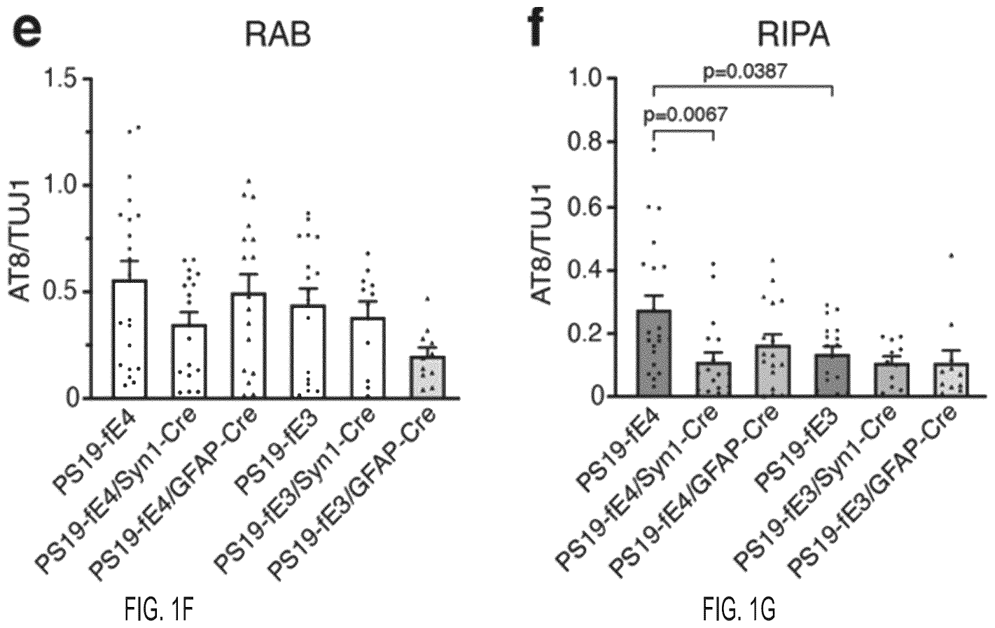

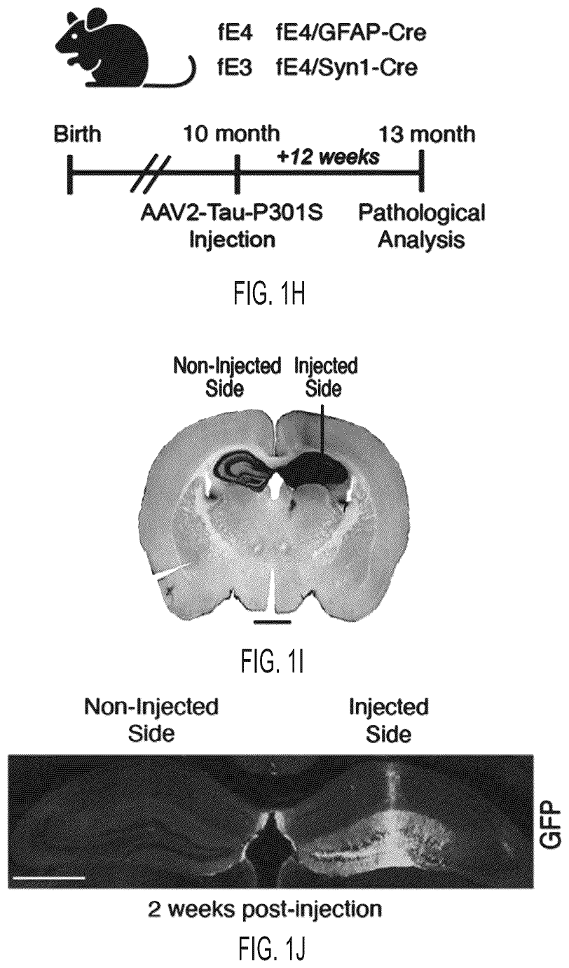

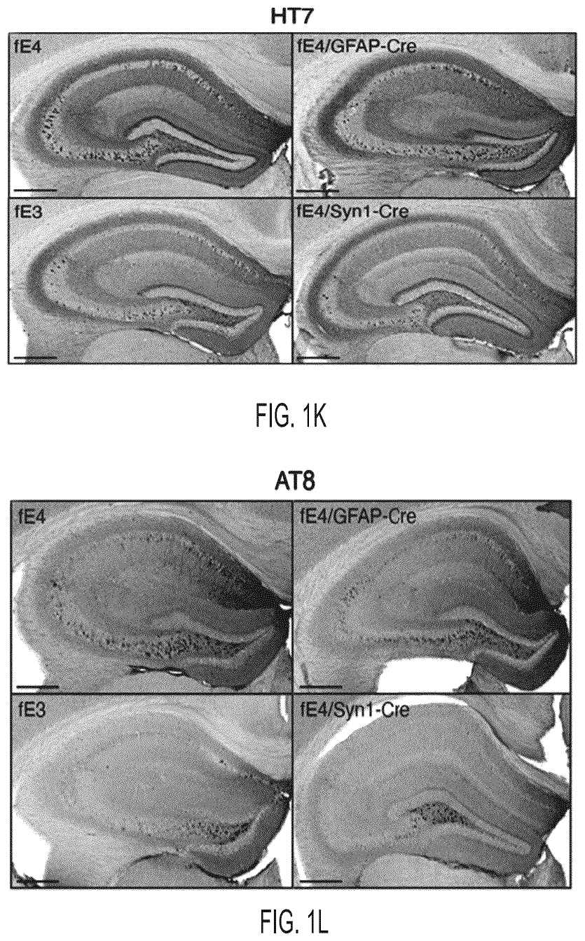

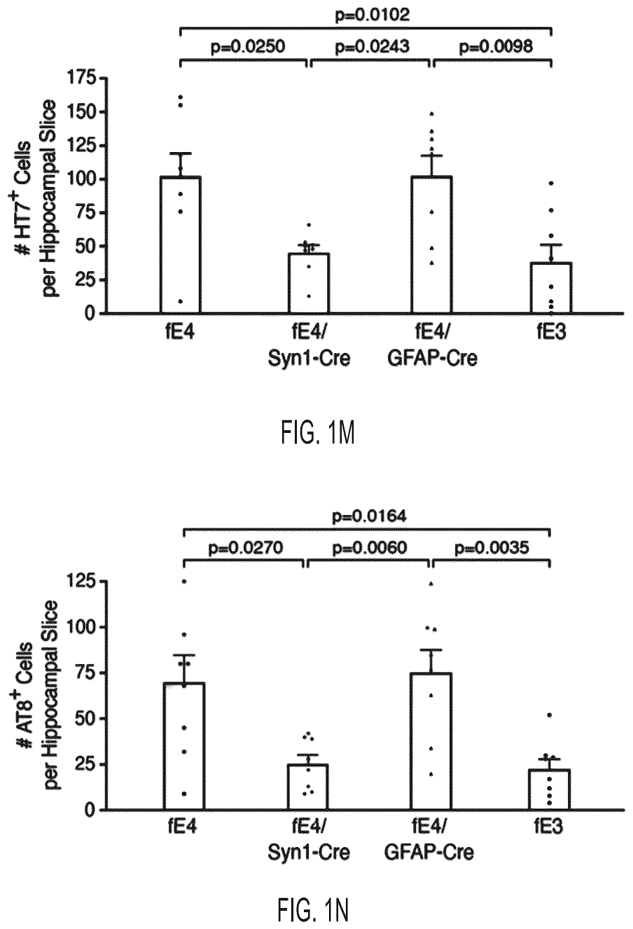

In order to quantitatively determine the levels of APOE protein within these various mouse models, hippocampal lysates were analyzed of 10-month-old mice by sandwich ELISA for human APOE. PS19-fE4/Syn1-Cre mice exhibited an approximate 20% decrease in APOE levels relative to PS19-fE4 mice (FIG. 1A), which aligns with previous data indicating that neuronal APOE contributes to about 20-30% of total APOE protein levels in the hippocampus and cortex. PS19-fE4/GFAP-Cre mice exhibited an approximate 70% decrease in APOE levels relative to PS19-fE4 mice, which agrees with the well-established role of astrocytes as being the main producers of APOE within the CNS. Similarly, PS19-fE3/Syn1-Cre mice exhibited an approximate 25% decrease and PS19-fE3/GFAP-Cre exhibited an approximate 67% decrease in APOE levels relative to PS19-fE3 mice (FIG. 1A). Taken together, these results provide strong evidence that APOE gene expression is eliminated in neurons or astrocytes when Cre recombinase expression is driven under a Syn1 or GFAP promoter, respectively, in these compound mouse models.

### Example 3: Removal of APOE4 from Neurons, but not Astrocytes, Drastically Reduces Tau Pathology

To determine whether the removal of neuronal or astrocytic APOE affects Tau pathology, the inventors assessed mice at 10 months of age, which is when PS19 mice exhibit extensive Tau pathology throughout the hippocampus (Yoshiyama et al., Neuron 53, 337-351 (2007)). The accumulation of pTau in the hippocampus was analyzed by immunohistochemical staining with the pTau-specific AT8 antibody and quantified Tau pathology as the percent of AT8 coverage area in the hippocampus.

PS19-fE4 mice presented extensive Tau pathology throughout the hippocampus and the extent of Tau pathology was significantly lower in PS19-fE3 mice (FIG. 1B-1C), as has been previously reported (Shi et al., Nature 549, 523-527 (2017)). Relative to PS19-fE4 mice, the PS19-fE4/Syn1-Cre mice exhibited a striking reduction of about 81% in Tau pathology, whereas PS19-fE4/GFAP-Cre mice had a minor reduction of about 30% that did not reach statistical significance. Interestingly, the reduction in pTau coverage area in PS19-fE4/Syn1-Cre mice resembled the extent of Tau pathology observed in PS19-fE3 mice (FIG. 1B-1C). There was no significant difference in Tau pathology between PS19-fE3 with and without Cre, likely because the Tau pathology in PS19-fE3 mice was already low.

Western blot analysis was also utilized to assess levels of pTau in mouse hippocampal tissue following sequential biochemical extraction with RAB and RIPA buffers, containing highly soluble and less soluble Tau proteins, respectively (Shi et al., Nature 549, 523-527 (2017); Ishihara et al., Neuron 24, 751-762 (1999)) (FIG. 1D-1G). There was no significant difference in pTau levels between the various genotype groups in the RAB fraction (FIG. 1D, 1F). However, PS19-fE4/Syn1-Cre and PS19-fE3 mice exhibited a significant reduction in pTau levels in the RIPA fraction relative to PS19-fE4 mice (FIG. 1E, 1G). PS19-fE4/GFAP-Cre mice showed a trend towards a reduction in pTau levels in the RIPA fraction of about 40%, relative to PS19-fE4 mice, which did not reach statistical significance. Taken together, these data indicate that neuronal APOE4 expression is a strong driver of Tau pathology, while astrocytic APOE4 exerts a minimal effect on Tau pathology.

### Example 4: Propagation of Tau Pathology is Reduced after Removal of APOE4 in Neurons, but not Astrocytes

To further investigate the mechanisms by which neuronal APOE4 drives Tau pathology, the effects of cell type-specific APOE4 expression on the propagation of Tau pathology was determined. Previous studies have illustrated that pathological Tau can propagate to anatomically connected brain regions after injecting various forms of Tau protein directly into mouse brains (Boluda et al., Acta Neuropathol. 129, 221-237 (2015); Rauch et al., Nature. 580, 381-385 (2020); Guo et al., J. Exp. Med. 213, 2635-2654 (2016); Kaufman et al., Neuron 92, 796-812 (2016)).

The extent of Tau propagation was analyzed after a single unilateral injection of an adeno-associated virus-2 encoding human P301S mutant Tau (AAV2-Tau-P301S) into the right dorsal hippocampus of fE mice with no Cre, Syn1-Cre, or GFAP-Cre (FIG. 1H-1I). The fE mice lack human P301S mutant Tau and instead express the endogenous mouse Mapt gene, allowing for a more accurate detection of human Tau spread since fE mice exhibit minimal Tau pathology. The mice were injected with the AAV2-Tau-P301S virus at 10 months of age and assessed 12 weeks post-injection at 13 months of age (FIG. 1H).

To provide evidence that the observed Tau propagation phenotype can truly be attributed to the spread of pathological human Tau between neurons as opposed to an unexpected consequence of the Tau-encoding virus itself traveling to the non-injected hippocampal side, unilateral injection into the right dorsal hippocampus of a 10-month-old fE4 mouse was evaluated of an AAV2 of the same serotype that encodes GFP (AAV2-GFP). Immunostaining with anti-GFP two weeks post-injection revealed that the GFP signal remains localized to neurons within the injected hippocampal side (FIG. 1J). The non-injected hippocampal side did not have any evidence of GFP signal in neuronal somas, although there were some GFP-positive neuronal projections, likely stemming from neurons residing on the injected hippocampal side. These results illustrate that the AAV2 itself does not spread between the right and left hippocampus following unilateral injection. Furthermore, for all immunohistochemical analyses, the number of soma-positive-Tau-containing neurons was quantified to more accurately reflect human mutant Tau spread between neurons and exclude confounding factors, such as Tau-positive commissural fibers from neurons originating from the injected side.

Immunohistochemical staining with a human Tau antibody (HT7) revealed robust human Tau propagation to the non-injected hippocampal side in PS19-fE4 mice and minimal Tau propagation in PS19-fE3 mice (FIG. 1K-1L), indicating that APOE4 promotes Tau spreading. Intriguingly, PS19-fE4/Syn1-Cre mice had limited human Tau propagation to the non-injected hippocampal side, while PS19-fE4/GFAP-Cre mice had extensive human Tau propagation that resembled the spread of Tau pathology observed in PS19-fE4 mice (FIG. 1K-1L). Notably, there was a 55% decrease in the number of HT7T cells with Tau propagation after removal of neuronal APOE4 and no discernable difference in the number of HT7⁺ cells with Tau propagation after the removal of astrocytic APOE4 (FIG. 1K-1N).

The extent of Tau propagation was further analyzed by immunostaining for pTau with the AT8 antibody. PS19-fE4 and PS19-fE4/GFAP-Cre mice both exhibited robust propagation of pTau to the non-injected hippocampal side, while PS19-fE4/Syn1-Cre and PS19-fE3 mice had drastically reduced pTau propagation (FIG. 1M-1N).

Taken together, these data indicate that one mechanism by which neuronal APOE4 drives Tau pathology is by stimulating the propagation of pathological Tau across synaptically connected neurons. This data also further supports the notion that astrocytic APOE4 is not a key mediator of Tau pathology, as evidenced by its lack of effect on Tau propagation.

### Example 5: Network Hyperexcitability is Eliminated after Removal of Neuronal, but not Astrocytic, APOE4

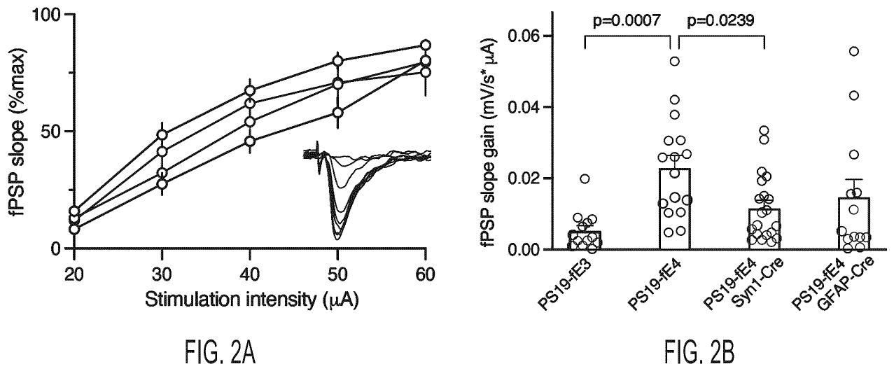

To determine the cell type-specific effects of APOE4 on neuronal function in the context of tauopathy, neuronal network excitability was measured in the hippocampal cornu ammonis (CA1) region of PS19-fE3 mice and PS19-fE4 mice with no Cre, Syn1-Cre, or GFAP-Cre by input-output curve analysis of network response to incremental stimulation of Schaffer collaterals (FIG. 2A)⁵⁴.

PS19-fE4 mice had notable CA1 neuron hyperexcitability as compared to PS19-fE3 mice (FIG. 2B). Removing neuronal APOE4 eliminated neuronal network hyperexcitability, while removing astrocytic APOE4 led to a minor reduction that did not reach significance (FIG. 2B), indicating that neuronal APOE4 drives neurodysfunction in the context of tauopathy.

### Example 6: Neurodegeneration is Reduced after Removal of Either Neuronal or Astrocytic APOE4

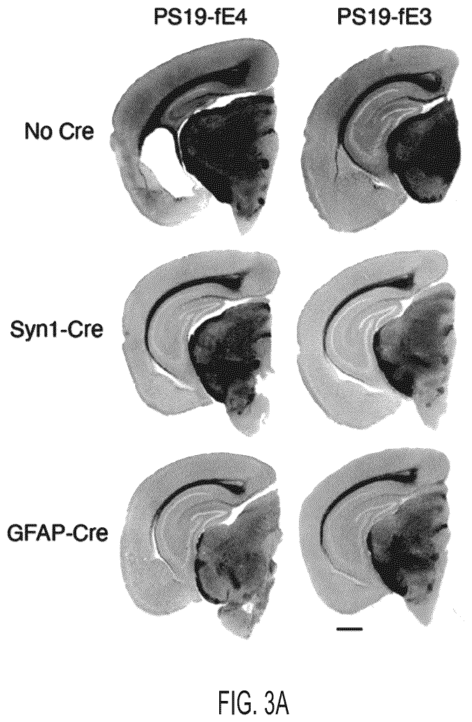

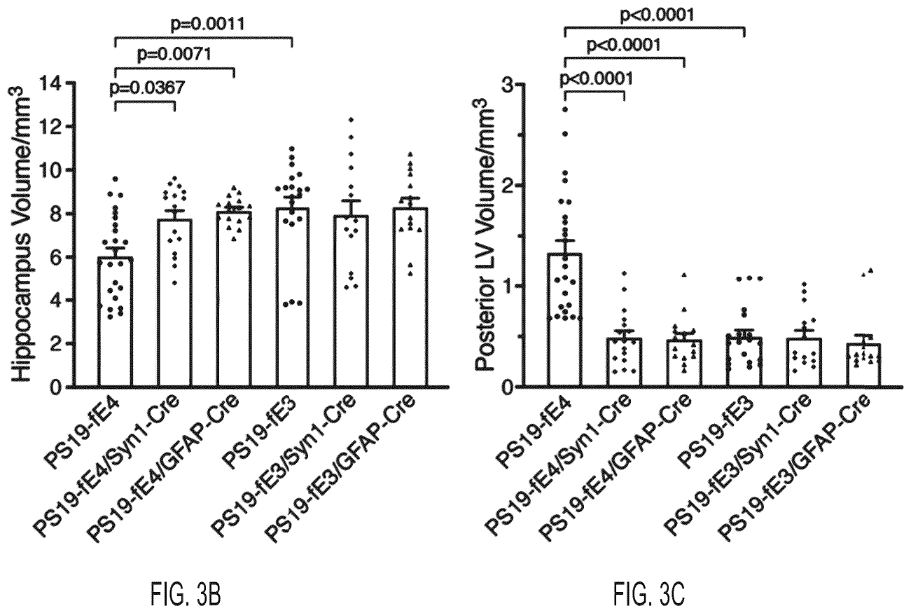

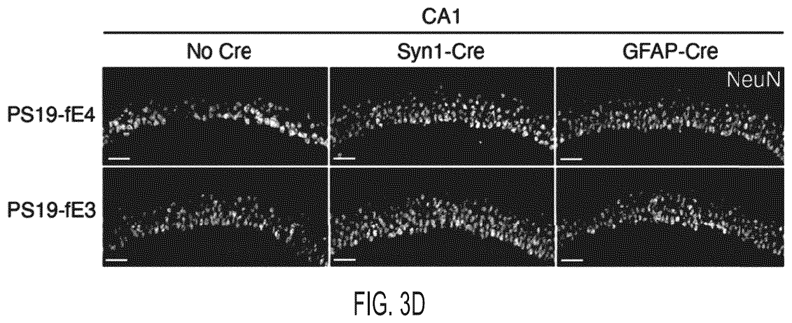

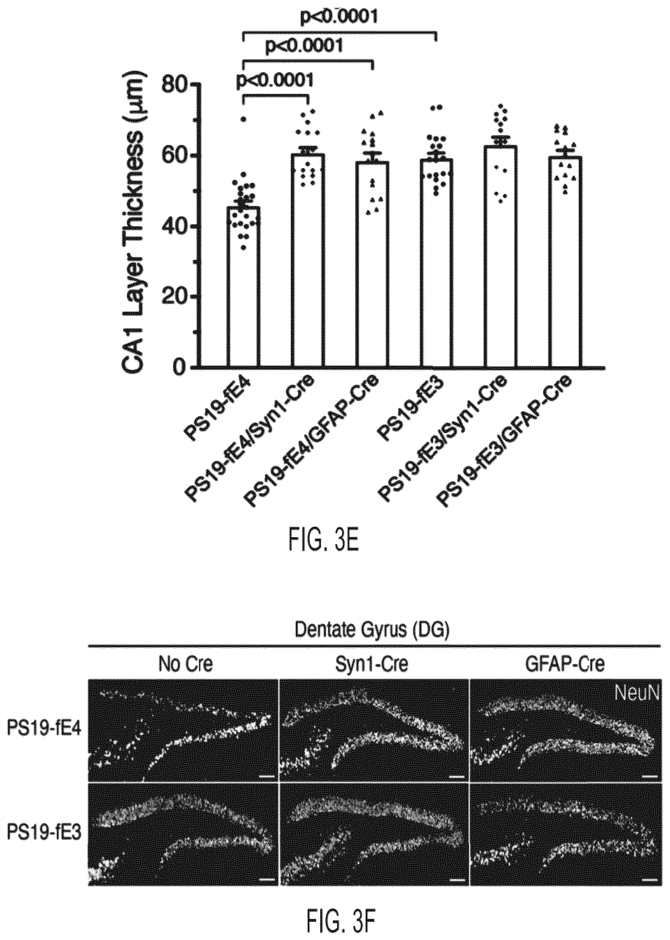

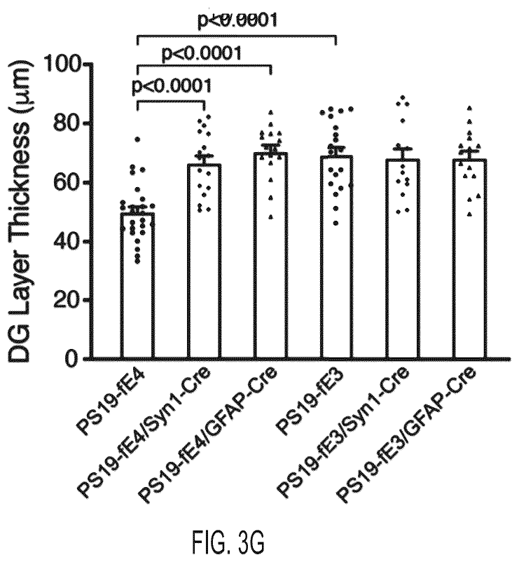

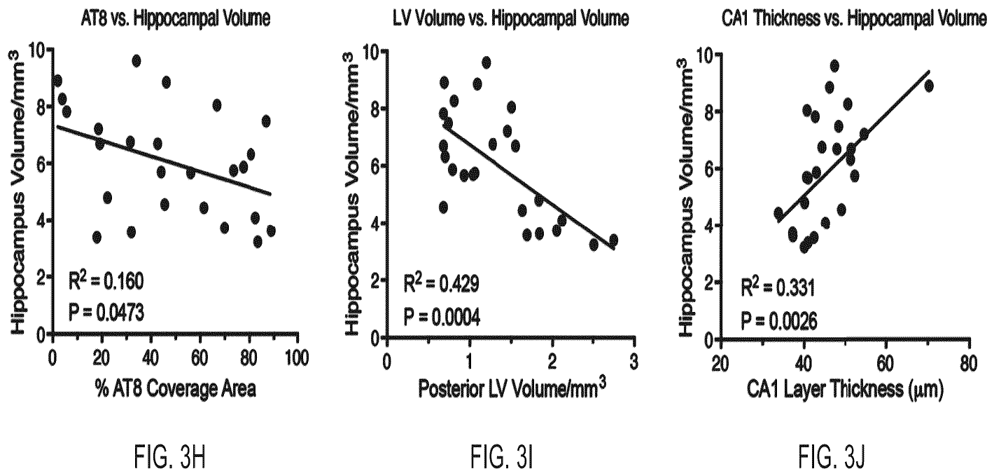

The extent of neurodegeneration in 10-month-old PS19-fE mice was evaluated after removal of APOE from neurons or astrocytes. Analyses of hippocampal and posterior lateral ventricle volumes revealed that PS19-fE4 mice exhibited extensive neurodegeneration relative to PS19-fE3 mice (FIG. 3A-3C). Neurodegeneration was significantly reduced in both PS19-fE4/Syn1-Cre and PS19-fE4/GFAP-Cre mice, as illustrated by increased hippocampal volume and decreased posterior lateral ventricle volume (FIG. 3A-3C), resembling those of PS19-fE3 mice. Removal of neuronal or astrocytic APOE3 did not significantly impact neurodegeneration (FIG. 3A-3C).

Quantification of the extent of neuronal cell loss within the various subfields of the hippocampus revealed that PS19-fE4 mice had extensive neuron loss in CA1 and the dentate gyrus, while PS19-fE4/Syn1-Cre and PS19-fE4/GFAP-Cre mice both had significantly reduced neuronal cell loss in both CA1 and dentate gyrus subfields of the hippocampus (FIG. 3D-3G). There was no significant difference in neuronal cell layer thickness in PS19-fE3 mice when APOE3 was removed from neurons or astrocytes (FIG. 3D-3G).

In PS19-fE4 mice, there was a weak, but significant, negative correlation between Tau pathology and hippocampal volume (FIG. 3H), suggesting that Tau pathology contributes to the neurodegeneration occurring in these mice, at least to some extent. There was also a strong negative correlation between hippocampal volume and the volume of the posterior lateral ventricle, and a strong positive correlation between the thickness of the CA1 cell layer and hippocampal volume (FIG. 3I-3J). Overall, these data illustrate that removal of either neuronal or astrocytic APOE4 is protective against Tau-mediated neurodegeneration and leads to reduced neuronal cell and hippocampal volume loss. This result is intriguing, considering the Tau pathology data showing that astrocytic APOE4 has minimal effects on Tau pathology accumulation and propagation.

### Example 7: Gliosis is Drastically Reduced after Removal of Either Neuronal or Astrocytic APOE4

The extent of microgliosis and astrogliosis was investigated within the genotype groups at 10 months of age to explore the roles of neuronal or astrocytic APOE4 in eliciting gliosis in the context of tauopathy. Immunohistochemical staining of microglia using an Iba1 antibody revealed that PS19-fE4 mice had extensive microgliosis in the hippocampus, as demonstrated by a high percent coverage area of microglia (FIG. 4A). PS19-fE4/Syn1-Cre and PS19-fE4/GFAP-Cre mice both exhibited drastic reductions in microgliosis (FIG. 4A). Additionally, PS19-fE3 mice with no Cre, Syn1-Cre, or GFAP-Cre all have greatly reduced microgliosis relative to PS19-fE4 mice (FIG. 4A). There was a strong negative correlation between Iba1 coverage area and hippocampal volume in PS19-fE4 mice, indicating that microgliosis is a good indicator and potential contributor to neurodegeneration (FIG. 4B).

This microgliosis phenotype was further defined by immunohistochemical staining of CD68, which is a marker of activated microglia. There was a dramatic difference in the coverage area of activated microglia between genotype groups, with PS19-fE4 mice exhibiting extensive microglial activation and all other genotype groups having minimal microglial activation (FIG. 4C). Interestingly, of all pathological correlations made in PS19-fE4 mice, the coverage area of CD68+ activated microglia had the strongest negative correlation with hippocampal volume (FIG. 4D), suggesting that the extent of microglial activation is the strongest indicator and potential contributor to APOE4-enhanced neurodegeneration in tauopathy. Taken together, these data indicate that microgliosis is strongly enhanced by APOE4 relative to APOE3 in tauopathy, and that removal of APOE4 from either neurons or astrocytes attenuates the extent of microgliosis.

The extent of astrogliosis was also assessed after removal of APOE from neurons or astrocytes. PS19-fE4 mice exhibited considerable astrogliosis in the hippocampus relative to PS19-fE3 mice, as detected by immunohistochemical staining with the astrocytic GFAP antibody (FIG. 4E). The extent of astrogliosis was greatly reduced after removal of either neuronal or astrocytic APOE4 (FIG. 4E). These observations were also supported by immunohistochemical staining for an activated astrocytic marker, S100β⁵⁶ (FIG. 4F). There was no obvious difference in the extent of astrogliosis after removal of APOE3 from either cell type relative to PS19-fE3 mice (FIG. 4E-4F). Strikingly, and in sharp contrast to microgliosis, neither the coverage area of astrocytes nor activated astrocytes was significantly correlated with hippocampal volume (FIG. 4G-4H). These data indicate that APOE4 strongly enhances astrogliosis relative to APOE3 in tauopathy, and that the removal of APOE4 from either neurons or astrocytes reduces astrocyte activation and/or recruitment. Furthermore, it also illustrates that the extent of astrogliosis might not be a valuable indicator or contributor to APOE4-enhanced neurodegeneration in tauopathy.

To further demystify the relationship between Tau pathology and gliosis, we investigated whether Tau propagation also elicits gliosis in the non-injected hippocampal side (refer to studies in FIG. 1H-1N). Indeed, fE4 mice, along with higher Tau spread, also exhibited higher microgliosis in the non-injected hippocampal side than fE3 mice (FIG. 4I). Fascinatingly, there was a significant decrease in microgliosis in the non-injected hippocampal side after removal of either neuronal or astrocytic APOE4. This indicates that removal of neuronal or astrocytic APOE4 reduces microgliosis regardless of the extent of Tau spread, as fE4/GFAP-Cre mice still had relatively high levels of Tau spread to the non-injected hippocampal side.

These findings indicate that astrocytic APOE4 acts downstream of Tau pathology to promote microgliosis. The extent of astrocyte coverage area in the non-injected hippocampal side was also assessed and no discernable differences in astrogliosis after removal of neuronal or astrocytic APOE4 were observed (FIG. 4I-4J), probably due to the relatively low astrogliosis in the non-injected hippocampal side even in fE4 mice in this Tau spreading model.

### Example 8: Myelin Deficits and Depletion of Oligodendrocyte Progenitor Cells are Reduced after Removal of APOE4 from Neurons, but not Astrocytes

Myelin degeneration and oligodendrocyte deficits have been observed in human AD brains (Benitez et al., Neuroimage Clin. 4: 64-71 (2013); Dean et al., JAMA Neurol. 74, 41-49 (2017); Nasrabady et al., Acta Neuro. Comms. 6, 22 (2018)) and in mouse models of Alzheimer's Disease and tauopathy (Desai et al., Glia 57, 54-65 (2009); Zhang et al., Sci. Adv. 6, eabb8680 (2020); Shi et al., Neuron 109, 2413-2426.e7 (2021)).

The inventors investigated the effects of neuronal and astrocytic APOE4 removal on the maintenance of myelin integrity and the density of hippocampal oligodendrocyte progenitor cells (OPCs). To determine myelin integrity in the hippocampus, myelin basic protein (MBP) was immunostained with an MBP-specific antibody. Quantification of the percent coverage area of MBP in the stratum radiatum underneath the pyramidal cell layer of CA1 revealed that PS19-fE4 mice had extensive myelin loss relative to PS19-fE3 mice (FIG. 4K). Strikingly, a significant reduction in myelin loss was observed in PS19-fE4/Syn1-Cre mice that resembled the phenotype of PS19-fE3 mice, while PS19-fE4/GFAP-Cre mice exhibited considerable myelin loss that resembled the phenotype of PS19-fE4 mice (FIG. 4K). These data indicate that neuronal APOE4 plays a primary detrimental role in promoting myelin deficits in the context of tauopathy, whereas astrocytic APOE4 does not impact this phenotype. There was no substantial difference in myelin integrity in PS19-fE3 mice after removal of neuronal or astrocytic APOE3.

To further characterize the effects of neuronal and astrocytic APOE4 on biological components involved in the myelination process, cells were immunostained with an NG2 antibody to probe for oligodendrocyte progenitor cells (OPCs), which have been suggested to aid the repair of damaged myelin in conditions of CNS injury and neurodegeneration (Shi et al. Glia 57, 54-65 (2009); Tripathi et al., J. Neurosci 30, 16383-16390 (2010)). A significant decrease was observed in the percent OPC coverage area in the hippocampus of PS19-fE4 mice, as compared to PS19-fE3 mice (FIG. 4L). Strikingly, removal of neuronal APOE4 significantly increased the percent OPC coverage area to levels that were similar to those in the PS19-fE3 mice (FIG. 4L). In contrast, PS19-fE4/GFAP-Cre mice had significantly lower OPC coverage area relative to PS19-fE4/Syn1-Cre mice and even exhibited a trend of decreased OPCs relative to PS19-fE4 mice, although it did not reach statistical significance (FIG. 4L). There were no obvious differences in OPC coverage area after removal of neuronal or astrocytic APOE3 in PS19-fE3 mice (FIG. 4L).

Immunohistochemical staining for myelin and OPCs in fE4 and fE3 mice revealed that APOE4 mice lacking the human mutant Tau-P301S do not exhibit myelin deficits and have similar OPC levels in the hippocampus as fE3 mice, illustrating that the effects of APOE4 on these phenotypes are specific to the setting of tauopathy.

Overall, these findings indicate that neuronal APOE4 plays a pivotal role in depleting the hippocampal OPC pool and causing myelin deficits in this compound tauopathy mouse model, while astrocytic APOE4 does not significantly contribute to either of these two pathological processes.

### Example 9: snRNA-Seq Identifies Neurodegenerative Disease-Associated Subpopulations of Neurons and Oligodendrocytes that are Largely Eliminated by Removing Neuronal, but not Astrocytic, APOE4

To gain an in-depth understanding of the cell type-specific effects of APOE4 at the transcriptomic level across different types of hippocampal cells, single-nucleus RNA sequencing (snRNA-seq) was performed on isolated hippocampi from 10-month-old PS19-fE4 mice with no Cre, Syn1-Cre, or GFAP-Cre and PS19-fE3 mice. The snRNA-seq dataset contained 119,317 nuclei covering 26,285 genes after normalization and filtering for quality control. Clustering by shared nearest neighbor (SNN) and visualization by uniform manifold approximation and projection (UMAP) revealed 33 distinct cell clusters. Based on their expression of marker genes, these clusters were assigned to:
16 excitatory (Ex) neuron clusters (3-5, 7, 12, 15, 16, 18-20, 23, 25-28, 33);
7 inhibitory (In) neuron clusters (6, 8, 9, 13, 22, 24, 30);
3 oligodendrocyte clusters (1, 2, 17);
one astrocyte cluster (10);
3 microglia clusters (11, 21 29);
2 oligodendrocyte progenitor cell (OPC) clusters (14, 32); and
1 unknown cluster (31)

APOE was highly expressed in astrocytes in PS19-fE4 and PS19-fE3 mice, and its expression was drastically reduced in astrocytes in PS19-fE4/GFAP-Cre mice. The inventors and coworkers have reported (Zalocusky et al., Nat. Neurosci. 24, 786-798 (2021)), some neurons also expressed APOE in PS19-fE4 and PS19-fE3 mice. As described herein, neuronal APOE expression was eliminated in PS19-fE4/Syn1-Cre mice.

Log odds ratio estimates from a Generalized Linear Mixed Effects Model to assess association with Animal Models (GLMM_AM) was used to identify cell clusters that were altered in PS19-fE3, PS19-fE4/Syn1-Cre, and PS19-fE4/GFAP-Cre mice versus PS19-fE4 mice. This analysis revealed that oligodendrocyte cluster 17 had lower odds of having cells from PS19-fE4/Syn1-Cre mice than from PS19-fE4 mice, with an almost complete elimination in PS19-fE4/Syn1-Cre mice. Based on differentially expressed (DE) gene analysis, cells in cluster 17 exhibited a drastic upregulated expression of three major heat shock protein genes and a significant downregulated expression of myelin basic protein (Mbp) and myelin associated oligodendrocyte basic protein (Mobp) genes relative to the other oligodendrocyte clusters. Differentially expressed (DE) pathway analysis revealed the enrichment of Kyoto Encyclopedia of Gene and Genomes (KEGG) pathways related to general neurodegeneration, AD, and other neurodegenerative diseases, indicating that cluster 17 represents neurodegenerative disease-associated oligodendrocytes.

Furthermore, excitatory neuron cluster 25 had significantly lower odds of having cells from PS19-fE4/Syn1-Cre and PS19-fE3 mice than from PS19-fE4 mice, with an almost complete elimination in PS19-fE4/Syn1-Cre mice. Differentially expressed (DE) gene analysis revealed that cells in cluster 25 had significant upregulated expression of three major heat shock protein genes, human MAPT gene, and amyloid precursor protein (App) gene. Differentially expressed (DE) pathway analysis revealed the enrichment of KEGG pathways related to general neurodegeneration, Alzheimer's Disease, and other neurodegenerative diseases, indicating that cluster 25 represents neurodegenerative disease-associated neurons.

Similarly, log odd ratio estimates from another Generalized Linear Mixed Effects Model to assess associations with histopathology (GLMM_histopathology) revealed that the proportion of cells in clusters 17 and 25 both exhibited significant negative associations with hippocampal volume and significant positive associations with the coverage area of pTau as well as astrogliosis and microgliosis. The associations were largely driven by the PS19-fE4 group and, to a lesser extent, by the PS19-fE4/GFAP-Cre group. The proportion of cells in cluster 17 also had a significant negative association with MBP coverage area.

Taken together, these findings illustrate that removal of neuronal, but not astrocytic APOE4, leads to a drastic reduction in the amount of neurodegenerative disease-associated oligodendrocytic and neuronal subpopulations within the hippocampus and that these two cell populations have strong associations with Tau pathology, gliosis, neurodegeneration, and oligodendrocyte deficits.

### Example 10: Neurodegenerative Disease-Associated Astrocyte and Microglia Subpopulations are Largely Eliminated by Removing Neuronal, but not Astrocytic, APOE4

Further subclustering of astrocytes identified 18 subpopulations. Log odds ratio estimates from a GLMM_AM revealed that astrocyte subclusters 4 and 7 had lower odds of having cells from PS19-fE4/Syn1-Cre mice than from PS19-fE4 mice, with a complete elimination of subcluster 7 in PS19-fE4/Syn1-Cre mice. Interestingly, astrocyte subclusters 4 and 7 highly expressed APOE. Differentially expressed (DE) gene and pathway analysis revealed the enrichment of KEGG pathways related to general neurodegeneration, Alzheimer's Disease, and other neurodegenerative diseases in subcluster 7, indicating that subcluster 7 represents neurodegenerative disease-associated astrocytes. Subcluster 4 showed an enrichment of KEGG pathways related to cAMP signaling, synaptic function, and long-term potentiation, indicating that subcluster 4 represents synaptic dysfunction-associated astrocytes.

Further subclustering of microglia also identified 18 subpopulations. Log odds ratio estimates from a GLMM_AM revealed that microglia subclusters 4 and 8 had lower odds of having cells from PS19-fE4/Syn1-Cre mice than from PS19-fE4 mice, with a complete elimination of subcluster 8 in PS19-fE4/Syn1-Cre mice. Interestingly, microglia subclusters 4 and 8 highly expressed APOE. Differentially expressed (DE) gene and pathway analysis revealed the enrichment of KEGG pathways related to general neurodegeneration, AD, and other neurodegenerative diseases in subcluster 8, indicating that subcluster 8 represents neurodegenerative disease-associated microglia. Subcluster 4 showed an enrichment of KEGG pathways related to synaptic function, cAMP signaling, and long-term potentiation, suggesting that subcluster 4 represents synaptic dysfunction-associated microglia.

Similarly, log odds ratio estimates from a GLMM_histopathology revealed that the proportion of cells in astrocyte subcluster 7 and microglia subcluster 8 both exhibited significant negative associations with hippocampal volume and significant positive associations with the coverage area of pTau as well as astrogliosis and microgliosis. The associations were largely driven by the PS19-fE4 group and, to a lesser extent, by the PS19-fE4/GFAP-Cre group. Interestingly, PCA clustering of oligodendrocyte cluster 17 and excitatory neuron cluster 25 together with all astrocyte and microglia subclusters illustrated that the disease-associated astrocyte subcluster 7 and microglia subcluster 8 have similar contributions to the eight pathological parameters as the two disease-associated clusters of oligodendrocytes (cluster 17) and neurons (cluster 25).

Taken together, these findings illustrate that removal of neuronal, but not astrocytic APOE4, leads to a drastic reduction in the amount of neurodegenerative disease-associated astrocyte and microglia subpopulations within the hippocampus and that these two glial subpopulations have strong associations with Tau pathology, gliosis, and neurodegeneration.

### Example 11: Removal of Astrocytic APOE4 Secondarily Reduces Neuronal APOE4 Expression

During analysis of snRNA-seq data, the inventors noticed that removal of astrocytic APOE also drastically reduced APOE expression in neurons in addition to its predicted elimination of APOE expression in astrocytes. To confirm this observation, a publicly available snRNA-seq dataset (GEO:GSE164507) was further analyzed that utilized a similar compound mouse model carrying a floxed-knock-in human APOE gene and an astrocyte-specific Cre (Aldh111-Cre/ERT2 BAC transgene) that deletes the human APOE gene in astrocytes in a tamoxifen-inducible manner during adulthood, along with either the presence or absence of P301S mutant human Tau (Wang et al., Neuron 109, 1657-1674.e7 (2021)).

In this snRNA-seq dataset, neuronal APOE expression was observed in both FE3 and FE4 mice, which was further increased in PS19-FE4 (TAFE4_oil) and PS19-FE3 (TAFE3_oil) mice, indicating that tauopathy increases neuronal APOE expression. Upon removal of astrocytic APOE induced by tamoxifen treatment during adulthood, a drastic reduction of neuronal APOE in both excitatory and inhibitory neurons was also clearly observed in PS19-fE4/Aldh111-Cre/ERT2 (TAFE4_tam) and PS19-fE3/Aldhlll-Cre/ERT2 (TAFE3 tam) mice relative to mice treated with oil. The effects of removing astrocytic APOE on reduction of neuronal APOE were observed in all subtypes of neurons, indicating a general regulation of neuronal APOE expression by astrocytic APOE.

Thus, the initial observation that removal of astrocytic APOE secondarily reduces neuronal APOE expression is confirmed in a different tauopathy model with human APOE, even when astrocytic APOE removal was driven under a different astrocyte-specific promoter and its removal was induced at a different timepoint in the lifespan of the mouse model.

To determine whether this observation can be validated at the protein level, the levels of APOE protein were quantitatively evaluated via sandwich ELISA in cultured primary neurons isolated from the cortex and hippocampus of prenatal pups. The analysis revealed that primary neurons from PS19-fE4/GFAP-Cre mice exhibited a significant 50% decrease in APOE protein levels relative to those from PS19-fE4 mice (FIG. 4M).

These data provide strong evidence that the removal of astrocytic APOE4 leads to a drastic decrease in neuronal APOE4 expression and protein production and solidifies this regulatory relationship between astrocytic and neuronal APOE4. Thus, the limited protective effects of removing astrocytic APOE4 are likely attributable to a secondary reduction, but not complete elimination, of neuronal APOE4.

Early studies by the inventors and coworkers reported that neuronal APOE expression is regulated by astrocyte-released factor(s) through modulation of the ERK pathway within neurons (Zhao et al., Nat. Commun. 11, 5540 (2020)). An in-depth comparison of snRNA-seq data between astrocytes from PS19-fE4/GFAP-Cre mice and those from PS19-fE4 mice revealed that some of the top down-regulated genes after astrocytic APOE4 removal were documented activators of the ERK/MAPK pathway, including dipeptidyl peptidase 10 (DPP10) and Neuregulin-3 (Nrg3).

The inventors hypothesized that astrocytic APOE4 may regulate neuronal APOE4 expression by promoting the release of these factors that activate the neuronal ERK pathway, leading to increased neuronal APOE4 expression. To test this possibility, primary neurons isolated from PS19-fE4 and PS19-fE4/GFAP-Cre mice were treated with 10 μg/mL of recombinant DPP10 or Nrg3 protein or a dPBS control. The APOE protein levels were then quantitatively evaluated in the primary neuronal cell lysates by sandwich ELISA.

PS19-fE4/GFAP-Cre primary neurons treated with DPP10 had a striking 98% increase in APOE protein levels relative to the dPBS control (FIG. 4N). On the other hand, there was no significant difference in APOE levels in PS19-fE4 primary neurons following DPP10 treatment (FIG. 4O). These data indicate that primary neurons from PS19-fE4/GFAP-Cre mice, in which the astrocytic APOE4 was removed, are sensitive to DPP10 upregulation of APOE expression, while primary neurons from PS19-fE4 mice are not. We did not observe differences in APOE levels in PS19-fE4/GFAP-Cre or PS19-fE4 primary neurons following treatment with Nrg3 (FIG. 4N-4O), indicating that Nrg3 is not involved in the regulation of neuronal APOE4 expression.

### Example 12: Neuronal Release of HMGB1 is Completely Eliminated by Removing Neuronal, but not Astrocytic, APOE4

In searching for pro-inflammation cytokines that could trigger Tau-induced and neurodegeneration-induced gliosis, the inventors observed that cells in the neurodegenerative disease-associated neuronal cluster 25 had drastically increased expression of the high mobility group box 1 (Hmgb1) gene. The HMGB1 protein is needed for glial cell activation (Paudel et al. Front. Neurosci. 12, 628 (2018)). Under pathological conditions, HMGB1 translocates from the nucleus to the cytoplasm of stressed or dying cells and it is then released to act as a proinflammatory cytokine.

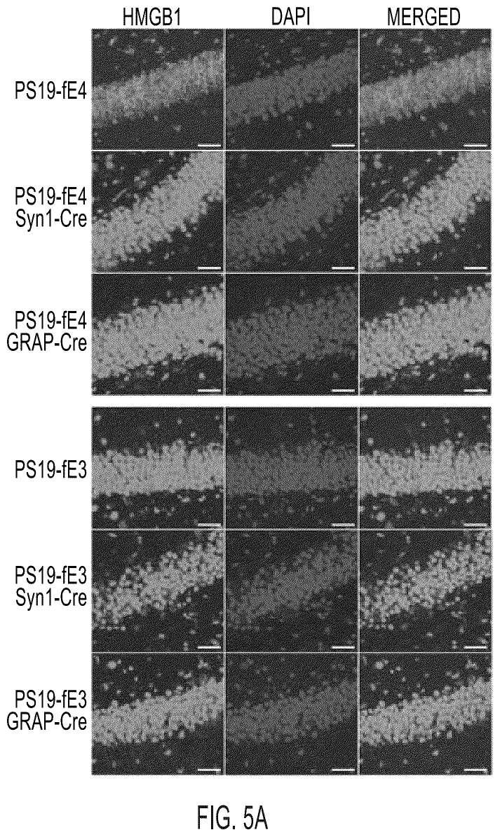

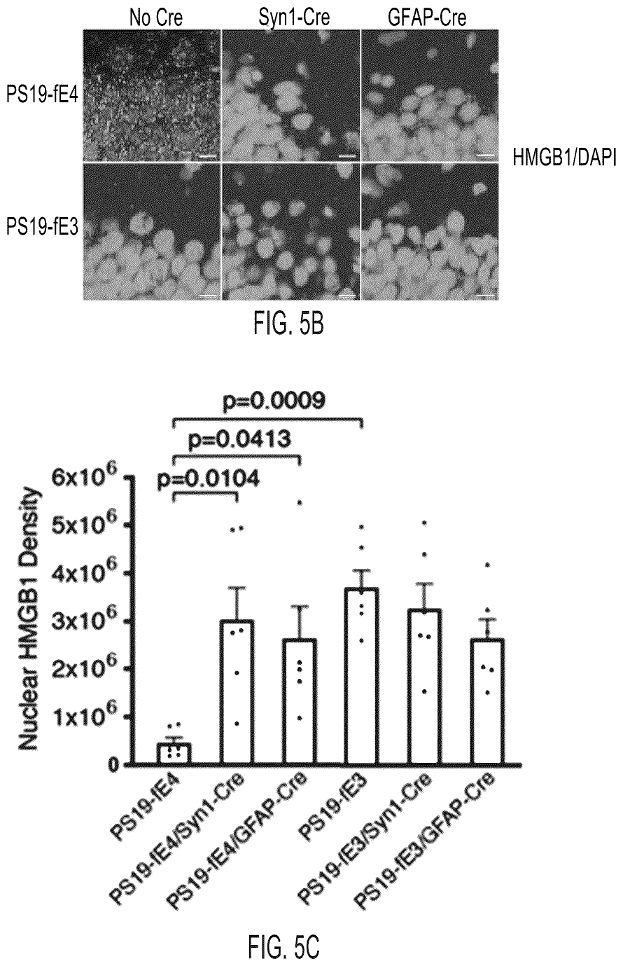

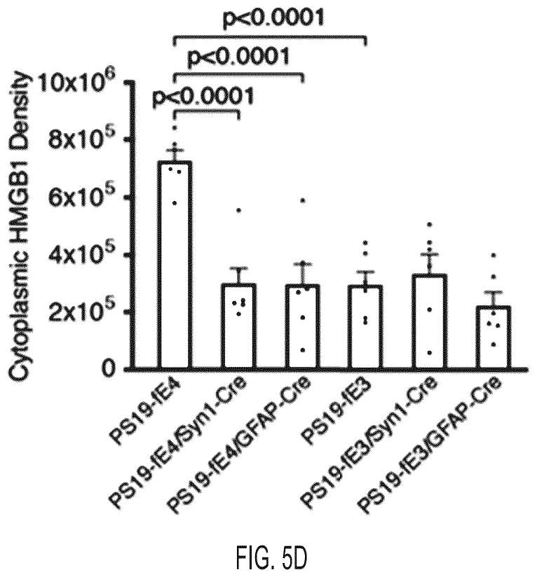

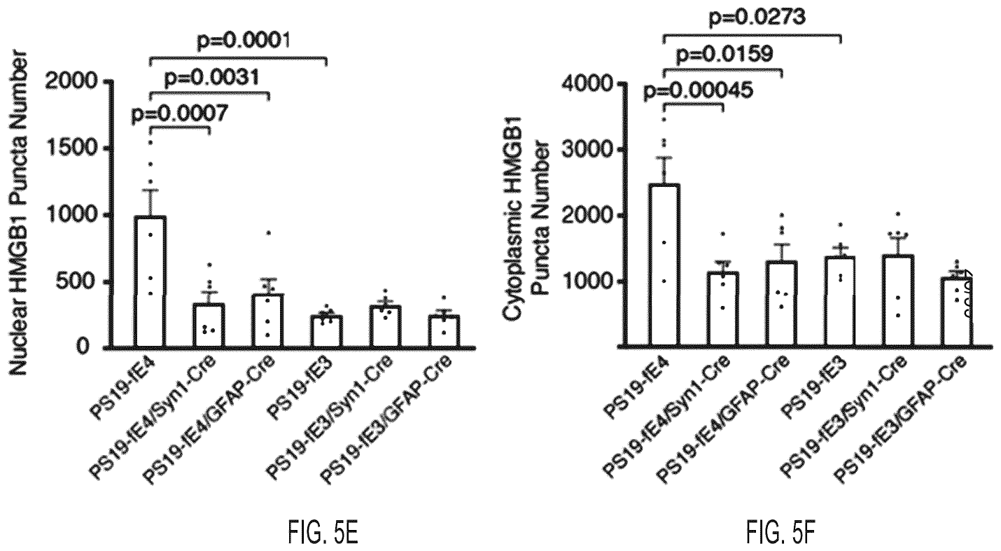

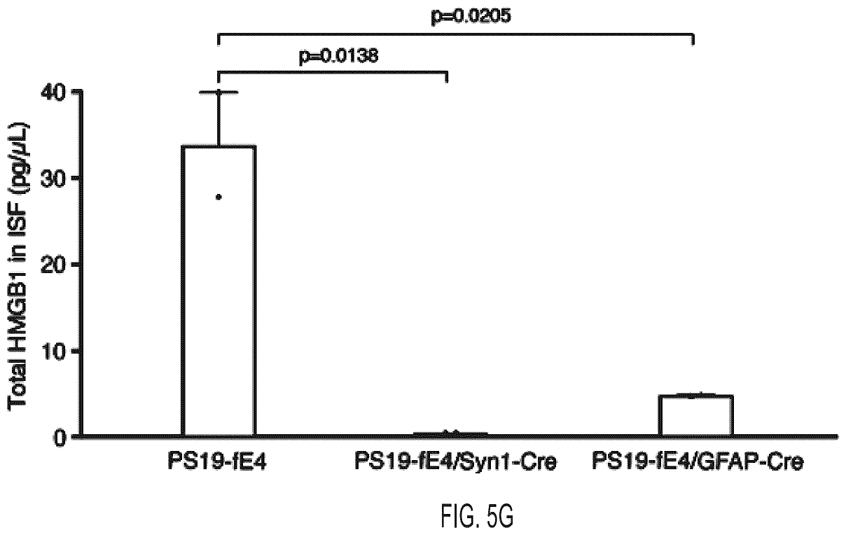

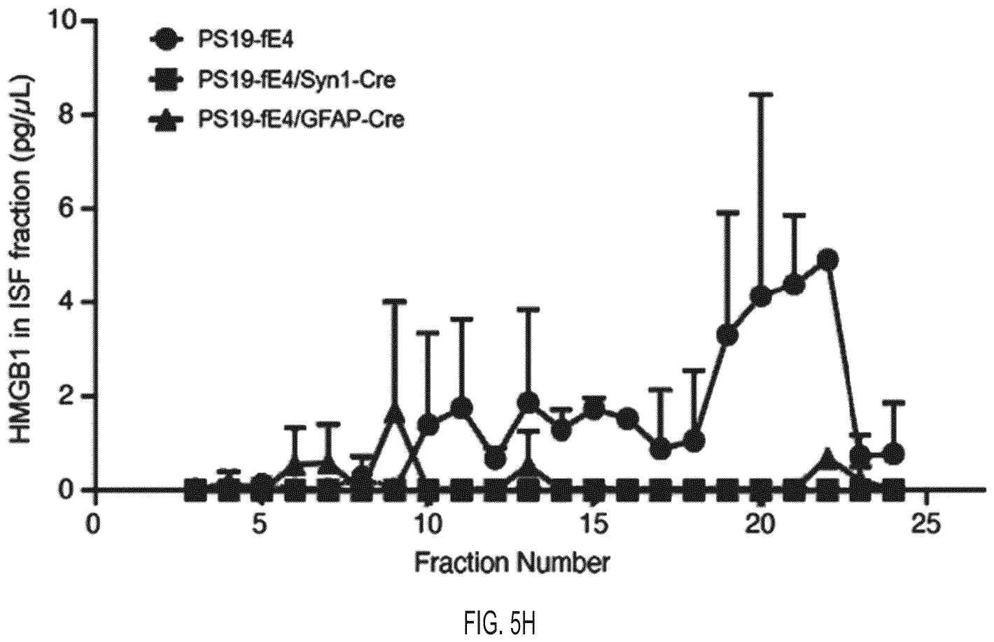

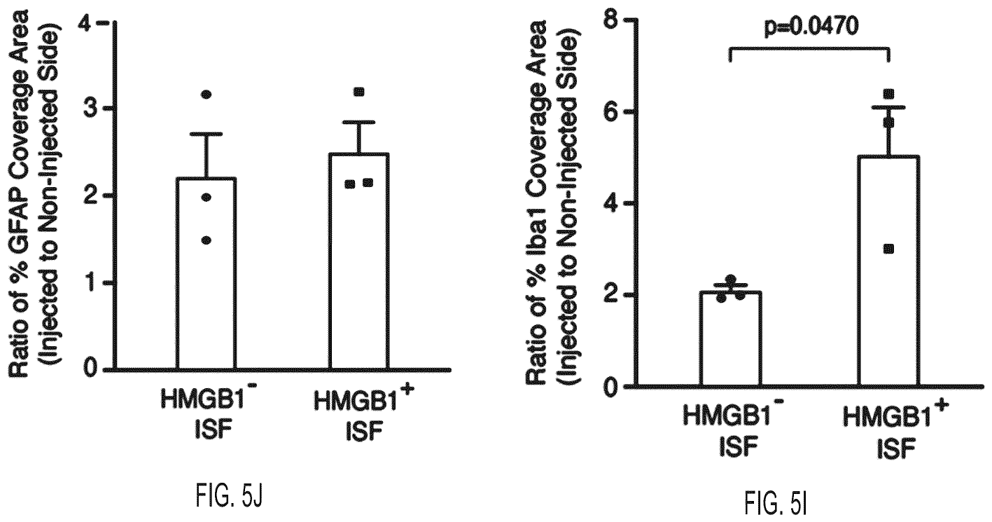

Immunohistochemical staining for HMGB1 protein and the nuclear marker DAPI in 10-month-old mice revealed that PS19-fE4 mice had a remarkably high amount of HMGB1 protein that translocated from the nucleus to the cytoplasm of neurons in the dentate gyrus of the hippocampus, as a significant majority of the protein can be found located outside of the nucleus of the hippocampal neurons (FIG. 5A-5D). On the other hand, HMGB1 protein in PS19-fE3 mice was largely retained within the nucleus and there was minimal translocation to the cytoplasm (FIG. 5A-5D). Interestingly, removal of APOE4 from either neurons or astrocytes attenuates this phenotype observed in PS19-fE4 mice, with the majority of HMGB1 protein remaining localized to the nucleus (FIG. 5A-5D).

Furthermore, PS19-fE4 mice contained a greater number of HMGB1-positive puncta in both the nucleus and the cytoplasm relative to the other genotype groups (FIG. 5E-5F). Notably, fE4 and fE3 mice that lacked human mutant Tau-P301S had HMGB1 protein localized to the nucleus, illustrating that the nucleo-cytoplasmic translocation of HMGB1 in APOE4 mice requires the coexistence of both APOE4 and tauopathy.

To evaluate the extent of disease-associated HMGB1 release from hippocampal neurons within these various genotype groups, the hippocampal interstitial fluid (ISF) from 8.5-month-old mice was collected over a 24 hour period using in vivo microdialysis (Yamada et al., J. Neurosci. 31: 13110-13117 (2011)). The levels of HMGB1 protein in the interstitial fluid (ISF) was quantitatively determined using sandwich ELISA. While PS19-fE4 mice exhibited high levels of HMGB1 protein within their hippocampal ISF, removal of neuronal APOE4 reduced HMGB1 protein levels down to an undetectable level in the ISF (FIG. 5G-5H), indicating that neuronal APOE4 plays a role in controlling HMGB1 release from neurons. Removal of astrocytic APOE4 also led to an 86% reduction, but not complete elimination, of HMGB1 protein in the ISF (FIG. 5G-5H), which is likely due to the secondary reduction, but not complete elimination, of neuronal APOE4 expression.

Experiments were also performed to determine whether HMGB1 is a potential modulatory factor of neuronal APOE4 expression. As such, primary neurons isolated from PS19-fE4/GFAP-Cre and PS19-fE4 mice were treated with 10 μg/mL of recombinant HMGB1 protein or a dPBS control and the APOE protein levels were quantitatively evaluated in cell lysates by sandwich ELISA. A significant difference was not observed in APOE protein levels of PS19-fE4/GFAP-Cre or PS19-fE4 primary neurons following treatment with HMGB1, indicating that HMGB1 is not involved in the regulation of neuronal APOE4 expression.

To evaluate the importance of neuronally released HMGB1 in triggering gliosis in APOE4-related tauopathies, interstitial fluid (ISF) collected from the hippocampus of an 8.5-month-old PS19-fE4 mouse was injected into the hippocampi of 8.5-month-old wildtype mice. The injected ISF fractions were either enriched with relatively high concentrations of HMGB1 protein (fractions 19-22 in FIG. 5H) or had undetectable levels of HMGB1 protein as a control (fractions 4-7 in FIG. 5H), as determined by sandwich ELISA. The wildtype mice received a unilateral injection into the right dorsal hippocampus of either HMGB1⁺ or control ISF and were analyzed 6 days post-injection to assess acute changes in gliosis.

Wildtype mice that were injected with the control (HMGB1⁻) ISF exhibited relatively low levels of microgliosis and astrogliosis on both the injected and non-injected hippocampal sides, illustrating that the majority of gliosis induced by the injection surgery itself subsided by 6 days post-injection (FIG. 5I-5J). On the contrary, wildtype mice injected with the HMGB1⁺ISF displayed significantly increased levels of microgliosis on the injected hippocampal side relative to the non-injected side (FIG. 5I). Interestingly, injection of HMGB1⁺ ISF does not lead to a significant increase in astrogliosis in the injected hippocampal side relative to the non-injected side (FIG. 5J). Taken together, these data indicate that the neuronal release of HMGB1 represents a novel mechanism by which neuronal APOE4 promotes glial cell activation.

### Example 13: Treatment with HMGB1 Inhibitors Substantially Reduces APOE4-Driven Gliosis, Tau Pathology, and Degeneration

Based on the findings that neuronal APOE4 is a potent driver of neuronal HMGB1 release, experiments were performed to test the therapeutic efficacy of HMGB1 inhibitors in combating APOE4-driven pathogenesis in the context of tauopathy.

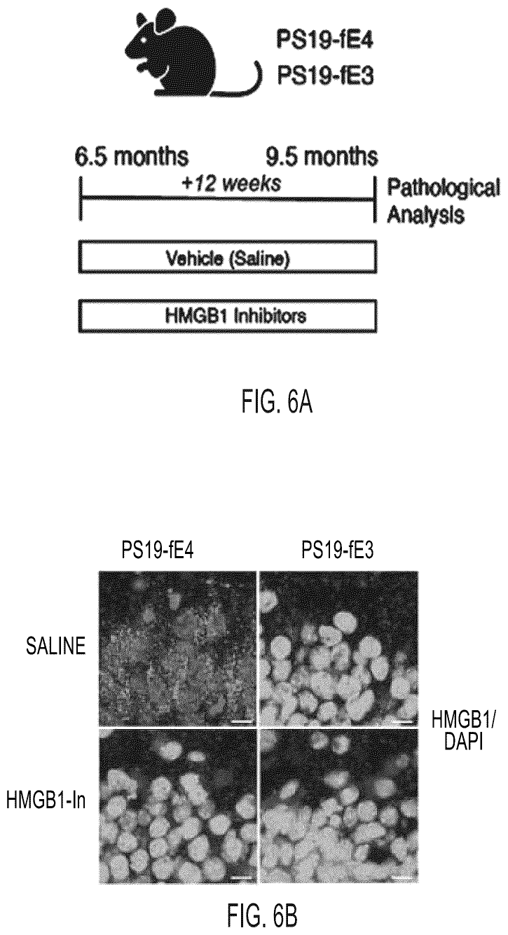

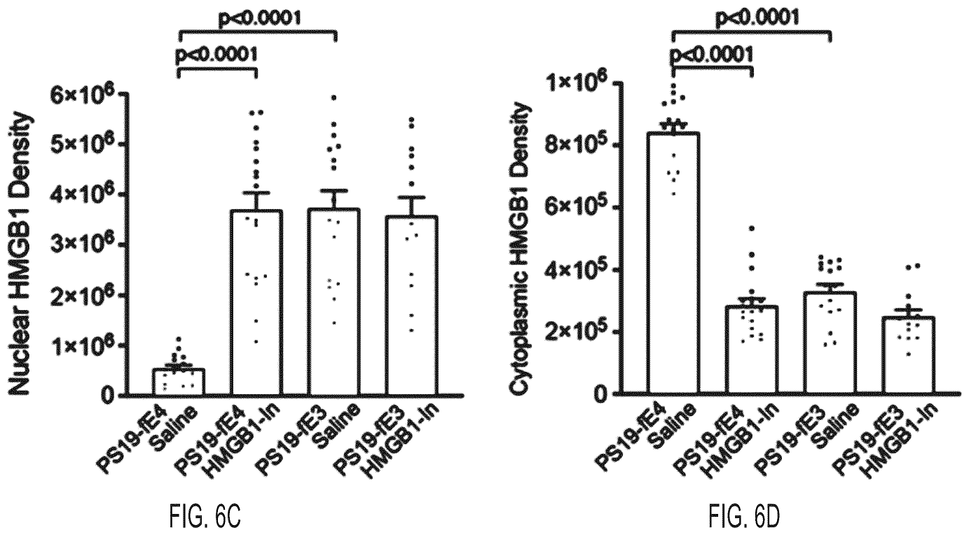

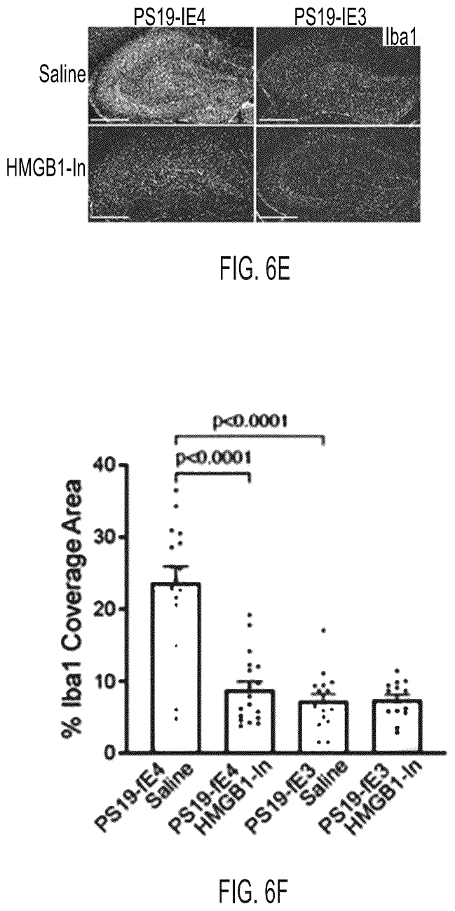

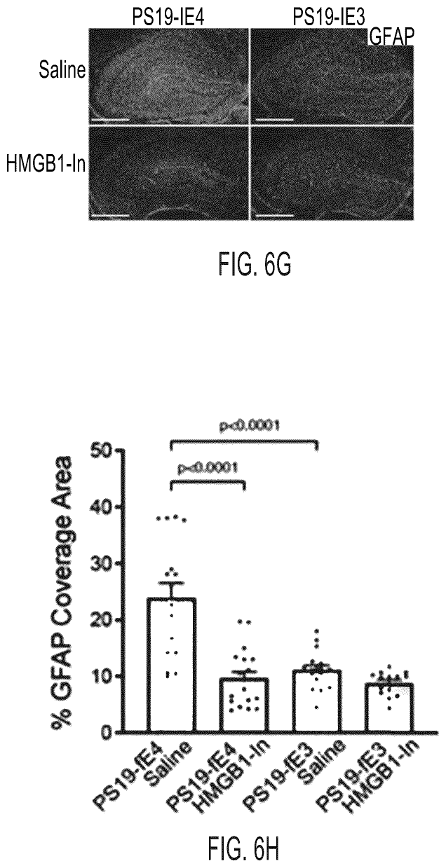

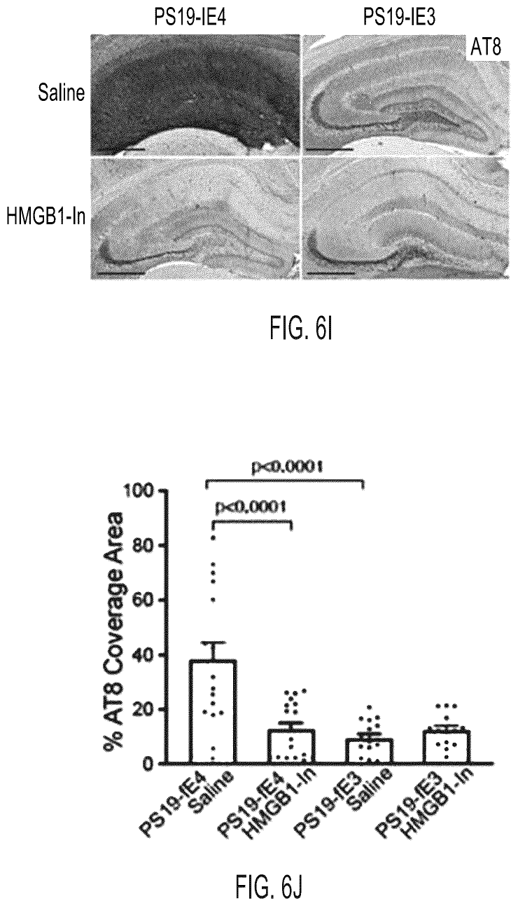

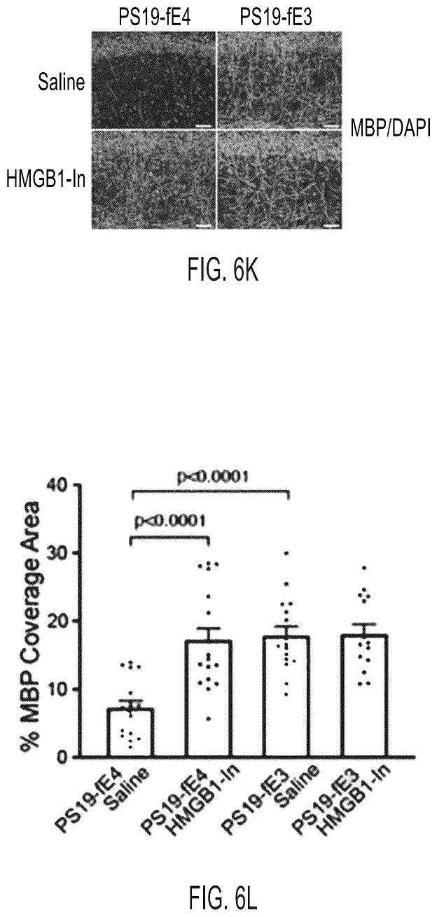

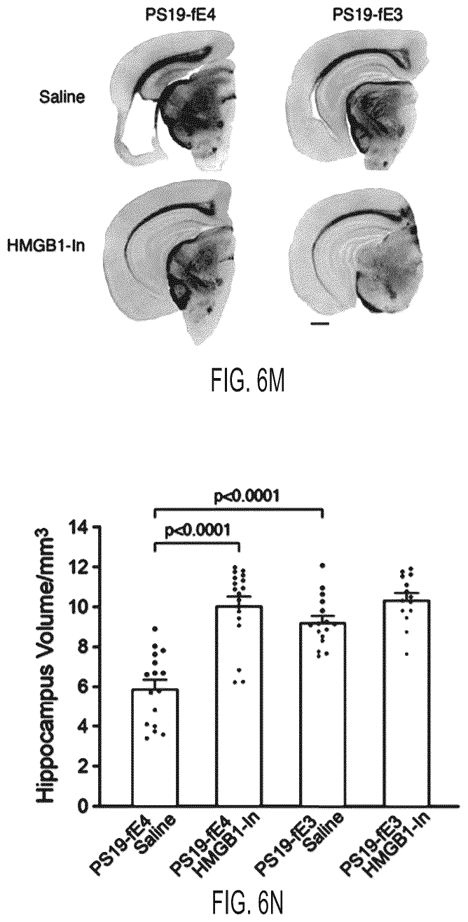

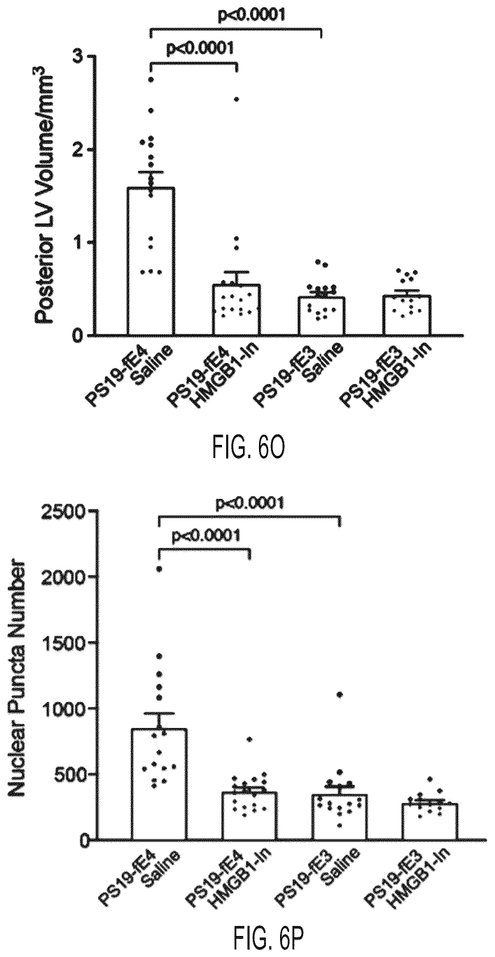

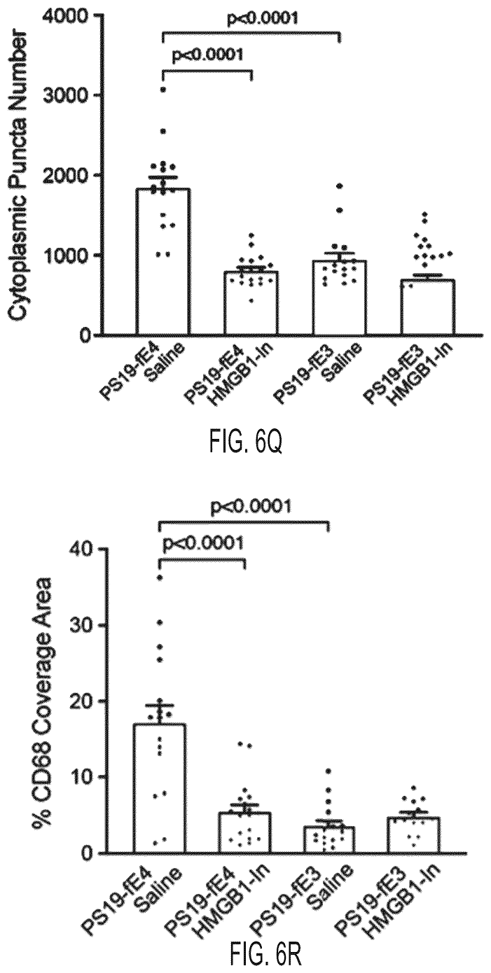

Two HMGB1 inhibitors were tested, ethyl pyruvate (EP) and glycyrrhizic acid (GA), which are selective inhibitors of HMGB1 translocation and release (Ulloa et al., Proc. Natl. Acad. Sci. USA 99: 12351-12356 (2002); Sun et al., Front. Immunol. 9, 1518 (2018); Mollica et al., Chem Biol. 14, 431-41 (2007); Dave et al., J. Leukoc. Biol. 86, 633-643 (2009)). A mixed solution of ethyl pyruvate (80 mg/kg) and glycyrrhizic acid (20 mg/kg) or saline vehicle was administered to PS19-fE4 and PS19-fE3 mice in a regimen of three doses per week for 12 weeks via intraperitoneal injections (FIG. 6A). Treatment began when the mice were 6.5 months of age, at about the onset of adverse pathology, and completed when the mice were 9.5 months of age, when severe neurodegeneration and pathological changes are typically present, as demonstrated in this and other studies (Shi et al., Nature 549, 523-527 (2017); Yoshiyama et al., Neuron 53: 337-351 (2007)).

The effectiveness of ethyl pyruvate (EP) and glycyrrhizic acid (GA) for blocking HMGB1 protein nucleo-cytoplasmic translocation was first confirmed by immunostaining with anti-HMGB1 and DAPI. Saline-treated PS19-fE4 mice exhibited extensive HMGB1 translocation out of the nucleus, whereas PS19-fE4 mice treated with the HMGB1 inhibitors had no discernable HMGB1 translocation (FIG. 6B-6D; 6P-6Q). In PS19-fE3 mice, there was no significant difference in HMGB1 translocation between saline- and inhibitor-treated groups.

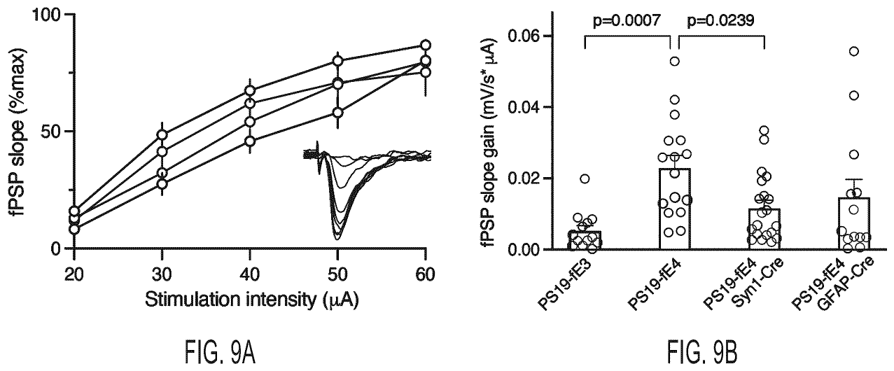

The extent of microgliosis and astrogliosis present in the mice following treatment was then evaluated. Saline-treated PS19-fE4 mice displayed considerable microgliosis throughout the hippocampus, as exemplified by a high coverage area of Iba1⁺ microglia (FIG. 6E-6F) and CD68⁺ activated microglia (FIG. 9R). whereas HMGB1 inhibitor-treated PS19-fE4 mice displayed a significant decrease in microgliosis. Furthermore, immunostaining with anti-GFAP revealed that saline-treated PS19-fE4 mice had extensive astrogliosis throughout the hippocampus, while HMGB1 inhibitor-treated PS19-fE4 mice had a significant reduction in astrogliosis (FIG. 6G-6H). PS19-fE3 mice did not display significant differences in microgliosis or astrogliosis whether treated with saline or HMGB1 inhibitors.

The extent of Tau pathology was then evaluated in these mice by immunostaining for pTau. PS19-fE4 mice treated with saline exhibited substantial Tau pathology throughout the hippocampus, whereas PS19-fE4 mice treated with HMGB1 inhibitors had a drastic reduction in Tau pathology (FIG. 6I-6J). Furthermore, the effectiveness of HMGB1 inhibitor administration was determined in combating myelin deficits by immunostaining for MBP. Saline-treated PS19-fE4 mice displayed severe myelin loss in the stratum radiatum of CA1, while HMGB1 inhibitor-treated PS19-fE4 mice displayed a rescue of the deficit with a high coverage area of MBP (FIG. 6K-6L). There were no discernable differences in myelin coverage area or Tau pathology between saline- and inhibitor-treated PS19-fE3 mice.

The impact of HMGB1 inhibitors on neurodegeneration was also determined by quantifying hippocampi and posterior lateral ventricle volumes. Saline-treated PS19-fE4 mice exhibited considerable neurodegeneration, whereas HMGB1 inhibitor-treated PS19-fE4 mice exhibited a rescue of neurodegeneration through a significant increase in hippocampal volume and a significant decrease in posterior lateral ventricle volume relative to saline-treated PS19-fE4 mice (FIG. 6M-6O). There were no obvious differences in neurodegeneration between saline- and HMGB1 inhibitor-treated PS19-fE3 mice.

Taken together, these findings illustrate that treatment of PS19-fE4 mice with HMGB1 inhibitors can effectively prevent the development of prominent APOE4-driven pathologies, including gliosis, Tau pathology, neurodegeneration, and myelin deficits, by blocking the nucleo-cytoplasmic translocation and subsequent release of HMGB1 protein. The data support a conclusion that HMGB1 translocation and release plays a role in the induction and exacerbation of these various APOE4-driven pathologies.

### Example 14

Apolipoprotein E4 (APOE4) is the major genetic risk factor for Alzheimer's disease (AD), however, its underlying cellular and molecular mechanisms remain elusive. In the brain, APOE is produced mainly by astrocytes and at lower levels by neurons. Described herein is a rigorous comparison of neuronal and astrocytic APOE4 effects on AD-related pathologies by selectively removing APOE4 from either cell type in mice carrying a human P301S mutant Tau transgene and a floxed-knock-in human APOE gene. The APOE4 mice had significantly more Tau pathology, gliosis, neurodegeneration, neurodysfunction, and myelin deficits than APOE3 mice. Single-nucleus RNA-sequencing identified an enrichment of neurodegenerative disease-associated subpopulations of neurons, oligodendrocytes, astrocytes, and microglia in APOE4 mice. Removing neuronal APOE4 drastically reduced all these pathologies and largely eliminated the disease-associated cell subpopulations, while removing astrocytic APOE4 only reduced gliosis and neurodegeneration. The limited protective effects of removing astrocytic APOE4 are attributable to a secondary reduction, but not complete elimination, of neuronal APOE4. Mechanistically, neuronal APOE4 promoted HMGB1 release from neurons to induce gliosis and subsequent neurodegeneration and myelin deficits, which were effectively blocked by treatment with HMGB1 inhibitors. Thus, neuronal APOE4 drives Tau-mediated inflammation and degeneration by promoting neuronal HMGB1 release, and HMGB1 inhibitors represent a novel approach for treating APOE4-related AD and other tauopathies.

### Introduction

Tauopathies are a class of neurodegenerative disorders defined by the abnormal intracellular accumulation of hyperphosphorylated Tau (pTau) protein. Alzheimer's disease (AD), which represents a major type of tauopathy, is a highly prevalent disorder that is characterized pathologically by the accumulation of amyloid plaques and Tau tangles and clinically by the loss of memory and other cognitive functions. Of these two major pathological hallmarks of AD, only Tau tangles strongly correlate with neurodegeneration and cognitive decline. Other AD pathological hallmarks that have been understudied include neuroinflammation and gliosis, which have recently been identified as major contributing factors to neurodegeneration. Additionally, oligodendrocyte deficits and myelin degeneration have been observed in human AD brains and in mouse models of AD and tauopathy. Thus, AD represents a complex set of pathologies, although the connections between these pathologies remain unclear. Understanding the relationships between pathologies and elucidating the underlying mechanisms responsible for their induction or exacerbation is important for developing better therapeutic strategies targeting these pathologies, either individually or in combination.

Epidemiological and genome-wide association studies have identified apolipoprotein E4 (APOE4) as the major genetic risk factor for AD. The human APOE gene exists as three common alleles, including E2, E3, and E4. APOE E4 is considered the most detrimental allele as it dose-dependently increases AD risk and decreases the age of disease onset. There have been great efforts to understand how APOE4 leads to increased AD risk, with an extensive body of work illustrating that APOE4 worsens many prominent AD-related pathologies relative to APOE. In particular, APOE4 has been shown to accelerate hippocampal volume loss in human patients and to increase neurodegeneration in mice with or without tauopathy. In addition to its well-studied roles in promoting amyloid pathology, recent studies have found that APOE4 also increases Tau burden in human brains and promotes the accumulation of pTau in mouse and human neuron models. Furthermore, APOE4 increases neuroinflammation and gliosis in human AD brains and in tauopathy mouse models. It has also been reported that APOE4 is associated with reduced myelination and white matter integrity in human brains. Together, these studies show clear evidence that APOE4 is implicated in promoting Tau pathology, gliosis, neurodegeneration, and myelin degeneration in AD and other tauopathies. Nonetheless, the underlying mechanisms responsible for APOE4's wide-ranging effects on these various pathologies remain unclear.

Recently, there has been increasing interest in establishing the cell type-specific effects of APOE4 in various aspects of AD pathogenesis. Within the central nervous system (CNS), APOE acts as the primary lipid transporter and is mainly produced by astrocytes. Conditions of stress or injury can induce APOE expression in neurons and microglia. Previous studies have indicated that the detrimental effects of APOE4 may depend on its cellular source. However, there is a gap in the understanding of the exact roles of neuronal and astrocytic APOE4 in triggering and/or exacerbating the various AD pathologies. In the current study, an extensive analysis was conducted directly comparing the impact of removing APOE4 from neurons or astrocytes on the development of major AD pathologies, including Tau pathology, gliosis, neurodegeneration, neurodysfunction, and myelin deficits, to better understand the cell type-specific roles of APOE4 in the pathogenesis of AD and other tauopathies. It was an aim to uncover key cellular and molecular mechanisms involved in triggering a cascade of AD-related pathologies and to determine whether these mechanisms are regulated by neuronal or astrocytic APOE4.

### Results

### Neuron- or Astrocyte-Specific Removal of the APOE Gene in Human APOE-KI Mice Expressing Human Mutant Tau

Mouse lines were generated expressing a floxed human APOE3 or APOE4 gene and a Cre recombinase gene under the control of a neuron-specific Synapsin-1 promoter (Syn1-Cre) or an astrocyte-specific Glial Fibrillary Acidic Protein promoter (GFAP-Cre). These floxed APOE-KI (fE) mice express homozygous human APOE3 or APOE4 in place of the endogenous mouse Apoe, and the human APOE gene is flanked by a pair of LoxP sites to allow for its precise excision in the presence of cell-type-specific Cre recombinase expression. The fE mice with no Cre, Syn1-Cre, or GFAP-Cre were crossbred with mice expressing mutant 1N4R human microtubule-associated protein Tau (MAPT) encoding the disease-associated P301S mutation (PS19 line), which has been widely utilized as a tauopathy mouse model. The resulting compound mice are referred to as PS19-fE, PS19-fE/Syn1-Cre or PS19-fE/GFAP-Cre mice.

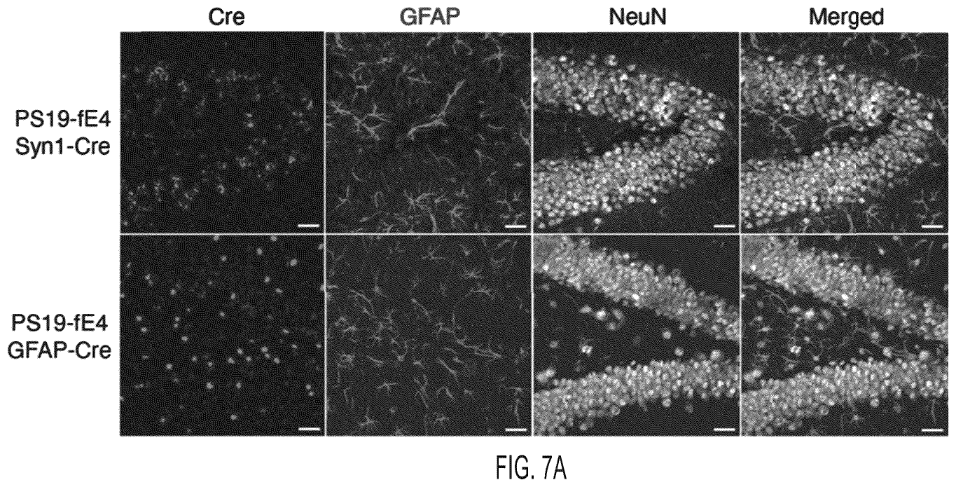

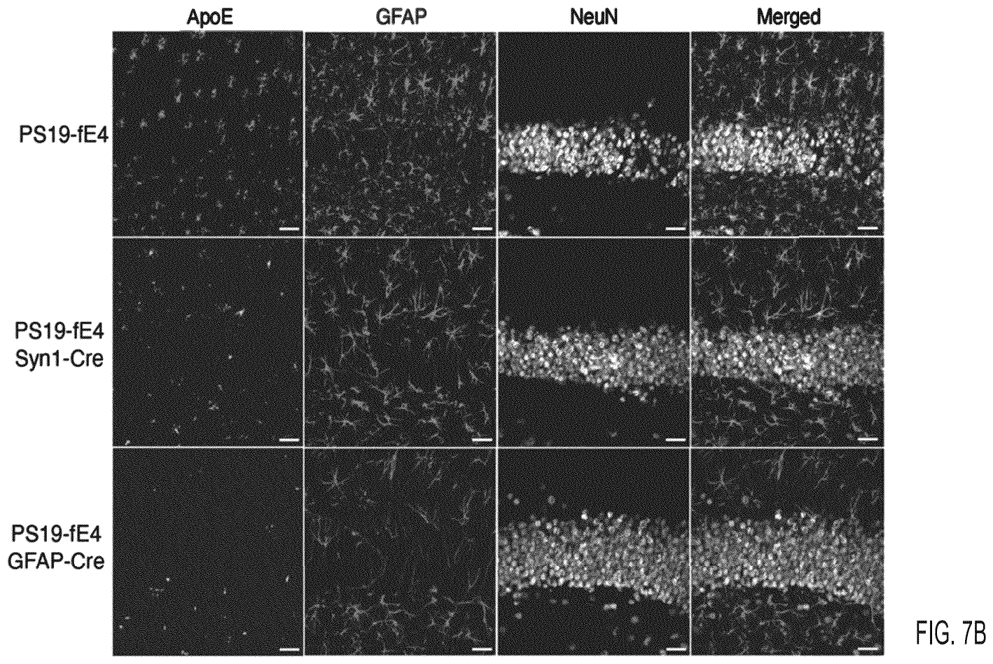

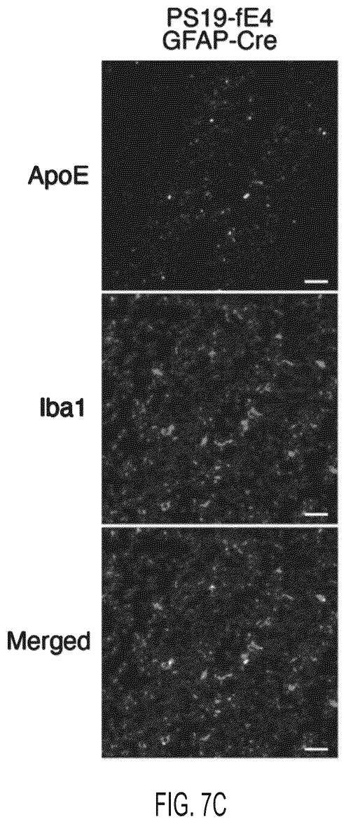

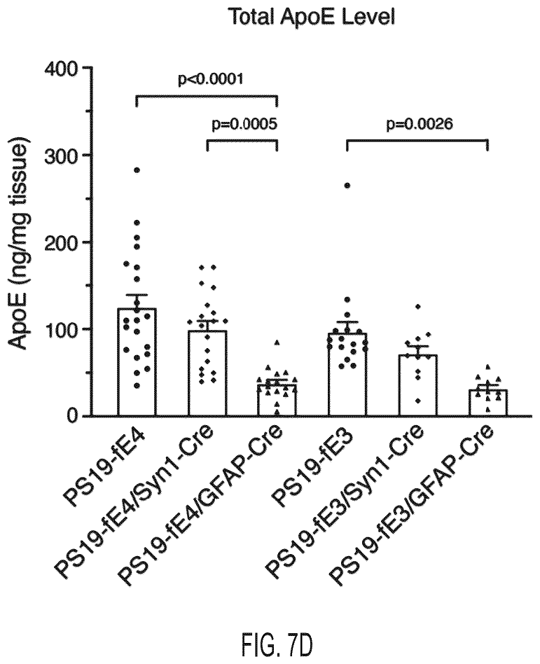

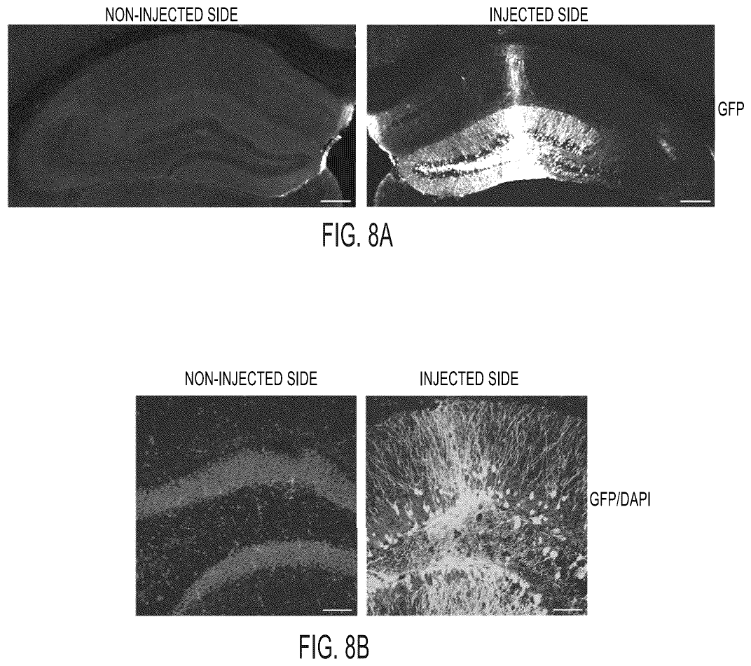

Rigorous characterization of the fE/Syn1-Cre and fE/GFAP-Cre mice had been conducted to validate the specificity of Cre recombinase expression under the neuron-specific Syn1 or the astrocyte-specific GFAP promoter. The PS19-fE/Cre mice were further evaluated by performing immunohistochemical analysis of brain sections from 10-month-old PS19-fE4/Syn1-Cre and PS19-fE4/GFAP-Cre mice. To confirm the specificity of Cre recombinase expression in hippocampal neurons and astrocytes when driven under the Syn1 or GFAP promoter, respectively, NeuN and GFAP antibodies along with a Cre recombinase antibody were employed. In PS19-fE4/Syn1-Cre mice, Cre recombinase was expressed exclusively in NeuN-positive neurons and was not expressed in GFAP-positive astrocytes (FIG. 7A). In PS19-fE4/GFAP-Cre mice, Cre recombinase was expressed exclusively in GFAP-positive astrocytes and was not expressed in NeuN-positive neurons (FIG. 7A). Co-immunostaining using antibodies against APOE, GFAP, and NeuN illustrates that PS19-fE4 mice without Cre have APOE expression in GFAP-positive astrocytes and some NeuN-positive neurons, whereas PS19-fE4/Syn1-Cre mice have APOE expression in astrocytes and lack APOE expression in neurons and PS19-fE4/GFAP-Cre mice have APOE expression in some neurons and lack APOE expression in astrocytes (FIG. 7B). The PS19-fE4/GFAP-Cre mice exhibited some APOE-positive cells that were negative for NeuN and GFAP (FIG. 7B) but positive for the microglial marker Iba1 (FIG. 7C), indicating that they were APOE-expressing microglia.

In order to quantitatively determine the levels of APOE protein within these various mouse models, hippocampal lysates of 10-month-old mice were analyzed by sandwich ELISA for human APOE. PS19-fE4/Syn1-Cre mice exhibited a ˜20% decrease in APOE levels relative to PS19-fE4 mice (FIG. 7D), which aligns with previous reports indicating that neuronal APOE contributes to ˜20-30% of total APOE protein levels in the hippocampus and cortex26,42. PS19-fE4/GFAP-Cre mice exhibited a drastic ˜70% decrease in APOE levels relative to PS19-fE4 mice, which agrees with the well-established role of astrocytes as being the main producers of APOE within the CNS. Similarly, PS19-fE3/Syn1-Cre mice exhibited a ˜25% decrease and PS19-fE3/GFAP-Cre exhibited a ˜67% decrease in APOE levels relative to PS19-fE3 mice (FIG. 7D). Taken together, these results provide strong evidence that APOE gene expression is eliminated in neurons or astrocytes when Cre recombinase expression is driven under a Syn1 or GFAP promoter, respectively, in these compound mouse models.

Removal of APOE4 from Neurons, but not Astrocytes, Drastically Reduces Tau Pathology

To determine whether the removal of neuronal or astrocytic APOE affects Tau pathology, mice at 10 months of age, which is when PS19 mice exhibit extensive Tau pathology throughout the hippocampus, were assessed. The accumulation of pTau in the hippocampus was assessed by immunohistochemical staining with the pTau-specific AT8 antibody and quantified Tau pathology as the percent of AT8 coverage area in the hippocampus. PS19-fE4 mice presented extensive Tau pathology throughout the hippocampus and the extent of Tau pathology was significantly lower in PS19-fE3 mice. Relative to PS19-fE4 mice, the PS19-fE4/Syn1-Cre mice exhibited a striking reduction (˜81%) in Tau pathology, whereas PS19-fE4/GFAP-Cre mice had a minor reduction (˜30%) that did not reach statistical significance. Interestingly, the reduction in pTau coverage area in PS19-fE4/Syn1-Cre mice resembles the extent of Tau pathology observed in PS19-fE3 mice. There was no significant difference in Tau pathology between PS19-fE3 with and without Cre, likely because the Tau pathology in PS19-fE3 mice was already low.

Western blot analysis was used to assess levels of pTau in mouse hippocampal tissue following sequential biochemical extraction with RAB and RIPA buffers, containing highly soluble and less soluble Tau proteins, respectively. There was no significant difference in pTau levels between the various genotype groups in the RAB fraction. However, PS19-fE4/Syn1-Cre and PS19-fE3 mice exhibited a significant reduction in pTau levels in the RIPA fraction relative to PS19-fE4 mice. PS19-fE4/GFAP-Cre mice showed a trend towards a reduction in pTau levels in the RIPA fraction (˜40%) relative to PS19-fE4 mice, which did not reach statistical significance. Taken together, these data indicate that neuronal APOE4 expression is a strong driver of Tau pathology, while astrocytic APOE4 exerts a minimal effect on Tau pathology.

Propagation of Tau Pathology is Reduced after Removal of APOE4 in Neurons, but not Astrocytes

To further investigate the mechanisms by which neuronal APOE4 drives Tau pathology, the effects of cell type-specific APOE4 expression on the propagation of Tau pathology were determined. Previous studies have illustrated that pathological Tau can propagate to anatomically connected brain regions after injecting various forms of Tau protein directly into mouse brains. The extent of Tau propagation after a single unilateral injection of an adeno-associated virus-2 encoding human P301S mutant Tau (AAV2-Tau-P301S) into the right dorsal hippocampus of fE mice with no Cre, Syn1-Cre, or GFAP-Cre was analyzed. The fE mice lack human P301S mutant Tau and instead express the endogenous mouse Mapt gene, allowing for a more accurate detection of human Tau spread since fE mice exhibit minimal Tau pathology. The mice were injected with the AAV2-Tau-P301S virus at 10 months of age and assessed 12 weeks post-injection at 13 months of age.

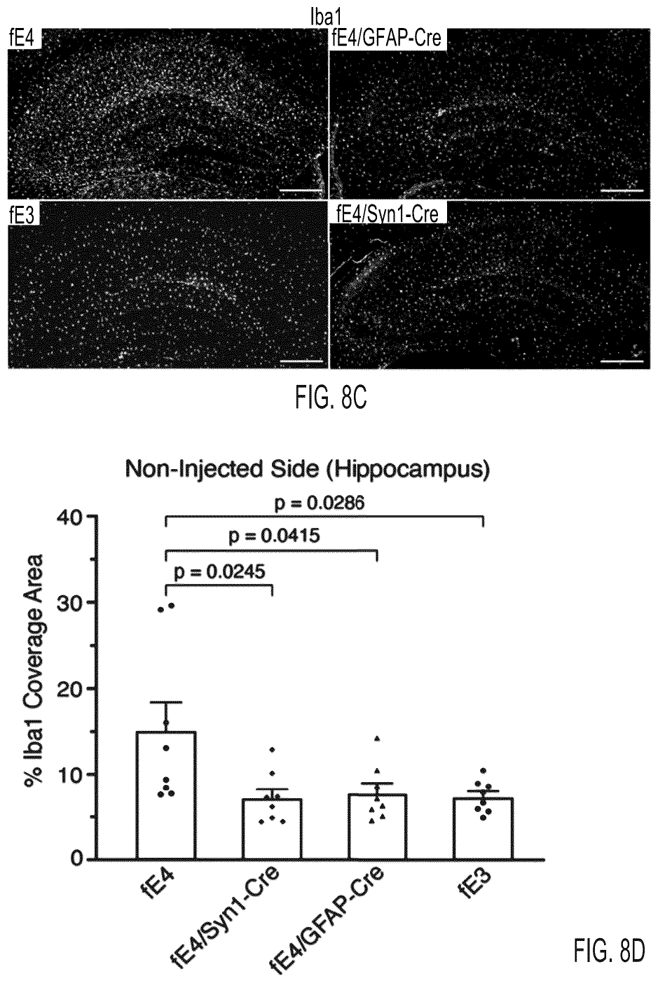

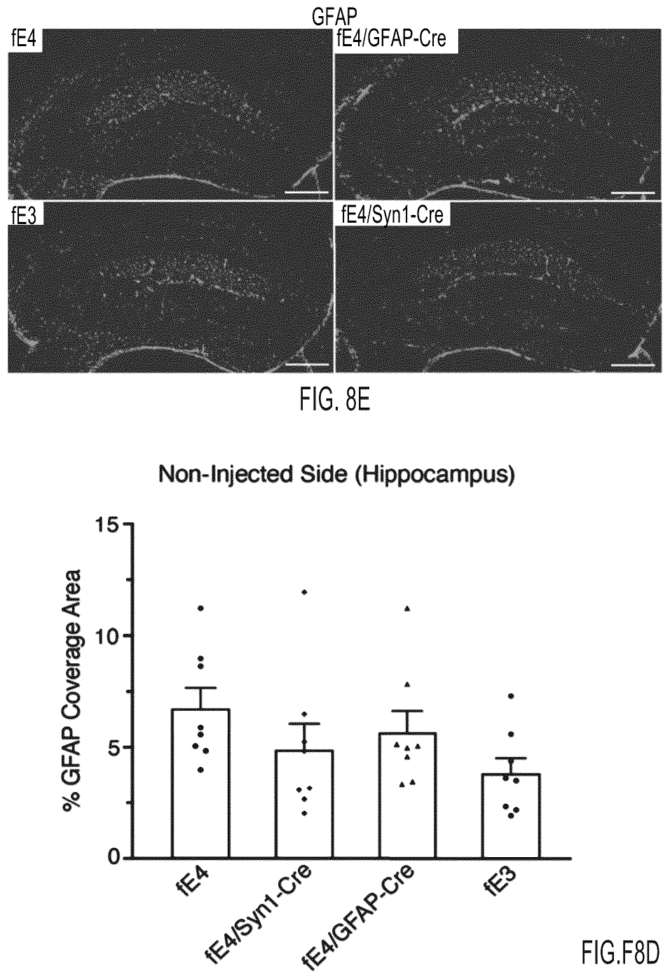

To provide evidence that the observed Tau propagation phenotype can truly be attributed to the spread of pathological human Tau between neurons as opposed to an unexpected consequence of the Tau-encoding virus itself traveling to the non-injected hippocampal side, the unilateral injection of an AAV2 of the same serotype that encodes GFP (AAV2-GFP) into the right dorsal hippocampus of a 10-month-old fE4 mouse was tested. Immunostaining with anti-GFP two weeks post-injection revealed that the GFP signal remains localized to neurons within the injected hippocampal side (FIG. 8A-B). The noninjected hippocampal side did not have any evident GFP signal in neuronal somas, although there were some GFP-positive neuronal projections, likely stemming from neurons residing on the injected hippocampal side. This illustrates that the AAV2 itself does not spread between the right and left hippocampus following unilateral injection. Furthermore, for all immunohistochemical analyses, the number of soma-positive-Tau-containing neurons was quantified to more accurately reflect human mutant Tau spread between neurons and exclude confounding factors, such as Tau-positive commissural fibers from neurons originating from the injected side.

Immunohistochemical staining with a human Tau antibody (HT7) revealed robust human Tau propagation to the non-injected hippocampal side in PS19-fE4 mice and minimal Tau propagation in PS19-fE3 mice, indicating that APOE4 promotes Tau spreading. Intriguingly, PS19-fE4/Syn1-Cre mice had limited human Tau propagation to the non-injected hippocampal side, while PS19-fE4/GFAP-Cre mice had extensive human Tau propagation that resembled the spread of Tau pathology observed in PS19-fE4 mice. Notably, there was a 55% decrease in the number of HT7+ cells with Tau propagation after removal of neuronal APOE4 and no discernable difference in the number of HT7+ cells with Tau propagation after the removal of astrocytic APOE4.

The extent of Tau propagation was analyzed by immunostaining for pTau with the AT8 antibody. PS19-fE4 and PS19-fE4/GFAP-Cre mice both exhibited robust propagation of pTau to the non-injected hippocampal side, while PS19-fE4/Syn1-Cre and PS19-fE3 mice had drastically reduced pTau propagation. Taken together, these data indicate that one mechanism by which neuronal APOE4 drives Tau pathology is by stimulating the propagation of pathological Tau across synaptically connected neurons. This data also further supports the notion that astrocytic APOE4 is not a key mediator of Tau pathology, as evidenced by its lack of effect on Tau propagation.

Network Hyperexcitability is Eliminated after Removal of Neuronal, but not Astrocytic, APOE4

To determine the cell type-specific effects of APOE4 on neuronal function in the context of tauopathy, neuronal network excitability in the hippocampal cornu ammonis (CA1) region of PS19-fE3 mice and PS19-fE4 mice with no Cre, Syn1-Cre, or GFAP-Cre was measured by input-output curve analysis of network response to incremental stimulation of Schaffer collaterals (FIG. 9A). PS19-fE4 mice had notable CA1 neuron hyperexcitability as compared to PS19-fE3 mice (FIG. 9B). Removing neuronal APOE4 eliminated neuronal network hyperexcitability, while removing astrocytic APOE4 led to a minor reduction that did not reach significance (FIG. 9B), indicating that neuronal APOE4 drives neurodysfunction in the context of tauopathy.

Neurodegeneration is Reduced after Removal of Either Neuronal or Astrocytic APOE4

Next, the extent of neurodegeneration in 10-month-old PS19-fE mice was evaluated after removal of APOE from neurons or astrocytes. Analyses of hippocampal and posterior lateral ventricle volumes revealed that PS19-fE4 mice exhibited extensive neurodegeneration relative to PS19-fE3 mice. Neurodegeneration was significantly reduced in both PS19-fE4/Syn1-Cre and PS19-fE4/GFAP-Cre mice, illustrated by increased hippocampal volume and decreased posterior lateral ventricle volume, resembling those of PS19-fE3 mice. Removal of neuronal or astrocytic APOE3 did not significantly impact neurodegeneration.

Quantification of the extent of neuronal cell loss within the various subfields of the hippocampus revealed that PS19-fE4 mice had extensive neuron loss in CA1 and the dentate gyrus, while PS19-fE4/Syn1-Cre and PS19-fE4/GFAP-Cre mice both had significantly reduced neuronal cell loss in both CA1 and dentate gyrus subfields of the hippocampus. There was no significant difference in neuronal cell layer thickness in PS19-fE3 mice when APOE3 was removed from neurons or astrocytes.

In PS19-fE4 mice, there was a weak, but significant, negative correlation between Tau pathology and hippocampal volume, suggesting that Tau pathology contributes to the neurodegeneration occurring in these mice, at least to some extent. There was also a strong negative correlation between hippocampal volume and the volume of the posterior lateral ventricle, and a strong positive correlation between the thickness of the CA1 cell layer and hippocampal volume. Overall, these data illustrate that removal of either neuronal or astrocytic APOE4 is protective against Tau-mediated neurodegeneration and leads to reduced neuronal cell and hippocampal volume loss. This result is intriguing, considering the Tau pathology data showing that astrocytic APOE4 has minimal effects on Tau pathology accumulation and propagation.

Gliosis is Drastically Reduced after Removal of Either Neuronal or Astrocytic APOE4

Next, the extent of microgliosis and astrogliosis within these various genotype groups at 10 months of age was investigated to explore the roles of neuronal or astrocytic APOE4 in eliciting gliosis in the context of tauopathy. Immunohistochemical staining of microglia using an Iba1 antibody revealed that PS19-fE4 mice had extensive microgliosis in the hippocampus, as demonstrated by a high percent coverage area of microglia. PS19-fE4/Syn1-Cre and PS19-fE4/GFAP-Cre mice both exhibited drastic reductions in microgliosis. Additionally, PS19-fE3 mice with no Cre, Syn1-Cre, or GFAP-Cre all have greatly reduced microgliosis relative to PS19-fE4 mice. There was a strong negative correlation between Iba1 coverage area and hippocampal volume in PS19-fE4 mice, indicating that microgliosis is a good indicator and potential contributor to neurodegeneration.

This microgliosis phenotype was further defined by immunohistochemical staining of CD68, which is a marker of activated microglia. There was a dramatic difference in the coverage area of activated microglia between genotype groups, with PS19-fE4 mice exhibiting extensive microglial activation and all other genotype groups having minimal microglial activation. Interestingly, of all pathological correlations made in PS19-fE4 mice, the coverage area of CD68+ activated microglia had the strongest negative correlation with hippocampal volume, suggesting that the extent of microglial activation is the strongest indicator and potential contributor to APOE4-enhanced neurodegeneration in tauopathy. Taken together, these data indicate that microgliosis is strongly enhanced by APOE4 relative to APOE3 in tauopathy, and that removal of APOE4 from either neurons or astrocytes attenuates the extent of microgliosis.

The extent of astrogliosis after removal of APOE from neurons or astrocytes was assessed. PS19-fE4 mice exhibited considerable astrogliosis in the hippocampus relative to PS19-fE3 mice, as detected by immunohistochemical staining with the astrocytic GFAP antibody. The extent of astrogliosis was greatly reduced after removal of either neuronal or astrocytic APOE4. These observations were also supported by immunohistochemical staining for an activated astrocytic marker, S100β. There was no obvious difference in the extent of astrogliosis after removal of APOE3 from either cell type relative to PS19-fE3 mice. Strikingly, and in sharp contrast to microgliosis, neither the coverage area of astrocytes nor activated astrocytes was significantly correlated with hippocampal volume. These data indicate that APOE4 strongly enhances astrogliosis relative to APOE3 in tauopathy, and that the removal of APOE4 from either neurons or astrocytes reduces astrocyte activation and/or recruitment. Furthermore, it also illustrates that the extent of astrogliosis might not be a valuable indicator or contributor to APOE4-enhanced neurodegeneration in tauopathy.

To further demystify the relationship between Tau pathology and gliosis, it was investigated whether Tau propagation also elicits gliosis in the non-injected hippocampal side. Indeed, we observed that fE4 mice, along with higher Tau spread, also exhibited higher microgliosis in the non-injected hippocampal side than fE3 mice (FIG. 8C-D). Fascinatingly, there was a significant decrease in microgliosis in the non-injected hippocampal side after removal of either neuronal or astrocytic APOE4. This indicates that removal of neuronal or astrocytic APOE4 reduces microgliosis regardless of the extent of Tau spread, as fE4/GFAP-Cre mice still had relatively high levels of Tau spread to the non-injected hippocampal side. These findings suggest that astrocytic APOE4 acts downstream of Tau pathology to promote microgliosis. Furthermore, we assessed the extent of astrocyte coverage area in the non-injected hippocampal side and observed no discernable differences in astrogliosis after removal of neuronal or astrocytic APOE4 (FIG. 8E-F), probably due to the relatively low astrogliosis in the non-injected hippocampal side even in fE4 mice in this Tau spreading model.

Myelin Deficits and Depletion of Oligodendrocyte Progenitor Cells are Reduced after Removal of APOE4 from Neurons, but not Astrocytes

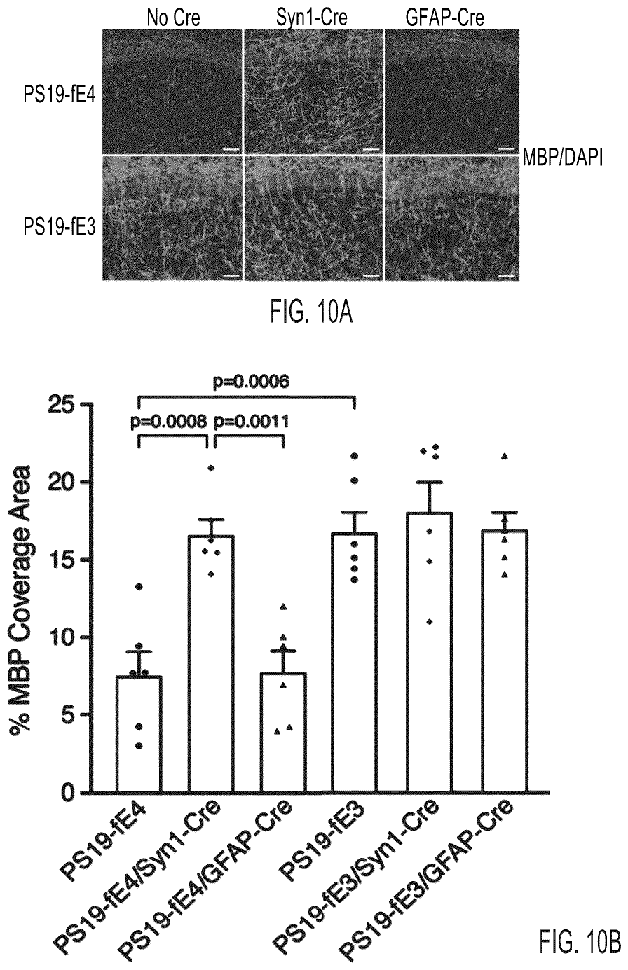

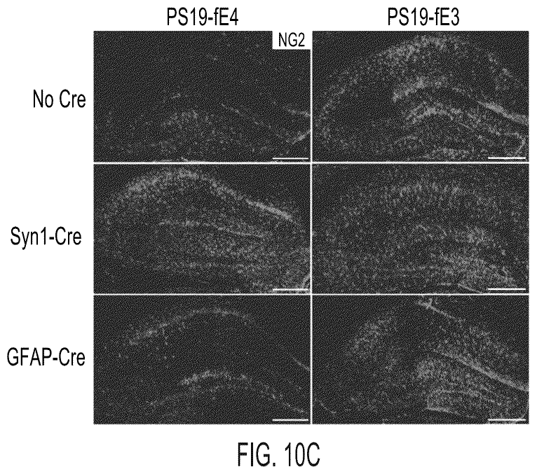

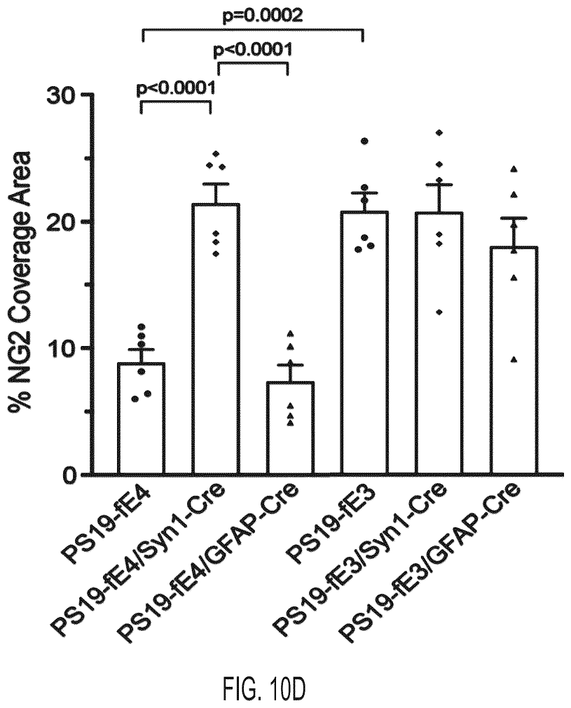

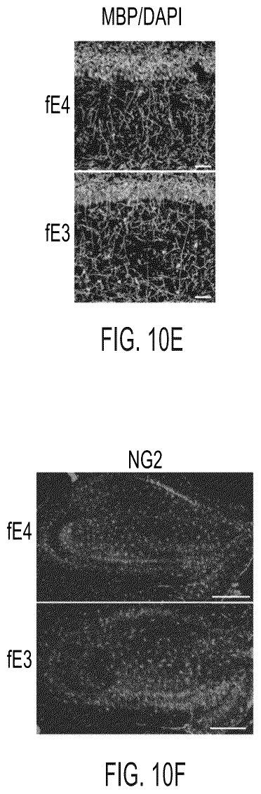

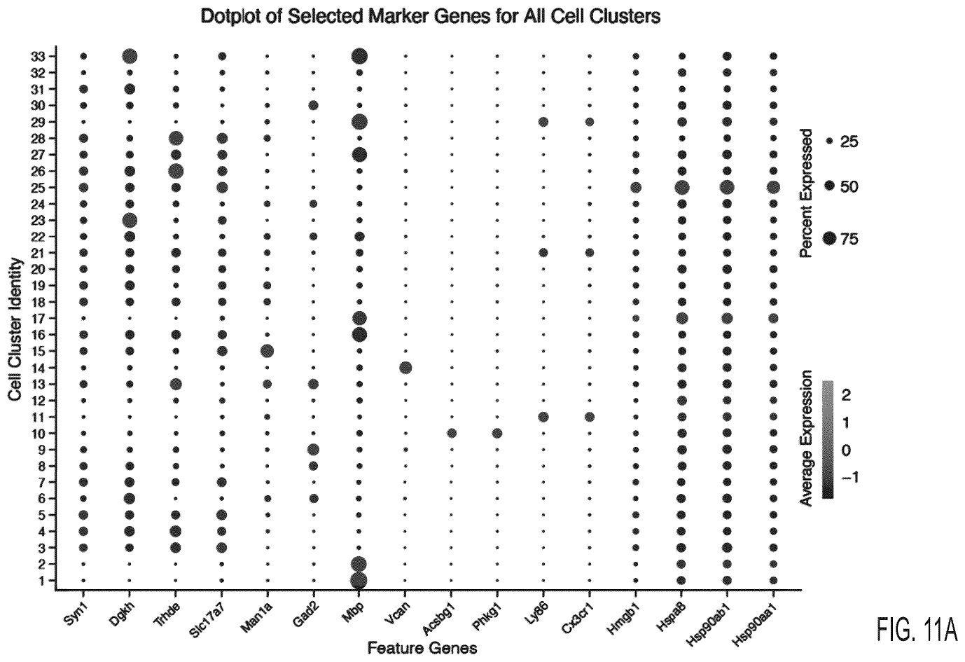

Since myelin degeneration and oligodendrocyte deficits have been observed in human AD brains and in mouse models of AD and tauopathy, the effects of neuronal and astrocytic APOE4 removal on the maintenance of myelin integrity and the density of hippocampal oligodendrocyte progenitor cells (OPCs) was investigated. To determine myelin integrity in the hippocampus, myelin basic protein (MBP) was stained with an MBP-specific antibody. Quantification of the percent coverage area of MBP in the stratum radiatum underneath the pyramidal cell layer of CA1 revealed that PS19-fE4 mice had extensive myelin loss relative to PS19-fE3 mice (FIG. 10A-B). Strikingly, a significant reduction in myelin loss in PS19-fE4/Syn1-Cre mice that resembled the phenotype of PS19-fE3 mice, while PS19-fE4/GFAP-Cre mice exhibited considerable myelin loss that resembled the phenotype of PS19-fE4 mice (FIG. 10A-B). This indicates that neuronal APOE4 plays a primary detrimental role in promoting myelin deficits in the context of tauopathy, whereas astrocytic APOE4 does not impact this phenotype. There was no substantial difference in myelin integrity in PS19-fE3 mice after removal of neuronal or astrocytic APOE3.

To further characterize the effects of neuronal and astrocytic APOE4 on biological components involved in the myelination process, an NG2 antibody was used to probe for OPCs, which have been suggested to aid the repair of damaged myelin in conditions of CNS injury and neurodegeneration. A significant decrease in the percent OPC coverage area in the hippocampus of PS19-fE4 mice, as compared to PS19-fE3 mice (FIG. 10C-D). Strikingly, removal of neuronal APOE4 significantly increased the percent OPC coverage area to levels that were similar to those in the PS19-fE3 mice (FIG. 10C-D). In contrast, PS19-fE4/GFAP-Cre mice had significantly lower OPC coverage area relative to PS19-fE4/Syn1-Cre mice and even exhibited a trend of decreased OPCs relative to PS19-fE4 mice, although it did not reach statistical significance (FIG. 10C-D). There were no obvious differences in OPC coverage area after removal of neuronal or astrocytic APOE3 in PS19-fE3 mice (FIG. 10C-D). Immunohistochemical staining for myelin and OPCs in fE4 and fE3 mice revealed that APOE4 mice lacking the human mutant Tau-P301S do not exhibit myelin deficits and have similar OPC levels in the hippocampus as fE3 mice, illustrating that the effects of APOE4 on these phenotypes are specific to the setting of tauopathy (FIG. 10E-F). Overall, these findings suggest that neuronal APOE4 plays a pivotal role in depleting the hippocampal OPC pool and causing myelin deficits in this compound tauopathy mouse model, while astrocytic APOE4 does not significantly contribute to either of these two pathological processes.

snRNA-Seq Identifies Neurodegenerative Disease-Associated Subpopulations of Neurons and Oligodendrocytes that are Largely Eliminated by Removing Neuronal, but not Astrocytic, APOE4

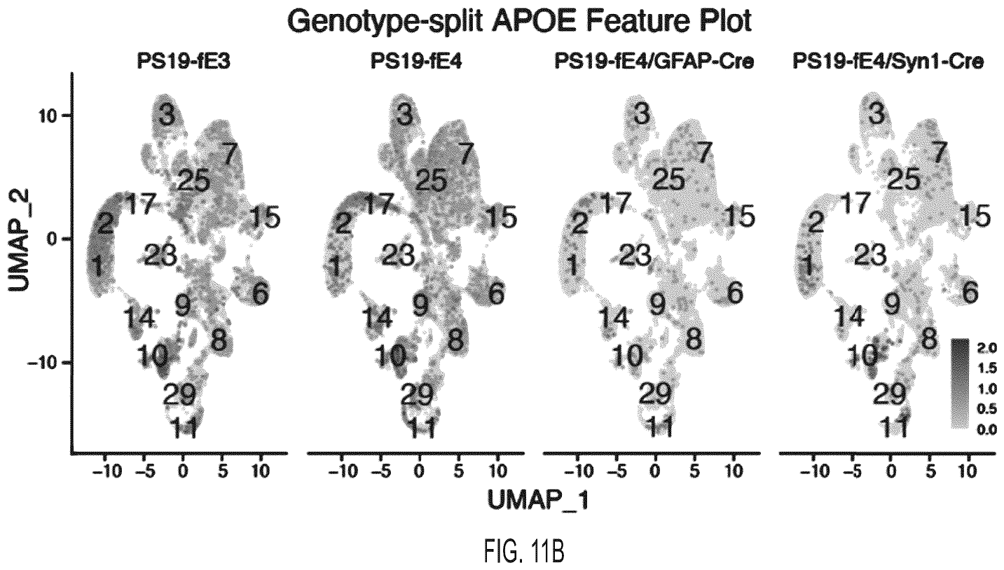

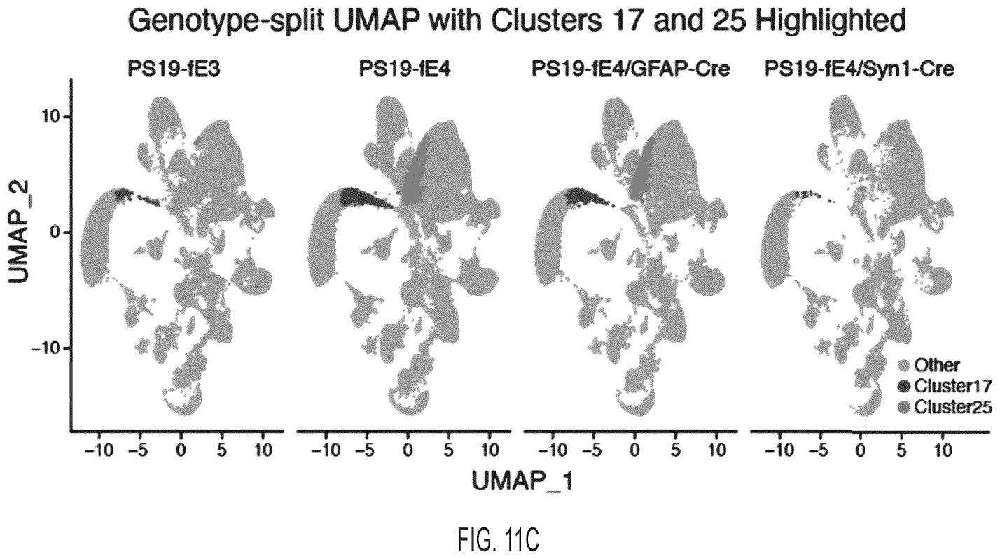

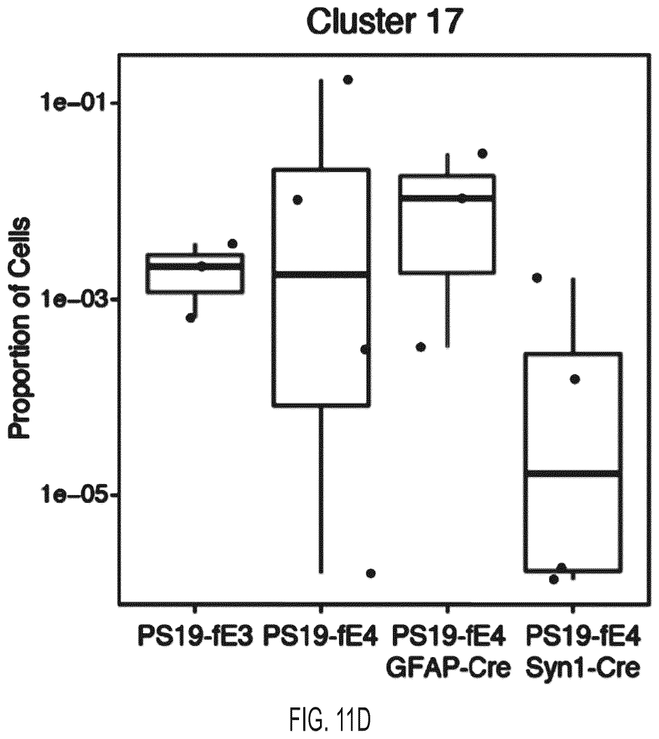

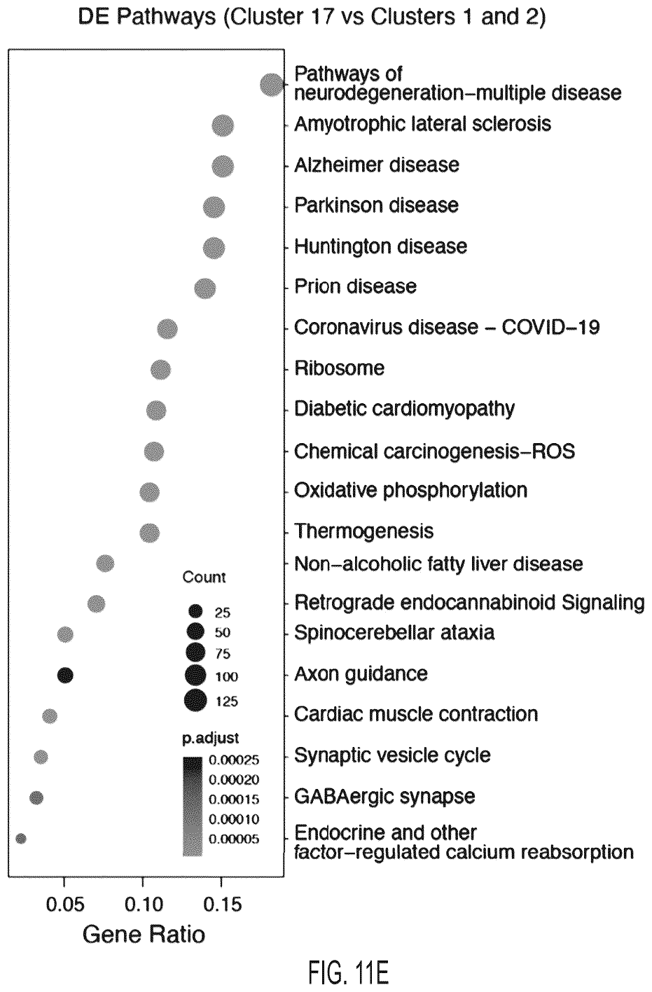

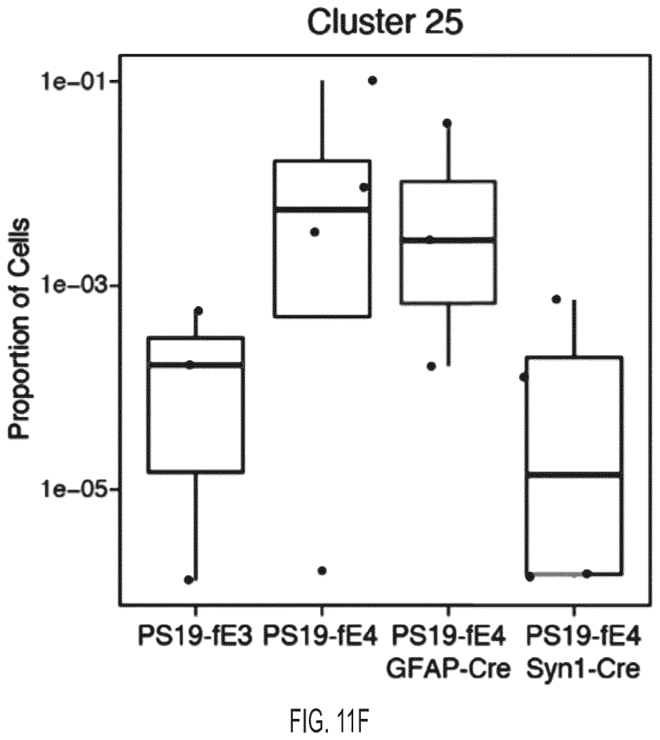

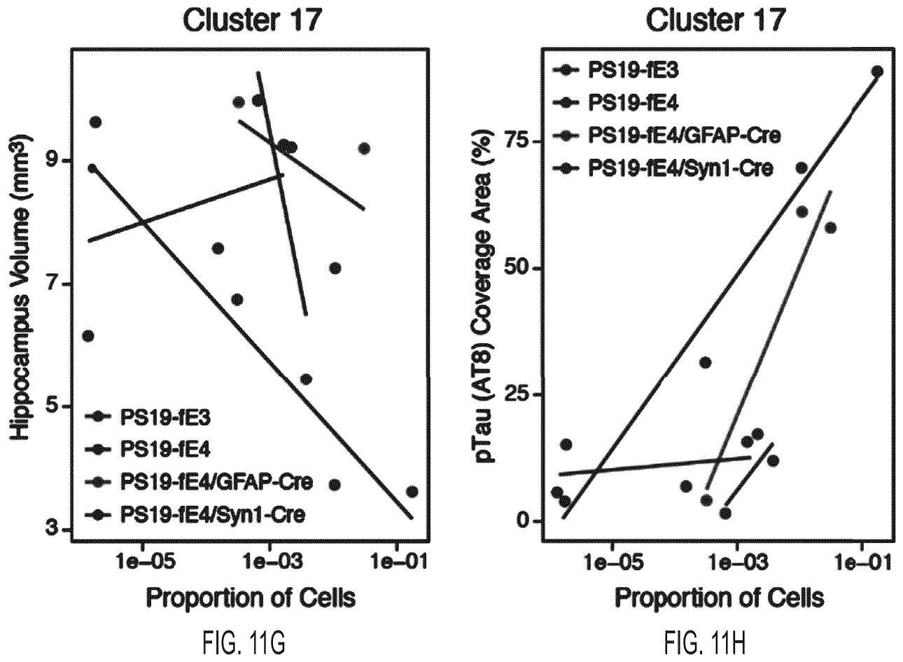

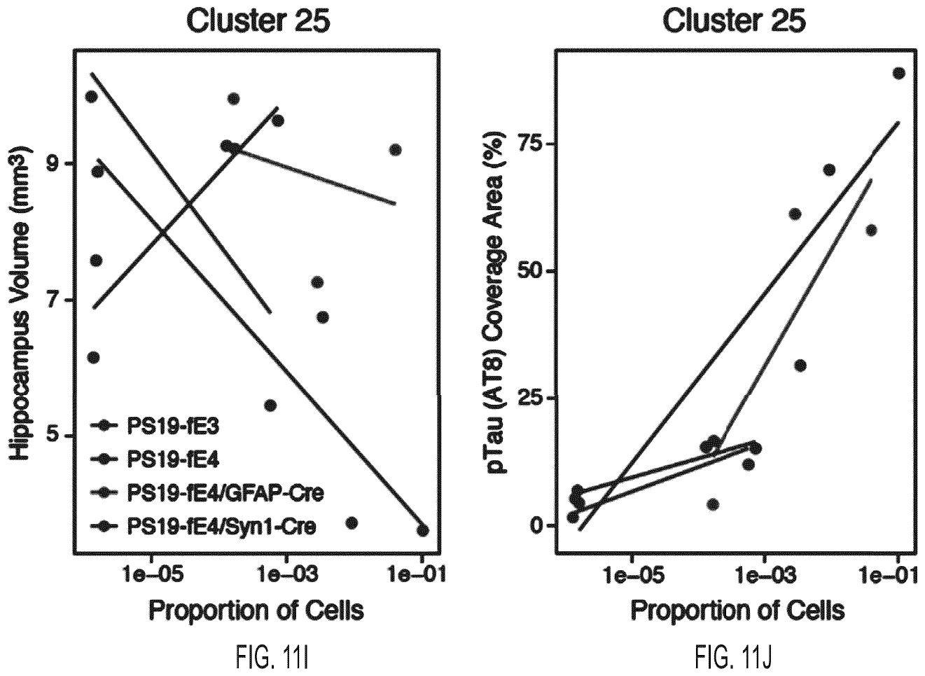

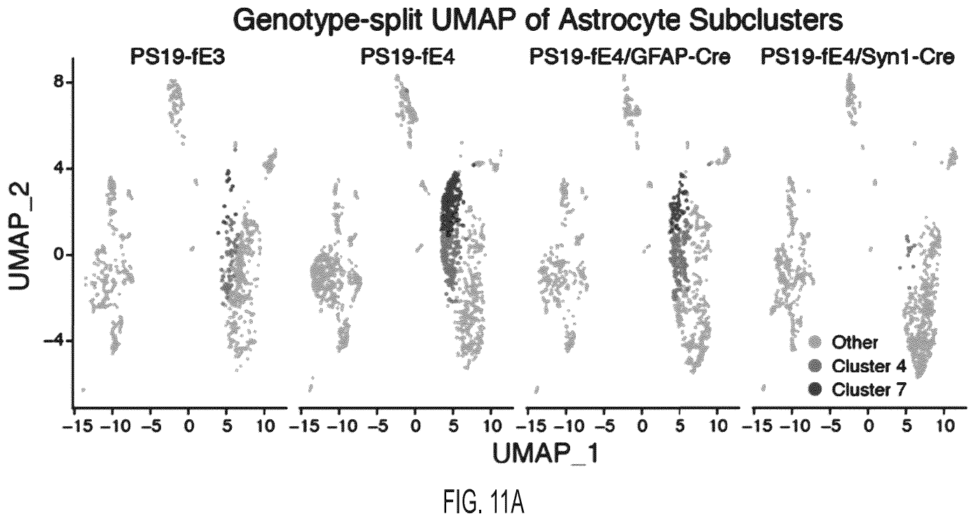

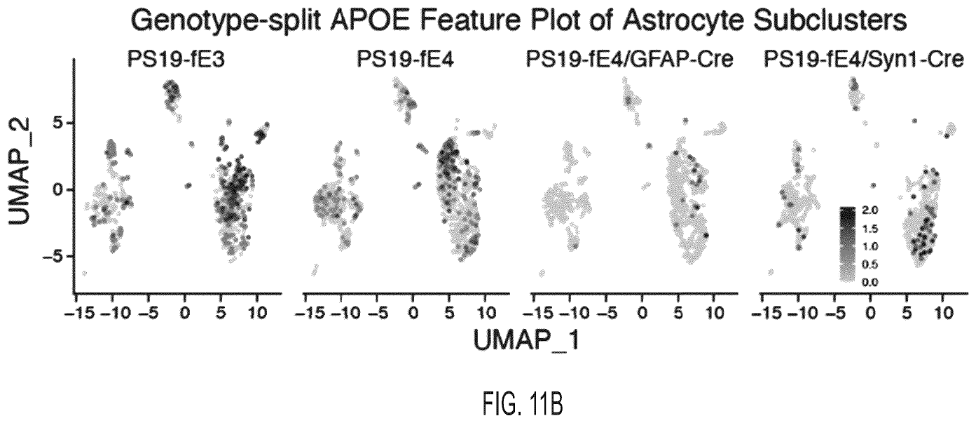

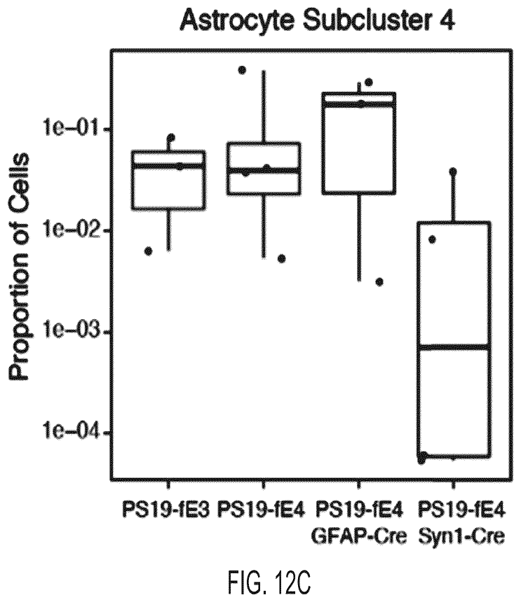

In order to gain an in-depth understanding of the cell type-specific effects of APOE4 at the transcriptomic level across different types of hippocampal cells, single-nucleus RNA sequencing (snRNA-seq) was performed on isolated hippocampi from 10-month-old PS19-fE4 mice with no Cre, Syn1-Cre, or GFAP-Cre and PS19-fE3 mice. The snRNA-seq dataset contained 119,317 nuclei covering 26,285 genes after normalization and filtering for quality control. Clustering by shared nearest neighbor (SNN) and visualization by uniform manifold approximation and projection (UMAP) revealed 33 distinct cell clusters. Based on their expression of marker genes, these clusters were assigned to 16 excitatory (Ex) neuron clusters (3-5, 7, 12, 15, 16, 18-20, 23, 25-28, 33), 7 inhibitory (In) neuron clusters (6, 8, 9, 13, 22, 24, 30), 3 oligodendrocyte clusters (1, 2, 17), one astrocyte cluster (10), 3 microglia clusters (11, 21 29), 2 oligodendrocyte progenitor cell (OPC) clusters (14, 32), and 1 unknown cluster (31) (FIG. 11A). As predicted, APOE was highly expressed in astrocytes in PS19-fE4 and PS19-fE3 mice, and its expression was drastically reduced in astrocytes in PS19-fE4/GFAP-Cre mice (FIG. 11B). As reported previously some neurons also expressed APOE in PS19-fE4 and PS19-fE3 mice, and neuronal APOE expression was eliminated in PS19-fE4/Syn1-Cre mice (FIG. 11B).

Log odds ratio estimates from a Generalized Linear Mixed Effects Model were used to assess association with Animal Models (GLMM_AM) was used to identify cell clusters that were altered in PS19-fE3, PS19-fE4/Syn1-Cre, and PS19-fE4/GFAP-Cre mice versus PS19-fE4 mice. This analysis revealed that oligodendrocyte cluster 17 had lower odds of having cells from PS19-fE4/Syn1-Cre mice than from PS19-fE4 mice, with an almost complete elimination in PS19-fE4/Syn1-Cre mice (FIG. 11C-D). Based on differentially expressed (DE) gene analysis, cells in cluster 17 exhibited a drastic upregulated expression of three major heat shock protein genes (FIG. 11A) and a significant downregulated expression of myelin basic protein (Mbp) and myelin associated oligodendrocyte basic protein (Mobp) genes relative to the other oligodendrocyte clusters. DE pathway analysis revealed the enrichment of Kyoto Encyclopedia of Gene and Genomes (KEGG) pathways related to general neurodegeneration, AD, and other neurodegenerative diseases (FIG. 711E), indicating that cluster 17 represents neurodegenerative disease-associated oligodendrocytes.

Furthermore, excitatory neuron cluster 25 had significantly lower odds of having cells from PS19-fE4/Syn1-Cre and PS19-fE3 mice than from PS19-fE4 mice, with an almost complete elimination in PS19-fE4/Syn1-Cre mice (FIG. 11C, F). DE gene analysis revealed that cells in cluster 25 had significant upregulated expression of three major heat shock protein genes (FIG. 11A), human MAPT gene, and amyloid precursor protein (App) gene. DE pathway analysis revealed the enrichment of KEGG pathways related to general neurodegeneration, AD, and other neurodegenerative diseases ( ), indicating that cluster 25 represents neurodegenerative disease-associated neurons.

Similarly, log odd ratio estimates from another Generalized Linear Mixed Effects Model to assess associations with histopathology (GLMM_histopathology) revealed that the proportion of cells in clusters 17 and 25 both exhibited significant negative associations with hippocampal volume and significant positive associations with the coverage area of pTau as well as astrogliosis and microgliosis. The associations were largely driven by the PS19-fE4 group and, to a lesser extent, by the PS19-fE4/GFAP-Cre group (FIG. 11G-J). The proportion of cells in cluster 17 also had a significant negative association with MBP coverage area (FIG. 11D). Taken together, these findings illustrate that removal of neuronal, but not astrocytic APOE4, leads to a drastic reduction in the amount of neurodegenerative disease-associated oligodendrocytic and neuronal subpopulations within the hippocampus and that these two cell populations have strong associations with Tau pathology, gliosis, neurodegeneration, and oligodendrocyte deficits.

### Neurodegenerative Disease-Associated Astrocyte and Microglia Subpopulations are Largely Eliminated by Removing Neuronal, but not Astrocytic, APOE4

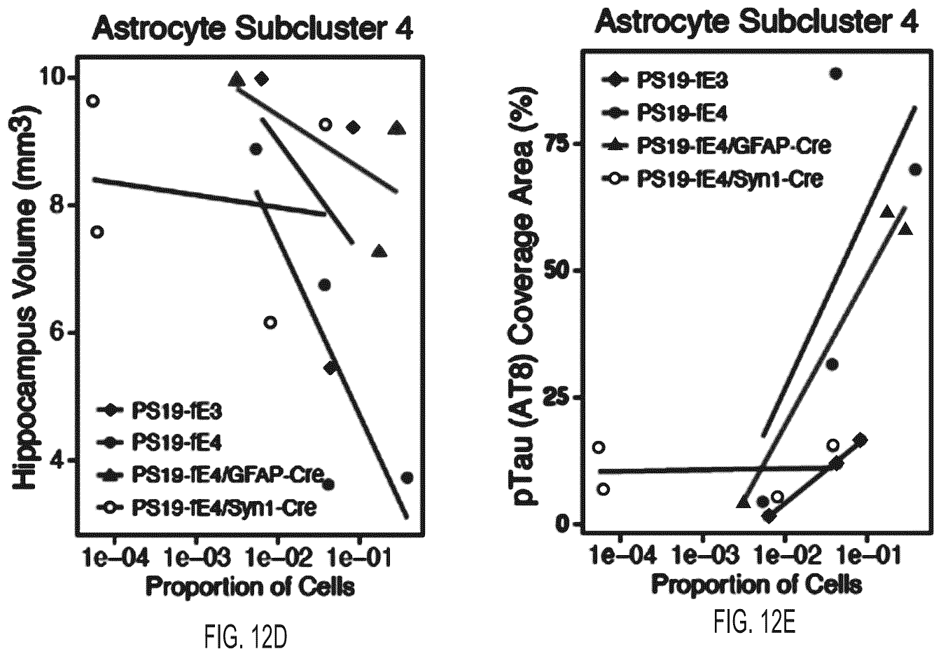

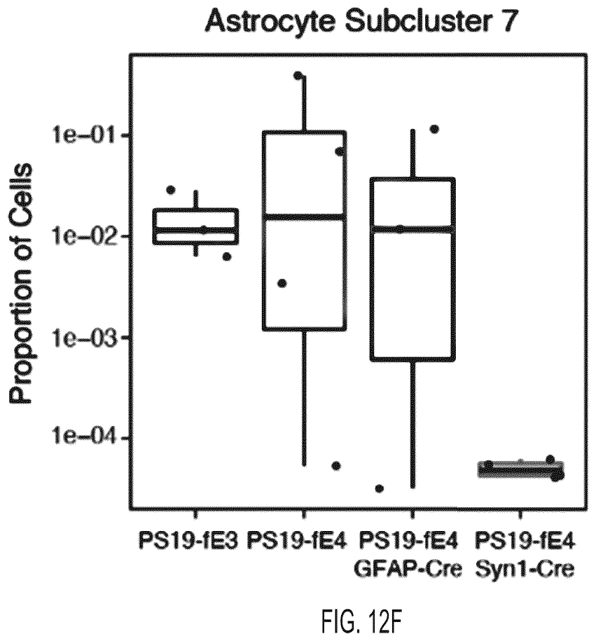

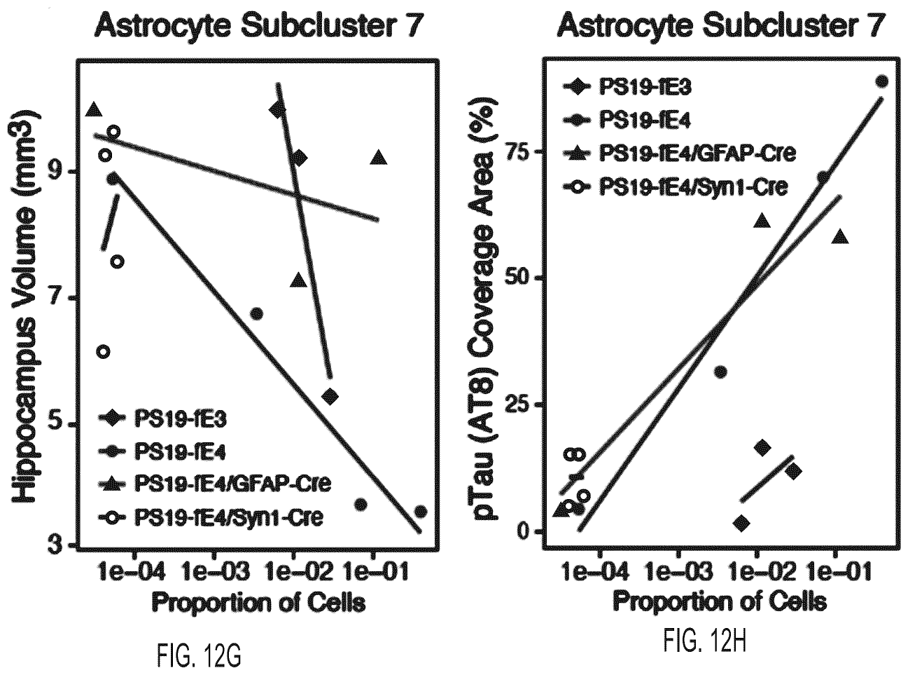

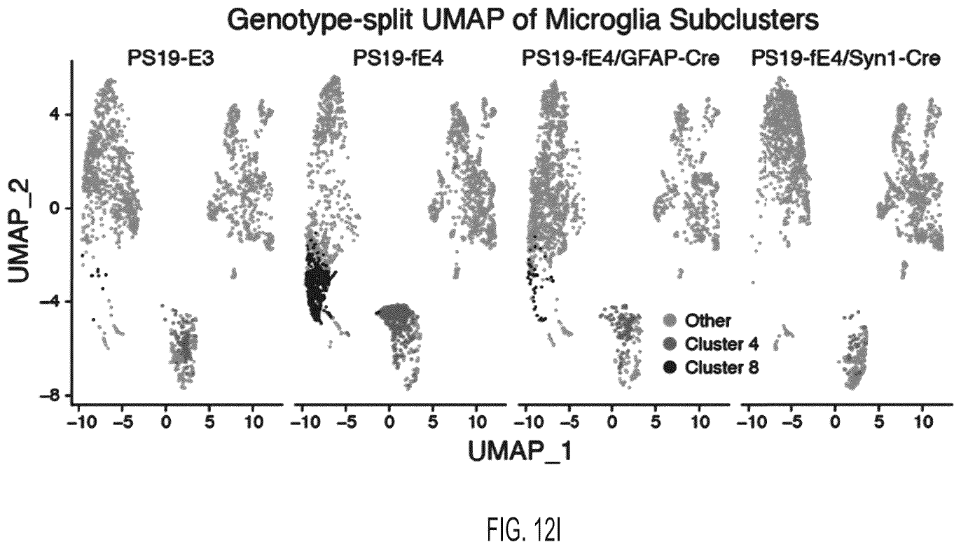

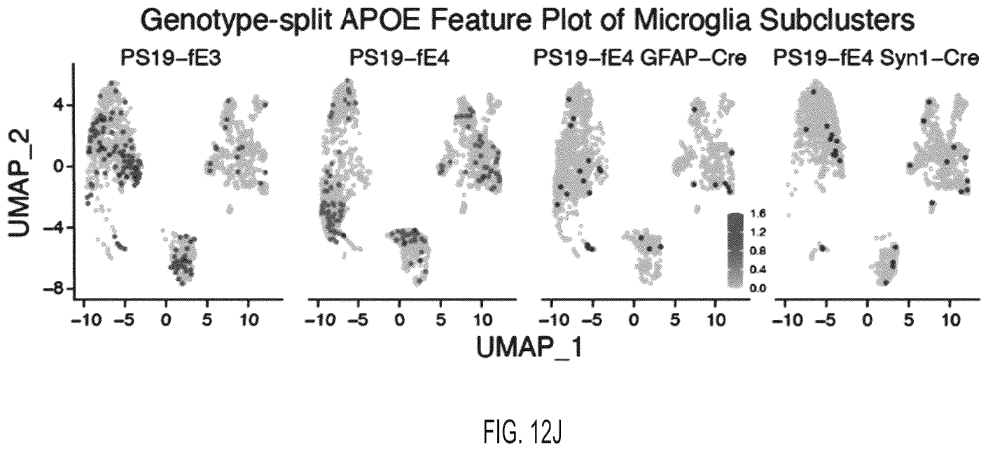

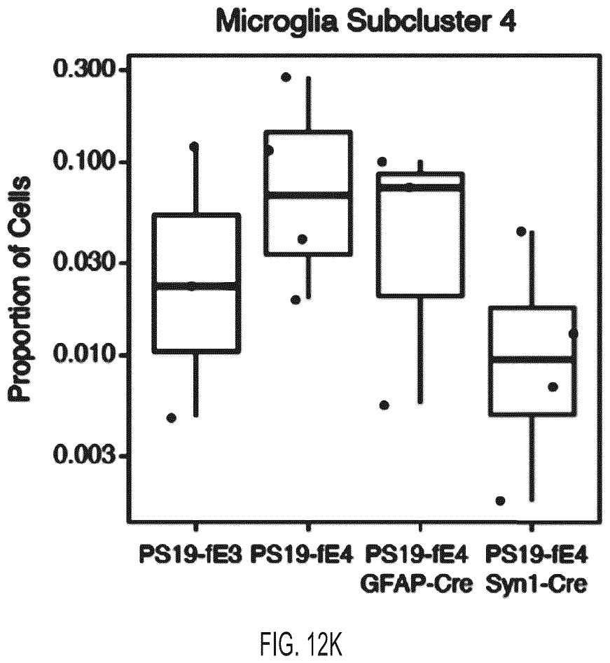

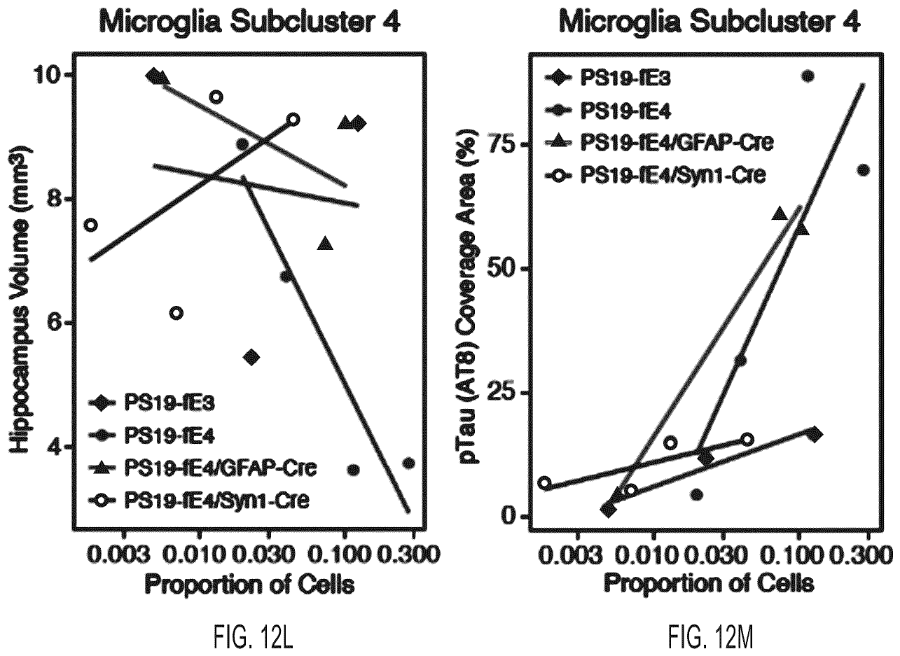

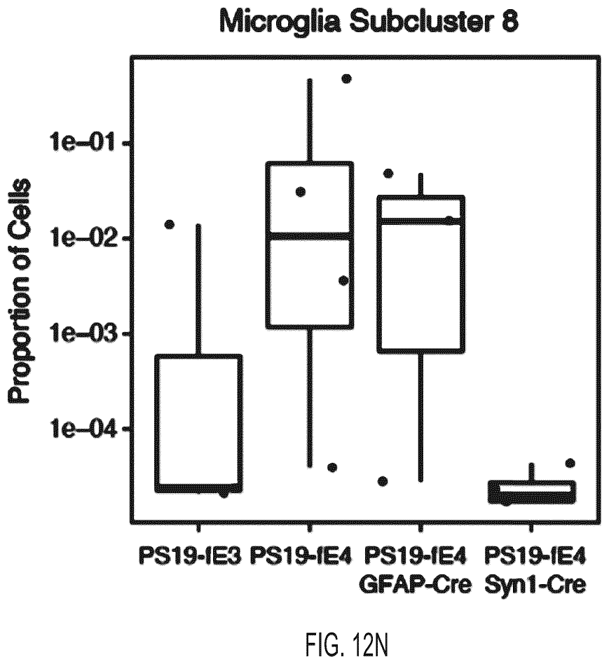

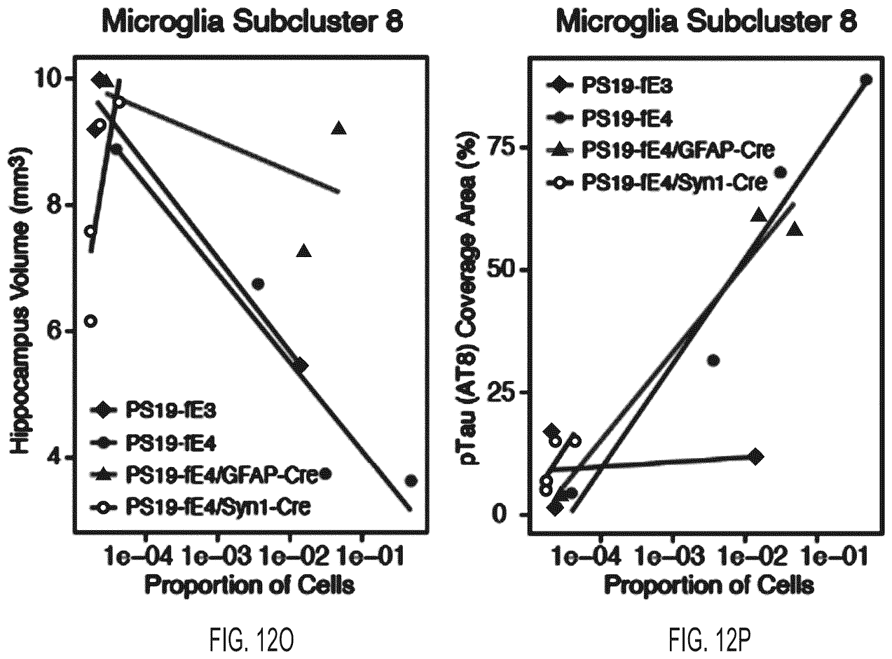

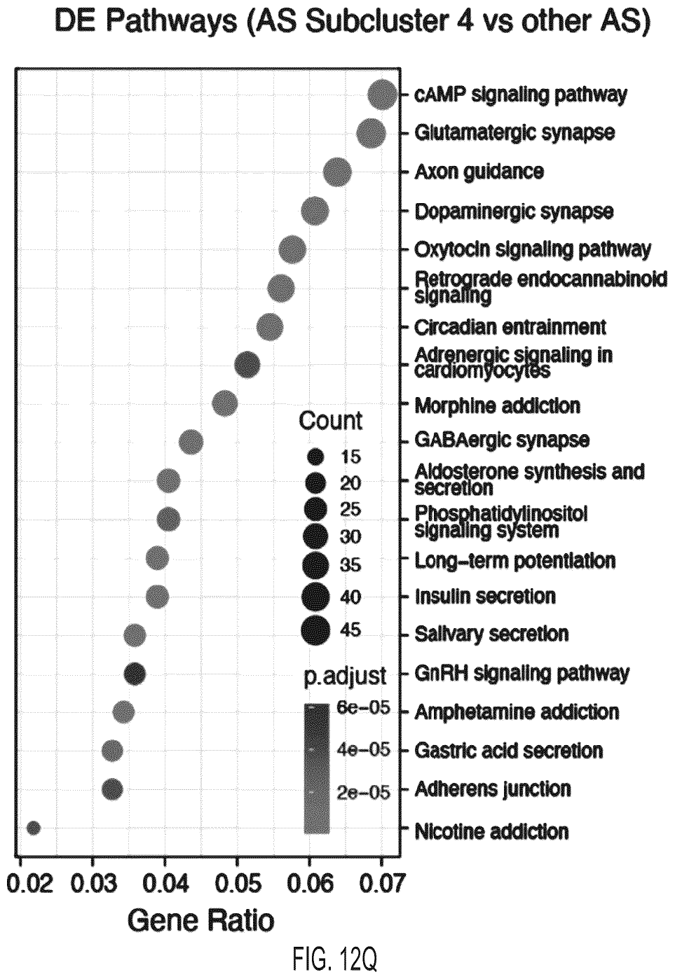

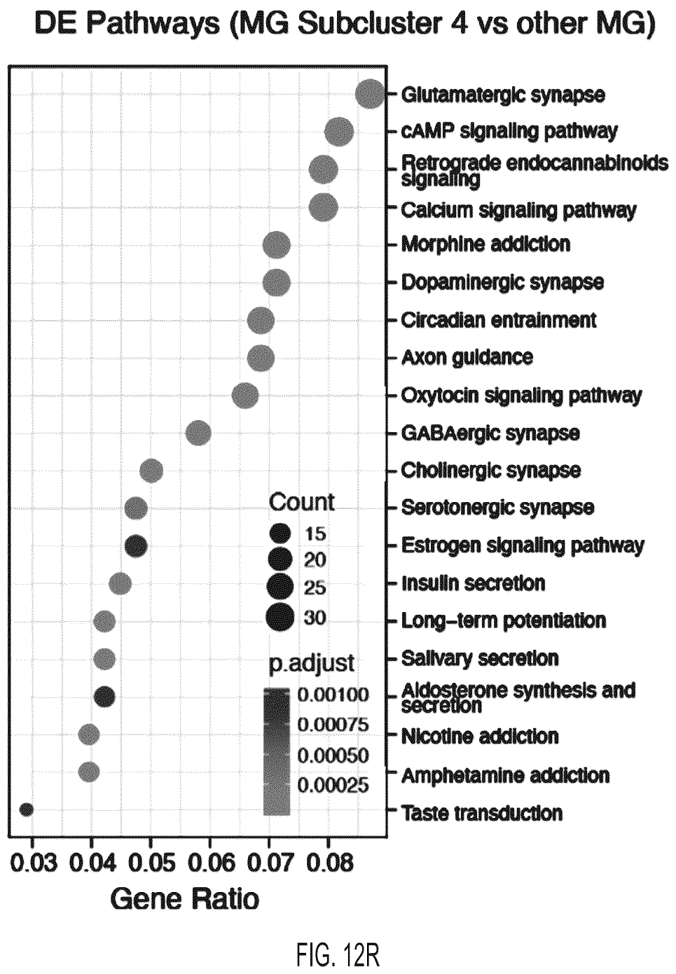

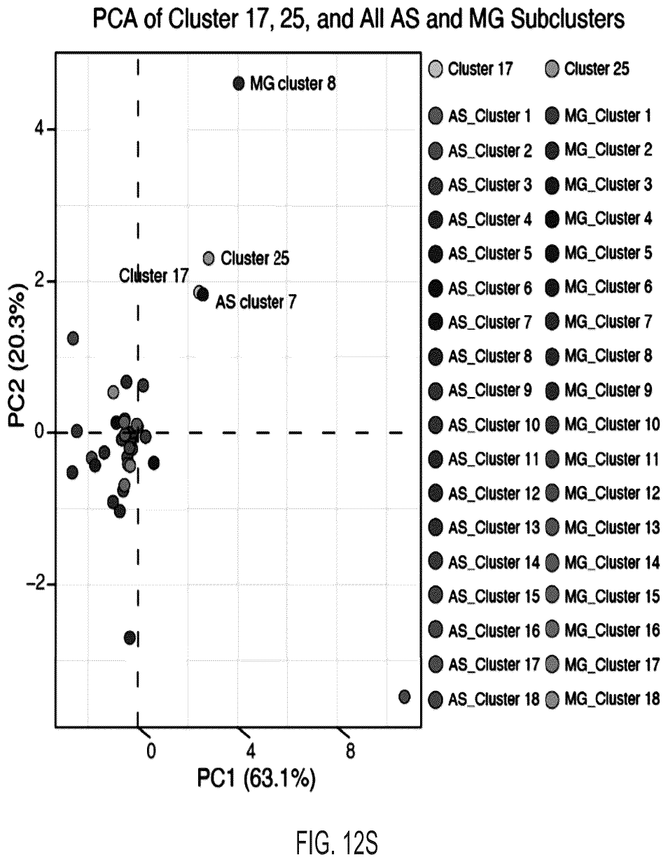

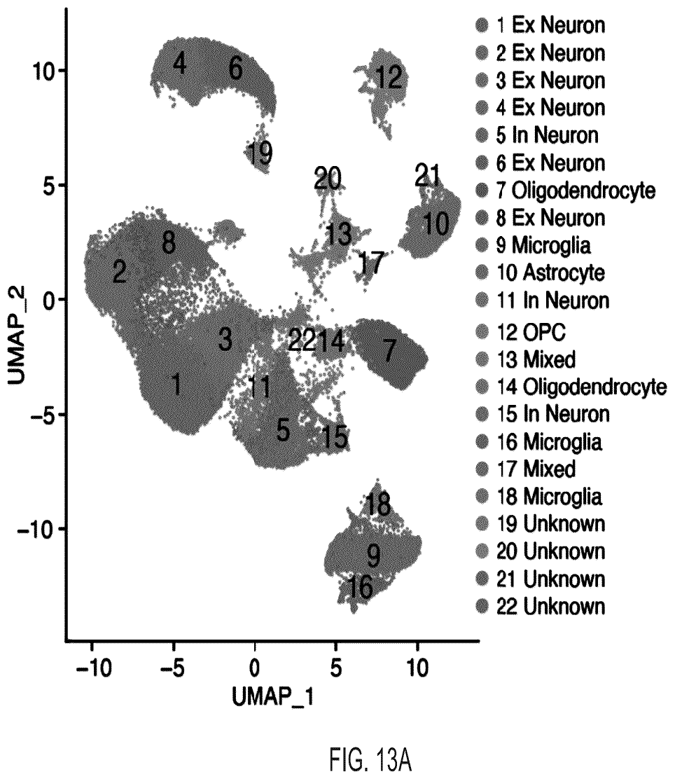

Further subclustering astrocytes identified 18 subpopulations. Log odds ratio estimates from a GLMM_AM revealed that astrocyte subclusters 4 and 7 had lower odds of having cells from PS19-fE4/Syn1-Cre mice than from PS19-fE4 mice, with a complete elimination of subcluster 7 in PS19-fE4/Syn1-Cre mice (FIG. 12A,C,F). Interestingly, astrocyte subclusters 4 and 7 highly expressed APOE (FIG. 12D). DE gene and pathway analysis revealed the enrichment of KEGG pathways related to general neurodegeneration, AD, and other neurodegenerative diseases in subcluster 7, indicating that subcluster 7 represents neurodegenerative disease-associated astrocytes. Subcluster 4 showed an enrichment of KEGG pathways related to cAMP signaling, synaptic function, and long-term potentiation FIG. 12Q), suggesting that subcluster 4 represents synaptic dysfunction-associated astrocytes.

Further subclustering microglia also identified 18 subpopulations. Log odds ratio estimates from a GLMM_AM revealed that microglia subclusters 4 and 8 had lower odds of having cells from PS19-fE4/Syn1-Cre mice than from PS19-fE4 mice, with a complete elimination of subcluster 8 in PS19-fE4/Syn1-Cre mice (FIG. 12I,K,N). Interestingly, microglia subclusters 4 and 8 highly expressed APOE (FIG. 12J). DE gene and pathway analysis revealed the enrichment of KEGG pathways related to general neurodegeneration, AD, and other neurodegenerative diseases in subcluster 8, indicating that subcluster 8 represents neurodegenerative disease-associated microglia. Subcluster 4 showed an enrichment of KEGG pathways related to synaptic function, cAMP signaling, and long-term potentiation (FIG. 12R), suggesting that subcluster 4 represents synaptic dysfunction-associated microglia.

Similarly, log odds ratio estimates from a GLMM_histopathology revealed that the proportion of cells in astrocyte subcluster 7 and microglia subcluster 8 both exhibited significant negative associations with hippocampal volume and significant positive associations with the coverage area of pTau as well as astrogliosis and microgliosis. The associations were largely driven by the PS19-fE4 group and, to a lesser extent, by the PS19-fE4/GFAP-Cre group (FIG. 12D-E,G-H,L-M,O-P). Interestingly, PCA clustering of oligodendrocyte cluster 17 and excitatory neuron cluster 25 together with all astrocyte and microglia subclusters illustrated that the disease-associated astrocyte subcluster 7 and microglia subcluster 8 have similar contributions to the eight pathological parameters as the two disease-associated clusters of oligodendrocytes (cluster 17) and neurons (cluster 25) (FIG. 12S). Taken together, these findings illustrate that removal of neuronal, but not astrocytic APOE4, leads to a drastic reduction in the amount of neurodegenerative disease-associated astrocyte and microglia subpopulations within the hippocampus and that these two glial subpopulations have strong associations with Tau pathology, gliosis, and neurodegeneration.

### Removal of Astrocytic APOE4 Secondarily Reduces Neuronal APOE4 Expression

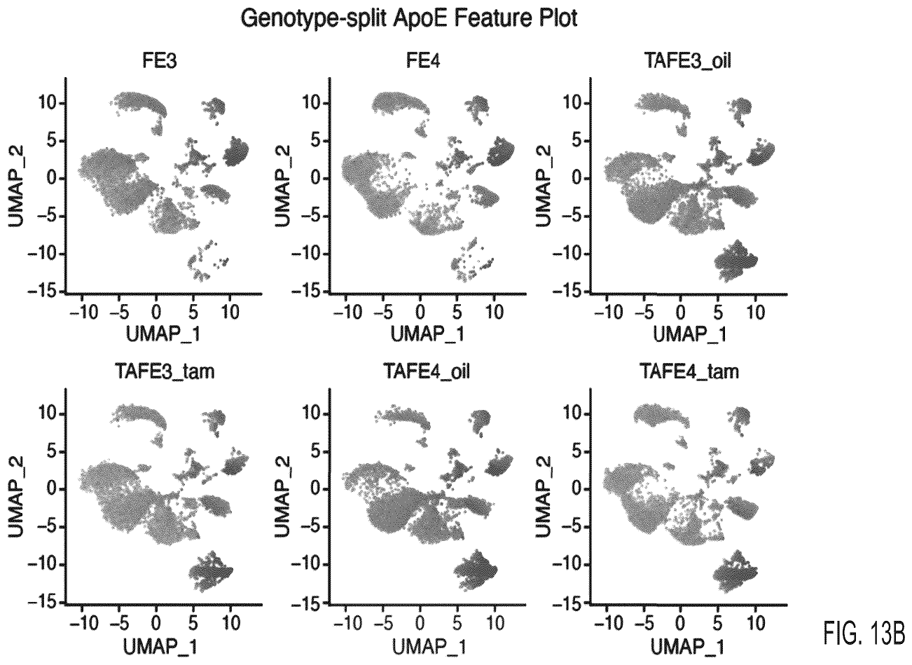

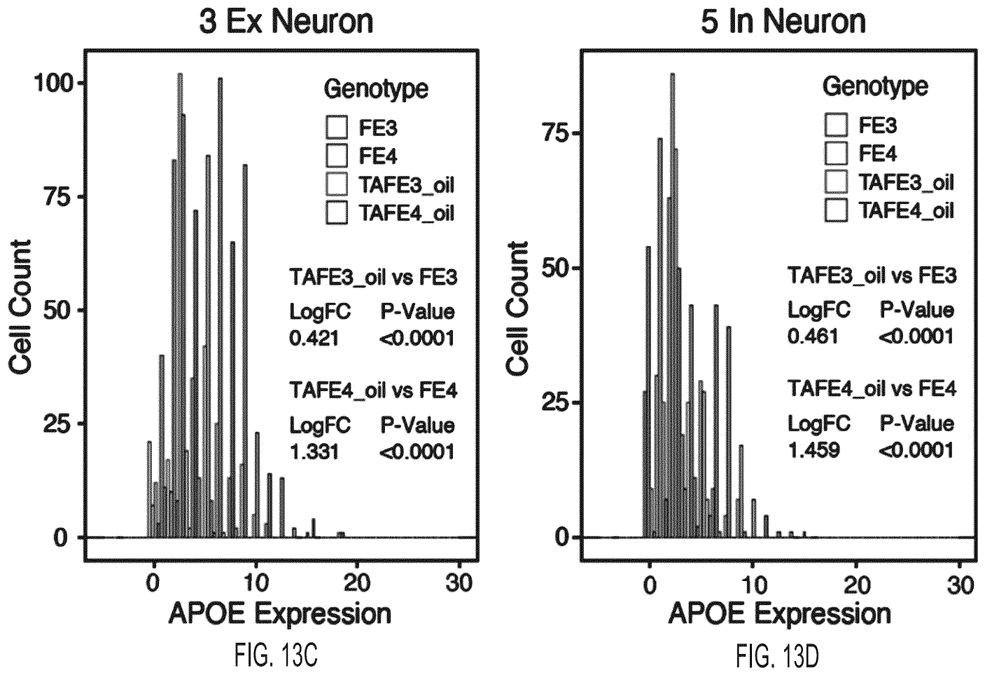

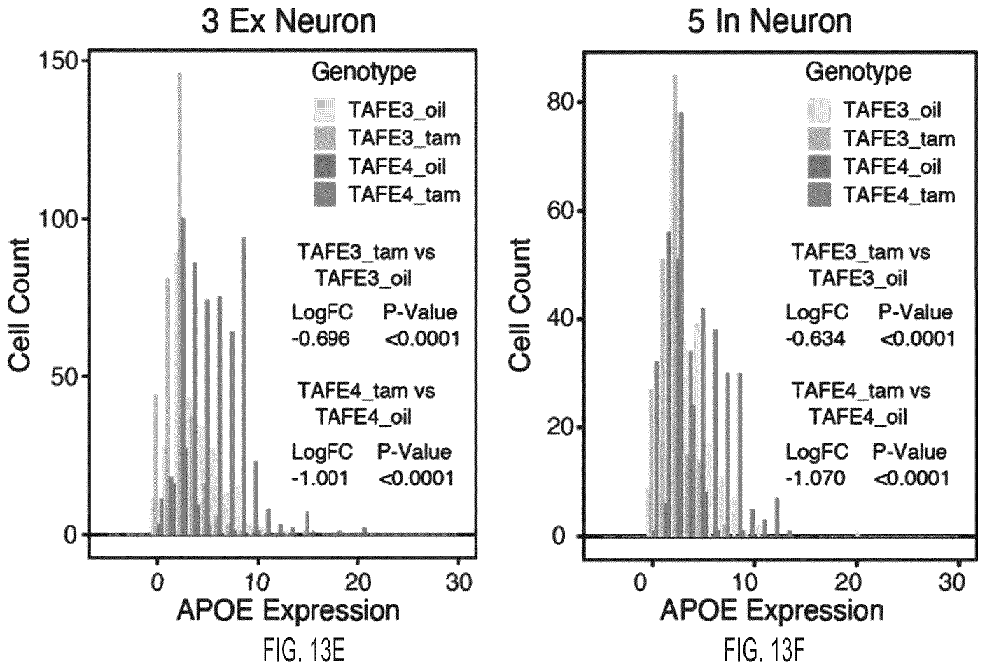

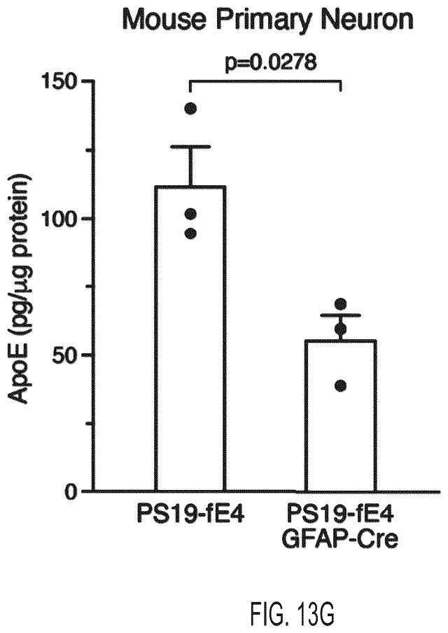

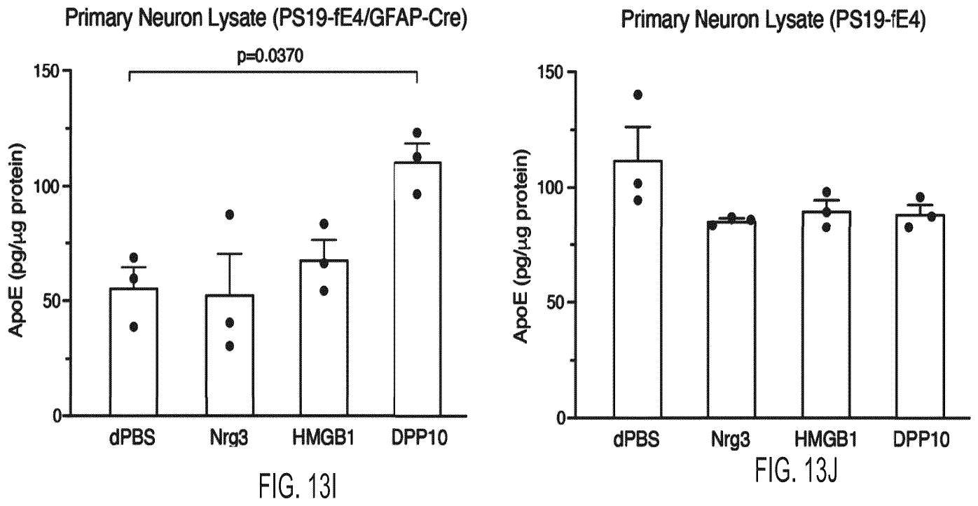

During analysis of snRNA-seq data, removal of astrocytic APOE also drastically reduced APOE expression in neurons in addition to its predicted elimination of APOE expression in astrocytes (FIG. 10B). To confirm this observation, a publicly available snRNA-seq dataset (GEO:GSE164507), was analyzed which utilized a similar compound mouse model carrying a floxed-knock-in human APOE gene and an astrocyte-specific Cre (Aldh111-Cre/ERT2 BAC transgene) that deletes the human APOE gene in astrocytes in a tamoxifen-inducible manner during adulthood, along with either the presence or absence of P301S mutant human Tau. In this snRNA-seq dataset (FIG. 13A), neuronal APOE expression was observed in both FE3 and FE4 mice (FIG. 13B), which was further increased in PS19-FE4 (TAFE4_oil) and PS19-FE3 (TAFE3_oil) mice (FIG. 13A,B), suggesting that tauopathy increases neuronal APOE expression. Upon removal of astrocytic APOE induced by tamoxifen treatment during adulthood, a drastic reduction of neuronal APOE in both excitatory and inhibitory neurons was also clearly observed in PS19-fE4/Aldh111-Cre/ERT2 (TAFE4 tam) and PS19-fE3/Aldh111-Cre/ERT2 (TAFE3 tam) mice relative to mice treated with oil (FIG. 13B,E,F). The effects of removing astrocytic APOE on reduction of neuronal APOE were observed in all subtypes of neurons, indicating a general regulation of neuronal APOE expression by astrocytic APOE. Thus, the initial observation that removal of astrocytic APOE secondarily reduces neuronal APOE expression is confirmed in a different tauopathy model with human APOE, even when astrocytic APOE removal was driven under a different astrocyte-specific promoter and its removal was induced at a different timepoint in the lifespan of the mouse model.

To determine whether this observation can be validated at the protein level, the levels of APOE protein were quantitatively evaluated via sandwich ELISA in cultured primary neurons isolated from the cortex and hippocampus of prenatal pups. The analysis revealed that primary neurons from PS19-fE4/GFAP-Cre mice exhibited a significant 50% decrease in APOE protein levels relative to those from PS19-fE4 mice (FIG. 13G). These data provide strong evidence that the removal of astrocytic APOE4 leads to a drastic decrease in neuronal APOE4 expression and protein production and solidifies this regulatory relationship between astrocytic and neuronal APOE4. Thus, the limited protective effects of removing astrocytic APOE4 are likely attributable to a secondary reduction, but not complete elimination, of neuronal APOE4.

Early studies had reported that neuronal APOE expression is regulated by astrocyte-released factor(s) through modulation of the ERK pathway within neurons. An in-depth comparison of snRNA-seq data between astrocytes from PS19-fE4/GFAP-Cre mice and those from PS19-fE4 mice revealed that some of the top down-regulated genes after astrocytic APOE4 removal were documented activators of the ERK/MAPK pathway, including dipeptidyl peptidase 10 (DPP10) and Neuregulin-3 (Nrg3). Accordingly, it was hypothesized that astrocytic APOE4 may regulate neuronal APOE4 expression by promoting the release of these factors that activate the neuronal ERK pathway, leading to increased neuronal APOE4 expression. To test this possibility, primary neurons isolated from PS19-fE4 and PS19-fE4/GFAP-Cre mice were treated with 10 g/mL of recombinant DPP10 or Nrg3 protein or a dPBS control and then quantitatively evaluated the APOE protein levels in cell lysates by sandwich ELISA. It was observed that PS19-fE4/GFAP-Cre primary neurons treated with DPP10 had a striking 98% increase in APOE protein levels relative to the dPBS control (FIG. 13H). On the other hand, there was no significant difference in APOE levels in PS19-fE4 primary neurons following DPP10 treatment (FIG. 13I). This data indicates that primary neurons from PS19-fE4/GFAP-Cre mice, in which the astrocytic APOE4 was removed, are sensitive to DPP10 upregulation of APOE expression, while primary neurons from PS19-fE4 mice are not. Differences in APOE levels in PS19-fE4/GFAP-Cre or PS19-fE4 primary neurons following treatment with Nrg3 (FIG. 13H,I) were not observed, indicating that Nrg3 is not involved in the regulation of neuronal APOE4 expression.

### Neuronal Release of HMGB1 is Completely Eliminated by Removing Neuronal, but not Astrocytic, APOE4

In searching for pro-inflammation cytokines that could trigger Tau- and neurodegeneration-induced gliosis, it was observed that cells in the neurodegenerative disease-associated neuronal cluster 25 had drastically increased expression of the high mobility group box 1 (Hmgbl) gene (FIG. 10A). The HMGB1 protein is widely recognized as a factor for glial cell activation. Under pathological conditions, HMGB1 translocates from the nucleus to the cytoplasm of stressed or dying cells and it is then released to act as a proinflammatory cytokine. Immunohistochemical staining for HMGB1 protein and the nuclear marker DAPI in 10-month-old mice revealed that PS19-fE4 mice had a remarkably high amount of HMGB1 protein that translocated from the nucleus to the cytoplasm of neurons in the dentate gyrus of the hippocampus, as a significant majority of the protein can be found located outside of the nucleus of the hippocampal neurons. On the other hand, HMGB1 protein in PS19-fE3 mice was largely retained within the nucleus and there was minimal translocation to the cytoplasm. Interestingly, removal of APOE4 from either neurons or astrocytes attenuates this phenotype observed in PS19-fE4 mice, with the majority of HMGB1 protein remaining localized to the nucleus.

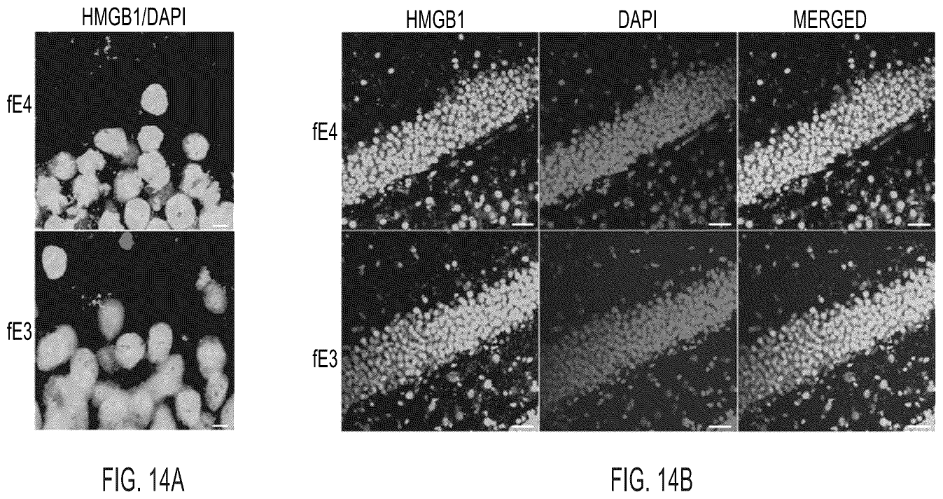

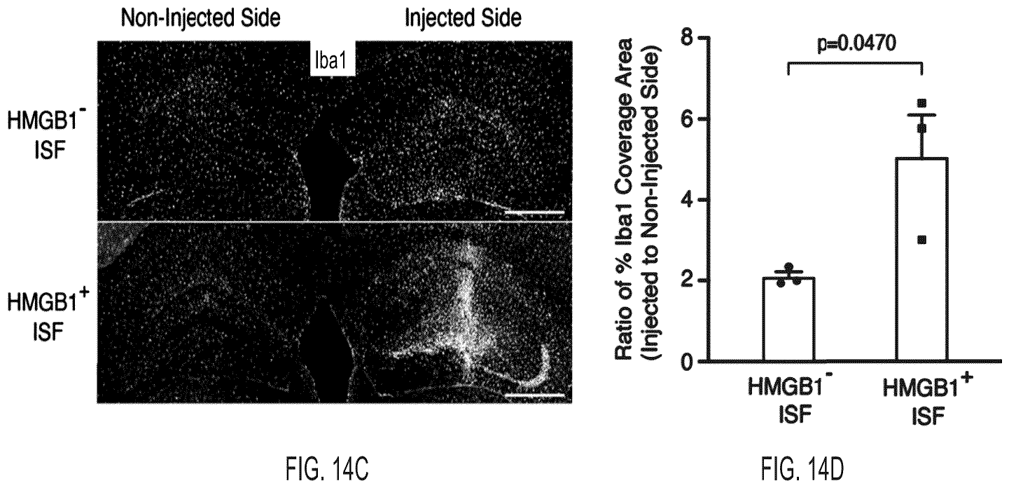

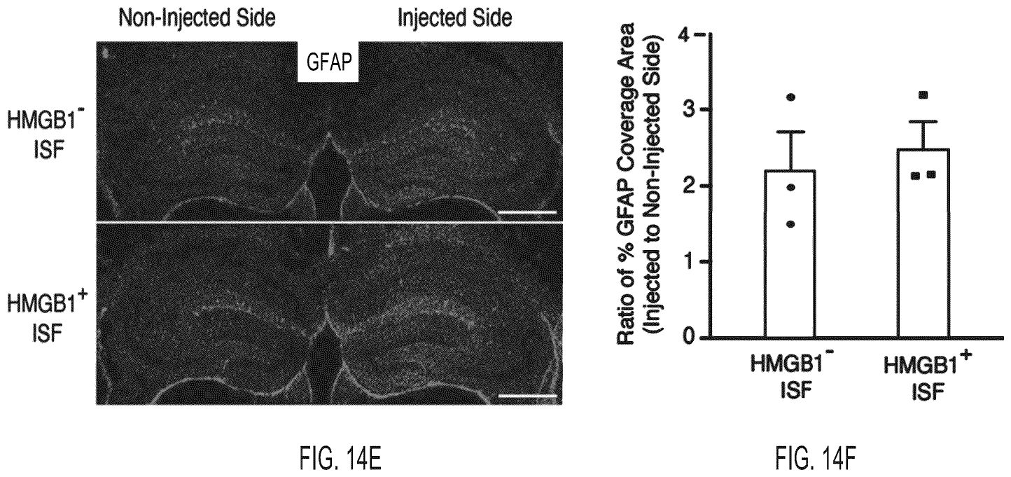

Furthermore, PS19-fE4 mice contained a greater number of HMGB1-positive puncta in both the nucleus and the cytoplasm relative to the other genotype groups. Notably, fE4 and fE3 mice that lacked human mutant Tau-P301S had HMGB1 protein localized to the nucleus, illustrating that the nucleo-cytoplasmic translocation of HMGB1 in APOE4 mice requires the coexistence of both APOE4 and tauopathy (FIG. 14).

To evaluate the extent of disease-associated HMGB1 release from hippocampal neurons within these various genotype groups, the hippocampal interstitial fluid (ISF) was collected from 8.5-month-old mice over a 24 hour period using in vivo microdialysis. The levels of HMGB1 protein in the ISF were quantitively determined using sandwich ELISA. While PS19-fE4 mice exhibited high levels of HMGB1 protein within their hippocampal ISF, removal of neuronal APOE4 reduced HMGB1 protein levels down to an undetectable level in the ISF, indicating that neuronal APOE4 plays a role in controlling HMGB1 release from neurons. Removal of astrocytic APOE4 also led to an 86% reduction, but not complete elimination, of HMGB1 protein in the ISF, which is likely due to the secondary reduction, but not complete elimination, of neuronal APOE4 expression (FIG. 13B,F,G).

To determine whether HMGB1 is a potential modulatory factor of neuronal APOE4 expression, primary neurons isolated from PS19-fE4/GFAP-Cre and PS19-fE4 mice were treated with 10 g/mL of recombinant HMGB1 protein or a dPBS control and quantitatively evaluated the APOE protein levels in cell lysates by sandwich ELISA. A significant difference in APOE protein levels in PS19-fE4/GFAP-Cre or PS19-fE4 primary neurons following treatment with HMGB1 was not observed (FIG. 13H,I), indicating that HMGB1 is not involved in the regulation of neuronal APOE4 expression.

To evaluate the importance of neuronally released HMGB1 in triggering gliosis in APOE4-related tauopathies, the ISF collected from the hippocampus of an 8.5-month-old PS19-fE4 mouse was injected into the hippocampi of 8.5-month-old wildtype mice. The injected ISF fractions were either enriched with relatively high concentrations of HMGB1 protein (fractions 19-22) or had undetectable levels of HMGB1 protein as a control (fractions 4-7), as determined by sandwich ELISA. The wildtype mice received a unilateral injection into the right dorsal hippocampus of either HMGB1⁺ or control ISF and were analyzed 6 days post-injection to assess acute changes in gliosis. Wildtype mice that were injected with the control (HMGB1⁻) ISF exhibited relatively low levels of microgliosis and astrogliosis on both the injected and noninjected hippocampal sides, illustrating that the majority of gliosis induced by the injection surgery itself subsided by 6 days post-injection (FIG. 14C-F). On the contrary, wildtype mice injected with the HMGB1⁺ ISF displayed significantly increased levels of microgliosis on the injected hippocampal side relative to the non-injected side (FIG. 14C-D). Interestingly, injection of HMGB1⁺ ISF does not lead to a significant increase in astrogliosis in the injected hippocampal side relative to the non-injected side (FIG. 14E-F). Taken together, these data suggest that the neuronal release of HMGB1 represents a novel mechanism by which neuronal APOE4 promotes glial cell activation.

Treatment with HMGB1 Inhibitors Substantially Reduces APOE4-Driven Gliosis, Tau Pathology, and Degeneration

Based on the findings that neuronal APOE4 is a potent driver of neuronal HMGB1 release, the therapeutic efficacy of HMGB1 inhibitors in combating APOE4-driven pathogenesis in the context of tauopathy was tested. To this end, two well-characterized HMGB1 inhibitors, ethyl pyruvate (EP) and glycyrrhizic acid (GA), which are selective inhibitors of HMGB1 translocation and release were tested A mixed solution of EP (80 mg/kg) and GA (20 mg/kg) or saline vehicle was administered to PS19-fE4 and PS19-fE3 mice at three doses per week for 12 weeks via intraperitoneal injections. Treatment began when the mice were 6.5 months of age, at about the onset of adverse pathology, and completed when the mice were 9.5 months of age, when severe neurodegeneration and pathological changes are typically present, as demonstrated in this and other studies.

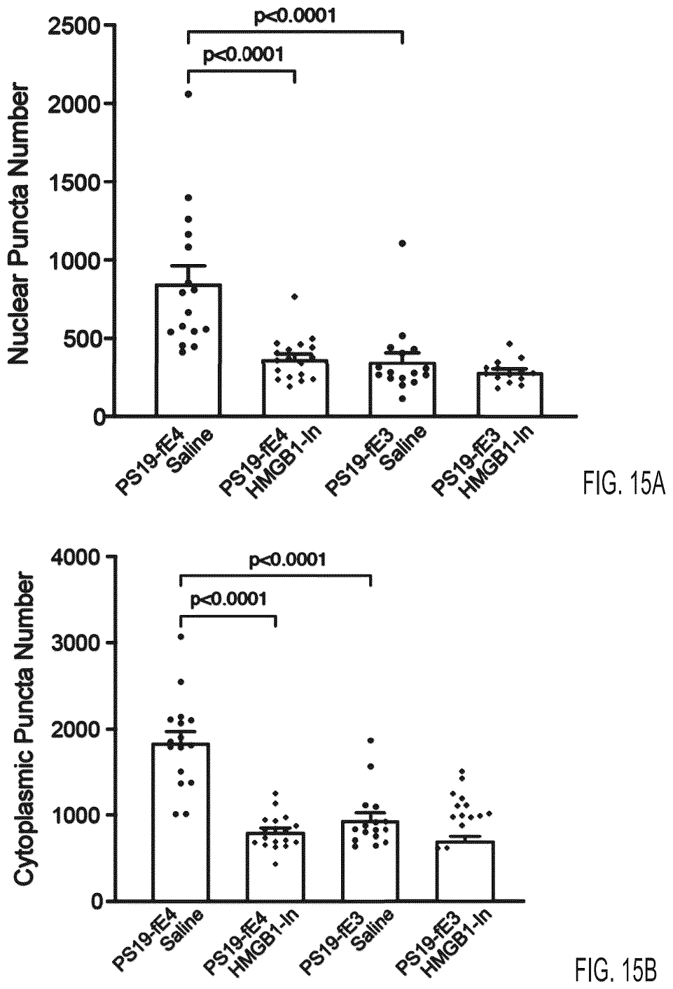

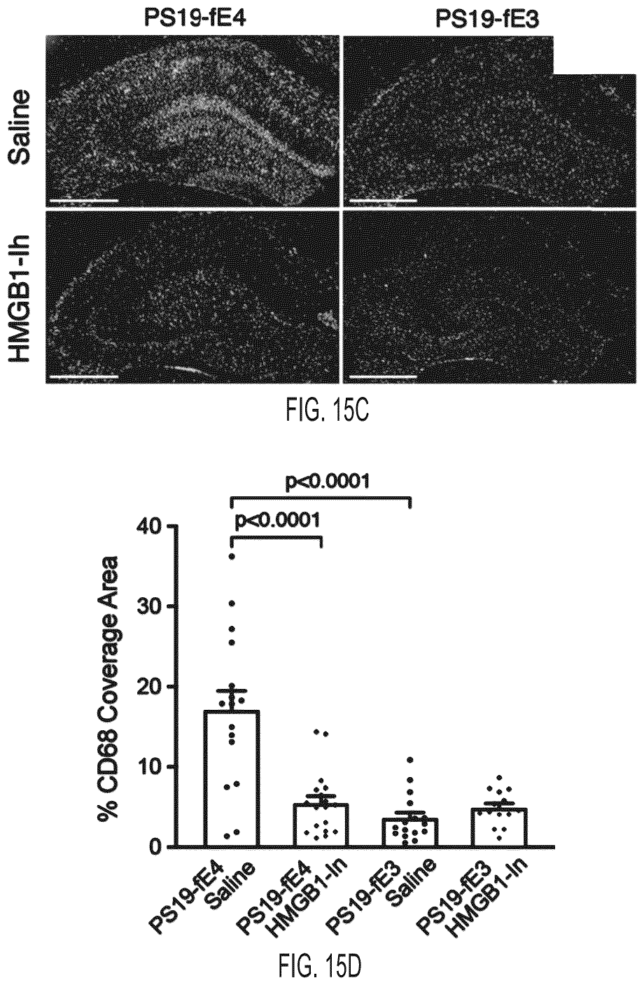

The effectiveness of EP and GA at blocking HMGB1 protein nucleo-cytoplasmic translocation was first confirmed by immunostaining with anti-HMGB1 and DAPI. Saline-treated PS19-fE4 mice exhibited extensive HMGB1 translocation out of the nucleus, whereas PS19-fE4 mice treated with the HMGB1 inhibitors had no discernable HMGB1 translocation (FIG. 15A,B). In PS19-fE3 mice, there was no significant difference in HMGB1 translocation between saline- and inhibitor-treated groups.

Next, the extent of microgliosis and astrogliosis present in the mice following treatment was evaluated. Saline-treated PS19-fE4 mice displayed considerable microgliosis throughout the hippocampus, as exemplified by a high coverage area of Iba1⁺ microglia and CD68⁺ activated microglia (FIG. 15C,D). whereas HMGB1 inhibitor-treated PS19-fE4 mice displayed a significant decrease in microgliosis. Furthermore, immunostaining with anti-GFAP revealed that saline-treated PS19-fE4 mice had extensive astrogliosis throughout the hippocampus, while HMGB1 inhibitor-treated PS19-fE4 mice had a significant reduction in astrogliosis. PS19-fE3 mice did not display significant differences in microgliosis or astrogliosis whether treated with saline or HMGB1 inhibitors.

The extent of Tau pathology in these mice was evaluated by immunostaining for pTau. PS19-fE4 mice treated with saline exhibited substantial Tau pathology throughout the hippocampus, whereas PS19-fE4 mice treated with HMGB1 inhibitors had a drastic reduction in Tau pathology. Furthermore, the effectiveness of HMGB1 inhibitor administration in combating myelin deficits was evaluated by immunostaining for MBP. Saline-treated PS19-fE4 mice displayed severe myelin loss in the stratum radiatum of CA1, while HMGB1 inhibitor-treated PS19-fE4 mice displayed a rescue of the deficit with a high coverage area of MBP. There were no discernable differences in myelin coverage area or Tau pathology between saline- and inhibitor-treated PS19-fE3 mice.

The impact of HMGB1 inhibitors on neurodegeneration by quantifying hippocampi and posterior lateral ventricle volumes was determined. Saline-treated PS19-fE4 mice exhibited considerable neurodegeneration, whereas HMGB1 inhibitor-treated PS19-fE4 mice exhibited a rescue of neurodegeneration through a significant increase in hippocampal volume and a significant decrease in posterior lateral ventricle volume relative to saline-treated PS19-fE4 mice. There were no obvious differences in neurodegeneration between saline- and HMGB1 inhibitor-treated PS19-fE3 mice.

Taken together, these findings illustrate that treatment of PS19-fE4 mice with HMGB1 inhibitors can effectively prevent the development of prominent APOE4-driven pathologies, including gliosis, Tau pathology, neurodegeneration, and myelin deficits, by blocking the nucleo-cytoplasmic translocation and subsequent release of HMGB1 protein. This supports the notion that HMGB1 translocation and release plays a role in the induction and exacerbation of these various APOE4-driven pathologies.

### Discussion

Within the central nervous system, APOE is mainly produced by astrocytes; however, conditions of stress or injury can induce neuronal APOE expression. In the present study, it was demonstrated that APOE4 leads to a significant increase in Tau pathology, gliosis, neurodegeneration, neurodysfunction, and myelin deficits and an enrichment of neurodegenerative disease-associated subpopulations of neurons, oligodendrocytes, astrocytes, and microglia. Removal of neuronal APOE4 drastically reduces all these observed pathologies and largely eliminates the disease-associated cell subpopulations, while removing astrocytic APOE4 only reduces gliosis and neurodegeneration, indicating that neuronal APOE4 is the main driver of many of these pathologies.

Strikingly, snRNA-seq and primary neuron culture studies revealed that removal of astrocytic APOE4 also leads to a considerable, but not complete, reduction in neuronal APOE4 expression and protein production. This suggests that the limited protective effects of removing astrocytic APOE4 on gliosis and neurodegeneration are likely attributable to this partial reduction of neuronal APOE4. The presence of Tau pathology, neurodysfunction, and myelin deficits after removal of astrocytic APOE4, and subsequent reduction of neuronal APOE4, likely suggests that these pathologies are sensitive to low levels of neuronal APOE4 and can only be fully rescued after its complete elimination. In addition, snRNA-seq analysis shows that even after astrocytic APOE4 removal, there is still an enrichment of disease-associated subpopulations of neurons, oligodendrocytes, astrocytes, and microglia. This finding illustrates that despite the overall reduction in gliosis and neurodegeneration after astrocytic APOE4 removal, these neurodegenerative disease-associated cell subpopulations are sensitive to low levels of neuronal APOE4 and they will still be present unless neuronal APOE4 is eliminated completely.

In addition, the neurodegenerative disease-associated neuron subpopulation exhibited a significant increase in expression of HMGB1, which is a nuclear protein known to induce neuroinflammation following its translocation to the cytoplasm and subsequent extracellular release. Mechanistically, it was demonstrated for the first time that neuronal APOE4 promotes HMGB1 release from neurons, as its removal leads to a complete elimination of HMGB1 release. Conversely, astrocytic APOE4 removal leads to a large decrease in HMGB1 release but fails to eliminate it completely, likely due to its partial yet incomplete reduction of neuronal APOE4. This neuronal APOE4-driven HMGB1 release induces gliosis and subsequent neurodegeneration and myelin deficits, all of which can be effectively blocked by a treatment with HMGB1 inhibitors.

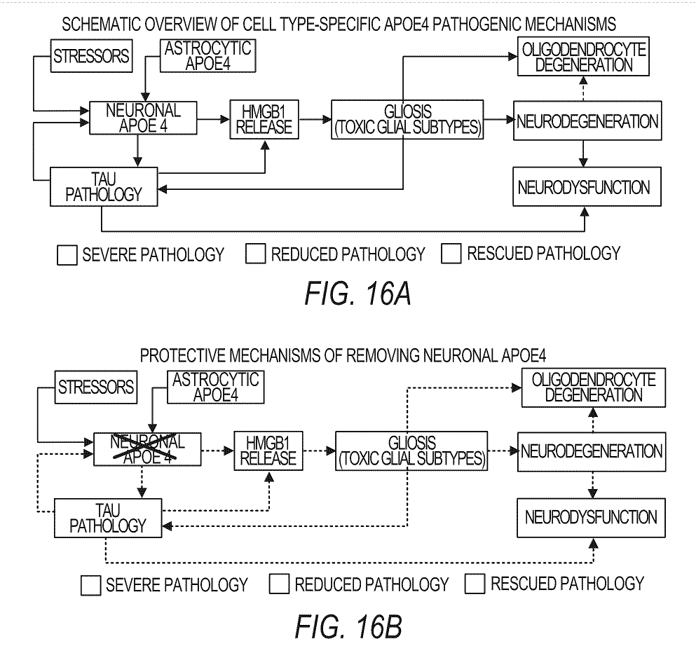

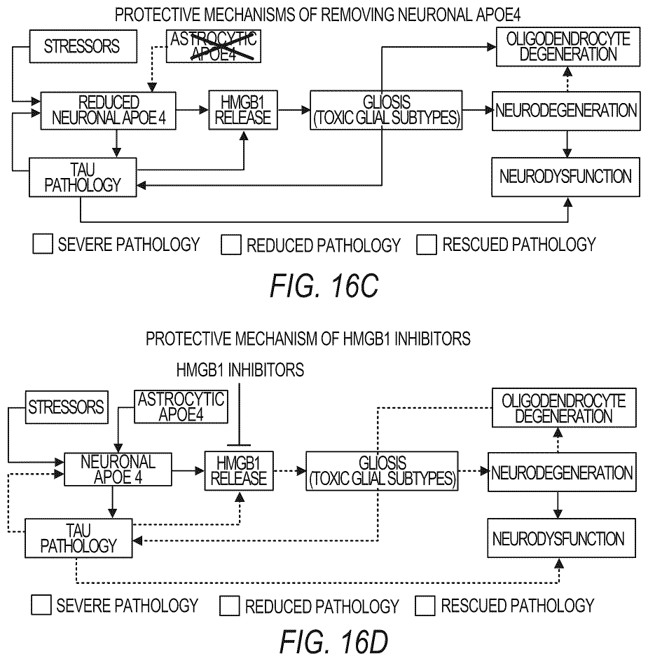

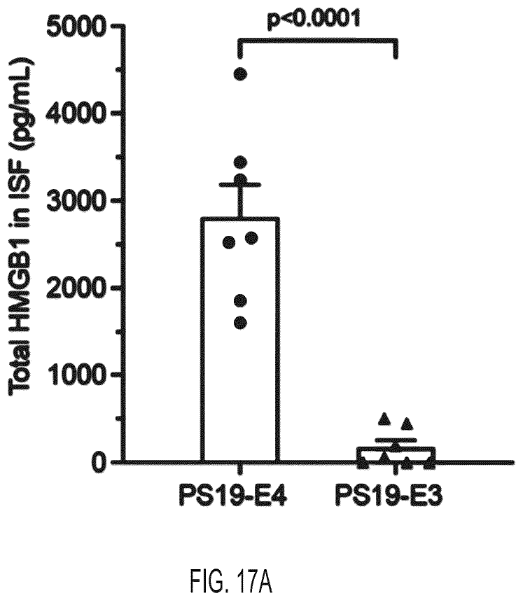

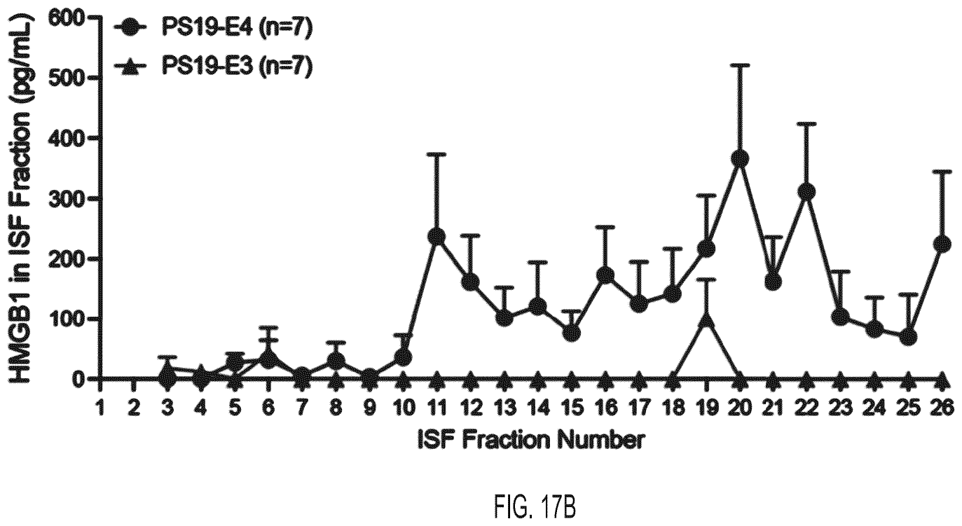

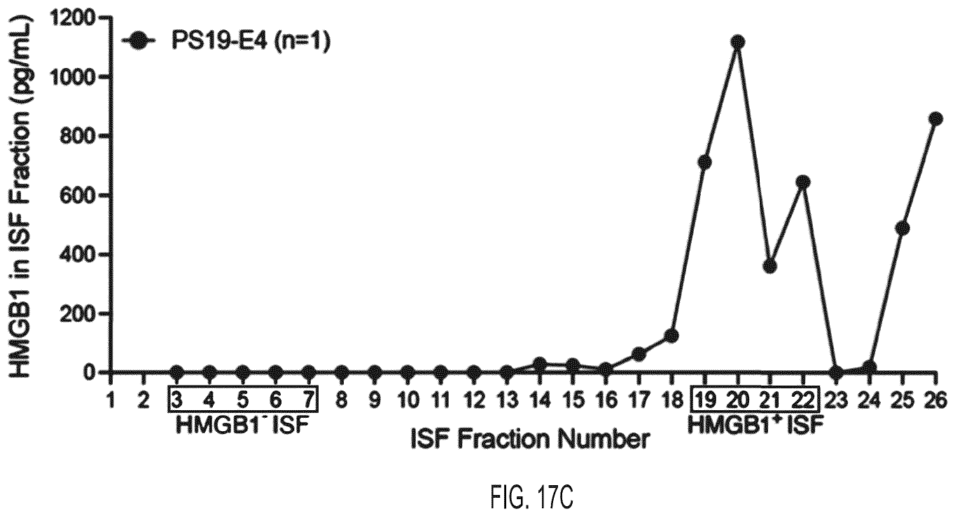

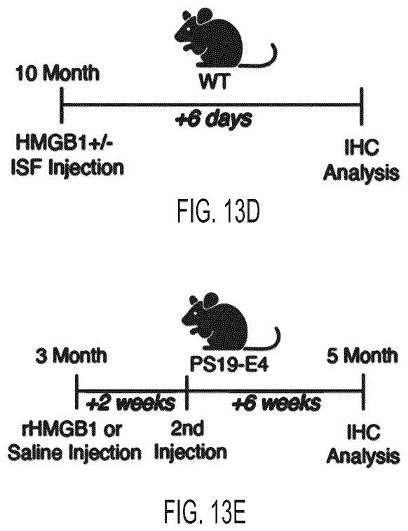

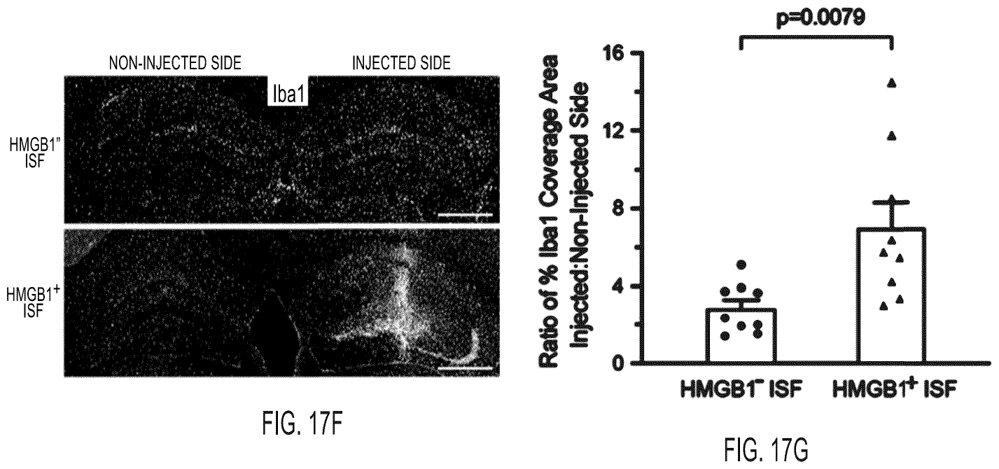

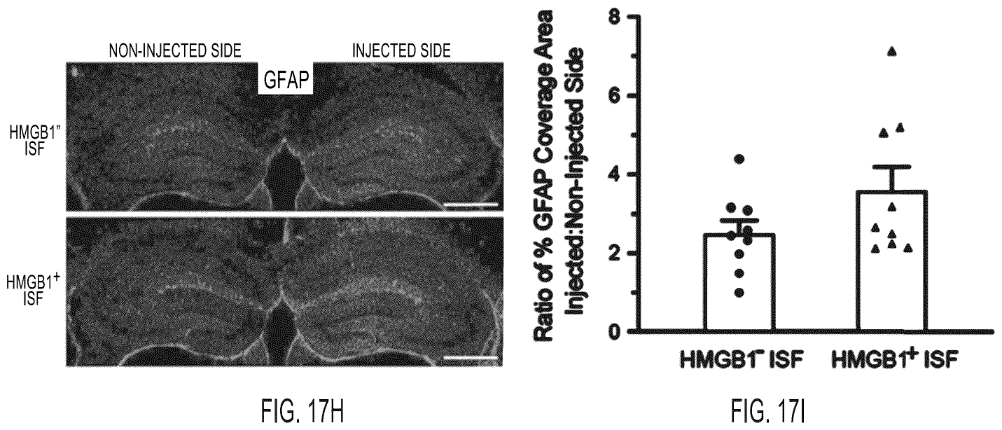

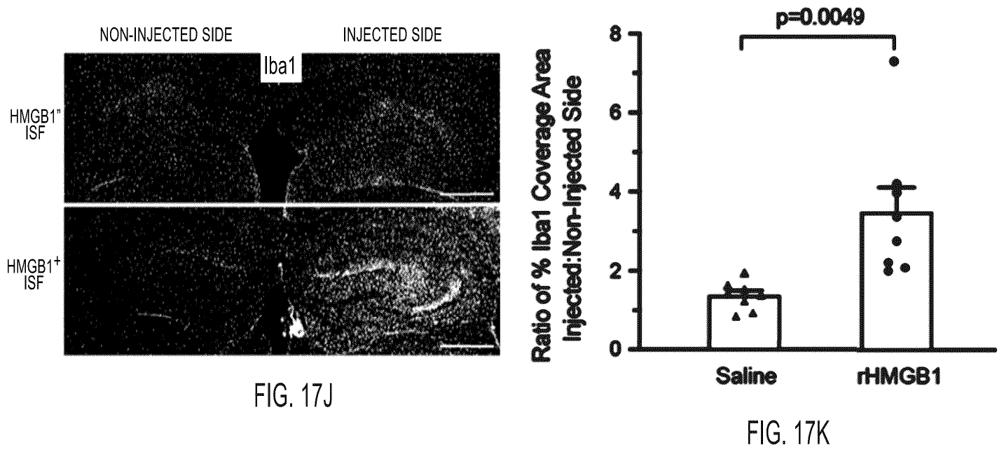

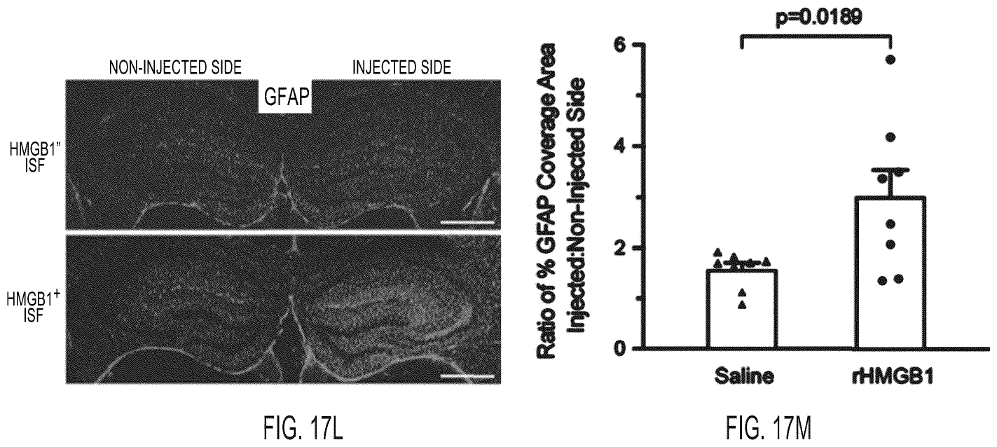

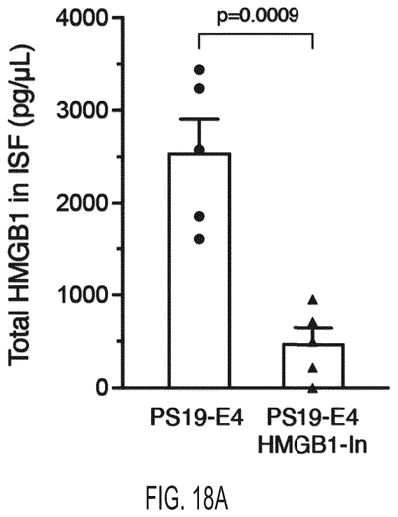

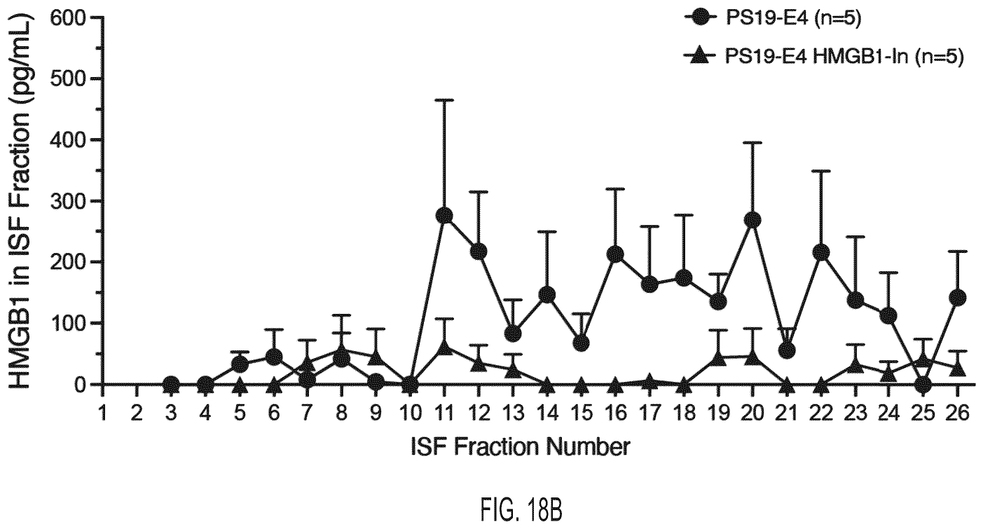

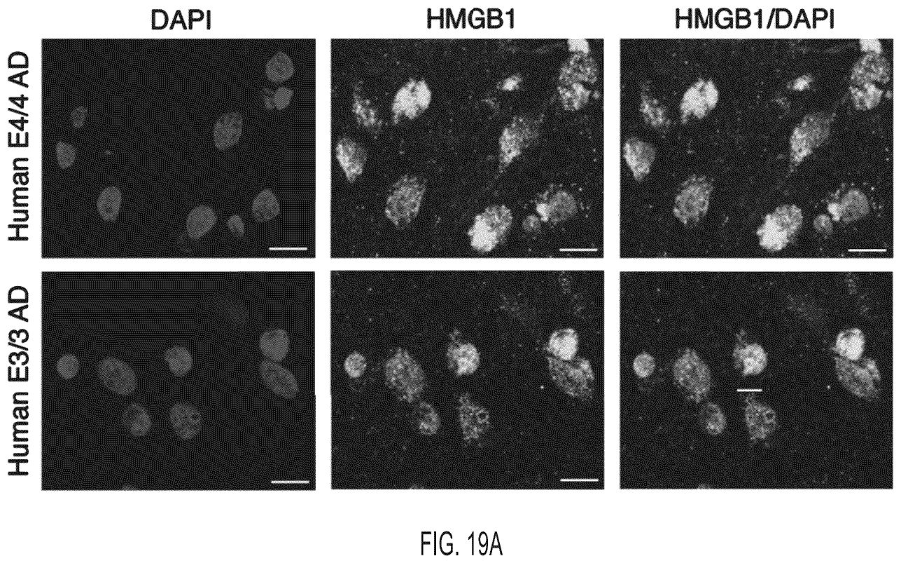

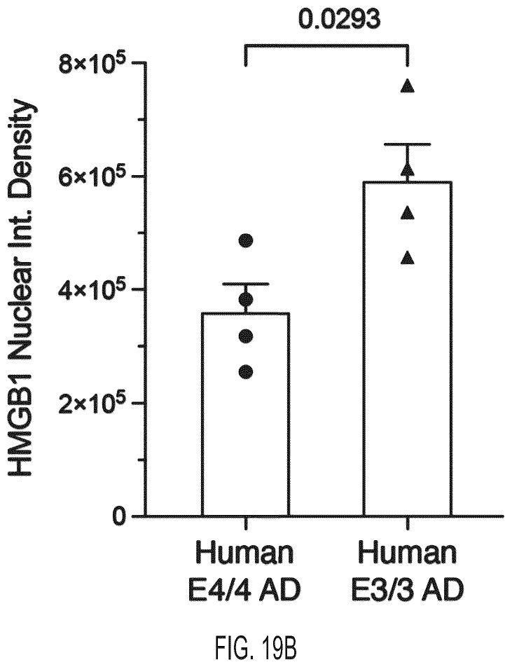

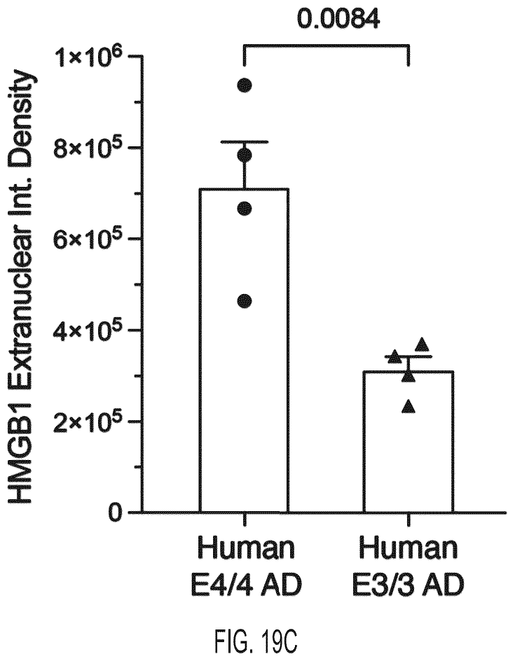

Based on all these findings, an “APOE4-HMGB1-inflammation-degeneration” cascade model of APOE4-related AD and other tauopathies is hypothesized (FIG. 16G). In the presence of both neuronal and astrocytic APOE4, as shown in PS19-fE4 mice (FIG. 16A), a pathogenic cascade initiates with neuronal expression of APOE4, which can be induced by various neuronal stressors. Neuronal APOE4 has a potent effect on the accumulation and propagation of Tau pathology, which can further induce neuronal APOE4 expression. Elevated neuronal APOE4, in concert with Tau pathology, triggers the nucleo-cytoplasmic translocation and release of HMGB1 from neurons. Astrocytic APOE4 does not have a direct effect on Tau pathology accumulation and propagation, however, it indirectly enhances APOE4 expression in neurons by promoting the release of astrocytic factors, such as DPP10, capable of regulating neuronal APOE4 expression, thus, secondarily promoting APOE4/Tau pathology-induced HMGB1 release from neurons. Upon its release from neurons, HMGB1 acts as an inflammatory cytokine that induces gliosis. The extensive gliosis, especially the accumulation of toxic astrocytic and microglial subtypes, results in the aberrant engulfment of neurons, synapses, and oligodendrocyte-derived myelin sheaths and subsequent neurodegeneration, neurodysfunction, and oligodendrocyte degeneration.

Removal of neuronal APOE4, as shown in PS19-fE4/Syn1-Cre mice (FIG. 16B), leads to a drastic reduction of the accumulation and propagation of Tau pathology in neurons. The reduced Tau pathology and the absence of neuronal APOE4 together present as a blockade for HMGB1 release from neurons. Since astrocytic APOE4 contributes to HMGB1 release by indirectly enhancing neuronal APOE4 expression, the lack of neuronal APOE4 blocks the indirect effect of astrocytic APOE4 on triggering neuronal HMGB1 release. Without adequate neuronal HMGB1 release, there is no induction of gliosis, particularly of the toxic microglial and astrocytic subtypes, consequently leading to reduced neurodegeneration, neurodysfunction, and oligodendrocyte degeneration. Thus, removal of neuronal APOE4 leads to a complete prevention or rescue of all observed pathologies.

Removal of astrocytic APOE4, as shown in the PS19-fE4/GFAP-Cre mice (FIG. 16C), leads to a significant, but not complete, reduction in neuronal APOE4 expression. The reduced levels of neuronal APOE4 are still sufficient to induce significant Tau pathology accumulation and propagation, which, together with the low levels of neuronal APOE4, triggers low levels of HMGB1 release. Without high enough levels of HMGB1 release, there is a large reduction in the overall levels of gliosis. Still, the low levels of neuronal APOE4 and HMGB1 release are sufficient to promote the accumulation of toxic astrocytic and microglial subtypes. The overall reduction of gliosis leads to reduced neurodegeneration, although a subpopulation of disease-associated neurons is still present due to the low levels of neuronal APOE4. The presence of toxic glial subtypes still leads to oligodendrocyte deficits, probably due to higher sensitivity of oligodendrocytes to lower levels of toxic glial subtypes. The high levels of Tau pathology and the presence of toxic glial subtypes may continuously contribute to neurodysfunction. Thus, removal of astrocytic APOE4 leads to a partial prevention or rescue of observed pathologies.

Importantly, the pharmacological study indicates that HMGB1 inhibitors represents a novel and effective approach for treating APOE4-related AD and other tauopathies. In the presence of HMGB1 inhibitors (FIG. 16D), the release of HMGB1 from neurons is blocked. Without released HMGB1 to act as an inflammatory cytokine, gliosis is significantly reduced, consequently leading to a significant reduction in neurodegeneration and oligodendrocyte degeneration. The lack of gliosis and potential decrease in toxic glial subtypes also lead to a significant reduction in Tau pathology, which, together with reduced neurodegeneration, likely ameliorates neurodysfunction. Thus, HMGB1 inhibitor treatment also leads to a complete prevention or rescue of all observed pathologies.

While inhibition of HMGB1 has been studied as a viable therapeutic option for a variety of neurological disorders, the present study provides the first evidence that treatment with HMGB1 inhibitors is a valuable and efficacious therapy for combating the APOE4-driven effects on many prominent AD pathologies. Furthermore, our findings also suggest that developing therapies targeting neuronal APOE4 removal are likely to provide higher therapeutic value than astrocytic APOE4 removal. Although removal of either neuronal or astrocytic APOE4 drastically reduces gliosis and neurodegeneration by the 10-month time point we analyzed, as demonstrated in our and other's studies, the long-term consequences of high levels of Tau pathology and the presence of neurodegenerative disease-associated glial subpopulations occurring in the absence of astrocytic APOE4 are unknown and may result in neurodegeneration at a later time point or disrupt other important processes. Furthermore, the removal of neuronal APOE4 provides the additional benefit of reducing Tau pathology, neurodysfunction, myelin degeneration, and OPC depletion. All in all, our study identifies neuronal APOE4 as a central player in triggering a pathogenic cascade in APOE4-related AD and other tauopathies and provides potential targets for anti-AD and tauopathy drug development.

### Example 15: Methods

Mice. Human LoxP-floxed APOE knock-in (fE) mice with conditional deletion of the human APOE gene were generated. Briefly, homozygous fE3 and fE4 mice44 were crossbred with Synapsin 1-Cre transgenic mice [B6.Cg-Tg(Syn1-Cre)671Jxm/J](The Jackson Laboratory)45 or GFAP-Cre transgenic mice [B6.Cg-Tg(GFAP-Cre)8Gtm](National Cancer Institute Mouse Repository). The fE/Cre mice were further crossbred with Tau-P301S (PS19) transgenic mice [B6;C3-Tg(Prnp-MAPT*P301S)PS19Vle/J](The Jackson Laboratory) expressing human P301S 1N4R Tau driven by the PrP promoter to generate PS19-fE4 and PS19-fE3 mice with no Cre, Syn1-Cre, or GFAP-Cre. Littermates that were negative for Syn1-Cre or GFAP-Cre were used as PS19-fE controls. For generation of the PS19-fE/Syn1-Cre line, only female Syn1-Cre mice were used for breeding purposes because germline recombination has been reported to occur in the progeny of male Syn1-Cre mice. Wildtype (WT) mice [C57BL/6J] were obtained from the Jackson Laboratory. All mice were on a pure C57BL/6 genetic background and were housed in a pathogen-free barrier facility on a 12 h light cycle at 19-23° C. and 30-70% humidity. Animals were identified by ear punch under brief isoflurane anesthesia and genotyped by polymerase chain reaction (PCR) of a tail clipping. All animals otherwise received no procedures except those reported in this study. All animal experiments were conducted in accordance with the guidelines and regulation of the National Institutes of Health, the University of California, and the Gladstone Institutes under the protocol AN176773.

For brain tissue collections, mice were deeply anesthetized with intraperitoneal injections of avertin (Henry Schein) and transcardially perfused for 1 min with 0.9% saline. Brains were either fixed as whole brains or hemi-brains, depending on the study. Right hemi-brains were drop-fixed for 48 h in 4% paraformaldehyde (16% PFA diluted in MilliQ H₂O) (Electron Microscopy Sciences), rinsed in 1×PBS (Corning) for 24 h, and cryoprotected in 30% sucrose (Sigma) for 48 h at 4° C. The fixed hemi-brains were cut into 30 m thick coronal sections on a freeze sliding microtome (Leica) and stored in cryoprotectant solution (30% Ethylene Glycol, 30% Glycerol, 40% 1×PBS) at −20° C. Left hemi-brains were snap frozen on dry ice and stored at −80° C.

Immunohistochemistry. For immunofluorescent staining, several sections from each mouse (˜300 m apart) were transferred to a 12-well plate in 1×PBS-T (PBS+0.1% Tween-20) (Millipore Sigma) and were washed 3×5 min in PBS-T to remove cryoprotectant solution. Sections were incubated for 5 min in boiling antigen retrieval buffer (Tris buffer, pH 7.6) (TEKNOVA) and washed 2×5 min in PBS-T. Sections were then incubated in blocking solution (5% normal donkey serum (Jackson Labs), 0.2% Triton-X (Millipore Sigma) in 1×PBS) for 1 h at room temperature to prevent non-specific antibody binding. After blocking, sections were washed 1×5 min in PBS-T and incubated in Mouse-on-Mouse (M.O.M.) Blocking Buffer (1 drop M.O.M IgG/4 mL PBS-T) (Vector Labs) for 1 h at room temperature. After M.O.M. block, sections were incubated in primary antibody at 4° C. overnight after being diluted to optimal concentrations (anti-APOE 1:200 (Cell Signaling); anti-CD68 1:100 (Bio-Rad); anti-Cre 1:800 (Cell Signaling); anti-GFAP 1:800 (Millipore Sigma); anti-GFP 1:5000 (Thermofischer); anti-HMGB1 1:100 (Abcam); anti-Iba1 (rbt) 1:200 (Wako); anti-Iba1 (gt) 1:200 (Abcam); anti-MBP 1:500 (Abcam); anti-NeuN 1:500 (Millipore Sigma); anti-NG2 1:500 (Abcam); anti-S100β 1:200 (Abcam)). Following primary antibody incubation, sections were washed 3×5 min in PBS-T and then incubated in fluorescence-labeled secondary antibodies (Abcam, Jackson Immuno, 1:1000 in PBS-T) for 1 h at room temperature protected from light after being diluted in PBS-T. Sections were then washed 2×5 min in PBS-T and incubated in DAPI (1:50,000 in PBS-T) (Thermofisher) for 8 min at room temperature protected from light. Sections were then washed 2×5 min in PBS-T, mounted onto microscope slides (Fisher Scientific), coverslipped with ProLong Gold mounting media (Vector Laboratories), and sealed with clear nail polish. Images were taken using an FV3000 confocal laser scanning microscope (Olympus) or Aperio VERSA slide scanning microscope (Leica) at 10×, 20×, 40×, or 60× magnifications depending on the stain. Image analyses of percent coverage area were performed using the open-source Fiji (ImageJ) software after setting a standard threshold value that is applied to all images. Researchers were also blinded to samples to exclude the possibility of bias.

For DAB (3,3′-diaminobenzidine) staining, several sections from each mouse (˜300 m apart) were transferred to a 12-well plate in 1×PBS-T and then washed 3×5 min in PBS-T to remove cryoprotectant solution. Sections were then incubated for 5 min in boiling antigen retrieval buffer (1×PBS, 0.1M sodium citrate, 0.1M citric acid) (Fisher Scientific, Fluka) and washed 2×5 min in PBS-T. Next, sections were incubated for 15 min in endogenous peroxidase buffer (1×PBS, 10% methanol (Fisher Scientific), 3% H₂O₂ (Sigma) and washed 3×5 min in PBS-T. Sections were then incubated in blocking solution (1×PBS-T, 5% normal donkey serum, 1% non-fat dry milk) for 1 h at room temperature. After blocking, sections were washed 2×5 min in PBS-T and then incubated in Avidin/Biotin blockage (4 drops of each block) (Vector Laboratories) for 15 min and then washed 2×5 min in PBS-T. Sections were incubated in M.O.M. Blocking Buffer (1 drop M.O.M IgG/4 mL PBS-T) (Vector Labs) for 1 h at room temperature. Following M.O.M. block, sections were washed 2×5 min and incubated in primary antibody at 4° C. overnight after being diluted in PBS-T to optimal concentrations (anti-pTau (AT8) 1:100 (Invitrogen); anti-HT7 1:200 (Peter Davies)). After primary antibody incubation, sections were washed 3×5 min in PBS-T and then incubated in biotinylated secondary antibody (1:200; Jackson Immuno) at room temperature for 1 h. Next, sections were washed 3×5 min in PBS-T and incubated in ABC buffer (Vector Laboratories) that was prepared 10 min prior to the incubation step. Sections were washed for 2×5 min in PBS-T and 1×5 min in Tris buffer (pH 7.6). Sections were incubated in DAB buffer (5 mL 1×PBS, 2 drops Buffer Stock Solution, 2 drops DAB, 2 drops H₂O₂) (Vector Laboratories) for precisely 2 minutes. Staining was halted by washing sections 3×5 min in Tris buffer (pH 7.6) and 2×5 min in PBS-T. Sections were mounted onto microscope slides and dried at room temperature overnight. Next, mounted sections were submerged into Xylene (Fisher Scientific) 2×5 min and coverslipped with DPX mounting media (Sigma-Aldrich). Images were taken using an Aperio VERSA slide scanning microscope (Leica) at 10× magnification.

Volumetric analysis. Serial coronal hippocampal brain sections (7 sections per mouse, 30 μm thick, 300 μm apart) were mounted onto microscope slides (Fisher Scientific) and dried at room temperature for 1 h. The 0.1% Sudan Black solution was prepared by adding the appropriate amount of Sudan Black powder (Sigma) to 70% ethanol (KOPTEC) and mixing the solution using a magnetic stirrer while and protected from light. The solution was then centrifuged at 3,000 RPM for 10 min and the collected supernatant was filtered using a 0.2 m filter syringe (Thermo Scientific) to remove undissolved dye. Sections were then stained with the 0.1% Sudan Black solution at room temperature for 10 min and washed 3×2 min in 70% ethanol and 3×5 min in Milli-Q water. Sections were then coverslipped with ProLong Gold mounting media (Invitrogen) and imaged on an Aperio VERSA slide scanning microscope (Leica) at 10× magnification. For hippocampal and posterior lateral ventricle volumetric analyses, the areas of interest were traced in ImageJ using the segmented line tool and the volume was calculated using the formula: volume=(sum of area)*0.3 mm23. The sum of area value was obtained by taking a sum of the quantified area measurements of all 7 brain sections per mouse, roughly between coordinates AP=−1.2 and AP=−3.4.

Neuronal layer thickness measurements. Two brain sections (30 μm thick, 300 μm apart) underwent immunofluorescence staining as described above using the primary antibody NeuN (1:500) to visualize the neuronal cell layers of the hippocampus. Sections were imaged at 20× magnification using an FV3000 confocal laser scanning microscope (Olympus). The thickness of the CA1 pyramidal cell layer and dentate gyrus granular cell layer of the hippocampus were measured on the Fiji (ImageJ) software by drawing a straight line perpendicular to the NeuN+ cell layers at two points per hippocampal subfield and taking the average value for each mouse.

Nuclear-cytoplasmic localization of HMGB1 measurements. Two brain sections (30 m thick, 300 m apart) were immunostained with anti-HMGB1 (1:100) and DAPI (1:50,000) as described above. Sections were imaged at 40× and 60× magnification using an FV3000 confocal laser scanning microscope (Olympus). All image processing and quantification was performed on the Fiji (ImageJ) software. Briefly, a 1-pixel median filter was applied to the DAPI channel and an appropriate threshold was set to create a mask of DAPI. The image calculator function was then used to overlay the DAPI mask and HMGB1 channel, which provided the HMGB1 staining that was only localized to the nucleus. After obtaining values for integrated density and particles, the image calculator was used to subtract the DAPI mask from HMGB1, which provided HMGB1 staining that was excluded from the nucleus.

Biochemical extraction of brain tissue. The hippocampus was dissected from snap frozen mouse hemibrains after thawing on ice. The hippocampal tissue was weighed and homogenized using a Polytron immersion disperser Polytron homogenizer (Kinematica AG) in ice-cold RAB buffer (G Biosciences) at 10 L/mg tissue, supplemented by phosphatase inhibitors (Roche) and protease inhibitors (Roche). Samples were then centrifuged using an Optima TLX ultracentrifuge (Beckman Coulter) at 50,000 g for 20 min at 4° C. and the supernatant was collected as the RAB-soluble fraction. The pellets were resuspended in ice-cold RIPA buffer (Thermo Scientific) at 10 L/mg tissue and centrifuged at 50,000 g for 20 min at 4° C. The supernatant was collected as the RIPA-soluble fraction and the pellet was stored at −80° C. for further use. All fractions were stored at −80° C. until further analyses.

Western blot analysis. Biochemically extracted mouse hippocampal tissue lysates were loaded onto 12% Bis-Tris SDS-PAGE gels (Invitrogen) and separated by gel electrophoresis at 160V using MOPS buffer. The separated proteins were transferred onto nitrocellulose membranes at 18V for 60 min (Trans-Blot Turbo Transfer System (Bio-rad). Membranes were washed 3×5 min in PBS-T and then incubated in Intercept blocking buffer (LI-COR) for 1 h at room temperature to block non-specific binding sites. After blocking, membranes were washed 3×5 min in PBS-T and incubated with primary antibody overnight at 4° C. (AT8 1:3,000 (Invitrogen), TUJ1 1:15,000 (Biolegend)). Membranes were washed 3×5 min in PBS-T and incubated in fluorescently-labeled secondary antibody (1:20,000; LI-COR) for 1 h in the dark at room temperature. Resulting bands were detected with the Odyssey CLx infrared imaging system (LI-COR), and the fluorescence intensity of the bands was quantified as a ratio of AT8:TUJ1 signal using the Image Studio software.

Sandwich ELISA. Biochemically extracted mouse hippocampal tissue lysates were diluted in Milli-Q H₂O to the appropriate concentration and were run according to the provided manufacturer protocols (human APOE (Abcam); mouse HMGB1 (Novus Biologicals). Reactions of samples were read on a SpectraMaX M5 spectrophotometer (Molecular Devices) and protein concentrations were determined after interpolating a standard curve and adjusting for dilutions.

Primary neuron cultures and recombinant protein treatment. Primary cultures of neurons were prepared from prenatal E20 pups of various genotypes. After collecting pup brains, the cortex plus hippocampus were isolated and placed in ice-cold dissociation media+kyneurenic acid media (DM/KY) (DM:Na₂SO₄ (81.8 mM); K₂SO₄ (30 mM); MgCl₂ (5.8 mM), CaCl₂) (0.25 mM); HEPES (1 mM); glucose (20 mM); phenol red (0.001%); NaOH (0.16 mM) in distilled H₂O)(KY: kynurenic acid (10 mM); phenol red (0.0025%); HEPES (5 mM); MgCl₂ (100 mM); NaOH (add dropwise until pH 7.4). The resulting DM/KY media was made by combining 90% DM with 10% KY media. The isolated tissue was finely minced and then submerged in pre-warmed Papain solution (1 mL per brain) for 13 min while gently inverting and then submerged in trypsin inhibitor solution for 5 min (5 mL for up to 10 brains) while gently inverting. The tissue pellet was washed with Optimem/Glucose solution (20 mM glucose, 1 mL per brain) while gently inverting. Then, fresh Optimem/Glucose solution was added and tissue was gently triturated until separated into single cells. After filtering the cells through a 40 um cell strainer, the dissociated cells were plated at 1×10⁶ cells/well of a 12-well plate or 3×10⁵ cells/well of a 24-well plate in Neurobasal medium supplemented with B27, 100 U/mL⁻¹ of penicillin G, 100 g/mL⁻¹ of streptomycin, and 1% GlutaMAX (B27/Neurobasal). Every 3-4 days, half of the media was removed and replaced with fresh B27/Neurobasal media. In some experiments, primary neurons were treated with either a Dulbecco's PBS (dPBS) vehicle or a recombinant protein of interest (10 g/mL for one well of a 12-well plate) for 24 hrs at day 14 in vitro. Following treatment, the media was collected and the cultures were harvested for analysis. Total protein levels present in cell lysates were obtained thru BCA analysis (Pierce).

Stereotaxic surgery on mice. Mice were anesthetized with an intraperitoneal injection of ketamine (60 mg/kg) and xylazine (30 mg/kg) and maintained on 0.8%-1.0% isofluorane (Henry Schein). Mice were secured in a stereotaxic alignment system model 940 using earbars and a tooth bar (Kopf Instruments). The scalp was prepared by removing hair using Nair and sterilizing with 70% ethanol. The scalp was then cut open using a scalpel and sterilized with 70% ethanol. The cranial sutures were better visualized using 3% hydrogen peroxide. Following identification of Bregma, a unilateral stereotaxic site was drilled with a 0.5 mm microburr (Fine Science Tools) using coordinates X=+1.5, Y=−2.1, Z=−2.1, with Z measured from the surface of the brain. Mice were injected with 2 μL of the respective virus (AAV2(Y444F)-smCBA-human_P301S_Tau-WPRE, 2.10E+13 vg/mL, Virovek); AAV2-Synapsin-GFP, 1.0E+13 vg/mL, SignaGen) or ISF fraction at a rate of 500 nL/min and allowed to diffuse for 3 min. Following surgery, mice were sutured with nylon monofilament nonabsorbable 6-0 sutures (Henry Schein), and administered analgesics buprenorphine (0.0375 mg/kg intraperitoneally), ketophen (5 mg/kg subcutaneously), and saline (500 μL intraperitoneally). Mice were monitored on a heating pad until ambulatory and provided Hydrogel for hydration.

Brain slices electrophysiological recordings and data analyses. For electrophysiological recording study, 8-month-old PS19-fE3 mice and PS19-fE4 mice with no Cre, Syn1-Cre, or GFAP-Cre were anaesthetized with isoflurane and decapitated. The brain was rapidly removed from the skull and placed in ice-cold (2-5° C.) slicing solution. Slicing solution contained (in mM): 110 choline chloride, 2.5 KCl, 26 NaHCO₃, 10 MgCl₂, 1.25 NaH₂PO₄, 0.5 CaCl₂), 10 glucose, 3 Na Pyruvate, 1 L-Ascorbic acid, pH 7.4. 350 m-thick sagittal slices were cut from both hemispheres using a vibratome (VT1200, Leica) and transferred to a 95% 02-C02 vapor interface holding chamber (BSK5, Scientific Systems Design) containing artificial cerebrospinal fluid (ACSF) where they were allowed to recover at 34° C. for one hour and held at room temperature (20-22° C.) afterwards. ACSF contained (in mM): 126 NaCl, 2.5 KCl, 1.5 CaCl₂), 1.5 MgCl₂, 26 NaHCO₃, 1.25 NaH₂PO₄, 10 glucose, and 1.5 L-Ascorbic acid, pH 7.4.

For input/output recording studies, local field post-synaptic potentials (fPSPs) were elicited by orthodromic stimulation of Schaffer collaterals by concentric bipolar stimulating electrode (FHC) connected to a constant voltage isolated stimulator (DS2A-MKII, Digitimer North America) and placed in CA2 stratum radiatum. fPSPs were recorded with a glass borosilicate microelectrode filled with ACSF and placed in CA1 stratum radiatum. Signals were sampled and digitized by MultiClamp 700B amplifier and Digidata 1550B1 acquisition system with pClamp10 software (Molecular Devices), and analyzed using IgorPro6 software (Wavemetrics) running custom macros. fPSP slopes were analyzed as the linear fit slope values between 10% and 90% of fPSP peak. Input-output relationships were recorded as the fPSP slope values in response to increasing stimulation intensity (20-60 μA), with fPSP slope gain calculated as the linear slope of the resulting input-output curve.

Microdialysis of mouse hippocampus. Brain interstitial fluid was collected using in vivo microdialysis of the hippocampus. Surgical procedures, including pre- and post-operative care, were conducted as described above for stereotaxic surgeries. During the surgery, a unilateral stereotaxic site was drilled with a 1.2 mm bone drill bit (BASi) and an AtmosLM guide cannula PEG-4 (Amuza) was stereotaxically implanted above the right hippocampus at coordinates X=+1.5, Y=−2.1, Z=−1.1. The cannula was secured in place using dental cement (GC America), and a temporary PEG-4 AtmosLM dummy probe (Amuza) was inserted and fixed with an AC-5 cap nut screw (Eicom). Two days post-surgery, mice were placed in a microdialysis stand-alone system (BASi) overnight to assimilate, and the following afternoon a 1000 kDa AtmosLM collection probe (Eicom) was inserted through the guide cannula into the hippocampus, which extends 1 mm father down to Z=−2.1 to target the dentate gyrus. Artificial CSF (Harvard Apparatus) made with 0.15% BSA (Thermo Scientific) was circulated through the system at a rate of 0.5 L/min using a push-pull method, and ISF was collected in a refrigerated fraction collector (BASI) each hour for roughly 24 hours. To prevent clogging of the tubing, pumps were operated at 10× collection speed for the first two hours before being adjusted to a 0.5 L/min flow rate. Following completion of ISF collection, mice were euthanized and perfused with 0.9% saline, as described above. The brain was dissected into hemispheres, with the right hemi-brain postfixed for 48 hours in 4% PFA and the left hemi-brain fresh frozen. ISF fractions were frozen at −80° C. for further analysis.

Treatment with HMGB1 inhibitors. At 6.5 months of age, male and female PS19-fE4 and PS19-fE3 mice were randomly assigned to the control or treatment group. Mice received intraperitoneal injections with either sterile grade 0.9% saline (Fisher Scientific) or a mixture of HMGB1 inhibitors: ethyl pyruvate (80 mg/kg) (Sigma-Aldrich) and glycyrrhizic acid (20 mg/kg) (Sigma-Aldrich) dissolved in 0.9% saline. The mice received three injections per week for 12 weeks, starting at 6.5 months of age until they reached 9.5 months of age. All mice were monitored for weight changes, grooming changes, and posture during the experiments and no changes were observed. Following treatment, the animals were perfused and their brain tissue was processed for histopathological analysis, as described above.

Single-nuclei preparation for 10× loading. The mouse hippocampus was dissected on ice and placed into a pre-chilled 2 mL Dounce with 1 mL of cold 1× Homogenization Buffer (1× HB) (250 mM Sucrose, 25 mM KCL, 5 mM MgCl2, 20 mM Tricine-KOH pH7.8, 1 mM DTT, 0.5 mM Sermidine, 0.15 mM Sermine, 0.3% NP40, 0.2 units/L RNase inhibitor, 0.2 units/μL Protease inhibitor). Dounce with “A” loose pestle (˜10 strokes) and then with “B” tight pestle (˜15 strokes). The homogenate was filtered using a 7 μM Flowmi strainer (Eppendorf) and transferred to a pre-chilled 2 mL LoBind tube (Fischer Scientific). Nuclei were pelleted by spinning for 5 min at 4° C. at 350 RCF. The supernatant was removed and the nuclei were resuspended in 400 L 1× HB. Next, 400 L of 50% Iodixanol solution was added to the nuclei and then slowly layered with 600 L of 30% Iodixanol solution under the 25% mixture, then layered with 600 μL of 40% Iodixanol solution under the 30% mixture. The nuclei were then spun for 20 min at 4° C. at 3,000 g in a pre-chilled swinging bucket centrifuge. 200 μL of the nuclei band at the 30%-40% interface was collected and transferred to a fresh tube. Then, 800 μL of 2.5% BSA in PBS plus 0.2 units/μL of RNase inhibitor was added to the nuclei and then were spun for 10 min at 500 RCF at 4 C. The nuclei were resuspended with 2% BSA in PBS plus 0.2 units/μL RNase inhibitor to reach ˜500 nuclei/μL. The nuclei were then filtered with a 40 μM Flowmi stainer. The nuclei were counted and then ˜13,000 nuclei per sample were loaded onto 10× Genomics Next GEM chip G. The snRNA-seq libraries were prepared using the Chromium Next GEM Single Cell 3′ Library and Gel Bead kit v3.1 (10× Genomics) according to the manufacturer's instructions. Libraries were sequenced on an Illumina NovaSeq 6000 sequencer at the UCSF CAT Core.

Custom reference genome. PS19 tau mutant floxed APOE knock-in mouse model was used for single-nucleus RNA-sequencing (snRNA-seq). The Homo sapiens microtubule associated protein tau (MAPT) (NCBI Reference Sequence: NM_001123066.4) and the Homo sapiens APOE are genes of interest for this study. These genes are not expected to be a part of the mouse reference genome, so to quantify the reads aligning to these genes of interest, a custom mouse reference genome was made using the reference mouse genome sequence (GRCm38) from Ensembl (release 98) and the mouse gene annotation file from GENCODE (release M23)⁷⁹, similar to those used in 10× Genomics Cell Ranger mouse reference package mm10 2020-A. The headers of the Ensembl reference mouse genome sequence fasta file with the chromosome names were modified to match the chromosome names in a fasta file from GENCODE. The annotation GTF file contains entries from non-polyA transcripts that overlap with the protein coding genes. These reads are flagged as multi-mapped and are not counted by the 10× Genomics Cell Ranger v6.1.1 count pipeline. To avoid this, the GTF file was modified to (1) remove version suffixes from transcript, gene, and exon ids to match the Cell Ranger reference packages, (2) remove non-polyA transcripts. The Homo sapiens MAPT sequence and Homo sapiens APOE sequence were appended as separate chromosomes to the end of the mouse reference genome sequence and the corresponding gene annotations were appended to the filtered mouse reference gene annotation GTF file. The 10× Genomics Cell Ranger v6.1.1 mkref pipeline was used to build the custom reference genome using the modified fasta and GTF file.

Pre-processing and clustering of mouse snRNA-seq samples. The snRNA-seq samples included a total of 16 samples with four mice from each of the four genotype groups (PS19-fE4, PS19-fE4 Syn1-Cre, PS19-fE4 GFAP-Cre, and PS19-fE3). Each group of four mice had two male and two female mice. The demultiplexed fastq files for these samples were aligned to the custom mouse reference genome (See custom reference genome methods for additional descriptions) using the 10× Genomics Cell Ranger v6.1.1 count pipeline, as described in the Cell Ranger documentation. The include-introns flag for the count pipeline was set to true to count the reads mapping to intronic regions. The Cell Ranger count web summaries showed a “Low Fraction Reads in Cells” error for two samples—one from the PS19-fE4 GFAP-Cre group and one from the PS19-fE3 group. These two samples had only ˜40% reads assigned to cell-associated barcodes and <80% reads mapped to the genome. These metrics were much higher for other 14 samples. Checking the experimental record indicated that these two samples had issues at the nuclear isolation step and lower cDNA was recovered due to the use of an expired old batch of sample preparation reagents. All other 14 samples were prepared with a new batch of sample preparation reagents. So, these two samples were excluded and only the remaining 14 samples were used for the downstream analyses with Seurat.

The filtered count matrices generated by the Cell Ranger count pipeline for 14 samples were processed using the R package for single-nucleus analysis Seurat v4.0.5⁸¹. Each sample was pre-processed as a Seurat object and the top 1% of cells per sample with a high number of unique genes, cells with <=200 unique genes, and cells >=0.25% mitochondrial genes were filtered out for each sample. The 14 samples were merged into a single Seurat object and normalization and variance stabilization was performed using sctransform with the “glmGamPoi” (Bioconductor package version 1.6.0) method for initial parameter estimation.

Graph-based clustering was performed using the Seurat v4.0.5 functions FindNeighbors and FindClusters. First, the cells were embedded in a k-nearest neighbor (KNN) graph based on the Euclidean distance in the PCA space. The edge weights between two cells were further modified using Jaccard similarity. Next, clustering was performed using the Louvain algorithm implementation in the FindClusters Seurat function. Clustering was performed for all combinations of 10, 15 and 20 PCs with 0.4, 0.5, 0.6, 0.7, 0.8 and 0.9 resolutions. Clustering with 15 PCs and 0.7 resolution resulted in 33 distinct biologically relevant clusters, which was used for further analyses.

Cell type assignment. Data visualization using Seurat v4.0.5 in the UMAP space for the 14 samples revealed no batch effects by age, sex, genotype, date of birth, or nuclear isolation date. The marker genes for each cluster were identified using the FindAllMarkers Seurat function on the SCT assay data. This algorithm uses the Wilcoxon Rank Sum test to iteratively identify differentially expressed genes in a cluster against all the other clusters. Marker genes were filtered to keep only positively expressed genes, detected in at least 25% of the cells in either population and with at least 0.5 log 2 fold change. Identities were assigned to cell clusters by matching the cell clusters to known cell types with the expression of canonical cell-type-specific genes, the expression of genes identified in publicly available mouse hippocampal single-cell RNA-seq datasets, and the expression of each cluster's marker genes in a publicly available resource of brain-wide in situ hybridization images.

Subclustering of astrocytic and microglial sn-RNA-seq data. The hippocampal cell cluster 10 was annotated as the astrocyte cells and hippocampal cell clusters 11, 21 and 29 were annotated as the microglial cells. Both these cell types were sub-clustered. Normalization and variance stabilization was performed using sctransform⁸² with the “glmGamPoi” (Bioconductor package version 1.6.0) method for initial parameter estimation. Graph-based clustering was performed using the Seurat v4.0.5 functions FindNeighbors and FindClusters. First, the cells were embedded in a k-nearest neighbor (KNN) graph based on the Euclidean distance in the PCA space. The edge weights between two cells were further modified using Jaccard similarity. Next, clustering was performed using the Louvain algorithm implementation in the FindClusters Seurat function. Clustering was performed for all combinations of 10, 15, 20, 25 and 30 PCs with 0.4, 0.5, 0.6, 0.7, 0.8 and 0.9 resolutions. Sub-clustering with 15 PCs and 0.9 resolution resulted in 18 distinct biologically relevant subclusters for astrocytes. Sub-clustering with 15 PCs and 0.9 resolution resulted in 18 distinct biologically relevant microglia subclusters.

Gene-set enrichment analysis. Differentially expressed genes between clusters of interest were identified using FindMarkers Seurat function on the SCT assay data. This algorithm uses the Wilcoxon Rank Sum test to identify differentially expressed genes between two populations. Differentially expressed genes were limited to genes detected in at least 10% of the cells in either population and with at least 0.1 log 2 fold change. Over-representation (or enrichment) analysis was performed using clusterProfiler v4.2.1 to find gene sets in the KEGG database for mouse associated with the differentially expressed genes. The p-values are based on a hypergeometric test and are adjusted for multiple testing using the Benjamini-Hochberg method. Significantly enriched gene sets were filtered to have an adjusted p-value less than 0.8 and at least 10 differentially expressed genes present in the gene set. The same method was used for gene-set enrichment analysis of astrocyte subclusters and microglia subclusters.

Association between clusters and genotype. A Generalized Linear Mixed-Effects Model to assess association with Animal Models (GLMM_AM) was implemented in the lme4 (v1.1-27.1) R package and used to estimate the associations between cluster membership and the mouse model. These models were run separately for each cluster of cells. The GLM model was performed with the family argument set to the binomial probability distribution and with the bobyqa control optimizer used for the maximum likelihood estimation. Cluster membership for each cell was modeled as a 0-1 response variable according to whether or not the cell belongs to the cluster under consideration. The corresponding mouse id from which the cell was derived was the random effect variable and the animal model for this mouse id was included as the fixed variable. The reference animal model was set to PS19 fE4. The resulting p-values for the estimated log odds ratio across the three animal models (with respect to the PS19 fE4) and clusters were adjusted for multiple testing using the Benjamini-Hochberg method. The same method was used for estimating the between cluster association with genotype for astrocyte subclusters and microglia subclusters.

Association between proportion of cell types and histopathological parameters. A Generalized Linear Mixed-Effects Model to assess association with histopathology (GLMM_histopathology) was implemented in the lme4 (v1.1-27.1) R package and used to identify cell types whose proportions are significantly associated with changes in histopathology across the samples. These models were performed separately for each combination of the cluster of cells and the eight histological parameters: hippocampal volume (mm³), the percent of AT8 coverage area, the percent of GFAP coverage area, the percent of S100β coverage area, the percent of IBA1 coverage area, the percent of CD68 coverage area, the percent of MBP coverage area, and the percent of OPC coverage area. The GLM model was performed with the family argument set to the binomial probability distribution family and with the ‘bobyqa’ control optimizer used for the maximum likelihood estimation. Cluster membership for each cell was modeled as a 0-1 response variable according to whether or not the cell belongs to the cluster under consideration. The corresponding mouse model from which the cell was derived was included as a random effect and further the mouse id within the given mouse model was modeled as a random effect as well. Note, this represents the hierarchical nature of this data for the GLMM, and the mouse models are first assumed to be sampled from an “universe” of mouse models, this is then followed by sampling mice within each mouse model. The modeling choice of including the mouse model as a random effect as opposed to a fixed effect is meant to increase the degrees of freedom (or maximize the statistical power) to detect the association of interest, particularly in light of the relatively small number of replicates (3-4) per animal model. The histological parameter under consideration was modeled as a fixed effect in this model.

A subset of cell types of interest was selected and the log Odds ratio estimates (derived from the GLMM fits) were visualized in a heatmap using pheatmap package 1.0.12 after adjusting the p-values distribution across histopathological parameters across cell types with Benjamini-Hochberg multiple testing correction. The pipeline was applied to the astrocyte and microglia subtypes and visualized the associations between astrocyte and microglia subtypes of interest and the eight histopathological parameters in FIG. 4h and FIG. 4i, respectively. The first 5 principal component coordinates were estimated using the eight log Odds ratio for unit change of the histopathological parameters for each of the cell types and astrocyte and microglia subcell types of interest. This was implemented using prcomp(scale=T, center=T) in stats R package. The first two PCs were visualized using fviz_pca_ind( ) implemented in factoextra 1.0.7 R package.

Mouse snRNA-seq dataset from GEO:GSE164507. Mouse snRNAseq data that are available on the Gene Expression Omnibus database: www.ncbi.nlm.nih.gov/geo (accession no. GSE164507) were reanalyzed. The complete publicly available data set which, for each sample, includes a filtered matrix of gene by cell expression, file with barcodes, and file with expressed genes, was downloaded. Briefly, the study examined P301S mutant Tau transgenic mice carrying floxed APOE-ε4 or APOE-ε3 alleles. These mice were crossed with mice expressing a Cre recombinase under the regulation of a tamoxifen-inducible ER element and the Aldh111 astrocyte-specific promoter. These Aldh111-CreERT2 mice were administered either tamoxifen, to induce Cre recombinase expression, or a vehicle at 5.5 months of age, after the onset of tau pathology. Isolated single nuclei from the hippocampus of these mice were sequenced using 10× Genomics Chromium Single Cell sequencing and the data was processed using Cell Ranger Single Cell Software Suite (v3.0.2). The filtered count matrices generated by the Cell Ranger count pipeline for all 8 samples were processed using Seurat v4.0.481. Samples were filtered to include only cells with 500-2,000 genes detected and <5% mitochondrial reads. The filtered samples were merged into a single Seurat object containing a matrix of 33,457 genes by 63,248 nuclei. Normalization and variance stabilization were performed using sctransform. Clustering was determined as implemented in Seurat v4.0.4 with the RunPCA( ), FindNeighbors( ), and FindClusters( ) functions. Nearest neighbor distances were computed using up to the first 15 PCs. This algorithm embeds cells in a k-nearest neighbor graph, based on Euclidean distance in PCA space. The edge weights between any two cells are further refined using Jaccard similarity. Clustering was implemented using the Louvain algorithm with the default settings and a resolution of 0.7, resulting in a set of 22 distinct clusters. Differential gene expression was detected using the Wilcoxon Rank Sum test in Seurat, implemented via the FindMarkers( ) function with min.pct=0.1, test.use=“wilcox”, and logfc.threshold=0.05.

General statistical analysis. The differences between genotype groups were evaluated by ordinary oneway ANOVA with Tukey's multiple comparisons test, where the mean of each column was compared with the mean of every other column. All plotted data are presented as the mean±SEM. The correlations between two data in the same genotype group were analyzed using simple linear regression and plotted as the mean±SEM. The analyses were performed and plots were created with GraphPad Prism version 9.2.0.

### REFERENCES

Agarwala et al., Nucleic Acids Res., 44:D7 (2016).
Ahlmann-Eltze et al., Bioinformatics, 36:5701 (2021).
Andrews-Zwilling et al., J. Neurosci., 30:13707 (2010).
Bajenaru et al., Mol. Cell Biol. 22:5100 (2002).
Bates et al., J. Stat. Soft., 67:1 (2015).
Benitez et al., Neuroimage Clin., 4:64 (2013).
Benjamini & Hochberg, J. R. Stat. Soc. Ser. B Stat. Methodol., 57:289 (1995).
Bien-Ly et al., J. Neurosci., 32:4803 (2012).
Boluda et al., Acta Neuropathol., 129:221 (2015).
Brecht et al., J. Neurosci., 24:2527 (2004).
Buttini et al., J. Neurosci.; 19:4867 (1999).
Cerutti & Chadi, Cell Biol. Int., 24:35 (2000).
Cho et al., Ann. Neurol., 80:247 (2016).
Corder et al., Science (80-.), 261:921 (1993).
Dave et al., J. Leukoc. Biol., 86:633 (2009).
Dean III et al., JAMA Neurol., 74:41 (2017).
Desai et al., Glia, 57:54 (2009).
Egensperger et al., Brain Pathol., 8:439 (1998).
Farrer et al., JAMA, 278:1349 (1997).
Frankish et al., Nucleic Acids Res., 49:D916 (2021).
Gale et al., J. Allergy Clin. Immunol., 134:127 (2014).
Gauley & Pisetsky, Autoimmunity, 42:299 (2009).
Guo et al., J. Exp. Med., 213:2635 (2016).
Hafemeister & Satija, Genome Biol., 20:296 (2019).
Hao et al., Cell, 184:3573 (2021).
Howe et al., Nucleic Acids Res., 49:D884 (2021).
Huang & Mahley, Neurobiol. Dis., 72:3 (2014).
Huang & Mucke, Cell, 148:1204 (2012).
Huang, Curr. Opin. Lipidol., 21:337 (2010).
Ishihara et al., Neuron, 24:751 (1999).
Josephs et al., Ann. Neurol., 63:204 (2008).
Kanehisa et al., Nucleic Acids Res., 44:D457 (2016).
Kaufman et al., Neuron, 92:796 (2016).
Knoferle et al., J. Neurosci., 34:14069 (2014).
Koutsodendris et al., Ann. Rev. Pathol., 17:73 (2022).
Lambert et al., Nat. Genet., 45:1452 (2013).
Leyns & Holtzman, Mol. Neurodegender., 12:50 (2017).
Lin et al., Neuron, 98:1141 (2018).
Luan et al., J. Surg. Res., 181:76 (2013).
Magna & Pisetsky, Mol. Med., 20:138 (2014).
Maroso et al., Nat. Med., 16:413 (2010).
Minett et al., J. Neuroinflammation, 13: (2016).
Mishra et al., Brain, 141:1828 (2018).
Mollica et al., Chem Biol., 14:431 (2007).
Moro et al., Exp. Neurol., 225:391 (2010).
Nasrabady et al., Acta Neuro. Comms., 6:22 (2018).
Operto et al., Neuroimage Clin., 24:101983 (2019).
Orr et al., Trends Pharmacol. Sci., 38:637 (2017).
Ossenkoppele et al., Brain, 139:1551 (2016).
Ou et al., Front. Aging Neurosci., 13:662474 (2021).
Overmyer et al., Demet. Geriatr. Cogn. Disord., 10:252 (1999).
Pang et al., Mediators Inflamm., 2016:4569521 (2016).
Paudel et al., Front. Neurosci., 12:628 (2018).
Pitas et al., Biochim. Biophys. Acta, 917:148 (1987).
Rajan et al., Alzheimers Dement., 17:1966 (2021).
Rangaraju et al., Mol. Neurodegender., 13:24 (2018).
Rauch et al., Nature, 580:381 (2020).
Rempe et al., Genesis, 44:44 (2006).
Rovere-Querini et al., EMBO Rep., 5:825 (2004).
Saunders et al., Neurology, 43:1467 (1993).
Scaffidi et al., Nature, 418:191 (2002).
Scholl et al., Neuron., 89:971 (2016).
Shi et al., J. Exp. Med., 216:2546 (2019).
Shi et al., Nature, 549:523 (2017).
Shi et al., Neuron., 109:2413 (2021).
Sim et al., Clin. Exp. Allergy, 52:115 (2022).
Sun et al., Front. Immunol., 9:1518 (2018).
Taubes et al., Nat. Aging, 1:932 (2021).
Tesseur et al., Am. J. Pathol., 156:951 (2000).
Tripathi et al., J. Neurosci., 30, 16383-16390 (2010).
Uhlmann et al., Ann. Neurol., 52:285 (2002).
Ulloa et al., Proc. Natl. Acad. Sci. USA, 99:12351 (2002).
Wang et al., Nat. Med., 24:647 (2018).
Wang et al., Neuron, 109:1657 (2021).
Wu et al., Innov. (N Y), 2:100141 (2021).
Xu et al., J. Neurosci., 26:4985 (2006).
Xu et al., J. Neurosci., 28:1452 (2008).
Yamada et al., J. Neurosci., 31:13110 (2011).
Yamazaki et al., Nature Reviews Neurology, 15:501 (2019).
Yang et al., J. Leukoc. Biol., 78:1 (2005).
Yoshiyama et al., Neuron, 53:337 (2007).
Zalocusky et al., Nat. Neurosci., 24:786 (2021).
Zhang et al., Sci. Adv., 6:eabb8680 (2020).
Zhao et al., Biomed Res. Int., 2017:9719647 (2017).
Zhao et al., Nat. Commun., 11:5540 (2020).
Zheng et al., Nat. Commun., 8:14049 (2017).
Zhu et al., Genes Dev., 15:859 (2001).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following Statements summarize aspects and features of the invention.

### Statements:

1. A method comprising administering one or more inhibitors of High mobility group box protein 1 (HMGB1) to a subject having at least one genomic APOE4 allele.

2. The method of statement 1, wherein the subject has two genomic APOE4 alleles.

3. The method of statement 1 or 2, wherein the subject expresses detectable levels of APOE4 protein.

4. The method of statement 1, 2 or 3, wherein the subject's cerebrospinal fluid has increased levels of High mobility group box protein 1 (HMGB1), dipeptidyl peptidase 10 (DPP10), or increased levels of both HMGB1 and DPP10 relative to a control.

5. The method of any one of statements 1-4, wherein the subject's cerebrospinal fluid has at least 25%, or at least 50%, or at least 90% increased levels of HMGB1 or DPP10 relative to a control.

6. The method of any one of statements 1-5, wherein one or more of the inhibitors of HMGB1 is glycyrrhizic acid, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies (m2G7 or #10-22), recombinant HMGB1 box A protein, acetylcholine, the nicotinic acetylcholine receptor subtype alpha 7 agonist GTS-21, Peptide P5779, resveratrol, metformin, or a combination thereof.

7. The method of any one of statements 1-6, wherein the subject exhibits symptoms of HMGB1 nucleo-cytosplasmic translocation, gliosis, neurodegeneration, tau pathology, or myelin deficit.

8. The method of any one of statements 1-7, wherein the subject exhibits symptoms of at least one tauopathy.

9. The method of any one of statements 7 or 8, wherein the tauopathy is a neurodegenerative disorder characterized by the deposition of abnormal tau protein in the brain.

10. The method of any one of statements 7-9, wherein the tauopathy is Alzheimer's disease, Pick disease, progressive supranuclear palsy, corticobasal degeneration, argyrophilic grain disease, primary age-related tauopathy, chronic traumatic encephalopathy, or frontotemporal dementia.

11. A method comprising measuring high mobility group box protein 1 (HMGB1) and dipeptidyl peptidase 10 (DPP10) levels in at least one subject's cerebrospinal fluid, and administering one or more HMGB1 inhibitors to any subject having increased levels of HMGB1 or DPP10 relative to a control.

12. The method of statement 11, wherein the at least subject has at least one genomic APOE4 allele.

13. The method of statement 11 or 12, wherein the subject has two genomic APOE4 alleles.

14. The method of any one of statements 11-13, wherein the subject expresses detectable levels of APOE4 protein.

15. The method of any one of statements 11-14, wherein increased levels of HMGB1 or DPP10 relative to a control is at least 25%, or at least 50%, or at least 90% increased levels of HMGB1 or DPP10 relative to a control.

16. The method of any one of statements 11-15, wherein one or more of the inhibitors of HMGB1 is glycyrrhizic acid, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies (m2G7 or #10-22), recombinant HMGB1 box A protein, acetylcholine, the nicotinic acetylcholine receptor subtype alpha 7 agonist GTS-21, Peptide P5779, resveratrol, metformin, or a combination thereof.

17. The method of any one of statements 11-16, wherein the subject exhibits symptoms of HMGB1 nucleo-cytosplasmic translocation, gliosis, neurodegeneration, tau pathology, or myelin deficit.

18. The method of any one of statements 11-17, wherein the subject exhibits symptoms of at least one tauopathy.

19. The method of any one of statements 17 or 18, wherein the tauopathy is a neurodegenerative disorder characterized by the deposition of abnormal tau protein in the brain.

20. The method of any one of statements 18-19, wherein the tauopathy is Alzheimer's disease, Pick disease, progressive supranuclear palsy, corticobasal degeneration, argyrophilic grain disease, primary age-related tauopathy, chronic traumatic encephalopathy, or frontotemporal dementia.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising administering one or more inhibitors of High mobility group box protein 1 (HMGB1) to a subject having at least one genomic APOE4 allele.

2. The method of claim 1, wherein the subject has two genomic APOE4 alleles.

3. The method of claim 1, wherein the subject expresses detectable levels of APOE4 protein.

4. The method of claim 1, wherein the subject's cerebrospinal fluid has increased levels of High mobility group box protein 1 (HMGB1), dipeptidyl peptidase 10 (DPP10), or increased levels of both HMGB1 and DPP10 relative to a control.

5. The method of claim 1, wherein the subject's cerebrospinal fluid has at least 25%, or at least 50%, or at least 90% increased levels of HMGB1 or DPP10 relative to a control.

6. The method of claim 1, wherein one or more of the inhibitors of HMGB1 is glycyrrhizic acid, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies, for example, m2G7 or #10-22, recombinant HMGB1 box A protein, acetylcholine, the nicotinic acetylcholine receptor subtype alpha 7 agonist GTS-21, Peptide P5779, resveratrol, metformin, or a combination thereof.

7. The method of claim 1, wherein the subject exhibits symptoms of HMGB1 nucleo-cytosplasmic translocation, gliosis, neurodegeneration, tau pathology, or myelin deficit.

8. The method of claim 1, wherein the subject exhibits symptoms of at least one tauopathy.

9. The method of claim 7, wherein the tauopathy is a neurodegenerative disorder characterized by the deposition of abnormal tau protein in the brain.

10. The method of claim 7, wherein the tauopathy is Alzheimer's disease, Pick disease, progressive supranuclear palsy, corticobasal degeneration, argyrophilic grain disease, primary age-related tauopathy, chronic traumatic encephalopathy, or frontotemporal dementia.

11. A method comprising measuring high mobility group box protein 1 (HMGB1) and dipeptidyl peptidase 10 (DPP10) levels in at least one subject's cerebrospinal fluid, and administering one or more HMGB1 inhibitors to any subject having increased levels of HMGB1 or DPP10 relative to a control.

12. The method of claim 11, wherein the at least subject has at least one genomic APOE4 allele.

13. The method of claim 11, wherein the subject has two genomic APOE4 alleles.

14. The method of claim 11, wherein the subject expresses detectable levels of APOE4 protein.

15. The method of claim 11, wherein increased levels of HMGB1 or DPP10 relative to a control is at least 25%, or at least 50%, or at least 90% increased levels of HMGB1 or DPP10 relative to a control.

16. The method of claim 11, wherein one or more of the inhibitors of HMGB1 is glycyrrhizic acid, ethyl pyruvate, nicotine, (−)-epigallocatechin gallate (EGCG), tanshinone, chlorogenic acid, emodin-6-O-β-D-glucoside, rosmarinic acid, isorhamnetin-3-O-galactoside, persicarin, forsythoside B, chloroquine, acteroside, shikonin, carbenoxolone, quercetin, lycopene, nafamostat mesilate, gabexate mesilate, sivelestat sodium, HMGB1 monoclonal antibodies, for example, m2G7 or #10-22, recombinant HMGB1 box A protein, acetylcholine, the nicotinic acetylcholine receptor subtype alpha 7 agonist GTS-21, Peptide P5779, resveratrol, metformin, or a combination thereof.

17. The method of claim 11, wherein the subject exhibits symptoms of HMGB1 nucleo-cytosplasmic translocation, gliosis, neurodegeneration, tau pathology, or myelin deficit.

18. The method of claim 11, wherein the subject exhibits symptoms of at least one tauopathy.

19. The method of claim 17, wherein the tauopathy is a neurodegenerative disorder characterized by the deposition of abnormal tau protein in the brain.

20. The method of claim 18, wherein the tauopathy is Alzheimer's disease, Pick disease, progressive supranuclear palsy, corticobasal degeneration, argyrophilic grain disease, primary age-related tauopathy, chronic traumatic encephalopathy, or frontotemporal dementia.
